(19) United States
(12) Patent Application Publication
(10) Pub. No.: US 2022/0401539 A1
(43) Pub. Date: Dec. 22, 2022

(54) Immunotherapy Targeting Tumor Neoantigenic Peptides

(71) Applicants: Institut Curie, Paris (FR); INSERM (Institut National de la Santé et de la Recherche Médicale), Paris (FR)

(72) Inventors: Olivier Delattre, Paris (FR); Olivier Saulnier, Paris (FR); Joshua Waterfall, Paris (FR); Céline Collin, Paris (FR); Julien Vibert, Saint Cloud (FR); Maud Gautier, Paris (FR)

(73) Assignees: Institut Curie, Paris (FR); Institut National de la Santé et de la Recherche Médicale, Paris (FR)

(21) Appl. No.: 17/770,304

(22) PCT Filed: Oct. 22, 2020
(86) PCT No.: PCT/EP2020/079832

§ 371 Date: Apr. 20, 2022

(30) Foreign Application Priority Data
Oct. 22, 2019 (EP) 19306370.8

(51) Int. Cl. A61K 39/00; C07K 16/32; C07K 14/725; A61P 35/00; G16B 30/20; C07K 14/47

(52) U.S. Cl. CPC A61K 39/001152; C07K 16/32; C07K 14/7051; A61P 35/00; G16B 30/20; C07K 14/4748; A61K 39/001196; A61K 2039/5158; C07K 2319/00; C07K 2317/92; C07K 2317/31

(57) ABSTRACT

The present disclosure relates to a tumor specific neoantigenic peptide, wherein said peptide (i) is encoded by a part of an (ORF) sequence from an unannotated transcript which transcription is positively regulated by an aberrant fusion protein, and (ii) is expressed at a higher level or frequency in a sample from said tumor compared to normal tissue sample. The present disclosure also relates to vaccine or immunogenic composition, antibodies and immune cells derived thereof and their use in therapy of cancer.

### FIELD OF THE DISCLOSURE

The present disclosure provides neoantigenic peptides encoded by a part of an (ORF) sequence from a transcript which transcription is regulated by an aberrant fusion transcription factor, such as the EWS-FLIT fusion protein, as well as nucleic acids, vaccines, antibodies and immune cells that can be used in cancer therapy.

### BACKGROUND

Harnessing the immune system to generate effective responses against tumors is a central goal of cancer immunotherapy. Part of the effective immune response involves T lymphocytes specific for tumor antigens. T cell activation requires their interaction with antigen-presenting cells (APCs), commonly dendritic cells (DCs), expressing TCR-cognate peptides presented in the context of a major histocompatibility molecule (MHC) and co-stimulation signals. Subsequently, activated T cells can recognize peptide-MHC complexes presented by all cell types, even malignant cells. Neoplasms often contain infiltrating T lymphocytes reactive with tumor cells.

However, the efficiency of immune responses against tumors is severely dampened by various immunosuppressive strategies developed by tumors; e.g, tumor cells express receptors that provide inhibitory signals to infiltrating T cells, or they secrete inhibitory cytokines. The development of checkpoint blockade therapy has provided means to bypass some of these mechanisms, leading to more efficient killing of cancer cells. The promising results yielded by this approach have opened new avenues for the development of T cell-based immunotherapy. Checkpoint inhibitors are, however, effective in a minority of patients and only in limited types of cancer.

A major goal in immunotherapy is to increase the proportion of responding patients and extend the cancer indications. Vaccination, administration of anti-tumor antibodies, or administration of immune cells specific for tumor antigens have all been proposed to increase the anti-tumor immune response, and can be administered alone, with other therapies such as chemotherapy or radiation, or as a combination therapy with checkpoint blockers. The selection of antigens able to trigger anti-tumor immunity without targeting healthy tissues has been a long-standing challenge.

Cancers are frequently characterized by abnormal transcription factors as a result of point mutation or gene fusion. These altered transcription factors may thus have gain-of-function, neomorphic DNA binding properties which lead to aberrant transcriptional activities which may impact tumor development/progression.

In particular, Ewing sarcoma is an aggressive tumor which mostly occurs in teenagers and young adults and which prognosis is still detrimental particularly in metastatic form of the disease or at relapse. It is characterized by specific gene fusions between members of the FET (FUS/TLS, EWS, TAF15) family of RNA binding proteins and members of the ETS family of transcription factors. The most frequent fusion is between EWS and FLI1.

The search for tumor neoantigens has mostly been focused on mutated sequences appearing as in cancer cells. These antigens are unique to each patient. Tumor antigens (the ones preferentially expressed in tumor cells) are, however, self-antigens that represent poor targets for vaccination (probably due to central tolerance). Identifying shared true neoantigens (absent from tissues) is a major challenge for the field.

New tumor neoantigens would be of interest and might improve or reduce the cost of cancer therapy in particular in the case of vaccination and adoptive cell therapy.

### SUMMARY

The present disclosure provides a method for identifying cancer specific neoantigenic peptides which comprises:
i. identifying transcripts from one or more samples isolated from a tumor driven by a fusion protein, notably a transcription factor fusion, obtained from one or more subjects,
   a. which transcription is specifically positively regulated by said fusion protein,
   b. which are specifically associated with the fusion tumor (e.g., the transcription factor fusion-driven tumor), and optionally
   c. which are encoded by neogenes originating from intergenic regions or intronic; and
ii. identifying among the transcripts identified at step (i) open reading frame (ORF) sequences, wherein typically said ORF sequences are expressed at higher level or frequency in a sample from said tumor as compared to a normal tissue sample.

In some embodiments, the fusion protein is a transcription factor fusion. Typically, said transcription factor fusion is encoded by a fusion gene selected from any one of PAX3-FOXO1, PAX7-FOXO1, ASPSCR1-TFE3, AHRR-NCOA2, EWSR1-CREB1, EWSR1-ATF1, FUS-ATF1, EWSR1-CREB1, COL1A1-PDGFB, EWSR1-WT1, WWTR1-CAMTA1, TFE3-YAP1, EWSR1-FLI1, EWSR1-ERG, EWSR1 fusion with various ETS partners such as ETV1 FEV and ETV4, FUS-ERG, EWSR1-NFATC2, CIC-DUX4, BCOR-CCNB3, EWSR1-NR4A3, TAF15-NR4A3, TCF12-NR4A3, TFG-NR4A3, ETV6-NTRK3, ALK-TPM4, ALK-TPM3, ALK-CLTC, ALK-RANBP2, ALK-ATIC, ALK-SEC31A, ALK-CARS, PLAF fusions, HMGA2 fusions, HMGA1 fusions, C-MKL2, f95-MKL2, FUS-CREB3L2, EWSR1-ZNF444, EWSR1-PBX1, EWSR1-POU5F1, FUS-DDIT3, EWSR1-DDIT3, EWS-CHOP, EWS-CHN, TGFBR3-MGEA5, TGFBR3-MGEA5, MYH9-USP6, PHF1 fusions, ACTB-GLI1, FUS-CREB3L1, NAB2-STAT6, NCOA2-SRF, NCOA2-TEAD1, SS18-SSX1, SS18-SSX2, SS18-SSX4, and CSF1-COL6A3. In more specific embodiments, the transcription factor fusion is encoded by an EWS (EWSR1) fusion gene (as defined notably in table 1 herein).

More specifically, the present disclosure provides a method for identifying Ewing-sarcoma specific neoantigenic peptides which comprises:
i. identifying unannotated transcripts among long read sequencing from one or more samples isolated from an Ewing Sarcoma tumor obtained from one or more subjects, which transcription is positively regulated by the EWS-FLI1 fusion protein or negatively regulated upon the EWS-FLI1 fusion protein depletion, and typically wherein the transcripts have no match on normal reference transcriptome;
ii. identifying among the unannotated transcripts of step (i) open reading frame (ORF) sequences, wherein said ORF sequences are expressed at higher level or frequency in an Ewing sarcoma sample as compared to a normal tissue sample.

The present disclosure also provides a tumor specific neoantigenic peptide, wherein the tumor is associated with a fusion protein and typically a transcription factor fusion, and wherein said peptide:
is encoded by a part of an (ORF) sequence from an unannotated transcript which transcription is positively regulated by said fusion protein (or in other words which transcription is negatively regulated upon said fusion protein depletion), and
is expressed at a higher level or frequency in a sample from said tumor compared to a normal tissue sample.

In some embodiments, the fusion protein is a transcription factor fusion which can be encoded by a fusion gene selected from any one of PAX3-FOXO1, PAX7-FOXO1, ASPSCR1-TFE3, AHRR-NCOA2, EWSR1-CREB1, EWSR1-ATF1, FUS-ATF1, EWSR1-CREB1, COL1A1-PDGFB, EWSR1-WT1, WWTR1-CAMTA1, TFE3-YAP1, EWSR1-FLI1, EWSR1-ERG, EWSR1 fusion with various ETS partners such as ETV1 FEV and ETV4, FUS-ERG, EWSR1-NFATC2, CIC-DUX4, BCOR-CCNB3, EWSR1-NR4A3, TAF15-NR4A3, TCF12-NR4A3, TFG-NR4A3, ETV6-NTRK3, ALK-TPM4, ALK-TPM3, ALK-CLTC, ALK-RANBP2, ALK-ATIC, ALK-SEC31A, ALK-CARS, PLAF fusions, HMGA2 fusions, HMGA1 fusions, C-MKL2, f95-MKL2, FUS-CREB3L2, EWSR1-ZNF444, EWSR1-PBX1, EWSR1-POU5F1, FUS-DDIT3, EWSR1-DDIT3, EWS-CHOP, EWS-CHN, TGFBR3-MGEA5, TGFBR3-MGEA5, MYH9-USP6, PHF1 fusions, ACTB-GLI1, FUS-CREB3L1, NAB2-STAT6, NCOA2-SRF, NCOA2-TEAD1, SS18-SSX1, SS18-SSX2, SS18-SSX4, and CSF1-COL6A3. In more specific embodiments, the fusion gene is an EWS (EWSR1) fusion gene as mentioned above or in the table 1 herein.

The present disclosure also encompasses a tumor specific neoantigenic peptide, wherein said tumor is associated with a fusion protein, notably a transcription factor fusion, and wherein said peptide
i) is encoded by a part of an (ORF) sequence from a neotranscript characterized in that:
   a. its expression is regulated by said fusion protein, as evidenced by expression in cell line wherein the expression of said fusion protein is made inducible,
   b. it is specifically associated with the fusion-driven tumor,
   c. optionally it is encoded by genome regions having binding motifs involved in promoter regulation, such as poly GGAA binding sites, and/or having binding sites for said transcription factor as determined by ChIP-seq experiments and/or histone marks activation, such as H3K27ac and H3K4me3 histone, at less than 5 kb of the TSS, and/or
   d. optionally it is encoded by intergenic or intronic regions of the genome;
ii) is expressed at a higher level or frequency in a sample from said tumor compared to normal tissue sample.

The present disclosure further provides an Ewing-sarcoma specific neoantigenic peptide, wherein said peptide:
is encoded by a part of an (ORF) sequence from a transcript
   which transcription is positively regulated by the EWS-FLI1 fusion protein (or in other words which transcription is negatively regulated upon the EWS-FLI1 fusion protein depletion), is specifically associated with the fusion-driven tumor type,
   optionally is encoded by genome regions having poly GGAA binding sites and/or histone marks activation, such as H3K27ac and H3K4me3 histone at no more than 500 bp of the TSS, and/or
   optionally it is encoded by intergenic or intronic regions of the genome.
      and
is expressed at a higher level or frequency in an Ewing sarcoma sample compared to normal tissue sample.

In one embodiment, the tumor neoantigenic peptide is 8 or 9 amino acids long, notably 8 to 11, and binds to at least one MHC class I molecule of said subject.

In another embodiment, the tumor neoantigenic peptide is from 13 to 25 amino acids long, and binds to at least one MHC class II molecule of said subject.

The present disclosure also encompasses peptides obtainable by the method as herein disclosed.

More particularly, the present disclosure provides Ewing sarcoma neoantigenic peptides, comprising at least 8 amino acids and encoded by an open reading frame (ORF) selected from the group comprising SEQ ID NO: 1-145 and the transcripts identified in table 9; optionally wherein the neoantigenic peptides are of SEQ ID NO:166-201.

Said neoantigenic peptides are typically expressed at higher levels, or higher frequency, in tumor cells (e.g., Ewing sarcoma cells) compared to normal healthy cells, optionally wherein said neoantigenic peptides are not expressed, or not detectably expressed in normal healthy cells.

In some embodiments said neoantigenic peptides are expressed in at least at least 5%, 6%, 7%, 8%, 9%, 10%, 15%, 20%, 25%, 30%, 40%, 50%, 60%, 70%, 80%, 90%, 95%, or even 99% of a population of subjects suffering from a cancer, notably from a bone or soft tissue tumor and more specifically from a population of subjects suffering from Ewing sarcoma.

Typically, the neoantigenic peptides bind MEW class I or class II with a binding affinity Kd of less than about 10⁻⁴, 10⁻⁵, 10⁻⁶, 10⁻⁷, 10⁻⁸ or 10⁻⁹ M (lower numbers indicating higher binding affinity).

The present disclosure also encompasses:
a population of autologous dendritic cells or antigen presenting cells that have been pulsed with one or more of the peptides as herein defined, or transfected with a polynucleotide encoding one or more of the peptides as herein described;
a vaccine or immunogenic composition, notably a sterile vaccine or immunogenic composition, capable of raising a specific T-cell response comprising
   a. one or more neoantigenic peptides as herein defined,
   b. one or more polynucleotides encoding a neoantigenic peptide as herein defined, optionally wherein the one or more polynucleotides are linked to a heterologous regulatory control nucleotide sequence; or
   c. a population of autologous dendritic cells or antigen presenting cells (notably artificial APC) that have been pulsed or loaded with one or more of the peptides as herein defined,
optionally in combination with a physiologically acceptable buffer, carrier, excipient, immunostimulant and/or adjuvant.
an antibody, or an antigen-binding fragment thereof, a T cell receptor (TCR), or a chimeric antigen receptor (CAR) that binds a neoantigenic peptide as herein defined, or a composition comprising thereof.
a polynucleotide encoding a neoantigenic peptide, an antibody, a CAR or a TCR as herein defined, typically operatively linked to a heterologous regulatory control nucleotide sequences, and a vector encoding such polynucleotide, or a vaccine or immunogenic composition comprising thereof, notably such polynucleotide or vector;
an immune cell, or a population or immune cells that targets one or more neoantigenic peptides, as herein defined, wherein the population of immune cells preferably targets a plurality of different tumor neoantigenic peptides as herein disclosed, or a composition comprising thereof.

Typically, the antibody or antigen-binding fragment thereof, TCR or CAR binds a neoantigenic peptide, optionally in association with an MEW molecule, with a K_{d} affinity of about 10⁻⁶ M or less.

In some embodiments, the T cell receptor can be made soluble and fused to an antibody fragment directed to a T cell antigen, optionally wherein the targeted antigen is CD3 or CD16.

In some embodiments, the antibody can be a multispecific antibody that further targets at least an immune cell antigen, optionally wherein the immune cell is a T cell, a NK cell or a dendritic cell, optionally wherein the targeted antigen is CD3, CD16, CD30 or a TCR. In any of the embodiments relating to an antibody, the antibody can be fusion, humanized, or human, and may be IgG, e.g. IgG1, IgG2, IgG3, IgG4.

The immune cell can be typically a T cell or a NK cell, a CD4+ and/or CD8+ cell, a TILs/tumor derived CD8 T cells, a central memory CD8+ T cells, a Treg, a MAIT, or a Yδ T cell. The cell can also be autologous or allogenic.

The T cell can comprises a recombinant antigen receptor selected from T cell receptor and chimeric antigen receptor as herein defined, wherein the antigen is a tumor neoantigenic receptor as herein disclosed.

The present disclosure also encompasses a method of producing an antibody, TCR or CAR that specifically binds a neoantigenic peptide as herein defined comprising the step of selecting an antibody, TCR or CAR that binds to a tumor neoantigen peptide of the present disclosure, optionally in association with an MEW or HLA molecule, with a K_{d} binding affinity of about 10⁻⁶ M or less. Antibodies, TCRs and CARs selected by said method are also part of the present application.

A polynucleotide encoding a neoantigenic peptide as herein defined, or an antibody, a CAR or a TCR as herein defined, optionally linked to a heterologous regulatory control sequence are also part of the present application.

As per the present disclosure, the neoantigenic peptide, the population of dendritic cells, the vaccine or immunogenic composition, the polynucleotide or the vector encoding the peptide can be used in cancer vaccination therapy of a subject; or for treating cancer in a subject suffering from cancer or at risk of cancer; or can be used for inhibiting proliferation of cancer cells. Typically, the peptide(s) bind at least one MHC molecule of said subject.

As per the present disclosure, the antibody or the antigen-binding fragment thereof, the multispecific antibody, the TCR, the CAR, the polynucleotide, or the vector encoding such antibody, TCR or CAR, as herein defined can be used in the treatment of cancer in a subject in need thereof, typically in a subject suffering from cancer or at risk of cancer, or can be used for inhibiting proliferation of cancer cells.

Still as per the present disclosure, the population of immune cells as herein defined can be used in cell therapy of a subject suffering from cancer or at risk of cancer, or can be used for inhibiting proliferation of cancer cells.

Typically, as per the present application, the cancer is Ewing sarcoma. Particularly, the various cancer therapies, or cancer therapeutic products of the present application (e.g., the neoantigenic peptide, the population of dendritic cells, the vaccine or immunogenic composition, the polynucleotide or the vector encoding the peptide, the antibody or the antigen-binding fragment thereof, the multispecific antibody, the TCR, the CAR, the polynucleotide, or the vector encoding such antibody, TCR or CAR or the population of immune cells (collectively referenced herein as the “Cancer Therapeutic Products”) are used in the treatment of a subject who is suffering from Ewing sarcoma or who is at risk of suffering from Ewing sarcoma.

Pharmaceutical compositions comprising any of the foregoing, optionally with a sterile pharmaceutically acceptable excipient(s), carrier, and/or buffer are also contemplated as well as methods of using them.

In any of the embodiments described herein, the cancer therapeutic products, as herein disclosed, can be administered in combination with at least one further therapeutic agent. Such further therapeutic agent can typically be a chemotherapeutic agent, or an immunotherapeutic agent.

For example, according to the present disclosure, any of the Cancer Therapeutic Products can be administered in combination with an anti-immunosuppressive/immunostimulatory agent. For example, the subject is further administered one or more checkpoint inhibitors typically selected from PD-1 inhibitors, PD-L1 inhibitors, Lag-3 inhibitors, Tim-3 inhibitors, TIGIT inhibitors, BTLA inhibitors, V-domain Ig suppressor of T-cell activation (VISTA) inhibitors and CTLA-4 inhibitors, IDO inhibitors.

Various embodiments of the methods, neoantigenic peptides and Cancer Therapeutic Products are described in detailed below. Except for alternatives clearly mentioned, combinations of such embodiments are encompassed by the present application.

### DETAILED DISCLOSURE

In contrast to the wild-type FLI1, the chimeric EWS-FLI1 has gain-of-function activities and, in particular, the ability to bind GGAA microsatellite sequences in the genome. It thus creates neomorphic enhancer regions that can interact with neighboring promoters and activate corresponding genes.

EWS-FLI1 can, not only activate known genes, but also lead to transcription activation of otherwise transcriptionally silent genome regions (in normal cells or tissues as compared to transcription factor fusion drive-tumors) generating neomorphic genes, also named “neogenes” or “new genes”. Such neogenes, or new genes, typically originate from intergenic or intronic regions of the genome and encode for neo-transcripts and have thus a standard gene structure (i.e. they contain exonic and intronic regions and they can be alternatively spliced and polyadenylated) and are typically not annotated in public databases, such as the public EBI (European Bioinformatic Institute) databases which reference is Ensembl database (https://www.ensembl.org/index.html) or the expression atlas, or the NCBI public databases such as the RefSeq (https://www.ncbi.nlm.nih.gov/refseq/about/) or GEO (https://www.ncbi.nlm.nih.gov/geo/) databases. Other databases can be used such as the GTEX database (https://gtexportal.org/home/). The neotranscripts encoded by this neogenes are thus typically unannotated in such public databases.

This mechanism is further shown, in the results included herein, to be prevalent in diverse transcription factor fusion-driven cancers, also generating tumor-specific neogenes in other tumor types such as alveolar rhabdomyosarcoma and desmoplastic small round cell tumor, driven by PAX-FOXO and EWS-WT1 (or EWSR1-WT1) chimeric fusion proteins respectively. Tumor-specific neogenes have thus also found in 16 additional fusion-driven cancers. Importantly, this observation in 19 transcription factor fusion-driven cancers can be applied to the hundreds of human cancers that are characterized by transcription factor fusions.

This finding has significant implications both for diagnosis of such tumor types as well as identifying potential therapeutic targets. Indeed, the inventors now provide demonstration that peptides encoded by the putative open reading frames of these neo-transcripts can be recognized by naïve T-cells from controls.

This discovery first provides tumor-specific biomarkers that can discriminate between tumors, for example that allows to discriminate Ewing sarcoma from other tumors, particularly from other sarcomas. Second, such neopeptides can lead to the expression of tumor-specific peptides, the presentation of which in an MHC-Class1 context can lead to a specific immune response thus constituting the basis for tumor-directed immunotherapies such as vaccination, development of neo-peptide specific antibodies or cytotoxic T-cell-based therapies.

The presently defined neoantigenic peptides are also expected to be highly immunogenic as they are derived from sequences absent from normal cells. Thus, the peptides of the present disclosure are expected to exhibit very low immunological tolerance.

The present disclosure also allows selecting peptides having shared tumor neoepitopes among a population of patients. Such shared tumor peptides are of high therapeutic interest since they may be used in immunotherapy for a large population of patients.

### Definitions

The term “fusion gene” as used herein is a hybrid or chimeric gene formed from two previously independent genes. It can occur as a result of translocation, interstitial deletion, or chromosomal inversion. Such fusion genes have been found to be prevalent in all main types of human tumors (Mitelman, F., Johansson, B. & Mertens, F. The impact of translocations and gene fusions on cancer causation. Nat Rev Cancer 7, 233-245 (2007)) and dan thus also be named oncogenic gene fusion. For example, the Ewing sarcoma is characterized by the reciprocal chromosomal translocation generating a fusion oncogene between the EWS gene (also named EWSR1) involved in various cellular processes, including gene expression, cell signaling, and RNA processing and transport, and an Ets family transcription factor, most commonly FLI-1. As used herein a transcription factor fusion is encoded by a fusion gene involving a gene coding for a transcription factor.

According to the present disclosure, the term “disease” refers to any pathological state, including cancer diseases, in particular those forms of cancer diseases described herein.

The term “normal” refers to the healthy state or the conditions in a healthy subject or tissue, i.e., non-pathological conditions, wherein “healthy” preferably means non-cancerous.

Cancer (medical term: malignant neoplasm) is a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor but some, like leukemia, do not.

Malignant tumor is essentially synonymous with cancer. Malignancy, malignant neoplasm, and malignant tumor are essentially synonymous with cancer.

As used herein, the term “tumor” or “tumor disease” refers to an abnormal growth of cells (called neoplastic cells, tumorigenous cells or tumor cells) preferably forming a swelling or lesion. By “tumor cell” is meant an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be either benign, pre-malignant or malignant.

A benign tumor is a tumor that lacks all three of the malignant properties of a cancer. Thus, by definition, a benign tumor does not grow in an unlimited, aggressive manner, does not invade surrounding tissues, and does not spread to non-adjacent tissues (metastasize).

Neoplasm is an abnormal mass of tissue as a result of neoplasia. Neoplasia (new growth in Greek) is the abnormal proliferation of cells. The growth of the cells exceeds, and is uncoordinated with that of the normal tissues around it. The growth persists in the same excessive manner even after cessation of the stimuli. It usually causes a lump or tumor. Neoplasms may be benign, pre-malignant or malignant.

“Growth of a tumor” or “tumor growth” according to the present disclosure relates to the tendency of a tumor to increase its size and/or to the tendency of tumor cells to proliferate.

For purposes of the present disclosure, the terms “cancer” and “cancer disease” are used interchangeably with the terms “tumor” and “tumor disease”.

Cancers are classified by the type of cell that resembles the tumor and, therefore, the tissue presumed to be the origin of the tumor. These are the histology and the location, respectively.

The term “cancer” according to the disclosure comprises leukemias, seminomas, melanomas, teratomas, lymphomas, neuroblastomas, gliomas, sarcomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer, soft tissue tumors and the metastases thereof. The term cancer according to the present disclosure also comprises cancer metastases and relapse of cancer.

Soft tissues include tendons, ligaments, fascia, skin, fibrous tissues, fat, and synovial membranes (which are connective tissue), and muscles, nerves and blood vessels (which are not connective tissue).

A “sarcoma” is a cancer that arises from transformed cells of mesenchymal (i.e., connective tissue) origin (see Yang J et al., October 2014 “The role of mesenchymal stem/progenitor cells in sarcoma: update and dispute”. Stem Cell Investigation; and Tobias J 2015 “Cancer and its Management” Seventh Edition. Chichester, West Sussex, P0198SQ, UK: John Wiley & Sons, Ltd. p. 446). Connective tissue is a broad term that includes bone, cartilage, fat, vascular, or hematopoietic tissues, and sarcomas can arise in any of these types of tissues. As a result, there are many subtypes of sarcoma, which are classified based on the specific tissue and type of cell from which the tumor originates (Amin M B 2017 AJCC “Cancer Staging Manual”, Eight Edition. Chicago, Ill. 60611, USA: Springer International Publishing AG Switzerland. pp. 471-548). Sarcomas are notably primary connective tissue tumors, meaning that they arise in connective tissues. An example of bone sarcoma is notably the Ewing sarcoma.

Typically, the cancers as per the present disclosure are associated with or driven by the expression of an aberrant transcription factor, also named herein transcription factor chimeric fusion or more shortly transcription factor fusion, having gain of function activity. Such transcription factor fusions are encoded by fusion genes (typically oncogenic fusion genes) involving fusion with a gene coding for a transcription factor. Example of such transcription factor fusions notably include those encoded by any one of the gene selected from PAX-FOXO fusions, such as PAX3-FOXO1, PAX7-FOXO1, ASPSCR1-TFE3, AHRR-NCOA2, fusion between the FET family of RNA-binding proteins (including the FUS, EWS and TAF15 proteins) and the ETS family of transcriptions factors (E26 transformation-specific or E-twenty-six, or Erythroblast Transformation Specific), such as EWSR1-CREB1, EWSR1-ATF1, FUS-ATF1, EWSR1-CREB1, COL1A1-PDGFB, EWSR1-WT1, WWTR1-CAMTA1, TFE3-YAP1, EWSR1-FLI1, EWSR1-ERG, EWSR1 fusion with various ETS partners such as ETV1, FEV and ETV4, FUS-ERG, EWSR1-NFATC2, CIC-DUX4, BCOR-CCNB3, EWSR1-NR4A3, EWSR1-PATZ1, TAF15-NR4A3, TCF12-NR4A3, TFG-NR4A3, ETV6-NTRK3, ALK-TPM4, ALK-TPM3, ALK-CLTC, ALK-RANBP2, ALK-ATIC, ALK-SEC31A, ALK-CARS, PLAF fusions, HMGA2 fusions, HMGA1 fusions, C-MKL2, f95-MKL2, FUS-CREB3L2, EWSR1-ZNF444, EWSR1-PBX1, EWSR1-POU5F1, FUS-DDIT3, EWSR1-DDIT3, EWS-CHOP, EWS-CHN, TGFBR3-MGEA5, TGFBR3-MGEA5, MYH9-USP6, PHF1 fusions, ACTB-GLI1, FUS-CREB3L1, NAB2-STAT6, NCOA2-SRF, NCOA2-TEAD1, SS18-SSX1, SS18-SSX2, SS18-SSX4, and CSF1-COL6A3. In some embodiments of the present disclosure, the transcription factor fusions are selected from PAX-FOXO, EWSR1-WT1 and EWSR1-FLI1 fusions (it is to be noted that EWSR1 can also be abbreviated in EWS).

In some embodiments of the present disclosure the term cancer include mesenchymatous tumors such as bone and soft tissue tumors or sarcomas, notably Alveolar rhabdomyosarcoma, Alveolar soft part sarcoma, Angiofibroma, Angiomatoid fibrous histiocytom, Clear cell sarcoma, extraskeletal myxoid chondrosarcoma (emCS), Dermatofibrosarcoma protuberans/giant cell fibroblastoma, Desmoplastic small round cell tumor, Epithelioid hemangioendothelioma, Ewing sarcoma, Ewing sarcoma-like such as EWSR1-NFATC2 sarcoma (NFAT), CIC-fused sarcoma (such as CIC-DUX4 sarcoma), EWSR1-PATZ1 sarcoma (PATZ1), or BCOR-rearranged sarcoma (BCOR-SSNB3 sarcoma), Infantile fibrosarcoma, inflammatory myofibroblastic tumor (TMFI), lipoblastoma, Lipoma, ordinary, Lipoma, chondroid, Low-grade fibromyxoid sarcoma, Mesenchymal chondrosarcoma (MCS), midline carcinoma (Midline), Myoepithelioma, soft tissue, Myxoid/round cell liposarcoma, Myxoinflammatory fibroblastic sarcoma/hemosiderotic fibrolipomatous tumor, Nodular fasciitis, Ossifying fibromyxoid tumor, pericytoma, myxoid liposarcoma (mLPS), solitary fibrous tumor (SFT), synovial sarcoma (SS), TFE3 renal cell carcinoma (TFE3, Sclerosing epithelioid fibrosarcoma, Spindle cell rhabdomyosarcoma, Solitary fibrous tumor, Tenosynovial giant cell tumor,

In some embodiments, the cancer is selected from Ewing sarcoma (Ew), alveolar rhabdomyosarcoma (aRMS), desmoplastic small round cell tumor (DSRCT), clear cell sarcoma (CCS), Ewing sarcoma-like such as EWSR1-NFATC2 sarcoma (NFAT), BCOR-rearranged sarcoma (BCOR-SSNB3), or CIC-fused sarcoma (CIC), such as CIC-DUX4 sarcoma synovial sarcoma (SS), angiomatoid fibrous histiocytoma (AFH), alveolar soft part sarcoma (ASPS), extraskeletal myxoid chondrosarcoma (emCS), low-grade fibromyxoid sarcoma (LGFS), mesenchymal chondrosarcoma (MCS), midline carcinoma (Midline), myxoid liposarcoma (mLPS), EWSR1-PATZ1 sarcoma (PATZ1), solitary fibrous tumor (SFT), TFE3 renal cell carcinoma (TFE3), and inflammatory myofibroblastic tumor (TMFI).

A “Neogene” or a “new gene”, as herein intended, corresponds to a region of the genome which is transcriptionally silent in normal cell or tissue, but which transcription is induced by a transcription factor fusion typically in a cancer cell wherein the cancer is driven by said transcription factor fusion. Neogenes, or new genes typically correspond to intergenic or intronic regions of the genome. A Neogene from a cancer cell as above defined therefore encodes a “neotranscript”, which is unannotated in a database referencing the transcriptome data of the corresponding normal cell from the same organism.

By “metastasis” is meant the spread of cancer cells from its original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor, i.e. a secondary tumor or metastatic tumor, at the target site depends on angiogenesis.

Tumor metastasis often occurs even after the removal of the primary tumor because tumor cells or components may remain and develop metastatic potential. In one embodiment, the term “metastasis” according to the present disclosure relates to “distant metastasis” which relates to a metastasis which is remote from the primary tumor and the regional lymph node system.

A relapse or recurrence occurs when a person is affected again by a condition that affected them in the past. For example, if a patient has suffered from a tumor disease, has received a successful treatment of said disease and again develops said disease said newly developed disease may be considered as relapse or recurrence. However, according to the present disclosure, a relapse or recurrence of a tumor disease may but does not necessarily occur at the site of the original tumor disease. Thus, for example, if a patient has suffered from ovarian tumor and has received a successful treatment a relapse or recurrence may be the occurrence of an ovarian tumor or the occurrence of a tumor at a site different to ovary. A relapse or recurrence of a tumor also includes situations wherein a tumor occurs at a site different to the site of the original tumor as well as at the site of the original tumor. Preferably, the original tumor for which the patient has received a treatment is a primary tumor and the tumor at a site different to the site of the original tumor is a secondary or metastatic tumor.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

By “being at risk” is meant a subject, i.e. a patient, that is identified as having a higher than normal chance of developing a disease, in particular cancer, compared to the general population. In addition, a subject who has had, or who currently has, a disease, in particular cancer, is a subject who has an increased risk for developing a disease, as such a subject may continue to develop a disease. Subjects who currently have, or who have had, a cancer also have an increased risk for cancer metastases.

The therapeutically active agents or product, vaccines and compositions described herein may be administered via any conventional route, including by injection or infusion.

The agents described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an agent described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on several of such parameters. In the case that a reaction in a patient is insufficient with an initial dose, higher doses (or effectively higher doses achieved by a different, more localized route of administration) may be used.

The pharmaceutical compositions as herein described are preferably sterile and contain an effective amount of the therapeutically active substance to generate the desired reaction or the desired effect.

The pharmaceutical compositions as herein described are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds. When used in medicine, the salts should be pharmaceutically compatible.

A “transcript” as herein intended is a messenger RNA (or mRNA) or a part of a mRNA which is expressed by an organism, notably in a particular tissue or even in a particular tissue. Expression of a transcript varies depending on many factors. In particular expression of a transcript may be modified in a cancer cell. Typically in a transcription factor fusion-driven tumor cell or tissue sample, expression of a transcript specifically associated to said transcription factor fusion-driven tumor is increased, as compared to any other sample (from another tumor cell or tissue, or from a normal healthy cell or tissue).

A “representative genome” (also known as reference genome or assembly) is a digital nucleic acid sequence database, assembled by scientists as a representative example of species set of genes. As they are often assembled from the sequencing of DNA from a number of donors, reference genomes do not accurately represent the set of genes of any single individual (animal or person). Instead a reference provides a haploid mosaic of different DNA sequences from each donor.

A “transcriptome” as herein intended is the full range of messenger RNA, or mRNA, molecules expressed by an organism. The term “transcriptome” can also be used to describe the array of mRNA transcripts produced in a particular cell or tissue type. In contrast with the genome, which is characterized by its stability, the transcriptome actively changes. In fact, an organism's transcriptome varies depending on many factors, including stage of development and environmental conditions. Typically, the transcriptome as herein intended is the human transcriptome.

A “normal representative transcriptome” is a digital database of mRNA in healthy subject, or tissue(s).

A reading frame is a way of dividing the sequence of nucleotides in a nucleic acid (DNA or RNA) molecule into a set of consecutive, non-overlapping triplets.

An open reading frame (ORF) is the part of a reading frame that has the ability to be translated. An ORF is a continuous stretch of codons that contain a start codon (usually AUG) at a transcription starting site (TSS) and a stop codon (usually UAA, UAG or UGA). An ATG codon within the ORF (not necessarily the first) may indicate where translation starts. The transcription termination site is located after the ORF, beyond the translation stop codon. In eukaryotic genes with multiple exons, ORFs span intron/exon regions, which may be spliced together after transcription of the ORF to yield the final mRNA for protein translation.

An exon is any part of a gene that will encode a part of the final mature RNA produced by that gene after introns have been removed by RNA splicing. The term exon refers to both the DNA sequence within a gene and to the corresponding sequence in RNA transcripts. In RNA splicing, introns are removed and exons are covalently joined to one another as part of generating the mature messenger RNA.

Thus, the untranslated sequences in 3′end and in 5′ (3′UTR and 5′UTR) present in mature RNA after splicing are exonic sequences, but are non-coding sequences because these sequences are located upstream of the start codon for the translation (5′UTR) or downstream the stop codon ending the translation (3′UTR).

“TPM” as used herein means “transcripts per million”. FPKM (fragments per kilobase of exon model per million reads mapped) and TPM (transcripts per million) are the most common units reported to estimate gene expression based on RNA-seq data. Both units are calculated from the number of reads that mapped to each particular gene sequence and both units are calculated taking into account two important factors in RNA-seq:
The number of reads from a gene depends on its length. One expects more reads to be produced from longer genes.
The number of reads from a gene depends on the sequencing depth that is the total number of reads you sequenced. One expects more reads to be produced from the sample that has been sequenced to a greater depth.

FPKM (introduced by Trapnell, C., Williams, B., Pertea, G. et al. Nat Biotechnol 28, 511-515 (2010).) are calculated with the following formula:

$$F P K {M}_{i} = \frac{{q}_{l}}{\frac{{l}_{i}}{{10}^{3}} * \frac{{\sum }_{j} {q}_{j}}{{10}^{6}}} = \frac{q}{{l}_{i} * {\sum }_{j} {q}_{j}} * 1 {0}^{9}$$

where qi are raw counts (number of reads that mapped for each gene), li is gene length and total number mapped reads is the total number of mapped reads. The interpretation of FPKM is that if you sequence your RNA sample again, you expect to see for gene i, FPKMi reads divided by gene i length over a thousand and divided by the total number of reads mapped over a million.

Li and Dewey, 2011 (Li, B., Dewey, C. N. RSEM: accurate transcript quantification from RNA-Seq data with or without a reference genome. BMC Bioinformatics 12, 323 (2011)) introduced the unit TPM and Pachter, 2011 (arXiv:1104.3889 [q-bio.GN] “Models for transcript quantification from RNA-Seq”) established the relationship between both units. It is possible to compute TPM from FPKM as follows:

$$T P {M}_{i} = ( \frac{F P F {M}_{i}}{{\sum }_{j} {FPKM}_{j}} ) * {10}^{6}$$

For TPM definition, the following definition can also be consulted: Wagner et al., Theory Biosci. 2012 December; 131(4):281-5.

For example, in the EMBL expression atlas database (which contains thousands of selected microarray and RNA-sequencing data that are manually curated and annotated with ontology terms Baseline expression results), baseline expression levels are set as follow and represented in different colours (see https://www.ebi.ac.uk/gxa/FAQ.html):
Grey box: expression level is below cutoff (0.5 FPKM or 0.5 TPM)
Light blue box: expression level is low (between 0.5 to 10 FPKM or 0.5 to 10 TPM)
Medium blue box: expression level is medium (between 11 to 1000 FPKM or 11 to 1000 TPM)
Dark blue box: expression level is high (more than 1000 FPKM or more than 1000 TPM)

If not otherwise specified, the above-mentioned reference expression levels can be used as reference, or thresholds, in the methods and definitions of the present disclosure. In some embodiments however, other threshold values can be used. For example, depending on the mean expression of the transcript in a sample from the disease of interest, the expression threshold or cut-off can be set at 7.5 TPM or 10 TPM.

The term “peptide or polypeptide,” is used interchangeably with “neoantigenic peptide or polypeptide” in the present specification to designate a series of residues, typically L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

A “tumor neoantigenic peptide”, as per the present application is a peptide that induces T cell reactivity.

In some embodiments, tumor neoantigenic peptides are entirely absent from the normal genome (in particular from the human genome). Such peptides are recognized as different from self and are presented by antigen-presenting cells (APC), such as dendritic cells (DC) and tumor cells themselves. Cross-presentation plays an important role as the APC is able to translocate exogenous antigens from the phagosome into the cytosol for proteolytic cleavage into the major histocompatibility complex I (MHC I) epitopes by the proteasome. Targeting such highly specific neoantigens (or neoantigenic peptides) enables immune cell to distinguish cancerous cells from normal cell avoiding the risk for autoimmunity. Typically, neoantigenic peptides-specific T cells possess functional avidity that may reach the avidity strength of anti-viral T cells (see Lennerz V et al., Cancer immunotherapy based on mutation-specific CD4+ T cells in human melanoma. Nat Med 2015; 21:81-5).

In some embodiments, tumor neoantigenic peptides as per the present application can also include peptides or proteins to which T cell tolerance is incomplete, typically because of restricted and/or low tissue expression pattern. Such neoantigenic peptides are typically highly and/or disproportionately expressed in tumor cells (i.e.: in tumor samples) as compared to normal (healthy) cells (i.e.: in normal samples). Tumor neoantigenic peptides may also be selectively expressed by the cell lineage from which the tumor evolved.

Ewing's sarcoma is a type of cancer that forms in bone or soft tissue. Ewing's sarcoma is the second most common malignant bone tumour occurring in children and young adults, and accounts for 10-15% of all primary bone tumours. Ewing's sarcoma can affect any bone but the most common sites are the lower extremity (45%), followed by the pelvis (20%), upper extremity (13%), axial skeleton and ribs (13%), and face (2%). 6 The femur is the most frequently affected bone, with the tumour usually arising in the midshaft. Typically, by light microscopy, the tumour consists of small round cells with regular round nuclei containing finely dispersed chromatin and inconspicuous nucleoli, and a narrow rim of clear or pale cytoplasm. Tumours with similar histology also arise in soft tissues. These include peripheral primitive neuroectodermal tumour (pPNET), neuroepithelioma, and Askin tumour. pPNET is the second most common soft tissue malignancy in childhood, accounting for 20% of sarcomas. 7 It is frequently found in the chest wall (Askin tumour), paraspinal tissues, abdominal wall, head and neck, and extremities. However, soft tissue extension is common in osseous Ewing's sarcoma and infiltration of adjacent bone is frequent in soft tissue pPNETs, which often makes it difficult to determine the primary site of tumour origin (Burchill S A. Ewing's sarcoma: diagnostic, prognostic, and therapeutic implications of molecular abnormalities. J Clin Pathol. 2003; 56(2):96-102) (Burchill S A. Ewing's sarcoma: diagnostic, prognostic, and therapeutic implications of molecular abnormalities. J Clin Pathol. 2003; 56(2): 96-102).

Typically, a subject of the present application is a mammal and notably a human. Thus, typically, the representative, or reference genome or transcriptome is the human genome or transcriptome.

Unless specifically stated or obvious from context, as used herein, the term “about” is to be understood as within a range of normal tolerance in the art, for example within 2 standard deviations of the mean. About can be understood as within 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Method for Selecting a Tumor Neoantigenic Peptide

The present disclosure provides a method for identifying tumor specific neoantigenic peptides which comprises:
i. identifying transcripts from one or more samples isolated from a tumor driven by a transcription factor fusion and obtained from one or more subjects,
   which transcription is specifically positively regulated by said transcription factor fusion,
   which are specifically associated with the transcription-fusion tumor type, and optionally
   which are encoded by neogenes, notably that originate from intergenic or intronic regions of the genome;
ii. identifying open reading frame (ORF) sequences from the transcripts of step (i), optionally wherein said ORF sequences are specifically expressed in a tissue (or cell) sample from said transcription factor fusion-driven tumor.

Neotranscripts as herein described typically align to the representative genome of the subject (e.g., typically the human genome), but do not align to the representative (e.g., typically the human) transcriptome (i.e. they have no match in the normal representative transcriptome). These transcripts are encoded from genome regions (intronic or intergenic) that are transcriptionally silent when the transcription factor fusion is not expressed. They can also be named unannotated transcripts (also named in the present application neo-transcripts) as they are typically unannotated in the databases referencing genes and their transcripts from normal cells or tissues. Therefore, the potential ORFs of the transcripts identified at step i) do not match any known protein in databases.

As previously defined, the normal representative transcriptome is the corresponding healthy cell or tissue transcriptome. Annotated sequences are sequences, which are typically described in databases such as Ensembl genome database or the expression atlas from EBI (typically the human assembly GRCh37, also known as hg19), the RefSeq human reference genome database, or the GEO databases from NCBI. The GENCODE transcriptome database (https://www.gencodegenes.org/) (typically the human GENCODE human transcriptome database, or the GTEX database (https://gtexportal.org/home/) are also usable according to the present method.

According to the present disclosure, the neotranscripts identified at step i) are specifically associated with the transcription-fusion tumor type. In other words, the expression of said transcripts is found only (or almost only) tumor cell wherein the corresponding transcription factor fusion is found. Expression of these transcripts can be quantified according to known methods in the field (see the results Section) and expression level compared to a threshold value. Threshold values can be set by default (see the values proposed in the EBIS databases), however in some embodiments of the present disclosure the threshold values can be set at 7.5 TPM at least or at 10 TPM at least. In some embodiments, sporadic low-level expression of said transcripts (as compared to their expression in the transcription factor fusion-driven tumor) can be found in a limited number of tissues or tumors (notably in germinal tissues such as placenta and testis), but these level are much lower, notably at least 10, at least 20, at least 50 or at least 100 times lower than the mean expression level observed in the transcription factor fusion-driven tumor.

By specifically expressed in a sample from said transcription factor fusion-driven tumor it is herein intended that the identified ORF sequence is expressed at higher level or frequency (typically disproportionally expressed) in the transcription factor fusion-driven tumor sample as compared to any other control samples (typically a corresponding healthy sample or another tumor sample) from the same type of subject. By disproportionally expressed it is intended that the expression is at least 10, at least 20, at least 50 or at least 100 times higher in the transcription factor fusion-driven tumor sample as compared to a control sample (i.e., a corresponding normal cell or tissue). Typically, the transcripts are expressed at a level of 7.5 TPM or more, notably at 10 TPM or more in the sample.

A transcription factor fusion as per the present disclosure is typically produced by gene fusions which are specific for a given tumor or group of tumors and has gain-of-function activities.

Gene fusions are hybrid genes formed when two previously independent genes become juxtaposed. The fusion can result from structural rearrangements like translocations, insertions, and deletions, transcription read-through of neighboring genes (see Nacu S et al., “Deep RNA sequencing analysis of readthrough gene fusions in human prostate adenocarcinoma and reference samples” BMC Med Genomics. 2011 Jan. 24; 4:11), or the trans- and cis-splicing of pre-mRNAs (Li H, Wang J, Ma X, Sklar J “Gene fusions and RNA trans-splicing in normal and neoplastic human cells” Cell Cycle. 2009 Jan. 15; 8(2):218-22). Gene fusions commonly exert their oncogenic influence by either deregulating one of the involved genes (e.g. by fusing a strong promoter to a proto-oncogene), forming a fusion protein with oncogenic functionality or inducing a loss of function. Gene fusion landscapes have now been studied in many cancer, or tumor, types, including breast, lung, prostate, lymphoid, soft tissue and gastric cancer (see Latysheva, N. S., & Babu, M. M. 2016 “Discovering and understanding oncogenic gene fusions through data intensive computational approaches” Nucleic acids research, 44(10), 4487-4503, for review).

Thus, according to the present application, additional fusion genes coding for aberrant fusion transcription factors (see notably Burchill S A. Ewing's sarcoma: diagnostic, prognostic, and therapeutic implications of molecular abnormalities. J Clin Pathol. 2003; 56(2):96-102; Mertens F, Antonescu C R, Mitelman F. Gene fusions in soft tissue tumors: Recurrent and overlapping pathogenetic themes, Genes Chromosomes Cancer. 2016; 55(4):291-310; and Enzinger and Weiss's Soft Tissue Tumors 6th Edition, John Goldblum Sharon Weiss Andrew L. Folpe) may be targeted as per the method as herein disclosed. For example, the fusion gene coding for fusion transcription factors and/or corresponding specific tumors, which are listed below in table 1, may be targeted:

TABLE 1
transcription factor fusion-driven tumors and
corresponding transcription factor fusions.

| SOFT TISSUE TUMOR | GENE FUSION |
|---|---|
| Alveolar rhabdomyosarcoma | PAX3-FOXO1 |
| | PAX7-FOXO1 |
| Alveolar soft part sarcoma | ASPSCR1-TFE3 |
| Angiofibroma | AHRR-NCOA2 |
| Angiomatoid fibrous histiocytoma | EWSR1-CREB1 |
| | EWSR1-ATF1 |
| | FUS-ATF1 |
| Clear cell sarcoma | EWSR1-ATF1 |
| | EWSR1-CREB1 |
| Dermatofibrosarcoma protuberans/ | COL1A1-PDGFB |
| giant cell fibroblastoma | |
| Desmoplastic small round cell | EWSR1-WT1 |
| tumor | |
| Epithelioid | WWTR1-CAMTA1 |
| hemangioendothelioma | TFE3-YAP1 |
| Ewing sarcoma | EWSR1-FLI1 |
| | EWSR1-ERG |
| | EWSR1 fusion with various |
| | ETS partners: ETV1 (7p22), |
| | FEV (2q36), ETV4 (17q21) |
| | FUS-ERG |
| Ewing sarcoma-like | EWSR1-NFATC2 |
| | CIC-DUX4 |
| | CIC-DUX4 |
| | BCOR-CCNB3 |
| EWSR1-PATZ1 sarcoma | EWSR1-PATZ1 |
| Extraskeletal myxoid | EWSR1-NR4A3 |
| chondrosarcoma | TAF15-NR4A3 |
| | TCF12-NR4A3 |
| | TFG-NR4A3 |
| Congenital Infantile | ETV6-NTRK3 or other |
| fibrosarcoma | NTRK fusions |
| Inflammatory myofibroblastic | ALK fusion with: |
| tumor | TPM4 (19p13.1), TPM3 (1q21), |
| | CLTC (17q23), RANBP2 (2q13), |
| | ATIC (2q35), SEC31A (4q21), |
| | CARS (11p15) |
| Lipoblastoma | PLAG1 fusions |
| Lipoma, ordinary | HMGA2 fusions |
| | HMGA1 fusions |
| Lipoma, chondroid | C11orf95-MKL2 |
| Low-grade fibromyxoid | FUS-CREB3L2 |
| sarcoma | FUS-CREB3L1 |
| Mesenchymal chondrosarcoma | HEY1-NCOA2 |
| Midline carcinoma | BRD4-NUTM1 |
| Myoepithelioma, soft tissue | EWSR1-ZNF444 |
| | EWSR1-PBX1 |
| | EWSR1-POU5F1 |
| Myxoid/round cell liposarcoma | FUS-DDIT3 |
| | EWSR1-DDIT3 |
| Myxoid liposarcoma | EWS-CHOP |
| Extraskeletal myxoid | EWS-CHN |
| chondrosarcoma | |
| Myxoinflammatory fibroblastic | TGFBR3-MGEA5 |
| sarcoma/hemosiderotic | |
| fibrolipomatous tumor | |
| Nodular fasciitis | MYH9-USP6 |
| Ossifying fibromyxoid tumor | PHF1 fusions |
| Pericytoma | ACTB-GLI1 |
| TFE3 Renal cell carcinoma | TFE3or TFEB fusions |
| Sclerosing epithelioid | FUS-CREB3L2 |
| fibrosarcoma | FUS-CREB3L1 |
| Solitary fibrous tumor | NAB2-STAT6 |
| Spindle cell | NCOA2 fusion with: |
| rhabdomyosarcoma | SRF(6p21), TEAD1(11p15) |
| Synovial sarcoma | SS18-SSX1 |
| | SS18-SSX2 |
| | SS18-SSX4 |
| Tenosynovial giant cell tumor | CSF1-COL6A3 |

In some embodiments of the method for identifying tumor neoantigenic peptides as defined above the transcription factor fusion-driven cancer (or tumor) and corresponding transcription factor fusion is selected from table 1. In some embodiments, the transcription factors fusion is a PAX-FOXO fusion, an EWS-FLI1 fusion or an EWS-WT1 fusion and the tumor is alveolar rhabdomyosarcoma, Desmoplastic small round cell tumor or Ewing sarcoma, respectively.

In more embodiments, the present disclosure provides a method for identifying Ewing-sarcoma specific neoantigenic peptides which comprises:
i. identifying transcripts from one or more samples isolated from an Ewing Sarcoma tumor obtained from one or more subjects, which transcription is positively regulated by the EWS-FLI1 fusion protein, which transcription is specifically associated with the Ewing sarcoma tumor and optionally which originate (i.e. is encoded) from intergenic or intronic regions of the genome;
ii. identifying open reading frame (ORF) sequences, among the transcripts of step (i) which are specifically expressed in an Ewing sarcoma sample.

Identification of transcripts, which are regulated by a transcription factor fusion and which originate (i.e., which are transcripted) from intergenic or intronic regions of the genome, according to step i) of the above defined method can be for example achieved by performing RNA (notably long read (5-15 kb)) sequencing using expression system (notably cell lines) wherein the expression of said transcription factor fusion is made inducible.

Identification of transcripts which originates from intergenic regions in the genome can be achieved practically by discarding transcripts which have a match in a reference database (see above for definition and suitable examples) of transcriptome from the representative healthy (i.e. normal) tissue or cell and retrieving only transcripts that have no match in said reference database (i.e., unannotated transcript).

The results included in the present application illustrate identification of unannotated transcripts that are regulated by EWS-FLI1 fusion protein by performing long reads (of about 10 kbases) sequencing (typically using the Pacific Biosciences plateform) using a cell line wherein expression of EWS-FLI1 has been down-regulated (using notably a DOX-inducible small hairpin RNA) and comparison with the human RefSeq database. Only unannotated transcripts that are negatively regulated upon EWS-FLI1 depletion were selected.

The results also illustrate a short-read strategy to explore in more depth the neotranscriptome of Ewing sarcoma based on genome-guided assembly (Shao and Kingsford, Nat Biotechnol. 2017 December; 35(12):1167-1169). Thus, typically, “Scallop” (Shao M, Kingsford; Nat Biotechnol. 2017), a reference-based transcript assembler, can be used to predict all transcript sequences based on aligned RNA-seq reads, independently of a reference transcriptome annotation (see also the results section relative to the experimental procedure. Transcripts that were not annotated in the reference humanGENCODE database (https://www.gencodegenes.org/human/release_19.html was used for example) were retrieved. The expression of transcripts detected specifically in various Ewing sarcoma samples (and not in other tumors or sarcomas) was explored across various databases of normal and tumor tissues. As mentioned above, some of the identified neogenes were moderately expressed (most less than 10 TPM) in germinal tissues (testis and placenta), and therefore allowed the few genes (less than 1.5% of neogenes identified) expressed in these tissues at more than 10 TPM to pass the filter nonetheless.

Typically, the transcripts of the present disclosure are further characterized in that they are encoded by neogenes having evidence of physical binding of the transcription factor fusion near their transcription star site (typically less than 5 kb), such as repeated (at least 2, 3, 4 or more) known binding motifs of transcription factors involved in promoter regulation, such as GGAA binding sites (Grunewald et al., Nat Rev Dis Primers. 2018 Jul. 5; 4(1):5) and/or chromatin activation marks. Typically, the binding motifs can be identified ChIP-Sequencing assays. By combining chromatin immunoprecipitation (ChIP) assays with sequencing, ChIP sequencing (ChIP-Seq) can thus be used for identifying genome-wide DNA binding sites for transcription factors. Following ChIP protocols, DNA-bound protein is immunoprecipitated using a specific antibody. The bound DNA is then coprecipitated, purified, and sequenced.

Typically, the EWS-FLI1 transcription factor fusion regulates of the transcription of the unannotated transcript upon binding on GGAA microsatellite sequences in the promoter region of said unannotated transcripts preferably also in the presence of activated histone marks, notably H3K27ac and/or H3K4me3 histone marks. Identifications of such binding domains and/or chromatin activation marks can be achieved for example using RNA seq data from Illumina Plateform and/or ChIP-seq data using appropriate filters, such as the presence of repeated (at least 2; 3; 4 times or more) GGAA motifs (poly GGAA motifs) and/or the presence of histone marks in the promoter or enhancer region of the transcript.

Cancer cells according to the present disclosure can be isolated from solid tumors or non-solid tumors as previously defined, notably bone tumors or soft tissue tumors. Typically, cancer cells are from an Ewing sarcoma tumor sample.

According to the present disclosure, the mRNA sequences can come from all types of cancer cell or tumor cell sample(s). The tumor may be a solid or a non-solid tumor, notably bone tumors or soft tissue tumors. In particular, the mRNA sequences come from an Ewing sarcoma sample from a primary or secondary tumor.

According to the present disclosure, the neo-transcript sequences are expressed at higher levels in tumor cells compared to normal healthy cells (e.g., disproportionally expressed in tumor cells as compared to normal healthy cells). Typically, the ratio of the median expression in the tumor sample over the median level of expression in all normal tissues is more than 5, notably more than 10 or more than 20. In some embodiments, the neo-transcript sequence is expressed in tumor cells and not in healthy cells, in particular not in thymus healthy cells. Such neo-transcript may be called tumor specific neo-transcripts as per the present disclosure. Neo-transcripts that are expressed at higher level(s) in tumor cells as compared to normal cell, typically that are disproportionally expressed in tumors cells as compared to normal cells as defined above may be called tumor associated neo-transcripts as per the present disclosure.

Typically said neo-transcripts are expressed in more than one representative Ewing sarcoma, notably more than 4 representative Ewing sarcoma.

In some embodiment, a neo-transcript as per the present disclosure is expressed in at least 1%, notably at least 5%, 6%, 7%, 8%, 9%, 10%, 15%, 20%, 25%, 30%, 40%, 50%, 60%, 70%, 80%, 90%, 95%, or even 99% of a population of subjects suffering from a tumor and more specifically from a population of subjects suffering from Ewing sarcoma. In some embodiments, a neotranscript according to the present disclosure is expressed in 100% of subjects suffering from a cancer, or tumor, and more specifically from a population of subjects suffering from Ewing sarcoma.

The neo-transcript sequence may be specific for a cancer, or tumor, cell type of shared between cancer, or tumor, cells. Typically, the neo-transcript is specific for Ewing sarcoma samples.

Open reading frame of the neo-transcripts can be then predicted, for example using ORF finder tools which are well-known in the field, notably by using ORFfinder (https://www.ncbi.nlm.nih.gov/orffinder/), in the three frames, with parameters “minimal ORF length”=75 nucleotides and “ORF start codon”=any.

The method as herein disclosed typically further comprises a step of determining the binding of putative neoantigen peptides to at least one MEW molecule of a subject or population of subjects suffering from the cancer, or tumor, driven by said transcription factor fusion (notably Ewing sarcoma). Typically, such step can be performed in silico.

MEW class I proteins form a functional receptor on most nucleated cells of the body. There are 3 major MEW class I genes in HLA: HLA-A, HLA-B, HLA-C and three minor genes HLA-E, HLA-F and HLA-G. 32-microglobulin binds with major and minor gene subunits to produce a heterodimer. MEW molecules of class I consist of a heavy chain and a light chain and are capable of binding a peptide of about 8 to 11 amino acids, but usually 8 or 9 amino acids, if this peptide has suitable binding motifs, and presenting it to cytotoxic T-lymphocytes. The binding of the peptide is stabilized at its two ends by contacts between atoms in the main chain of the peptide and invariant sites in the peptide-binding groove of all MEW class I molecules. There are invariant sites at both ends of the groove which bind the amino and carboxy termini of the peptide. Variations in peptide length are accommodated by a kinking in the peptide backbone, often at proline or glycine residues that allow the required flexibility. The peptide bound by the MEW molecules of class I usually originates from an endogenous protein antigen. As an example, the heavy chain of the MHC molecules of class I is typically an HLA-A, HLA-B or HLA-C monomer, and the light chain is β-2-microglobulin, in humans.

There are 3 major and 2 minor MHC class II proteins encoded by the HLA. The genes of the class II combine to form heterodimeric (αβ) protein receptors that are typically expressed on the surface of antigen-presenting cells. The peptide bound by the MHC molecules of class II usually originates from an extracellular or exogenous protein antigen. As an example, the α-chain and the β-chain are in particular HLA-DR, HLA-DQ and HLA-DP monomers, in humans. MHC class II molecules are capable of binding a peptide of about 8 to 20 amino acids, notably from 10 to 25 or from 13 to 25 if this peptide has suitable binding motifs, and presenting it to T-helper cells. These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

In the present application, “MHC molecule” refers to at least one MHC class I molecule or at least one MHC Class II molecule.

When carried out on human samples, the method may comprise a step of determining the patient's class I or class I Major Histocompatibility Complex (MHC, aka human leukocyte antigen (HLA) alleles).

A MHC allele database is carried out by analyzing known sequences of MHC I and MHC II and determining allelic variability for each domain. This can be typically determined in silico using appropriate software algorithms well-known in the field. Several tools have been developed to obtain HLA allele information from genome-wide sequencing data (whole-exome, whole-genome, and RNA sequencing data), including OptiType, Polysolver, PHLAT, HLAreporter, HLAforest, HLAminer, and seq2HLA (see Kiyotani K et al., Immunopharmacogenomics towards personalized cancer immunotherapy targeting neoantigens; Cancer Science 2018; 109:542-549). For example, the seq2hla tool (see Boegel S, Lower M, Schafer M, et al. HLA typing from RNA-Seq sequence reads. Genome Med. 2012; 4:102), which is well designed to perform the method as herein disclosed is an in silico method written in python and R, which takes standard RNA-Seq sequence reads in fastq format as input, uses a bowtie index (Langmead B, et al., Ultrafast and memory-efficient alignment of short DNA sequences to the human genome. Genome Biol. 2009, 10: R25-10.1186/gb-2009-10-3-r25) comprising all HLA alleles and outputs the most likely HLA class I and class II genotypes (in 4 digit resolution), a p-value for each call, and the expression of each class.

The affinity of all possible peptides encoded by each neo-transcript sequence for each MEW allele from the subject can be determined in silico using computational methods to predict peptide binding-affinity to HLA molecules. Indeed, accurate prediction approaches are based on artificial neural networks with predicted IC₅₀. For example, NetMHCpan software which has been modified from NetMHC to predict peptides binding to alleles for which no ligands have been reported, is well appropriate to implement the method as herein disclosed (Lundegaard C et al., NetMHC-3.0: accurate web accessible predictions of human, mouse and monkey MEW class I affinities for peptides of length 8-11; Nucleic Acids Res. 2008; 36:W509-W512; Nielsen M et al. NetMHCpan, a method for quantitative predictions of peptide binding to any HLA-A and -B locus protein of known sequence. PLoS One. 2007; 2:e796, but see also Kiyotani K et al., Immunopharmacogenomics towards personalized cancer immunotherapy targeting neoantigens; Cancer Science 2018; 109:542-549 and Yarchoan M et al., Nat rev. cancer 2017; 17(4):209-222). NetMHCpan software predicts binding of peptides to any MHC molecule of known sequence using artificial neural networks (ANNs). The method is trained on a combination of more than 180,000 quantitative binding data and MS derived MEW eluted ligands. The binding affinity data covers 172 MEW molecules from human (HLA-A, B, C, E), mouse (H-2), cattle (BoLA), primates (Patr, Mamu, Gogo) and swine (SLA). The MS eluted ligand data covers 55 HLA and mouse alleles.

In example embodiments, neoantigenic peptides encoded by neo-transcripts as herein disclosed and having a Kd affinity of predicted peptides for MHC alleles with a score less than 10⁻⁴. 10⁻⁵, 10⁻⁶, 10⁻⁷, or less than 500 nM or a rank less than 2% (typically depending on netMHCpan version) are selected as tumor neoantigenic peptides.

In a particular embodiment, neoantigenic peptides, and a Kd affinity of predicted peptides for MHC alleles with a score less than 50 nM or a rank less than 0.5% (typically depending on netMHCpan version) are selected as tumor neoantigenic peptides.

Thus, the neoantigenic peptide as per the present disclosure, and typically obtainable as per the present method, binds at least one HLA/MHC molecule with an affinity sufficient for the peptide to be presented on the surface of a cell as an antigen. Generally, the neoantigenic peptide has an IC50 affinity of less than 10⁻⁴. or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less for at least one HLA/MHC molecule (lower numbers indicating greater binding affinity), typically a molecule of said subject suffering from a cancer, or a tumor.

Peptides binding to MHC Class 1 molecules can thus be predicted using for example the NetMHCpan 4.1 suite (http://www.cbs.dtu.dk/services/NetMHCpan/), using “HLA allele”=A2, “peptide length”=8-11 and “rank threshold for strong binding”=0.5%.

In some embodiments, the present method may thus independently include:
a step of exclusion of fusion transcripts or predicted peptides expressed at high levels or high frequency on healthy cells. An alignment of the neo-transcript sequence against the Refseq data (Reference Sequence collection from NCBI providing integrated set of annotated sequences, including genomic DNA, transcripts, and proteins) of healthy cells, allow determining the relative amount of fusion transcript sequence(s) present in healthy cells; In one embodiment, fusion transcripts or predicted peptides expressed on healthy cells are discarded.
a step to confirm that a selected tumor neoantigenic peptide is not expressed in healthy cells of the subject. This step can be carried out in silico using typically the Basic local alignment search tool (BLAST) and performing alignment of the sequence of the neoantigenic peptide against the proteome of healthy cells; and/or
a step to confirm that the fusion transcript or predicted peptide is expressed in cancer, or tumor, cells of the subject. The presence of the selected fusion transcript sequence in cancer, or tumor, cells can be checked typically by RT-PCR in mRNA extracted from said cells (see for example WO93/23549).

Neoantigenic Peptides, Polynucleotides and Vectors

The present disclosure also encompasses neotranscripts having at least the following characteristics:
i) their expression is regulated by a transcription factor fusion as evidenced by expression in cell line wherein the expression of said transcription factor fusion is made inducible,
ii) they are specifically associated with the fusion-driven tumor type,
iii) they are encoded by genome regions having binding motifs involved in promoter regulation, such as poly GGAA, binding sites and/or binding sites for said transcription factor as determined by ChIP-seq experiments, and/or histone activation marks, such as H3K27ac and H3K4me3 histone at less than 5 kb of the TSS.

In some embodiments, the transcripts are further selected according to one or more of the following properties:
their mean expression in the disease of interest (i.e., the transcription factor fusion-driven tumor) of more than 7.5 TPM,
Logarithm of (mean expression in other samples/mean expression in disease of interest) of less than 3,
mean expression in any other samples (i.e., any sample that is not a sample from the disease of interest) of less than 2 TPM, notably between 0 and 2 TPM.
99% quantile of expression in other samples comprised between 0 and 10 TPM,
maximum mean expression in another tumor or tissue of less than 10 TPM, notably of less than, typically comprised between 0 and 10 TMP (excluding testis and placenta),

The present disclosure also relates to an isolated tumor neoantigenic peptide comprising at least 8, 9, 10, 11, or 12 amino acids, encoded by a portion of an open reading frame (ORF) from a neo-transcript sequence as herein disclosed, and/or obtained according to a method of the present disclosure. The peptide may be 8-9, 8-10, 8-11, 12-25, 13-25, 12-20, or 13-20 amino acids in length.

The present disclosure also more specifically encompasses an Ewing sarcoma isolated tumor neoantigenic peptide encoded by a portion of a human mRNA neo-transcript sequence from an Ewing sarcoma cell, which transcription is positively regulated by the EWS-FLI1 fusion protein.

In example embodiments the neoantigenic peptide comprising at least 8, 9, 10, 11 or 12 amino acids is encoded by a part of an open reading frame (ORF) of any of the neo-transcript sequences as defined in SEQ ID NO:1-145.

The N-terminus of the peptides of at least 8 amino acids may be encoded by the triplet codon starting at any of nucleotide positions 1, 4, 7, 10, 13, 16, 19.

A peptide as above defined is typically obtainable according to the method of the present disclosure and thus encompasses one or more of the characteristics as previously described. In particular a neoantigenic peptide as per the present disclosure may exhibit one or more of a combination of the following characteristics:
It binds or specifically binds MEW class I of a subject and is 8 to 11 amino acids, notably 8, 9, 10, or 11 amino acids. Typically, the neoantigenic peptide is 8 or 9 amino acids long, and binds to at least one MEW class I molecule of the subject; or alternatively, it binds to at least one MEW class II molecule of said subject and contains from 12 to 25 amino acids, notably is 12, 13, 14, 15, 16, 17, 18, 19, 20, 21, 22, 23, 24 or 25 amino acids long.
It binds at least one HLA/MHC molecule of said subject suffering from a cancer, or a tumor (notably a transcription factor fusion-riven cancer or tumor as herein described) with an affinity sufficient for the peptide to be presented on the surface of a cell as an antigen. Typically, the neoantigenic peptide has an IC50 of less than 10⁻⁴. or 10⁻⁵, or 10⁻⁶, or 10⁻⁷ or less than 500 nM, at least less than 250 nM, at least less than 200 nM, at least less than 150 nM, at least less than 100 nM, at least less than 50 nM or less (lower numbers indicating greater binding affinity).
It does not induce an autoimmune response and/or invoke immunological tolerance when administered to a subject.
It is expressed at higher levels in tumor cells compared to normal healthy cells. Typically, the ratio of its median expression in the tumor sample over the median level of expression in all normal tissues is more than 5, notably more than 10 or more than 20. In another embodiment, the neoantigenic peptide is a tumor specific antigen (TSA), i.e.: it is only expressed in cancer, or tumor cells and not in healthy cells (e.g., not detectably expressed). Lack of expression of a neoantigenic peptide in healthy cell mays be tested using notably the Basic local alignment search tool (BLAST) and performing alignment of the sequence of the neoantigenic peptide against the proteome of healthy cells.

A tumor neoantigenic peptide may first be validated by RT transcription analysis of fusion transcripts sequence in tumors cell from a subject. Typically also, immunization with a tumor neoantigenic peptide as per the present disclosure elicits a T cell response

In a particular embodiment, the present disclosure encompasses an Ewing sarcoma specific neoantigenic peptide comprising at least 8 amino acids. Typically, said neoantigenic peptides binds to HLA-A02 with an affinity sufficient for the peptide to be presented on the surface of cells as an antigen. Kd affinity can be determined or predicted by using tetramer preparation as illustrated in the examples. Briefly, HLA 02:01/peptide tetramers can be prepared using adapted commercial kits and incubated with human CD8+ prepared from healthy controls. Tetramer-CD8+ cell binding can be assessed by flow cytometry.

In a particular embodiment, a tumor neoantigenic peptide as per the present disclosure binds to a MHC molecule present in at least 1%, 5%, 10%, 15%, 20%, 25% or more of subjects. Notably, a tumor neoantigenic peptide as herein disclosed is expressed in at least 1%, 5%, 10%, 15%, 20%, 25% of subjects from a population of subjects suffering from a cancer or a tumor, notably suffering from Ewing sarcoma.

More particularly, a tumor neoantigenic peptide of the present disclosure is capable of eliciting an immune response against a tumor present in at least 5%, 6%, 7%, 8%, 9%, 10%, 15%, 20%, 25%, 30%, 40%, 50%, 60%, 70%, 80%, 90%, 95%, or even 99% of a population of subjects suffering from a cancer, or a tumor, and more specifically from a population of subjects suffering from Ewing sarcoma. In some embodiments, a tumor neoantigenic peptide according to the present disclosure is expressed in 100% of subjects suffering from a cancer, or a tumor, and more specifically from a population of subjects suffering from Ewing sarcoma.

The neoantigenic peptide can also be modified by extending or decreasing the compound's amino acid sequence, e.g., by the addition or deletion of amino acids. The peptides can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MEW molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions are generally made in accordance with the following Table 2 when it is desired to finely modulate the characteristics of the peptide.

TABLE 2

| Original residue | Exemplary substitution |
|---|---|
| Ala | Ser |
| Arg | Lys, His |
| Asn | Gln |
| Asp | Glu |
| Cys | Ser |
| Gln | Asn |
| Glu | Asp |
| Gly | Pro |
| His | Lys; Arg |
| Ile | Leu; Val |
| Leu | Ile; Val |
| Lys | Arg; His |
| Met | Leu; Ile |
| Phe | Tyr; Trp |
| Ser | Thr |
| Thr | Ser |
| Trp | Tyr, Phe |
| Tyr | Trp, Phe |
| Val | Ile, Leu |
| Pro | Gly |

Substantial changes in function (e.g., affinity for MHC molecules or T cell receptors) are made by selecting substitutions that are less conservative than those in above Table, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl, is substituted for (or by) a hydrophobic residue, e.g. leucyl, isoleucyl, phenylalanyl, valyl or alanyl; (b) a residue having an electropositive side chain, e.g., lysl, arginyl, or histidyl, is substituted for (or by) an electronegative residue, e.g. glutamyl or aspartyl; or (c) a residue having a bulky side chain, e.g. phenylalanine, is substituted for (or by) one not having a side chain, e.g., glycine.

The peptides and polypeptides may also comprise isosteres of two or more residues in the neoantigenic peptide or polypeptides. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

In addition, the neoantigenic peptide may be conjugated to a carrier protein, a ligand, or an antibody. Half-life of the peptide may be improved by PEGylation, glycosylation, polysialylation, HESylation, recombinant PEG mimetics, Fc fusion, albumin fusion, nanoparticle attachment, nanoparticulate encapsulation, cholesterol fusion, iron fusion, or acylation.

Modifications of peptides and polypeptides with various amino acid mimetics or unnatural amino acids are particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half life of the peptides of the present disclosure is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides and polypeptides may be modified to provide desired attributes other than improved serum half-life. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the peptide may be linked to the T helper peptide without a spacer.

The neoantigenic peptide may be linked to the T helper peptide either directly or via a spacer either at the amino or carboxy terminus of the peptide. The amino terminus of either the neoantigenic peptide or the T helper peptide may be acylated. Exemplary T helper peptides include tetanus toxoid 830-843, influenza 307-319, malaria circumsporozoite 382-398 and 378-389.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Infornation's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

In a further aspect, the present disclosure provides a nucleic acid (e.g. polynucleotide) encoding a neoantigenic peptide as herein disclosed. The polynucleotide may be selected from DNA, cDNA, PNA, CNA, RNA, either single- and/or double-stranded, or native or stabilized forms of polynucleotides, such as for example polynucleotides with a phosphorothiate backbone, or combinations thereof and it may or may not contain introns so long as it codes for the peptide. Only peptides that contain naturally occurring amino acid residues joined by naturally occurring peptide bonds are encodable by a polynucleotide. In some embodiments, the polynucleotide may be linked to a heterologous regulatory control sequence (e.g., heterologous transcriptional and/or translational regulatory control nucleotide sequences as well-known in the field).

A still further aspect of the disclosure provides an expression vector capable of expressing a neoantigenic peptide as herein disclosed. Expression vectors for different cell types are well known in the art and can be selected without undue experimentation. Generally, the DNA is inserted into an expression vector, such as a plasmid, in proper orientation and correct reading frame for expression. The expression vector will comprise the appropriate heterologous transcriptional and/or translational regulatory control nucleotide sequences recognized by the desired host. The polynucleotide encoding the tumor neoantigenic peptide may be linked to such heterologous regulatory control nucleotide sequences or may be non-adjacent yet operably linked to such heterologous regulatory control nucleotide sequences. The vector is then introduced into the host through standard techniques. Guidance can be found for example in Sambrook et al. (1989) Molecular Cloning, A Laboratory Manual, Cold Spring Harbor Laboratory, Cold Spring Harbor, N.Y.

Antigen Presenting Cells (APCs)

The present disclosure also encompasses a population of antigen presenting cells that have been pulsed with one or more of the peptides as previously defined and/or obtainable in a method as previously described. Preferably, the antigen presenting cells are dendritic cell (DCs) or artificial antigen presenting cells (aAPCs) (see Neal, Lillian R et al. “The Basics of Artificial Antigen Presenting Cells in T Cell-Based Cancer Immunotherapies.” Journal of immunology research and therapy vol. 2,1 (2017): 68-79). Dendritic cells (DC) are professional antigen-presenting cells (APC) that have an extraordinary capacity to stimulate naive T-cells and initiate primary immune responses to pathogens. Indeed, the main role of mature DCs are to sense antigens and produce mediators that activate other immune cells, particularly T cells. DCs are potent stimulators for lymphocyte activation as they express MHC molecules that trigger TCRs (signal 1) and co-stimulatory molecules (signal 2) on T cells. Additionally, DCs also secrete cytokines that support T cell expansion. T cells require presented antigen in the form of a processed peptide to recognize foreign pathogens or tumor. Presentation of peptide epitopes derived from pathogen/tumor proteins is achieved through MHC molecules. MHC class I (MHC-I) and MHC class II (MHC-II) molecules present processed peptides to CD8+ T cells and CD4+ T cells, respectively. Importantly, DCs home to inflammatory sites containing abundant T cell populations to foster an immune response. Thus, DCs can be a crucial component of any immunotherapeutic approach, as they are intimately involved with the activation of the adaptive immune response. In the context of vaccines, DC therapy can enhance T cell immune responses to a desired target in healthy volunteers or patients with infectious disease or cancer. In one embodiment, APCS are artificial APC, which are genetically modified to express the desired T-cell co-stimulatory molecules, human HLA alleles and/or cytokines. Such artificial antigen presenting cells (aAPC) are able to provide the requirements for adequate T-cell engagement, co-stimulation, as well as sustained release of cytokines that allow for controlled T-cell expansion. These cells are not subject to the constraints of time and limited availability and can be stored in small aliquots for subsequent use in generating T-cell lines from different donors, thus representing an off the shelf reagent for immunotherapy applications. Expression of potent co-stimulatory signals on these aAPC endows this system with higher efficiency lending to increased efficacy of adoptive immunotherapy. Furthermore, aAPC can be engineered to express genes directing release of specific cytokines to facilitate the preferential expansion of desirable T-cell subsets for adoptive transfer; such as long lived memory T-cells (see for review Hasan A H et al., Artificial Antigen Presenting Cells: An Off the Shelf Approach for Generation of Desirable T-Cell Populations for Broad Application of Adoptive Immunotherapy; Adv Genet Eng. 2015; 4(3): 130, Kim J V, Latouche J B, Rivière I, Sadelain M. The ABCs of artificial antigen presentation. Nat Biotechnol. 2004; 22:403-410 or Wang C, Sun W, Ye Y, Bomba H N, Gu Z. Bioengineering of Artificial Antigen Presenting Cells and Lymphoid Organs. Theranostics 2017; 7(14):3504-3516.).

Typically, the dendritic cells are autologous dendritic cells that are pulsed with a neoantigenic peptide as herein disclosed. The peptide may be any suitable peptide that gives rise to an appropriate T-cell response. The antigen-presenting cell (or stimulator cell) typically has an MHC class I or II molecule on its surface, and in one embodiment is substantially incapable of itself loading the MHC class I or II molecule with the selected antigen. The MHC class I or II molecule may readily be loaded with the selected antigen in vitro.

As an alternative the antigen presenting cell may comprise an expression construct encoding a tumor neoantigenic peptide as herein disclosed. The polynucleotide may be any suitable polynucleotide as previously defined and it is preferred that it is capable of transducing the dendritic cell, thus resulting in the presentation of a peptide and induction of immunity.

Thus the present disclosure encompasses a population of APCs than can be pulsed or loaded with the neoantigenic peptide as herein disclosed, genetically modified (via DNA or RNA transfer) to express at least one neoantigenic peptide as herein disclosed, or that comprise an expression construct encoding a tumor neoantigenic peptide of the present disclosure. Typically the population of APCs is pulsed or loaded, modified to express or comprises at least one, at least 5, at least 10, at least 15, or at least 20 different neoantigenic peptide or expression construct encoding it.

The present disclosure also encompasses compositions comprising APCs as herein disclosed. APCs can be suspended in any known physiologically compatible pharmaceutical carrier, such as cell culture medium, physiological saline, phosphate-buffered saline, cell culture medium, or the like, to form a physiologically acceptable, aqueous pharmaceutical composition. Parenteral vehicles include sodium chloride solution, Ringer's dextrose, dextrose and sodium chloride, lactated Ringer's. Other substances may be added as desired such as antimicrobials. As used herein, a “carrier” refers to any substance suitable as a vehicle for delivering an APC to a suitable in vitro or in vivo site of action. As such, carriers can act as an excipient for formulation of a therapeutic or experimental reagent containing an APC. Preferred carriers are capable of maintaining an APC in a form that is capable of interacting with a T cell. Examples of such carriers include, but are not limited to water, phosphate buffered saline, saline, Ringer's solution, dextrose solution, serum-containing solutions, Hank's solution and other aqueous physiologically balanced solutions or cell culture medium. Aqueous carriers can also contain suitable auxiliary substances required to approximate the physiological conditions of the recipient, for example, enhancement of chemical stability and isotonicity. Suitable auxiliary substances include, for example, sodium acetate, sodium chloride, sodium lactate, potassium chloride, calcium chloride, sorbitan monolaurate, triethanolamine oleate, and other substances used to produce phosphate buffer, Tris buffer, and bicarbonate buffer.

Vaccine Compositions

The present disclosure further encompasses a vaccine or immunogenic composition capable of raising a specific T-cell response comprising:
one or more neoantigenic peptides as herein defined,
one or more polynucleotides encoding a neoantigenic peptide as herein defined; and/or
a population of antigen presenting cells (such as autologous dendritic cells or artificial APC) as described above.

A suitable vaccine or immunogenic composition will preferably contain between 1 and 20 neoantigenic peptides, more preferably 2, 3, 4, 5, 6, 7, 8, 9, 10, 11, 12, 13, 14, 15, 16, 17, 18, 19, 20, 21, 22, 23, 24, 25 different neoantigenic peptides, further preferred 6, 7, 8, 9, 10 11, 12, 13, or 14 different neoantigenic peptides, and most preferably 12, 13 or 14 different neoantigenic peptides.

The neoantigenic peptide(s) may be linked to a carrier protein. Where the composition contains two or more neoantigenic peptides, the two or more (e.g. 2-25) peptides may be linearly linked by a spacer molecule as described above, e.g. a spacer comprising 2-6 nonpolar or neutral amino acids.

In one embodiment of the present disclosure the different neoantigenic peptides, encoding polynucleotides, vectors, or APCs are selected so that one vaccine or immunogenic composition comprises neoantigenic peptides capable of associating with different MEW molecules, such as different MEW class I molecules. Preferably, such neoantigenic peptides are capable of associating with the most frequently occurring MEW class I molecules, e.g. different fragments capable of associating with at least 2 preferred, more preferably at least 3 preferred, even more preferably at least 4 preferred MEW class I molecules. In some embodiments, the compositions comprise peptides, encoding polynucleotides, vectors, or APCs capable of associating with one or more MEW class II molecules. The MEW is optionally HLA-A, -B, -C, -DP, -DQ, or -DR.

The vaccine or immunogenic composition is capable of raising a specific cytotoxic T-cells response and/or a specific helper T-cell response.

Thus in a particular embodiment, the present disclosure also relates to a neoantigenic peptide as described above, wherein the neoantigenic peptide has a tumor specific neoepitope and is included in a vaccine or immunogenic composition. A vaccine composition is to be understood as meaning a composition for generating immunity for the prophylaxis and/or treatment of diseases. Accordingly, vaccines are medicines which comprise or generate antigens and are intended to be used in humans or animals for generating specific defense and protective substance by vaccination. An “immunogenic composition” is to be understood as meaning a composition that comprises or generates antigen(s) and is capable of eliciting an antigen-specific humoral or cellular immune response, e.g. T-cell response.

In a preferred embodiment, the neoantigenic peptide according to the disclosure is 8 or 9 residues long, or from 13 to 25 residues long. When the peptide is less than 20 residues, in order to have a peptide better suited for in vivo immunization, said neoantigenic peptide, is optionally flanked by additional amino acids to obtain an immunization peptide of more amino acids, usually more than 20.

Pharmaceutical compositions (i.e., the vaccine or immunogenic composition) comprising a peptide as herein described may be administered to an individual already suffering from a cancer or a tumor. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific CTL activity in the patient's blood. It must be kept in mind that the peptide and compositions of the present invention may generally be employed in serious disease states, that is, life-threatening or potentially life threatening situations, especially when the cancer has metastasized. In such cases, in view of the minimization of extraneous substances and the relative nontoxic nature of the peptide, it is possible and may be felt desirable by the treating physician to administer substantial excesses of these peptide compositions.

For therapeutic use, administration should begin at the detection or surgical removal of tumors. This is followed by boosting doses until at least symptoms are substantially abated and for a period thereafter.

The vaccine or immunogenic compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor.

The vaccine or immunogenic composition may be a pharmaceutical composition which additionally comprises a pharmaceutically acceptable adjuvant, immunostimulatory agent, stabilizer, carrier, diluent, excipient and/or any other materials well known to those skilled in the art. Such materials should be non-toxic and should not interfere with the efficacy of the active ingredient. The carrier is preferably an aqueous carrier but its precise nature of the carrier or other material will depend on the route of administration. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may further contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. See, for example, Butterfield, B M J. 2015 22; 350 for a discussion of cancer vaccines.

Example adjuvants that increase or expand the immune response of a host to an antigenic compound include emulsifiers, muramyl dipeptides, avridine, aqueous adjuvants such as aluminum hydroxide, chitosan-based adjuvants, saponins, oils, Amphigen, LPS, bacterial cell wall extracts, bacterial DNA, CpG sequences, synthetic oligonucleotides, cytokines and combinations thereof. Emulsifier include, for example, potassium, sodium and ammonium salts of lauric and oleic acid, calcium, magnesium and aluminum salts of fatty acids, organic sulfonates such as sodium lauryl sulfate, cetyltrhethylammonlum bromide, glycerylesters, polyoxyethylene glycol esters and ethers, and sorbitan fatty acid esters and their polyoxyethylene, acacia, gelatin, lecithin and/or cholesterol. Adjuvants that comprise an oil component include mineral oil, a vegetable oil, or an animal oil. Other adjuvants include Freund's Complete Adjuvant (FCA) or Freund's Incomplete Adjuvant (FIA). Cytokines useful as additional immunostimulatory agents include interferon alpha, interleukin-2 (IL-2), and granulocyte macrophage-colony stimulating factor (GM-CSF), or combinations thereof.

The concentration of peptides as herein described in the vaccine or immunogenic formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The peptides as herein described may also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871; 4,501,728; 4,837,028; and 5,019,369.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

Cytotoxic T-cells (CTLs) recognize an antigen in the form of a peptide bound to an MEW molecule rather than the intact foreign antigen itself. The MEW molecule itself is located at the cell surface of an antigen presenting cell. Thus, an activation of CTLs is only possible if a trimeric complex of peptide antigen, MEW molecule, and antigen presenting cell (APC) is present. Correspondingly, it may enhance the immune response if not only the peptide is used for activation of CTLs, but if additionally APCs with the respective MEW molecule are added. Therefore, in some embodiments the vaccine or immunogenic composition according to the present disclosure alternatively or additionally contains at least one antigen presenting cell, preferably a population of APCs.

The vaccine or immunogenic composition may thus be delivered in the form of a cell, such as an antigen presenting cell, for example as a dendritic cell vaccine. The antigen presenting cells such as a dendritic cell may be pulsed or loaded with a neoantigenic peptide as herein disclosed, may comprise an expression construct encoding a neoantigenic peptide as herein disclosed, or may be genetically modified (via DNA or RNA transfer) to express one, two or more of the herein disclosed neoantigenic peptides, for example at least 2, 3, 4, 5, 6, 7, 8, 9, or 10 neoantigenic peptides.

Suitable vaccines or immunogenic compositions may also be in the form of DNA or RNA relating to neoantigenic peptides as described herein. For example, DNA or RNA encoding one or more neoantigenic peptides or proteins derived therefrom may be used as the vaccine, for example by direct injection to a subject. For example, DNA or RNA encoding at least 2, 3, 4, 5, 6, 7, 8, 9, 10, 11, 12, 13, 14, 15, 16, 17, 18, 19, 20, 21, 22, 23, 24 or 25 neoantigenic peptides or proteins derived therefrom.

A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833; WO 91/06309; and Felgner et al., Proc. Natl. Acad. Sci. USA 84: 7413-7414 (1987).

Delivery systems may optionally include cell-penetrating peptides, nanoparticulate encapsulation, virus like particles, liposomes, or any combination thereof. Cell penetrating peptides include TAT peptide, herpes simplex virus VP22, transportan, Antp. Liposomes may be used as a delivery system. Listeria vaccines or electroporation may also be used.

The one or more neoantigenic peptides may also be delivered via a bacterial or viral vector containing DNA or RNA sequences which encode one or more neoantigenic peptides. The DNA or RNA may be delivered as a vector itself or within attenuated bacteria virus or live attenuated virus, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host CTL response. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhivectors and the like, will be apparent to those skilled in the art from the description herein.

An appropriate mean of administering nucleic acids encoding the peptides as herein described involves the use of minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MEW presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Thus, the DNA or RNA encoding the neoantigenic peptide(s) may typically be operably linked to one or more of:
a promoter that can be used to drive nucleic acid molecule expression. AAV ITR can serve as a promoter and is advantageous for eliminating the need for an additional promoter element. For ubiquitous expression, the following promoters can be used: CMV (notably human cytomegalovirus immediate early promoter (hCMV-IE)), CAG, CBh, PGK, SV40, RSV, Ferritin heavy or light chains, etc. For brain expression, the following promoters can be used: Synapsin1 for all neurons, CaMKIIalpha for excitatory neurons, GAD67 or GAD65 or VGAT for GABAergic neurons, etc. Promoters used to drive RNA synthesis can include: Pol III promoters such as U6 or HI. The use of a Pol II promoter and intronic cassettes can be used to express guide RNA (gRNA). Typically, the promoter includes a down-stream cloning site for minigene insertion. For examples of suitable promoters sequences, see notably U.S. Pat. Nos. 5,580,859 and 5,589,466.
Transcriptional transactivators or other enhancer elements, which can also increase transcription activity, e.g. the regulatory R region from the 5′ long terminal repeat (LTR) of human T-cell leukemia virus type 1 (HTLV-1) (which when combined with a CMV promoter has been shown to induce higher cellular immune response).
Translation optimizing sequences e.g. a Kozak sequence flanking the AUG initiator codon (ACCAUGG) within mRNA, and codon optimization.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immunostimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used.

DNA vaccines or immunogenic compositions as herein described can be enhanced by co-delivering cytokines that promote cell-mediated immune responses, such as IL-2, IL-12, IL-18, GM-CSF and IFNγ. CXC chemokines such as IL-8, and CC chemokines such as macrophage inflammatory protein (MIP)-1α, MIP-3α, MIP-3β, and RANTES, may increase the potency of the immune response. DNA vaccine immunogenicity can also be enhanced by co-delivering plasmid-encoded cytokine-inducing molecules (e.g. LeIF), co-stimulatory and adhesion molecules, e.g. B7-1 (CD80) and/or B7-2 (CD86). Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Vaccines or immunogenic compositions comprising peptides may be administered in combination with vaccines or immunogenic compositions comprising polynucleotide encoding the peptides. For example, administration of peptide vaccine and DNA vaccine may be alternated in a prime-boost protocol. For example, priming with a peptide immunogenic composition and boosting with a DNA immunogenic composition is contemplated, as is priming with a DNA immunogenic composition and boosting with a peptide immunogenic composition.

The present disclosure also encompasses a method for producing a vaccine composition comprising the steps of:
a) Optionally, identifying at least one neoantigenic peptide according to the method as previously described;
b) producing said at least one neoantigenic peptide, at least one polypeptide encoding neoantigenic peptide(s), or at least a vector comprising said polypeptide(s) as described herein; and
c) optionally adding physiologically acceptable buffer, excipient and/or adjuvant and producing a vaccine with said at least one neoantigenic peptide, polypeptide or vector.

Another aspect of the present disclosure is a method for producing a DC vaccine, wherein said DCs present at least one neoantigenic peptide as herein disclosed.

Antibodies TCRs, CARs and Derivatives Thereof

The present disclosure also relates to an antibody or an antigen-binding fragment thereof that specifically binds a neoantigenic peptide as herein defined.

In some embodiments, the neoantigenic peptide is in association with an MHC or HLA molecule.

Typically, said antibody, or antigen-binding fragment thereof binds a neoantigenic peptide as herein defined, alone or optionally in association with an MHC or HLA molecule, with a Kd binding affinity of 10⁻⁷ M or less, 10⁻⁸ M or less, 10⁻⁹ M or less, 10⁻¹⁰ M or less, or 10⁻¹¹ M or less.

To promote the infiltration and recognition of tumor cells by lymphocytes T (LT), another strategy consists in using antibodies capable of recognizing more than one antigenic target simultaneously and more particularly two antigenic targets simultaneously. There are many formats of bispecific antibodies. BiTE (bi-specific T-cell engager) are the first to have been developed. These are proteins of fusion consisting of two scFvs (variable domains heavy VH and light VL chains) from two antibodies linked by a binding peptide: one recognizes the LT marker (CD3+) and the other a tumor antigen. The goal is to favor recruitment and activation of LTs in contact with tumor, thus leading to cell lysis tumor (See for review Patrick A. Baeuerle and Carsten Reinhardt; Bispecific T-Cell Engaging Antibodies for Cancer Therapy; Cancer Res 2009; 69: (12). Jun. 15, 2009; and Galaine et al., Innovations & Thérapeutiques en Oncologie, vol. 3-n° 3-7, mai-août 2017).

In a particular embodiment, said antibody is a bi-specific T-cell engager that targets a tumor neoantigenic peptide as herein defined, optionally in association with a MHC or an HLA molecule and which further targets at least an immune cell antigen. Typically, the immune cell is a T cell, a NK cell or a dendritic cell. In this context, the targeted immune cell antigen may be for example CD3, CD16, CD30 or a TCR.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, variable heavy chain (VH) regions capable of specifically binding the antigen, single chain antibody fragments, including single chain variable fragments (scFv), and single domain antibodies (e.g., VHH antibodies, sdAb, sdFv, nanobody) fragments. The term encompasses genetically engineered and/or otherwise variants modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody and fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments, the antibody comprises a light chain variable domain and a heavy chain variable domain, e.g. in an scFv format.

Antibodies include variant polypeptide species that have one or more amino acid substitutions, insertions, or deletions in the native amino acid sequence, provided that the antibody retains or substantially retains its specific binding function. Conservative substitutions of amino acids are well known and described above.

The present disclosure further includes a method of producing an antibody, or antigen-binding fragment thereof, comprising a step of selecting antibodies that bind to a tumor neoantigen peptide as herein defined, optionally in association with an MHC or HLA molecule, with a Kd binding affinity of about 10⁻⁶ M or less, 10⁻⁷ M or less, 10⁻⁸ M or less, 10⁻⁹ M or less, 10⁻¹⁰ M or less, or 10⁻¹¹ M or less.

In some embodiments, the antibodies are selected from a library of human antibody sequences. In some embodiments, the antibodies are generated by immunizing an animal with a polypeptide comprising the neoantigenic peptide, optionally in association with an MHC or HLA molecule, followed by the selection step.

Antibodies including chimeric, humanized or human antibodies can be further affinity matured and selected as described above. Humanized antibodies contain rodent-sequence derived CDR regions; typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence, and are typically produced via phage display technologies of human antibody libraries, or immunization of transgenic mice whose native immunoglobin loci have been replaced with segments of human immunoglobulin loci.

Antibodies produced by said method, as well as immune cells expressing such antibodies or fragments thereof are also encompassed by the present disclosure.

The present disclosure also encompasses pharmaceutical compositions comprising one or more antibodies as herein disclosed alone or in combination with at least one other agent, such as a stabilizing compound, which may be administered in any sterile, biocompatible pharmaceutical carrier and optionally formulated with formulated with sterile pharmaceutically acceptable buffer(s), diluent(s), and/or excipient(s). Pharmaceutically acceptable carriers typically enhance or stabilize the composition, and/or can be used to facilitate preparation of the composition. Pharmaceutically acceptable carriers include solvents, dispersion media, coatings, antibacterial and antifungal agents, isotonic and absorption delaying agents, and the like that are physiologically compatible and in some embodiments pharmaceutically inert.

Administration of pharmaceutical composition comprising antibodies as herein disclosed can be accomplished orally or parenterally. Methods of parenteral delivery include topical, intra-arterial (directly to the tumor), intramuscular, spinal, subcutaneous, intramedullary, intrathecal, intraventricular, intravenous, intraperitoneal, or intranasal administration.

Thus, in addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Depending on the route of administration, the active compound, i.e., antibody, bispecific and multispecific molecule, may be coated in a material to protect the compound from the action of acids and other natural conditions that may inactivate the compound.

The composition is typically sterile and preferably fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical compositions of the disclosure can be prepared in accordance with methods well known and routinely practiced in the art. See. e.g., Remington: The Science and Practice of Pharmacy, Mack Publishing Co., 20th ed., 2000; and Sustained and Controlled Release Drug Delivery Systems, J R. Robinson, ed., Marcel Dekker, Inc., New York, 1978. Pharmaceutical compositions are preferably manufactured under GMP conditions.

The present disclosure also encompasses a T cell receptor (TCR) that targets a neoantigenic peptide as herein defined in association with an MHC or HLA molecule.

The present disclosure further includes a method of producing a TCR, or an antigen-binding fragment thereof, comprising a step of selecting TCRs that bind to a tumor neoantigen peptide as herein defined, optionally in association with an MHC or HLA molecule, optionally with a Kd binding affinity of about 10⁻⁶ M or less, 10⁻⁷ M or less, 10⁻⁸ M or less, 10⁻⁹ M or less, 10⁻¹⁰ M or less, or 10⁻¹¹M or less.

Nucleic acid encoding the TCR can be obtained from a variety of sources, such as by polymerase chain reaction (PCR) amplification of naturally occurring TCR DNA sequences, followed by expression of antibody variable regions, followed by the selecting step described above. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable α and β chains (also known as TCRa and TCRp, respectively) or a variable γ and δ chains (also known as TCRy and TCRS, respectively) and that is capable of specifically binding to an antigen peptide bound to a MHC receptor. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3 rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable 0 chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) {see, e.g., Jores et al., Pwc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains {e.g., a-chain, β-chain) can contain two immunoglobulin domains, a variable domain {e.g., Va or Vp; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain {e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3y chain, a CD35 chain, two CD3s chains, and a homodimer of CD3ζ chains. The CD3y, CD35, and CD3s chains are highly related cell surface proteins of the immunoglobulin superfamily containing a single immunoglobulin domain. The transmembrane regions of the CD3y, CD35, and CD3s chains are negatively charged, which is a characteristic that allows these chains to associate with the positively charged T cell receptor chains. The intracellular tails of the CD3y, CD35, and CD3s chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

While T-cell receptors (TCRs) are transmembrane proteins and do not naturally exist in soluble form, antibodies can be secreted as well as membrane bound. Importantly, TCRs have the advantage over antibodies that they in principle can recognize peptides generated from all degraded cellular proteins, both intra- and extracellular, when presented in the context of MHC molecules. Thus TCRs have important therapeutic potential.

The present disclosure also relates to soluble T-cell receptors (sTCRs) that contain the antigen recognition part directed against a tumor neoantigenic peptide as herein disclosed (see notably Walseng E, Wälchli S, Fallang L-E, Yang W, Vefferstad A, Areffard A, et al. (2015) Soluble T-Cell Receptors Produced in Human Cells for Targeted Delivery. PLoS ONE 10(4): e0119559). In a particular embodiment, the soluble TCR can be fused to an antibody fragment directed to a T cell antigen, optionally wherein the targeted antigen is CD3 or CD16 (see for example Boudousquie, Caroline et al. “Polyfunctional response by ImmTAC (IMCgp100) redirected CD8+ and CD4+ T cells.” Immunology vol. 152, 3 (2017): 425-438. doi:10.1111/imm.12779).

The present disclosure also encompasses a chimeric antigen receptor (CAR) which is directed against a tumor neoantigenic peptide as herein disclosed. CARs are fusion proteins comprising an antigen-binding domain, typically derived from an antibody, linked to the signalling domain of the TCR complex. CARs can be used to direct immune cells such T-cells or NK cells against a tumor neoantigenic peptide as previously defined with a suitable antigen-binding domain selected.

The antigen-binding domain of a CAR is typically based on a scFv (single chain variable fragment) derived from an antibody. In addition to an N-terminal, extracellular antibody-binding domain, CARs typically may comprise a hinge domain, which functions as a spacer to extend the antigen-binding domain away from the plasma membrane of the immune effector cell on which it is expressed, a transmembrane (TM) domain, an intracellular signalling domain (e.g. the signalling domain from the zeta chain of the CD3 molecule (CD3) of the TCR complex, or an equivalent) and optionally one or more co-stimulatory domains which may assist in signalling or functionality of the cell expressing the CAR. Signalling domains from co-stimulatory molecules including CD28, OX-40 (CD134), and 4-1BB (CD137) can be added alone (second generation) or in combination (third generation) to enhance survival and increase proliferation of CAR modified T cells. Potential co-stimulatory domains also include ICOS-1, CD27, GITR, CD28, and DAP10.

Thus, the CAR may include
(1) In its extracellular portion, one or more antigen binding molecules, such as one or more antigen-binding fragment, domain, or portion of an antibody, or one or more antibody variable domains, and/or antibody molecules.
(2) In its transmembrane portion, a transmembrane domain derived from human T cell receptor-alpha or -beta chain, a CD3 zeta chain, CD28, CD3-epsilon, CD45, CD4, CD5, CD8, CD9, CD16, CD22, CD33, CD37, CD64, CD80, CD86, CD134, CD137, ICOS, CD154, or a GITR. In some embodiments, the transmembrane domain is derived from CD28, CD8 or CD3-zeta.
(3) One or more co-stimulatory domains, such as co-stimulatory domains derived from human CD28, 4-1BB (CD137), ICOS-1, CD27, OX 40 (CD137), DAP10, and GITR (AITR). In some embodiments, the CAR comprises co-stimulating domains of both CD28 and 4-1BB.
(4) In its intracellular signalling domain, an intracellular signalling domain comprising one or more ITAMs, for example, the intracellular signalling domain is CD3-zeta, or a variant thereof lacking one or two ITAMs (e.g. ITAM3 and ITAM2), or the intracellular signalling domain is derived from FcεRIγ.

The CAR can be designed to recognize tumor neoantigenic peptide alone or in association with an HLA or MEW molecule.

Exemplary antigen receptors, including CARs and recombinant TCRs, as well as methods for engineering and introducing the receptors into cells, include those described, for example, in international patent application publication numbers WO2000/14257, WO2013/126726, WO2012/129514, WO2014/031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Pat. No. 7,446,190, and those described in International Patent Application Publication No.: WO2014/055668.

The present disclosure also encompasses polynucleotides encoding antibodies, antigen-binding fragments or derivatives thereof, TCRs and CARs as previously described as well as vector comprising said polynucleotide(s).

Immune Cells

The present disclosure further encompasses immune cells which target one or more tumor neoantigenic peptides as previously described.

As used herein, the term “immune cell” includes cells that are of hematopoietic origin and that play a role in the immune response. Immune cells include lymphocytes, such as B cells and T cells, natural killer cells, myeloid cells, such as monocytes, macrophages, eosinophils, mast cells, basophils, and granulocytes.

As used herein, the term “T cell” includes cells bearing a T cell receptor (TCR), in particular TCR directed against a tumor neoantigenic peptide as herein disclosed. T-cells according to the present disclosure can be selected from the group consisting of inflammatory T-lymphocytes, cytotoxic T-lymphocytes, regulatory T-lymphocytes, Mucosal-Associated Invariant T cells (MAIT), Yδ T cell, tumour infiltrating lymphocyte (TILs) or helper T-lymphocytes included both type 1 and 2 helper T cells and Th17 helper cells. In another embodiment, said cell can be derived from the group consisting of CD4+T-lymphocytes and CD8+T-lymphocytes. Said immune cells may originate from a healthy donor or from a subject suffering from a cancer, or a tumor.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation.

In another embodiment, said cell can be derived from a healthy donor, from a subject diagnosed with cancer or tumor, notably with Ewing sarcoma. The cell can be autologous or allogeneic.

In allogeneic immune cell therapy, immune cells are collected from healthy donors, rather than the patient. Typically these are HLA matched to reduce the likelihood of graft vs. host disease. Alternatively, universal ‘off the shelf’ products that may not require HLA matching comprise modifications designed to reduce graft vs. host disease, such as disruption or removal of the TCRαβ receptor. See Graham et al., Cells. 2018 October; 7(10): 155 for a review. Because a single gene encodes the alpha chain (TRAC) rather than the two genes encoding the beta chain, the TRAC locus is a typical target for removing or disrupting TCRαβ receptor expression. Alternatively, inhibitors of TCRαβ signalling may be expressed, e.g. truncated forms of CD3ζ can act as a TCR inhibitory molecule. Disruption or removal of HLA class I molecules has also been employed. For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression. Ren et al. simultaneously knocked out TCRαβ, B2M and the immune-checkpoint PD1. Generally, the immune cells are activated and expanded to be utilized in the adoptive cell therapy. The immune cells as herein disclosed can be expanded in vivo or ex vivo. The immune cells, in particular T-cells can be activated and expanded generally using methods known in the art. Generally the T-cells are expanded by contact with a surface having attached thereto an agent that stimulates a CD3/TCR complex associated signal and a ligand that stimulates a co-stimulatory molecule on the surface of the T cells.

In one embodiment of the present disclosure, the immune cell can be modified to be directed to tumor neoantigenic peptides as previously defined. In a particular embodiment, said immune cell may express a recombinant antigen receptor directed to said neoantigenic peptide its cell surface. By “recombinant” is meant an antigen receptor which is not encoded by the cell in its native state, i.e. it is heterologous, non-endogenous. Expression of the recombinant antigen receptor can thus be seen to introduce new antigen specificity to the immune cell, causing the cell to recognise and bind a previously described peptide. The antigen receptor may be isolated from any useful source. In some embodiments, the cells comprise one or more nucleic acids introduced via genetic engineering that encode one or more antigen receptors, wherein the antigen include at least one tumor neoantigenic peptide as per the present disclosure.

Among the antigen receptors as per the present disclosure are genetically engineered T cell receptors (TCRs) and components thereof, as well as functional non-TCR antigen receptors, such as chimeric antigen receptors (CAR) as previously described.

Methods by which immune cells can be genetically modified to express a recombinant antigen receptor are well known in the art. A nucleic acid molecule encoding the antigen receptor may be introduced into the cell in the form of e.g. a vector, or any other suitable nucleic acid construct. Vectors, and their required components, are well known in the art. Nucleic acid molecules encoding antigen receptors can be generated using any method known in the art, e.g. molecular cloning using PCR. Antigen receptor sequences can be modified using commonly-used methods, such as site-directed mutagenesis.

The present disclosure also relates to a method for providing a T cell population which targets a tumor neoantigenic peptide as herein disclosed.

The T cell population may comprise CD8+ T cells, CD4+ T cells or CD8+ and CD4+ T cells.

T cell populations produced in accordance with the present disclosure may be enriched with T cells that are specific to, i.e. target, the tumor neoantigenic peptide of the present disclosure. That is, the T cell population that is produced in accordance with the present disclosure will have an increased number of T cells that target one or more tumor neoantigenic peptide. For example, the T cell population of the disclosure will have an increased number of T cells that target a tumor neoantigenic peptide compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a tumor neoantigenic peptide will be increased.

T cell populations produced in accordance with the present disclosure may be enriched with T cells that are specific to, i.e. target, tumor neoantigenic peptide. That is, the T cell population that is produced in accordance with the present disclosure will have an increased number of T cells that target one or more tumor neoantigenic peptide of the present disclosure. For example, the T cell population of the present disclosure will have an increased number of T cells that target a tumor neoantigenic peptide compared with the T cells in the sample isolated from the subject. That is to say, the composition of the T cell population will differ from that of a “native” T cell population (i.e. a population that has not undergone the identification and expansion steps discussed herein), in that the percentage or proportion of T cells that target a tumor neoantigenic peptide will be increased.

The T cell population according to the present disclosure may have at least about 0.2, 0.3, 0.4, 0.5, 0.6, 0.7, 0.8, 0.9, 1, 2, 3, 4, 5, 6, 7, 8, 9, 10, 1 1, 12, 13, 14, 15, 16, 17, 18, 19, 20, 25, 30, 35, 40, 45, 50, 55, 60, 65, 70, 75, 80, 85, 90, 95 or 100% T cells that target a tumor neoantigenic peptide as herein disclosed. For example, the T cell population may have about 0.2%-5%, 5%-10%, 10-20%, 20-30%, 30-40%, 40-50%, 50-70% or 70-100% T cells that target a tumor neoantigenic peptide of the present disclosure.

An expanded population of tumor neoantigenic peptide-reactive T cells may have a higher activity than a population of T cells not expanded, for example, using a tumor neoantigenic peptide. Reference to “activity” may represent the response of the T cell population to restimulation with a tumor neoantigenic peptide, e.g. a peptide corresponding to the peptide used for expansion, or a mix of tumor neoantigenic peptide. Suitable methods for assaying the response are known in the art. For example, cytokine production may be measured (e.g. IL2 or IFNy production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

In a preferred embodiment present disclosure provides a plurality or population, i.e. more than one, of T cells wherein the plurality of T cells comprises a T cell which recognizes a clonal tumor neoantigenic peptide and a T cell which recognizes a different clonal tumor neoantigenic peptide. As such, the present disclosure provides a plurality of T cells which recognize different clonal tumor neoantigenic peptide. Different T cells in the plurality or population may alternatively have different TCRs which recognize the same tumor neoantigenic peptide.

In a preferred embodiment the number of clonal tumor neoantigenic peptide recognized by the plurality of T cells is from 2 to 1000. For example, the number of clonal neo-antigens recognized may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, preferably 2 to 100. There may be a plurality of T cells with different TCRs but which recognize the same clonal neo-antigen.

The T cell population may be all or primarily composed of CD8+ T cells, or all or primarily composed of a mixture of CD8+ T cells and CD4+ T cells or all or primarily composed of CD4+ T cells.

In particular embodiments, the T cell population is generated from T cells isolated from a subject with a tumor. For example, the T cell population may be generated from T cells in a sample isolated from a subject with a tumor. The sample may be a tumor sample, a peripheral blood sample or a sample from other tissues of the subject.

In a particular embodiment the T cell population is generated from a sample from the tumor in which the tumor neoantigenic peptide is identified. In other words, the T cell population is isolated from a sample derived from the tumor of a patient to be treated. Such T cells are referred to herein as ‘tumor infiltrating lymphocytes’ (TILs).

T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

Cancer Therapeutic and Diagnostic Methods

In any of the embodiments, the Cancer Therapeutic Products described herein may be used in methods for inhibiting proliferation of cancer cells. The Cancer Therapeutic Products described herein may also be used in the treatment of cancer or tumor, typically a cancer driven (or associated with) by a transcription factor fusion as notably listed in table 1 in patients suffering from such cancer or tumor, or for the prophylactic treatment of such cancer, in patients at risk of such cancer or tumor.

Cancers that can be treated using the therapy described herein include any solid or non-solid tumors, typically the tumors are driven (or associated with) by a transcription factor fusion as notably listed in table 1, typically the tumor is a mesenchymatous tumor, or a sarcoma, as listed in table 1. In a specific embodiment of the present disclosure, the cancer is Ewing sarcoma.

Cancers includes also the cancers which are refractory to treatment with other chemotherapeutics. The term “refractory”, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

A subject as per the present disclosure is typically a patient in need thereof that has been diagnosed with tumor, typically a tumor driven (or associated with) by a transcription factor fusion as notably listed in table 1, notably Ewing sarcoma or is at risk of developing tumor, notably Ewing sarcoma. The subject is typically a mammal, notably a human, dog, cat, horse or any animal in which a tumor specific immune response is desired.

The present disclosure also pertains to a neoantigenic peptide, a population of APCs, a vaccine or immunogenic composition, a polynucleotide encoding a neoantigenic peptide or a vector as previously defined for use in cancer vaccination therapy of a subject or for treating cancer in a subject, typically a tumor driven (or associated with) by a transcription factor fusion as notably listed in table 1, notably for treating Ewing sarcoma, wherein the peptide(s) binds at least one MHC molecule of said subject.

The present disclosure also provides a method for treating cancer, typically a tumor driven (or associated with) by a transcription factor fusion as notably listed in table 1, notably Ewing sarcoma, in a subject comprising administering a vaccine or immunogenic composition as described herein to said subject in a therapeutically effective amount to treat the subject. The method may additionally comprise the step of identifying a subject who has a cancer or a tumor, notably who is suffering from Ewing sarcoma.

The present disclosure also relates to a method of treating cancer, typically a tumor driven (or associated with) by a transcription factor fusion as notably listed in table 1, notably Ewing sarcoma, comprising producing an antibody or antigen-binding fragment thereof by the method as herein described and administering to a subject with cancer, or tumor said antibody or antigen-binding fragment thereof, or with an immune cell expressing said antibody or antigen-binding fragment thereof, in a therapeutically effective amount to treat said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MEW or HLA molecule, for use in cancer therapy of a subject, notably Ewing sarcoma therapy, wherein the tumor neoantigenic peptide binds at least one MHC molecule of said subject.

The present disclosure also relates to an antibody (including variants and derivatives thereof), a T cell receptor (TCR) (including variants and derivatives thereof), or a CAR (including variants and derivatives thereof) which are directed against a tumor neoantigenic peptide as herein described, optionally in association with an MEW or HLA molecule, or an immune cell which targets a neoantigenic peptide, as previously defined, for use in adoptive cell or CAR-T cell therapy in a subject, wherein the tumor neoantigenic peptide binds at least one MEW molecule of said subject.

Typically, the skilled person is able to select an appropriate antigen receptor which binds and recognizes a tumor neoantigenic peptide as previously defined with which to redirect an immune cell to be used for use in cancer cell therapy, notably Ewing sarcoma cell therapy. In a particular embodiment, the immune cell for use in the method of the present disclosure is a redirected T-cell, e.g. a redirected CD8+ and/or CD4+ T-cell.

The inventors herein provide demonstration that peptides which expression is positively regulated by EWS-FLI1 can be recognized by naïve T-cells from controls. This discovery has strong potentials in diagnostic. Indeed, it provides tumor-specific biomarkers that can discriminate Ewing sarcoma from other tumors, particularly from other sarcomas.

The present disclosure therefore also encompasses a method for the diagnostic of a tumor associated with transcription factor fusion in a patient. Said method comprises the identification, as per the method as herein disclosed, in a tumor sample obtained from said patient of one or more unannotated transcripts which transcription is positively regulated by said transcription factor fusion or negatively regulated upon depletion of said transcription factor fusion. As previously defined an transcription factor fusion is typically a fusion transcription factor having gain-of-function activities. Typically, said one or more transcripts have no match on the representative transcriptome.

More specifically, the present disclosure provides a method for the diagnostic of Ewing sarcoma tumor in a patient, notably for discriminating Ewing sarcoma from other sarcomas. Said method comprises a step of identifying, in an Ewing sarcoma tumor sample obtained from a patient one or more unannotated transcript which transcription is positively regulated by EWS-FLI1, or negatively regulated upon EW-FLI1 depletion. Typically, said one or more transcripts have no match on the representative transcriptome. Detection and identification of transcript can be achieved as described in the present application. Typically also a neoantigenic peptide encoded by a part of an (ORF) sequence from such an unannotated trancript is expressed at a higher level or frequency in an Ewing sarcoma sample compared to normal tissue sample. Typically also, such neoantigenic peptide can be recognized by naïve T-cells from said patient.

The present application also encompasses a method for treating a patient suffering from a tumor associated with transcription factor fusion (also named herein transcription factor fusion) in a patient as herein disclosed, notably suffering from an Ewing's sarcoma tumor comprising a step of diagnosing said tumor as per the method as above defined and a step of administering an treatment dedicated to the identified tumor.

In some embodiment, the present application relates to a method for treating a patient suffering from a tumor associated with transcription factor fusion in a patient, notably suffering from an Ewing's sarcoma tumor, comprising (i) a step of diagnosing said tumor as per the method as above defined and (ii) a step of administering any one or a combination of the cancer therapeutic products described herein.

In some embodiments, cancer treatment, vaccination therapy and/or adoptive cell cancer therapy as above described are administered in combination with additional cancer therapies; In some embodiments, cancer treatment, vaccination therapy and/or adoptive cell cancer therapy as above described are administered in combination with targeted therapy, immunotherapy such as immune checkpoint therapy and immune checkpoint inhibitor, co-stimulatory antibodies, chemotherapy and/or radiotherapy.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Co-stimulatory antibodies deliver positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350.

Example of anti-PD-L1 antibodies include, but are not limited to, LY3300054, atezolizumab, durvalumab and avelumab.

Example of anti-CTLA-4 antibodies include, but are not limited to, ipilimumab (see, e.g., US patents U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in U.S. Pat. No. 8,491,895.

Example of anti-VISTA antibodies are described in US patent application US20130177557.

Example of inhibitors of the LAG3 receptor are described in U.S. Pat. No. 5,773,578.

Example of KIR inhibitor is IPH4102 targeting KIR3DL2.

As used herein, the term “chemotherapy” has its general meaning in the art and refers to the treatment that consists in administering to the patient a chemotherapeutic agent. A chemotherapeutic entity as used herein refers to an entity which is destructive to a cell, that is the entity reduces the viability of the cell. The chemotherapeutic entity may be a cytotoxic drug. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; biological response modifiers such as IFNa, IL-2, G-CSF and GM-CSF; and pharmaceutically acceptable salts, acids or derivatives of any of the above.

Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

“In combination” may refer to administration of the additional therapy before, at the same time as or after administration of the T cell composition according to the present disclosure.

In addition or as an alternative to the combination with checkpoint blockade, the T cell composition of the present disclosure may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells (see the above listed checkpoint inhibitors) and more particularly but not limited to PD-1, Lag-3, Tim-3, TIGIT, BTLA CTLA-4 and combinations of these. The T cell as discussed here may be modified by any of these methods.

The T cell according to the present disclosure may also be genetically modified to express molecules increasing homing into tumors and or to deliver inflammatory mediators into the tumor microenvironment, including but not limited to cytokines, soluble immune-regulatory receptors and/or ligands.

In a particular embodiment, said tumor neoantigenic peptide is used in cancer vaccination therapy in combination with another immunotherapy such as immune checkpoint therapy, more particularly in combination with anti-checkpoint antibodies such as the above exemplified antibodies and notably but not limited to the anti-PD1, anti-PDL1, anti-CTLA-4, anti-TIM-3, anti-LAG3, anti-GITR antibodies.

TABLE 3
description of neotranscripts of the present disclosure

JUJE1|chr1: 73, 171, 787-73, 178, 490|exon1a-2-3
JUJE1|chr1: 73, 171, 787-73, 178, 490|exon1b-2-3
JUJE2|chr2: 4633219-4637217|exon1-2-3
JUJE3|chr3: 197839238-197842858|exon1-2-3
JUJE10|chr10: 19203764-19206511|exon 1-2-3-4
JUJE10|chr10: 19203764-19206511|exon 1-2-3-5
JUJE10|chr10: 19203764-19206511|exon 1-3-4
JUJE10|chr10: 19203764-19206511|exon 1-3-5

TABLE 4
Description of the sequences

| SEQ ID NO: | Sequence type/name |
|---|---|
| 1-145 | EWS neotranscripts |
| 146 | JUJE1\|chr1: 73, 171, 787-73, 178, 490\|exon1a-2-3 |
| 147 | JUJE1\|chr1: 73, 171, 787-73, 178, 490\|exon1b-2-3 |
| 148 | JUJE2\|chr2: 4633219-4637217\|exon1-2-3 |
| 149 | JUJE3\|chr3: 197839238-197842858\|exon1-2-3 |
| 150 | JUJE10\|chr10: 19203764-19206511\|exon 1-2-3-4 |
| 151 | JUJE10\|chr10: 19203764-19206511\|exon 1-2-3-5 |
| 152 | JUJE10\|chr10: 19203764-19206511\|exon 1-3-4 |
| 153 | JUJE10\|chr10: 19203764-19206511\|exon 1-3-5 |
| 154 | EWS-FLI1 primers F |
| 155 | EWS-FLI1 primers R |
| 156 | RPLP0 primer F |
| 157 | RPLP0 primer R |
| 158 | JUJE1 primer F |
| 159 | JUJE1 primer R |
| 160 | JUJE2 primer F |
| 161 | JUJER primer |
| 162 | JUJE3 primer F |
| 163 | JUJE3 primer R |
| 164 | JUJE10 primer F |
| 165 | JUJE 10 primer R |
| 166-201 | neopeptides |

TABLE 5
neogenes classifications

| | Cell_line | ChIP-seq | Type |
|---|---|---|---|
| Ewing_neogene | | | |
| Ew_NG1 | 1 | 1 | 2 |
| Ew_NG2 | 1 | 1 | 2 |
| Ew_NG3 | 1 | 1 | 2 |
| Ew_NG4 | 1 | 1 | 2 |
| Ew_NG5 | 1 | 0 | 1 |
| Ew_NG6 | 1 | 0 | 1 |
| Ew_NG7 | 1 | 0 | 1 |
| Ew_NG8 | 1 | 1 | 2 |
| Ew_NG9 | 0 | 0 | 0 |
| Ew_NG10 | 1 | 1 | 2 |
| Ew_NG11 | 0 | 0 | 0 |
| Ew_NG12 | 1 | 1 | 2 |
| Ew_NG13 | 1 | 1 | 2 |
| Ew_NG14 | NA | 0 | 0 |
| Ew_NG15 | NA | 0 | 0 |
| Ew_NG16 | 1 | 0 | 1 |
| Ew_NG17 | 0 | 0 | 0 |
| Ew_NG18 | 1 | 0 | 1 |
| Ew_NG19 | 0 | 0 | 0 |
| Ew_NG20 | 1 | 0 | 1 |
| Ew_NG21 | 1 | 1 | 2 |
| Ew_NG22 | 1 | 1 | 2 |
| Ew_NG23 | 1 | 0 | 1 |
| Ew_NG24 | 1 | 1 | 2 |
| Ew_NG25 | 1 | 1 | 2 |
| aRMS_neogene | | | |
| aRMS_NG1 | NA | 0 | 0 |
| aRMS_NG2 | 0 | 0 | 0 |
| aRMS_NG3 | 0 | 0 | 0 |
| aRMS_NG4 | 0 | 0 | 0 |
| aRMS_NG5 | 0 | 1 | 1 |
| aRMS_NG6 | 0 | 1 | 1 |
| aRMS_NG7 | 0 | 1 | 1 |
| aRMS_NG8 | 0 | 1 | 1 |
| aRMS_NG9 | 0 | 0 | 0 |
| aRMS_NG10 | 0 | 1 | 1 |
| aRMS_NG11 | 0 | 0 | 0 |
| aRMS_NG12 | 0 | 0 | 0 |
| aRMS_NG13 | NA | 1 | 1 |
| aRMS_NG14 | 0 | 0 | 0 |
| aRMS_NG15 | NA | 0 | 0 |
| aRMS_NG16 | 1 | 0 | 1 |
| aRMS_NG17 | 1 | 1 | 2 |
| aRMS_NG18 | 0 | 1 | 1 |
| aRMS_NG19 | NA | 0 | 0 |
| aRMS_NG20 | NA | 0 | 0 |
| aRMS_NG21 | NA | 0 | 0 |
| aRMS_NG22 | 1 | 0 | 1 |
| aRMS_NG23 | 1 | 1 | 2 |
| aRMS_NG24 | NA | 0 | 0 |
| aRMS_NG25 | NA | 0 | 0 |
| aRMS_NG26 | 0 | 0 | 0 |
| aRMS_NG27 | 0 | 0 | 0 |
| aRMS_NG28 | 0 | 1 | 1 |
| aRMS_NG29 | 1 | 1 | 2 |
| aRMS_NG30 | 1 | 0 | 1 |
| aRMS_NG31 | 0 | 1 | 1 |
| aRMS_NG32 | 0 | 0 | 0 |
| aRMS_NG33 | 0 | 1 | 0 |
| aRMS_NG34 | 0 | 0 | 0 |
| aRMS_NG35 | 1 | 0 | 1 |
| aRMS_NG36 | NA | 0 | 0 |
| DSRCT_neogene | | | |
| DSRCT_NG1 | 1 | 1 | 2 |
| DSRCT_NG2 | 0 | 0 | 0 |
| DSRCT_NG3 | 0 | 1 | 0 |
| DSRCT_NG4 | 1 | 1 | 2 |
| DSRCT_NG5 | 1 | 1 | 2 |
| DSRCT_NG6 | 0 | 1 | 1 |
| DSRCT_NG7 | 0 | 1 | 1 |
| DSRCT_NG8 | 0 | 1 | 1 |
| DSRCT_NG9 | 1 | 0 | 1 |
| DSRCT_NG10 | 1 | 1 | 2 |
| DSRCT_NG11 | 1 | 1 | 2 |
| DSRCT_NG12 | 0 | 0 | 0 |
| DSRCT_NG13 | 1 | 1 | 2 |
| DSRCT_NG14 | 1 | 1 | 2 |
| DSRCT_NG15 | NA | 0 | 0 |
| DSRCT_NG16 | 1 | 1 | 2 |
| DSRCT_NG17 | 0 | 1 | 1 |
| DSRCT_NG18 | 1 | 1 | 2 |
| DSRCT_NG19 | 1 | 0 | 1 |
| DSRCT_NG20 | 1 | 1 | 2 |
| DSRCT_NG21 | 1 | 1 | 2 |
| DSRCT_NG22 | 0 | 0 | 0 |
| DSRCT_NG23 | 1 | 0 | 1 |
| DSRCT_NG24 | 1 | 1 | 2 |
| DSRCT_NG25 | 1 | 1 | 2 |
| DSRCT_NG26 | 0 | 1 | 1 |
| DSRCT_NG27 | 0 | 0 | 0 |
| DSRCT_NG28 | 1 | 1 | 2 |
| DSRCT_NG29 | 1 | 1 | 2 |
| DSRCT_NG30 | 0 | 1 | 1 |
| DSRCT_NG31 | 1 | 1 | 2 |
| DSRCT_NG32 | 0 | 1 | 1 |
| DSRCT_NG33 | 0 | 1 | 1 |
| DSRCT_NG34 | 0 | 0 | 0 |
| DSRCT_NG35 | 0 | 0 | 0 |
| DSRCT_NG36 | 1 | 0 | 1 |
| DSRCT_NG37 | 1 | 0 | 1 |

TABLE 6
numbers of neogenes/neotranscripts identified by
transcription factor fusion-driven tumor type

| | Fusion gene (most | Number of | Number of | Number | Number of |
|---|---|---|---|---|---|
| Diagnosis | commmon if several) | neogenes | neotranscripts | of ORFs | strong binders |
| AFH | EWSR1-ATF1/CREB1 | 20 | 28 | 1917 | 1536 |
| aRMS | PAX3/7-FOXO1 | 36 | 72 | 6743 | 6105 |
| ASPS | ASPSCR1-TFE3 | 33 | 103 | 3841 | 3281 |
| BCOR | BCOR-CCNB3 | 6 | 6 | 160 | 78 |
| CCS | EWSR1-ATF1/CREB1 | 15 | 24 | 1956 | 2543 |
| CIC | CIC-DUX4 | 8 | 16 | 1639 | 1312 |
| DSRCT | EWSR1-WT1 | 37 | 105 | 8558 | 6850 |
| emCS | EWSR1-NR4A3 | 2 | 2 | 71 | 72 |
| Ew | EWSR1-FLI1 | 25 | 61 | 4839 | 4355 |
| LGFS | FUS-CREB3L2 | 44 | 70 | 3443 | 2657 |
| MCS | HEY-NCOA2 | 11 | 21 | 733 | 527 |
| Midline | BRD-NUT | 29 | 64 | 4300 | 3566 |
| mLPS | FUS-DDIT3 | 9 | 14 | 832 | 652 |
| NFAT | EWSR1-NFATC2 | 47 | 88 | 4510 | 3769 |
| PATZ1 | EWSR1-PATZ1 | 22 | 30 | 997 | 720 |
| SFT | NAB2-STAT6 | 35 | 54 | 2962 | 2511 |
| SS | SS18-SSX | 3 | 16 | 1157 | 1106 |
| TFE3 | ASPSCR1-TFE3 | 15 | 32 | 1987 | 1643 |
| TMFI | ALK fusions | 8 | 13 | 1100 | 1006 |

TABLE 7
peptides and Net MHCpan values: SB: Strong Binder; WB: Weak Binder;
Score EL: The raw prediction score. %Rank_EL: Rank of the predicted binding score
compared to a set of random natural peptides. This measure is not affected by inherent
bias of certain molecules towards higher or lower mean predicted affinities. Strong
binders are defined as having %rank <0.5, and weak binders with %rank <2. In grey,
tested SB peptides.

| ID | MHC | Peptide | Score_EL | %Rank_EL | BindLevel |
|---|---|---|---|---|---|
| Chr2_lcl_ORF2 | HLA-A*02:01 | VLYPGYYSL | 0.9689120 | 0.014 | SB |
| Chr2_lcl_ORF2 | HLA-A*02:01 | LQLAISSAV | 0.1288180 | 1.566 | WB |
| Chr10_lcl_ORF19 | HLA-A*02:01 | FLSQELLPA | 0.6954240 | 0.186 | SB |
| Chr10_lcl_ORF25 | HLA-A*02:01 | YISRWSLPI | 0.0531370 | 2.832 | |
| Chr10_lcl_ORF13 | HLA-A*02:01 | SLCFCNHML | 0.1462430 | 1.450 | WB |
| Chr10_lcl_ORF13 | HLA-A*02:01 | FCNHMLVYI | 0.0221690 | 4.520 | |
| Chr10_lcl_ORF22 | HLA-A*02:01 | KLAESLWIA | 0.8668710 | 0.066 | SB |
| Chr10_lcl_ORF22 | HLA-A*02:01 | SLWIAILHL | 0.7126450 | 0.170 | SB |
| Chr10_lcl_ORF22 | HLA-A*02:01 | LWIAILHLV | 0.0080400 | 4.014 | |
| Chr10_lcl_ORF17 | HLA-A*02:01 | YVWHKHQKV | 0.7674120 | 0.130 | SB |
| Chr10_lcl_ORF17 | HLA-A*02:01 | RLKNVLLHI | 0.4692910 | 0.433 | SB |
| Chr10_lcl_ORF13 | HLA-A*02:01 | AIIAFMTSL | 0.4662990 | 0.437 | SB |
| Chr10_lcl_ORF21 | HLA-A*02:01 | RLYDQSVFL | 0.9858280 | 0.006 | SB |
| Chr10_lcl_ORF23 | HLA-A*02:01 | YMLAYFRGL | 0.3824480 | 0.564 | WB |
| Chr10_lcl_ORF14 | HLA-A*02:01 | KLAFENVFA | 0.7895910 | 0.114 | SB |
| ChrlexA_lcl_ORF | HLA-A*02:01 | MQFSLFVFV | 0.4407340 | 0.472 | SB |
| ChrlexA_lcl_ORF | HLA-A*02:01 | ALVCLIYLI | 0.0999940 | 1.887 | WB |
| ChrlexB_lcl_ORF | HLA-A*02:01 | MQFSLFVFV | 0.4407340 | 0.472 | SB |
| ChrlexB_lcl_ORF | HLA-A*02:01 | IITDSSTTV | 0.4765820 | 0.423 | SB |
| ChrlexB_lcl_ORF | HLA-A*02:01 | ALVCLIYLI | 0.0999940 | 1.887 | WB |
| CHR3_lcl_ORF4 | HLA-A*02:01 | SMIPHRYKL | 0.6821530 | 0.202 | SB |
| CHR3_lcl_ORF6 | HLA-A*02:01 | YLSRPSPGV | 0.8840530 | 0.057 | SB |
| CHR3_lcl_ORF8 | HLA-A*02:01 | ILSYFHMEL | 0.5895040 | 0.291 | SB |
| CHR3_lcl_ORF7 | HLA-A*02:01 | KLNPIVSYT | 0.5575100 | 0.326 | SB |
| CHR3_lcl_ORF4 | HLA-A*02:01 | FLCHPNPDL | 0.5040700 | 0.388 | SB |
| CHR3_lcl_ORF1 | HLA-A*02:01 | YLHCSSIQC | 0.0684900 | 2.430 | |
| CHR3_lcl_ORF8 | HLA-A*02:01 | YLYFCVILT | 0.0492490 | 2.944 | |
| CHR3_lcl_ORF7 | HLA-A*02:01 | QLIVLYCRI | 0.0099370 | 6.762 | |
| CHR3_lcl_ORF3 | HLA-A*02:01 | VLLVQTEFC | 0.0222770 | 4.508 | |
| CHR3_lcl_ORF2 | HLA-A*02:01 | GLSQPHCLV | 0.2341620 | 0.987 | WB |
| CHR3_lcl_ORF11 | HLA-A*02:01 | KLFCKRPML | 0.4024460 | 0.530 | WB |
| Chr2_lcl_ORF2 | HLA-A*02:01 | FLHHGMERV | 0.9328920 | 0.030 | SB |
| ChrlexA_lcl_ORF4 | HLA-A*02:01 | LLGELAHMV | 0.8720010 | 0.063 | SB |
| CHR3_lcl_ORF12 | HLA-A*02:01 | FLFFFFFCI | 0.0932250 | 1.978 | WB |
| CHR3_lcl_ORf4 | HLA-A*02:01 | VLLSYGISV | 0.8889580 | 0.055 | SB |
| CHR3_lcl_ORF8 | HLA-A*02:01 | RMTYLYFCV | 0.0569350 | 2.724 | |

TABLE 8
Synthetized and tested peptide sequences

| | Numéro | | | | |
|---|---|---|---|---|---|
| MHC | peptide | Peptide | Score_EL | %Rank_EL | BindLevel |
| HLA-A*02:01 | Chr1_A2 | LLGELAHMV | 0.8720010 | 0.063 | SB |
| HLA-A*02:01 | Chr3_A2_1 | VLLSYGISV | 0.8889580 | 0.055 | SB |
| HLA-A*02:01 | Chr3_A2_2 | FLCHPNPDL | 0.5040700 | 0.388 | SB |
| HLA-A*02:01 | Chr3_A2_3 | SMIPHRYKL | 0.6821530 | 0.202 | SB |
| HLA-A*02:01 | Chr3_A2_4 | YLSRPSPGV | 0.8840530 | 0.057 | SB |
| HLA-A*02:01 | Chr3_A2_5 | ILSYFHMEL | 0.5895040 | 0.291 | SB |
| HLA-A*02:01 | Chr3_A2_6 | KLNPIVSYT | 0.5575100 | 0.326 | SB |
| HLA-A*02:01 | Chr10_A2_1 | FLSQELLPA | 0.6954240 | 0.186 | SB |
| HLA-A*02:01 | Chr10_A2_2 | AIIAFMTSL | 0.4662990 | 0.437 | SB |
| HLA-A*02:01 | Chr10_A2_3 | KLAESLWIA | 0.8668710 | 0.066 | SB |
| HLA-A*02:01 | Chr10_A2_4 | SLWIAILHL | 0.7126450 | 0.170 | SB |
| HLA-A*02:01 | ChrlO_A2_5 | RLYDQSVFL | 0.9858280 | 0.006 | SB |
| HLA-A*02:01 | Chr10_A2_6 | YMLAYFRGL | 0.3824480 | 0.564 | WB |
| HLA-A*02:01 | Chr10_A2_7 | YVWHKHQKV | 0.7674120 | 0.130 | SB |
| HLA-A*02:01 | Chr10_A2_8 | KLAFENVFV | 0.7895910 | 0.114 | SB |

TABLE 9
identified transcripts sequence as per the present disclosure.

| Chromosome | feature | start | end | strand | attributes |
|---|---|---|---|---|---|
| chr1 | transcript | 22870455 | 22885380 | + | gene_id “aRMS_NG1”; transcript_id |
| | | | | | “aRMS_NG1.2”; RPKM “0.7620”; cov |
| | | | | | “10.5076”; |
| chr1 | exon | 22870455 | 22872138 | + | gene_id “aRMS_NG1”; transcript_id |
| | | | | | “aRMS_NG1.2”; exon “1”; |
| chr1 | exon | 22878552 | 22879901 | + | gene_id “aRMS_NG1”; transcript_id |
| | | | | | “aRMS_NG1.2”; exon “2”; |
| chr1 | exon | 22882622 | 22885380 | + | gene_id “aRMS_NG1”; transcript_id |
| | | | | | “aRMS_NG1.2”; exon “3”; |
| chr1 | transcript | 22882093 | 22889845 | + | gene_id “aRMS_NG1”; transcript_id |
| | | | | | “aRMS_NG1.1”; RPKM “1.8775”; cov |
| | | | | | “25.8906”; |
| chr1 | exon | 22882093 | 22889845 | + | gene_id “aRMS_NG1”; transcript_id |
| | | | | | “aRMS_NG1.1”; exon “1”; |
| chr1 | transcript | 178523288 | 178528839 | − | gene_id “aRMS_NG2”; transcript_id |
| | | | | | “aRMS_NG2.1”; RPKM “2.3424”; cov |
| | | | | | “32.3011”; |
| chr1 | exon | 178523288 | 178528839 | − | gene_id “aRMS_NG2”; transcript_id |
| | | | | | “aRMS_NG2.1”; exon “1”; |
| chr1 | transcript | 178523288 | 178531111 | − | gene_id “aRMS_NG2”; transcript_id |
| | | | | | “aRMS_NG2.2”; RPKM “1.2211”; cov |
| | | | | | “16.8393”; |
| chr1 | exon | 178523288 | 178524819 | − | gene_id “aRMS_NG2”; transcript_id |
| | | | | | “aRMS_NG2.2”; exon “1”; |
| chr1 | exon | 178528991 | 178531111 | − | gene_id “aRMS_NG2”; transcript_id |
| | | | | | “aRMS_NG2.2”; exon “2”; |
| chr10 | transcript | 113233117 | 113319297 | + | gene_id “aRMS_NG21”; transcript_id |
| | | | | | “aRMS_NG21.1”; RPKM “2.1273”; cov |
| | | | | | “29.3359”; |
| chr10 | exon | 113233117 | 113239865 | + | gene_id “aRMS_NG21”; transcript_id |
| | | | | | “aRMS_NG21.1”; exon “1”; |
| chr10 | exon | 113241588 | 113241647 | + | gene_id “aRMS_NG21”; transcript_id |
| | | | | | “aRMS_NG21.1”; exon “2”; |
| chr10 | exon | 113315484 | 113319297 | + | gene_id “aRMS_NG21”; transcript_id |
| | | | | | “aRMS_NG21.1”; exon “3”; |
| chr10 | transcript | 113233117 | 113319297 | + | gene_id “aRMS_NG21”; transcript_id |
| | | | | | “aRMS_NG21.2”; RPKM “3.4666”; cov |
| | | | | | “47.8042”; |
| chr10 | exon | 113233117 | 113239865 | + | gene_id “aRMS_NG21”; transcript_id |
| | | | | | “aRMS_NG21.2”; exon “1”; |
| chr10 | exon | 113241588 | 113241647 | + | gene_id “aRMS_NG21”; transcript_id |
| | | | | | “aRMS_NG21.2”; exon “2”; |
| chr10 | exon | 113316935 | 113319297 | + | gene_id “aRMS_NG21”; transcript_id |
| | | | | | “aRMS_NG21.2”; exon “3”; |
| chr10 | transcript | 113233117 | 113319297 | + | gene_id “aRMS_NG21”; transcript_id |
| | | | | | “aRMS_NG21.3”; RPKM “2.5307”; cov |
| | | | | | “34.8990”; |
| chr10 | exon | 113233117 | 113239865 | + | gene_id “aRMS_NG21”; transcript_id |
| | | | | | “aRMS_NG21.3”; exon “1”; |
| chr10 | exon | 113241588 | 113241647 | + | gene_id “aRMS_NG21”; transcript_id |
| | | | | | “aRMS_NG21.3”; exon “2”; |
| chr10 | exon | 113298069 | 113298124 | + | gene_id “aRMS_NG21”; transcript_id |
| | | | | | “aRMS_NG21.3”; exon “3”; |
| chr10 | exon | 113316935 | 113319297 | + | gene_id “aRMS_NG21”; transcript_id |
| | | | | | “aRMS_NG21.3”; exon “4”; |
| chr10 | transcript | 113233117 | 113319297 | + | gene_id “aRMS_NG21”; transcript_id |
| | | | | | “aRMS_NG21.4”; RPKM “1.1643”; cov |
| | | | | | “16.0552”; |
| chr10 | exon | 113233117 | 113239865 | + | gene_id “aRMS_NG21”; transcript_id |
| | | | | | “aRMS_NG21.4”; exon “1”; |
| chr10 | exon | 113241588 | 113241647 | + | gene_id “aRMS_NG21”; transcript_id |
| | | | | | “aRMS_NG21.4”; exon “2”; |
| chr10 | exon | 113298069 | 113298124 | + | gene_id “aRMS_NG21”; transcript_id |
| | | | | | “aRMS_NG21.4”; exon “3”; |
| chr10 | exon | 113315484 | 113319297 | + | gene_id “aRMS_NG21”; transcript_id |
| | | | | | “aRMS_NG21.4”; exon “4”; |
| chr11 | transcript | 13049308 | 13076552 | + | gene_id “aRMS_NG22”; transcript_id |
| | | | | | “aRMS_NG22.1”; RPKM “1.8322”; cov |
| | | | | | “25.2665”; |
| chr11 | exon | 13049308 | 13049463 | + | gene_id “aRMS_NG22”; transcript_id |
| | | | | | “aRMS_NG22.1”; exon “1”; |
| chr11 | exon | 13073165 | 13076552 | + | gene_id “aRMS_NG22”; transcript_id |
| | | | | | “aRMS_NG22.1”; exon “2”; |
| chr11 | transcript | 20183702 | 20222468 | + | gene_id “aRMS_NG23”; transcript_id |
| | | | | | “aRMS_NG23.2”; RPKM “1.7210”; cov |
| | | | | | “23.7326”; |
| chr11 | exon | 20183702 | 20183916 | + | gene_id “aRMS_NG23”; transcript_id |
| | | | | | “aRMS_NG23.2”; exon “1”; |
| chr11 | exon | 20209051 | 20209129 | + | gene_id “aRMS_NG23”; transcript_id |
| | | | | | “aRMS_NG23.2”; exon “2”; |
| chr11 | exon | 20220812 | 20222468 | + | gene_id “aRMS_NG23”; transcript_id |
| | | | | | “aRMS_NG23.2”; exon “3”; |
| chr11 | transcript | 20184380 | 20222468 | + | gene_id “aRMS_NG23”; transcript_id |
| | | | | | “aRMS_NG23.1”; RPKM “1.2683”; cov |
| | | | | | “17.4895”; |
| chr11 | exon | 20184380 | 20185174 | + | gene_id “aRMS_NG23”; transcript_id |
| | | | | | “aRMS_NG23.1”; exon “1”; |
| chr11 | exon | 20209051 | 20209129 | + | gene_id “aRMS_NG23”; transcript_id |
| | | | | | “aRMS_NG23.1”; exon “2”; |
| chr11 | exon | 20220812 | 20222468 | + | gene_id “aRMS_NG23”; transcript_id |
| | | | | | “aRMS_NG23.1”; exon “3”; |
| chr11 | transcript | 71384875 | 71391010 | − | gene_id “aRMS_NG24”; transcript_id |
| | | | | | “aRMS_NG24.1”; RPKM “20.7618”; cov |
| | | | | | “286.3067”; |
| chr11 | exon | 71384875 | 71391010 | − | gene_id “aRMS_NG24”; transcript_id |
| | | | | | “aRMS_NG24.1”; exon “1”; |
| chr11 | transcript | 71391032 | 71395304 | − | gene_id “aRMS_NG25”; transcript_id |
| | | | | | “aRMS_NG25.1”; RPKM “3.3159”; cov |
| | | | | | “45.7260”; |
| chr11 | exon | 71391032 | 71395304 | − | gene_id “aRMS_NG25”; transcript_id |
| | | | | | “aRMS_NG25.1”; exon “1”; |
| chr13 | transcript | 27573827 | 27580633 | − | gene_id “aRMS_NG26”; transcript_id |
| | | | | | “aRMS_NG26.1”; RPKM “1.5525”; cov |
| | | | | | “21.4097”; |
| chr13 | exon | 27573827 | 27580633 | − | gene_id “aRMS_NG26”; transcript_id |
| | | | | | “aRMS_NG26.1”; exon “1”; |
| chr13 | transcript | 27573827 | 27581460 | − | gene_id “aRMS_NG26”; transcript_id |
| | | | | | “aRMS_NG26.2”; RPKM “1.7013”; cov |
| | | | | | “23.4610”; |
| chr13 | exon | 27573827 | 27575530 | − | gene_id “aRMS_NG26”; transcript_id |
| | | | | | “aRMS_NG26.2”; exon “1”; |
| chr13 | exon | 27576270 | 27576468 | − | gene_id “aRMS_NG26”; transcript_id |
| | | | | | “aRMS_NG26.2”; exon “2”; |
| chr13 | exon | 27581224 | 27581460 | − | gene_id “aRMS_NG26”; transcript_id |
| | | | | | “aRMS_NG26.2”; exon “3”; |
| chr13 | transcript | 104658677 | 104674357 | − | gene_id “aRMS_NG27”; transcript_id |
| | | | | | “aRMS_NG27.2”; RPKM “0.9192”; cov |
| | | | | | “12.6757”; |
| chr13 | exon | 104658677 | 104664120 | − | gene_id “aRMS_NG27”; transcript_id |
| | | | | | “aRMS_NG27.2”; exon “1”; |
| chr13 | exon | 104664379 | 104667144 | − | gene_id “aRMS_NG27”; transcript_id |
| | | | | | “aRMS_NG27.2”; exon “2”; |
| chr13 | exon | 104667854 | 104667921 | − | gene_id “aRMS_NG27”; transcript_id |
| | | | | | “aRMS_NG27.2”; exon “3”; |
| chr13 | exon | 104668785 | 104668908 | − | gene_id “aRMS_NG27”; transcript_id |
| | | | | | “aRMS_NG27.2”; exon “4”; |
| chr13 | exon | 104674255 | 104674357 | − | gene_id “aRMS_NG27”; transcript_id |
| | | | | | “aRMS_NG27.2”; exon “5”; |
| chr13 | transcript | 104667962 | 104674357 | − | gene_id “aRMS_NG27”; transcript_id |
| | | | | | “aRMS_NG27.1”; RPKM “1.0791”; cov |
| | | | | | “14.8805”; |
| chr13 | exon | 104667962 | 104668908 | − | gene_id “aRMS_NG27”; transcript_id |
| | | | | | “aRMS_NG27.1”; exon “1”; |
| chr13 | exon | 104674255 | 104674357 | − | gene_id “aRMS_NG27”; transcript_id |
| | | | | | “aRMS_NG27.1”; exon “2”; |
| chr17 | transcript | 76541142 | 76549365 | + | gene_id “aRMS_NG28”; transcript_id |
| | | | | | “aRMS_NG28.1”; RPKM “6.8005”; cov |
| | | | | | “93.7787”; |
| chr17 | exon | 76541142 | 76541264 | + | gene_id “aRMS_NG28”; transcript_id |
| | | | | | “aRMS_NG28.1”; exon “1”; |
| chr17 | exon | 76542082 | 76549365 | + | gene_id “aRMS_NG28”; transcript_id |
| | | | | | “aRMS_NG28.1”; exon “2”; |
| chr17 | transcript | 76541142 | 76549365 | + | gene_id “aRMS_NG28”; transcript_id |
| | | | | | “aRMS_NG28.2”; RPKM “1.4157”; cov |
| | | | | | “19.5220”; |
| chr17 | exon | 76541142 | 76541264 | + | gene_id “aRMS_NG28”; transcript_id |
| | | | | | “aRMS_NG28.2”; exon “1”; |
| chr17 | exon | 76541689 | 76549365 | + | gene_id “aRMS_NG28”; transcript_id |
| | | | | | “aRMS_NG28.2”; exon “2”; |
| chr17 | transcript | 76541142 | 76576389 | + | gene_id “aRMS_NG28”; transcript_id |
| | | | | | “aRMS_NG28.3”; RPKM “19.7431”; cov |
| | | | | | “272.2587”; |
| chr17 | exon | 76541142 | 76541264 | + | gene_id “aRMS_NG28”; transcript_id |
| | | | | | “aRMS_NG28.3”; exon “1”; |
| chr17 | exon | 76562497 | 76576389 | + | gene_id “aRMS_NG28”; transcript_id |
| | | | | | “aRMS_NG28.3”; exon “2”; |
| chr17 | transcript | 76541142 | 76576389 | + | gene_id “aRMS_NG28”; transcript_id |
| | | | | | “aRMS_NG28.4”; RPKM “15.7457”; cov |
| | | | | | “217.1337”; |
| chr17 | exon | 76541142 | 76552531 | + | gene_id “aRMS_NG28”; transcript_id |
| | | | | | “aRMS_NG28.4”; exon “1”; |
| chr17 | exon | 76562497 | 76576389 | + | gene_id “aRMS_NG28”; transcript_id |
| | | | | | “aRMS_NG28.4”; exon “2”; |
| chr17 | transcript | 76549408 | 76560136 | + | gene_id “aRMS_NG28”; transcript_id |
| | | | | | “aRMS_NG28.5”; RPKM “1.3354”; cov |
| | | | | | “18.4157”; |
| chr17 | exon | 76549408 | 76554529 | + | gene_id “aRMS_NG28”; transcript_id |
| | | | | | “aRMS_NG28.5”; exon “1”; |
| chr17 | exon | 76554552 | 76560136 | + | gene_id “aRMS_NG28”; transcript_id |
| | | | | | “aRMS_NG28.5”; exon “2”; |
| chr17 | transcript | 76549408 | 76560136 | + | gene_id “aRMS_NG28”; transcript_id |
| | | | | | “aRMS_NG28.6”; RPKM “1.7127”; cov |
| | | | | | “23.6176”; |
| chr17 | exon | 76549408 | 76560136 | + | gene_id “aRMS_NG28”; transcript_id |
| | | | | | “aRMS_NG28.6”; exon “1”; |
| chr19 | transcript | 57417319 | 57431910 | + | gene_id “aRMS_NG29”; transcript_id |
| | | | | | “aRMS_NG29.1”; RPKM “1.9098”; cov |
| | | | | | “26.3369”; |
| chr19 | exon | 57417319 | 57431910 | + | gene_id “aRMS_NG29”; transcript_id |
| | | | | | “aRMS_NG29.1”; exon “1”; |
| chr19 | transcript | 57598646 | 57609882 | + | gene_id “aRMS_NG30”; transcript_id |
| | | | | | “aRMS_NG30.2”; RPKM “0.7978”; cov |
| | | | | | “11.0013”; |
| chr19 | exon | 57598646 | 57598863 | + | gene_id “aRMS_NG30”; transcript_id |
| | | | | | “aRMS_NG30.2”; exon “1”; |
| chr19 | exon | 57599589 | 57599885 | + | gene_id “aRMS_NG30”; transcript_id |
| | | | | | “aRMS_NG30.2”; exon “2”; |
| chr19 | exon | 57601082 | 57609882 | + | gene_id “aRMS_NG30”; transcript_id |
| | | | | | “aRMS_NG30.2”; exon “3”; |
| chr19 | transcript | 57599288 | 57609882 | + | gene_id “aRMS_NG30”; transcript_id |
| | | | | | “aRMS_NG30.1”; RPKM “3.1613”; cov |
| | | | | | “43.5951”; |
| chr19 | exon | 57599288 | 57599885 | + | gene_id “aRMS_NG30”; transcript_id |
| | | | | | “aRMS_NG30.1”; exon “1”; |
| chr19 | exon | 57601082 | 57609882 | + | gene_id “aRMS_NG30”; transcript_id |
| | | | | | “aRMS_NG30.1”; exon “2”; |
| chr2 | transcript | 28563554 | 28568661 | − | gene_id “aRMS_NG3”; transcript_id |
| | | | | | “aRMS_NG3.1”; RPKM “1.3596”; cov |
| | | | | | “18.7490”; |
| chr2 | exon | 28563554 | 28567345 | − | gene_id “aRMS_NG3”; transcript_id |
| | | | | | “aRMS_NG3.1”; exon “1”; |
| chr2 | exon | 28567828 | 28568661 | − | gene_id “aRMS_NG3”; transcript_id |
| | | | | | “aRMS_NG3.1”; exon “2”; |
| chr2 | transcript | 28569716 | 28575724 | + | gene_id “aRMS_NG4”; transcript_id |
| | | | | | “aRMS_NG4.1”; RPKM “0.9699”; cov |
| | | | | | “13.3743”; |
| chr2 | exon | 28569716 | 28569913 | + | gene_id “aRMS_NG4”; transcript_id |
| | | | | | “aRMS_NG4.1”; exon “1”; |
| chr2 | exon | 28572231 | 28572389 | + | gene_id “aRMS_NG4”; transcript_id |
| | | | | | “aRMS_NG4.1”; exon “2”; |
| chr2 | exon | 28573133 | 28575724 | + | gene_id “aRMS_NG4”; transcript_id |
| | | | | | “aRMS_NG4.1”; exon “3”; |
| chr2 | transcript | 28581222 | 28588798 | + | gene_id “aRMS_NG4”; transcript_id |
| | | | | | “aRMS_NG4.2”; RPKM “0.8424”; cov |
| | | | | | “11.6167”; |
| chr2 | exon | 28581222 | 28585068 | + | gene_id “aRMS_NG4”; transcript_id |
| | | | | | “aRMS_NG4.2”; exon “1”; |
| chr2 | exon | 28585795 | 28586098 | + | gene_id “aRMS_NG4”; transcript_id |
| | | | | | “aRMS_NG4.2”; exon “2”; |
| chr2 | exon | 28587040 | 28588798 | + | gene_id “aRMS_NG4”; transcript_id |
| | | | | | “aRMS_NG4.2”; exon “3”; |
| chr2 | transcript | 28581222 | 28588798 | + | gene_id “aRMS_NG4”; transcript_id |
| | | | | | “aRMS_NG4.3”; RPKM “0.7655”; cov |
| | | | | | “10.5566”; |
| chr2 | exon | 28581222 | 28584621 | + | gene_id “aRMS_NG4”; transcript_id |
| | | | | | “aRMS_NG4.3”; exon “1”; |
| chr2 | exon | 28584947 | 28586098 | + | gene_id “aRMS_NG4”; transcript_id |
| | | | | | “aRMS_NG4.3”; exon “2”; |
| chr2 | exon | 28586955 | 28588798 | + | gene_id “aRMS_NG4”; transcript_id |
| | | | | | “aRMS_NG4.3”; exon “3”; |
| chr20 | transcript | 46428924 | 46474114 | − | gene_id “aRMS_NG31”; transcript_id |
| | | | | | “aRMS_NG31.1”; RPKM “1.7745”; cov |
| | | | | | “24.4700”; |
| chr20 | exon | 46428924 | 46452080 | − | gene_id “aRMS_NG31”; transcript_id |
| | | | | | “aRMS_NG31.1”; exon “1”; |
| chr20 | exon | 46469306 | 46474114 | − | gene_id “aRMS_NG31”; transcript_id |
| | | | | | “aRMS_NG31.1”; exon “2”; |
| chr20 | transcript | 46428924 | 46475805 | − | gene_id “aRMS_NG31”; transcript_id |
| | | | | | “aRMS_NG31.2”; RPKM “0.9184”; cov |
| | | | | | “12.6647”; |
| chr20 | exon | 46428924 | 46452080 | − | gene_id “aRMS_NG31”; transcript_id |
| | | | | | “aRMS_NG31.2”; exon “1”; |
| chr20 | exon | 46469306 | 46471101 | − | gene_id “aRMS_NG31”; transcript_id |
| | | | | | “aRMS_NG31.2”; exon “2”; |
| chr20 | exon | 46471923 | 46472032 | − | gene_id “aRMS_NG31”; transcript_id |
| | | | | | “aRMS_NG31.2”; exon “3”; |
| chr20 | exon | 46473327 | 46473445 | − | gene_id “aRMS_NG31”; transcript_id |
| | | | | | “aRMS_NG31.2”; exon “4”; |
| chr20 | exon | 46474829 | 46474940 | − | gene_id “aRMS_NG31”; transcript_id |
| | | | | | “aRMS_NG31.2”; exon “5”; |
| chr20 | exon | 46475236 | 46475805 | − | gene_id “aRMS_NG31”; transcript_id |
| | | | | | “aRMS_NG31.2”; exon “6”; |
| chr20 | transcript | 46428924 | 46475805 | − | gene_id “aRMS_NG31”; transcript_id |
| | | | | | “aRMS_NG31.3”; RPKM “4.0069”; cov |
| | | | | | “55.2560”; |
| chr20 | exon | 46428924 | 46452080 | − | gene_id “aRMS_NG31”; transcript_id |
| | | | | | “aRMS_NG31.3”; exon “1”; |
| chr20 | exon | 46469306 | 46472035 | − | gene_id “aRMS_NG31”; transcript_id |
| | | | | | “aRMS_NG31.3”; exon “2”; |
| chr20 | exon | 46473327 | 46473445 | − | gene_id “aRMS_NG31”; transcript_id |
| | | | | | “aRMS_NG31.3”; exon “3”; |
| chr20 | exon | 46474829 | 46474940 | − | gene_id “aRMS_NG31”; transcript_id |
| | | | | | “aRMS_NG31.3”; exon “4”; |
| chr20 | exon | 46475236 | 46475805 | − | gene_id “aRMS_NG31”; transcript_id |
| | | | | | “aRMS_NG31.3”; exon “5”; |
| chr20 | transcript | 46428924 | 46474114 | − | gene_id “aRMS_NG31”; transcript_id |
| | | | | | “aRMS_NG31.4”; RPKM “0.7667”; cov |
| | | | | | “10.5722”; |
| chr20 | exon | 46428924 | 46452080 | − | gene_id “aRMS_NG31”; transcript_id |
| | | | | | “aRMS_NG31.4”; exon “1”; |
| chr20 | exon | 46469306 | 46471101 | − | gene_id “aRMS_NG31”; transcript_id |
| | | | | | “aRMS_NG31.4”; exon “2”; |
| chr20 | exon | 46471923 | 46472035 | − | gene_id “aRMS_NG31”; transcript_id |
| | | | | | “aRMS_NG31.4”; exon “3”; |
| chr20 | exon | 46473327 | 46474114 | − | gene_id “aRMS_NG31”; transcript_id |
| | | | | | “aRMS_NG31.4”; exon “4”; |
| chr20 | transcript | 46428924 | 46475805 | − | gene_id “aRMS_NG31”; transcript_id |
| | | | | | “aRMS_NG31.5”; RPKM “6.3456”; cov |
| | | | | | “87.5059”; |
| chr20 | exon | 46428924 | 46452080 | − | gene_id “aRMS_NG31”; transcript_id |
| | | | | | “aRMS_NG31.5”; exon “1”; |
| chr20 | exon | 46469306 | 46471101 | − | gene_id “aRMS_NG31”; transcript_id |
| | | | | | “aRMS_NG31.5”; exon “2”; |
| chr20 | exon | 46471923 | 46472035 | − | gene_id “aRMS_NG31”; transcript_id |
| | | | | | “aRMS_NG31.5”; exon “3”; |
| chr20 | exon | 46473327 | 46473445 | − | gene_id “aRMS_NG31”; transcript_id |
| | | | | | “aRMS_NG31.5”; exon “4”; |
| chr20 | exon | 46474829 | 46474940 | − | gene_id “aRMS_NG31”; transcript_id |
| | | | | | “aRMS_NG31.5”; exon “5”; |
| chr20 | exon | 46475236 | 46475805 | − | gene_id “aRMS_NG31”; transcript_id |
| | | | | | “aRMS_NG31.5”; exon “6”; |
| chr21 | transcript | 38665986 | 38668816 | + | gene_id “aRMS_NG32”; transcript_id |
| | | | | | “aRMS_NG32.1”; RPKM “1.0943”; cov |
| | | | | | “15.0911”; |
| chr21 | exon | 38665986 | 38667410 | + | gene_id “aRMS_NG32”; transcript_id |
| | | | | | “aRMS_NG32.1”; exon “1”; |
| chr21 | exon | 38668375 | 38668816 | + | gene_id “aRMS_NG32”; transcript_id |
| | | | | | “aRMS_NG32.1”; exon “2”; |
| chr22 | transcript | 21386459 | 21391479 | + | gene_id “aRMS_NG33”; transcript_id |
| | | | | | “aRMS_NG33.1”; RPKM “2.3515”; cov |
| | | | | | “32.4278”; |
| chr22 | exon | 21386459 | 21391479 | + | gene_id “aRMS_NG33”; transcript_id |
| | | | | | “aRMS_NG33.1”; exon “1”; |
| chr22 | transcript | 26445016 | 26464871 | + | gene_id “aRMS_NG34”; transcript_id |
| | | | | | “aRMS_NG34.1”; RPKM “0.8321”; cov |
| | | | | | “11.4742”; |
| chr22 | exon | 26445016 | 26446624 | + | gene_id “aRMS_NG34”; transcript_id |
| | | | | | “aRMS_NG34.1”; exon “1”; |
| chr22 | exon | 26447440 | 26447531 | + | gene_id “aRMS_NG34”; transcript_id |
| | | | | | “aRMS_NG34.1”; exon “2”; |
| chr22 | exon | 26452329 | 26452488 | + | gene_id “aRMS_NG34”; transcript_id |
| | | | | | “aRMS_NG34.1”; exon “3”; |
| chr22 | exon | 26452588 | 26464871 | + | gene_id “aRMS_NG34”; transcript_id |
| | | | | | “aRMS_NG34.1”; exon “4”; |
| chr3 | transcript | 174019088 | 174026584 | − | gene_id “aRMS_NG5”; transcript_id |
| | | | | | “aRMS_NG5.1”; RPKM “4.3565”; cov |
| | | | | | “60.0765”; |
| chr3 | exon | 174019088 | 174020623 | − | gene_id “aRMS_NG5”; transcript_id |
| | | | | | “aRMS_NG5.1”; exon “1”; |
| chr3 | exon | 174026000 | 174026584 | − | gene_id “aRMS_NG5”; transcript_id |
| | | | | | “aRMS_NG5.1”; exon “2”; |
| chr3 | transcript | 174019088 | 174057277 | − | gene_id “aRMS_NG5”; transcript_id |
| | | | | | “aRMS_NG5.2”; RPKM “1.2396”; cov |
| | | | | | “17.0947”; |
| chr3 | exon | 174019088 | 174020623 | − | gene_id “aRMS_NG5”; transcript_id |
| | | | | | “aRMS_NG5.2”; exon “1”; |
| chr3 | exon | 174057129 | 174057277 | − | gene_id “aRMS_NG5”; transcript_id |
| | | | | | “aRMS_NG5.2”; exon “2”; |
| chr4 | transcript | 8396124 | 8396972 | + | gene_id “aRMS_NG6”; transcript_id |
| | | | | | “aRMS_NG6.1”; RPKM “2.5108”; cov |
| | | | | | “34.6243”; |
| chr4 | exon | 8396124 | 8396972 | + | gene_id “aRMS_NG6”; transcript_id |
| | | | | | “aRMS_NG6.1”; exon “1”; |
| chr4 | transcript | 8397151 | 8402660 | + | gene_id “aRMS_NG7”; transcript_id |
| | | | | | “aRMS_NG7.1”; RPKM “3.2380”; cov |
| | | | | | “44.6528”; |
| chr4 | exon | 8397151 | 8402660 | + | gene_id “aRMS_NG7”; transcript_id |
| | | | | | “aRMS_NG7.1”; exon “1”; |
| chr4 | transcript | 129503931 | 129554649 | + | gene_id “aRMS_NG8”; transcript_id |
| | | | | | “aRMS_NG8.1”; RPKM “1.7869”; cov |
| | | | | | “24.6409”; |
| chr4 | exon | 129503931 | 129504083 | + | gene_id “aRMS_NG8”; transcript_id |
| | | | | | “aRMS_NG8.1”; exon “1”; |
| chr4 | exon | 129551125 | 129554649 | + | gene_id “aRMS_NG8”; transcript_id |
| | | | | | “aRMS_NG8.1”; exon “2”; |
| chr4 | transcript | 129532346 | 129537825 | + | gene_id “aRMS_NG9”; transcript_id |
| | | | | | “aRMS_NG9.1”; RPKM “1.6663”; cov |
| | | | | | “22.9788”; |
| chr4 | exon | 129532346 | 129537825 | + | gene_id “aRMS_NG9”; transcript_id |
| | | | | | “aRMS_NG9.1”; exon “1”; |
| chr4 | transcript | 129537851 | 129540993 | + | gene_id “aRMS_NG10”; transcript_id |
| | | | | | “aRMS_NG10.1”; RPKM “2.7696”; cov |
| | | | | | “38.1925”; |
| chr4 | exon | 129537851 | 129540993 | + | gene_id “aRMS_NG10”; transcript_id |
| | | | | | “aRMS_NG10.1”; exon “1”; |
| chr4 | transcript | 129548522 | 129551102 | + | gene_id “aRMS_NG11”; transcript_id |
| | | | | | “aRMS_NG11.1”; RPKM “2.0706”; cov |
| | | | | | “28.5537”; |
| chr4 | exon | 129548522 | 129551102 | + | gene_id “aRMS_NG11”; transcript_id |
| | | | | | “aRMS_NG11.1”; exon “1”; |
| chr5 | transcript | 177515541 | 177542258 | − | gene_id “aRMS_NG12”; transcript_id |
| | | | | | “aRMS_NG12.1”; RPKM “1.3290”; cov |
| | | | | | “18.3274”; |
| chr5 | exon | 177515541 | 177515705 | − | gene_id “aRMS_NG12”; transcript_id |
| | | | | | “aRMS_NG12.1”; exon “1”; |
| chr5 | exon | 177518090 | 177518189 | − | gene_id “aRMS_NG12”; transcript_id |
| | | | | | “aRMS_NG12.1”; exon “2”; |
| chr5 | exon | 177520226 | 177520390 | − | gene_id “aRMS_NG12”; transcript_id |
| | | | | | “aRMS_NG12.1”; exon “3”; |
| chr5 | exon | 177542062 | 177542258 | − | gene_id “aRMS_NG12”; transcript_id |
| | | | | | “aRMS_NG12.1”; exon “4”; |
| chr6 | transcript | 23228518 | 23408352 | + | gene_id “aRMS_NG13”; transcript_id |
| | | | | | “aRMS_NG13.4”; RPKM “1.0148”; cov |
| | | | | | “13.9941”; |
| chr6 | exon | 23228518 | 23229280 | + | gene_id “aRMS_NG13”; transcript_id |
| | | | | | “aRMS_NG13.4”; exon “1”; |
| chr6 | exon | 23304101 | 23304154 | + | gene_id “aRMS_NG13”; transcript_id |
| | | | | | “aRMS_NG13.4”; exon “2”; |
| chr6 | exon | 23395032 | 23395165 | + | gene_id “aRMS_NG13”; transcript_id |
| | | | | | “aRMS_NG13.4”; exon “3”; |
| chr6 | exon | 23397482 | 23397706 | + | gene_id “aRMS_NG13”; transcript_id |
| | | | | | “aRMS_NG13.4”; exon “4”; |
| chr6 | exon | 23399158 | 23399350 | + | gene_id “aRMS_NG13”; transcript_id |
| | | | | | “aRMS_NG13.4”; exon “5”; |
| chr6 | exon | 23403169 | 23403351 | + | gene_id “aRMS_NG13”; transcript_id |
| | | | | | “aRMS_NG13.4”; exon “6”; |
| chr6 | exon | 23404092 | 23408352 | + | gene_id “aRMS_NG13”; transcript_id |
| | | | | | “aRMS_NG13.4”; exon “7”; |
| chr6 | transcript | 23228518 | 23408352 | + | gene_id “aRMS_NG13”; transcript_id |
| | | | | | “aRMS_NG13.3”; RPKM “1.3118”; cov |
| | | | | | “18.0896”; |
| chr6 | exon | 23228518 | 23229280 | + | gene_id “aRMS_NG13”; transcript_id |
| | | | | | “aRMS_NG13.3”; exon “1”; |
| chr6 | exon | 23304101 | 23304154 | + | gene_id “aRMS_NG13”; transcript_id |
| | | | | | “aRMS_NG13.3”; exon “2”; |
| chr6 | exon | 23395032 | 23395182 | + | gene_id “aRMS_NG13”; transcript_id |
| | | | | | “aRMS_NG13.3”; exon “3”; |
| chr6 | exon | 23397482 | 23397706 | + | gene_id “aRMS_NG13”; transcript_id |
| | | | | | “aRMS_NG13.3”; exon “4”; |
| chr6 | exon | 23398137 | 23398343 | + | gene_id “aRMS_NG13”; transcript_id |
| | | | | | “aRMS_NG13.3”; exon “5”; |
| chr6 | exon | 23399158 | 23399350 | + | gene_id “aRMS_NG13”; transcript_id |
| | | | | | “aRMS_NG13.3”; exon “6”; |
| chr6 | exon | 23403169 | 23403351 | + | gene_id “aRMS_NG13”; transcript_id |
| | | | | | “aRMS_NG13.3”; exon “7”; |
| chr6 | exon | 23404092 | 23408352 | + | gene_id “aRMS_NG13”; transcript_id |
| | | | | | “aRMS_NG13.3”; exon “8”; |
| chr6 | transcript | 23228518 | 23408352 | + | gene_id “aRMS_NG13”; transcript_id |
| | | | | | “aRMS_NG13.2”; RPKM “0.7512”; cov |
| | | | | | “10.3586”; |
| chr6 | exon | 23228518 | 23229280 | + | gene_id “aRMS_NG13”; transcript_id |
| | | | | | “aRMS_NG13.2”; exon “1”; |
| chr6 | exon | 23304101 | 23304154 | + | gene_id “aRMS_NG13”; transcript_id |
| | | | | | “aRMS_NG13.2”; exon “2”; |
| chr6 | exon | 23395032 | 23395165 | + | gene_id “aRMS_NG13”; transcript_id |
| | | | | | “aRMS_NG13.2”; exon “3”; |
| chr6 | exon | 23399158 | 23399350 | + | gene_id “aRMS_NG13”; transcript_id |
| | | | | | “aRMS_NG13.2”; exon “4”; |
| chr6 | exon | 23403169 | 23403351 | + | gene_id “aRMS_NG13”; transcript_id |
| | | | | | “aRMS_NG13.2”; exon “5”; |
| chr6 | exon | 23404092 | 23408352 | + | gene_id “aRMS_NG13”; transcript_id |
| | | | | | “aRMS_NG13.2”; exon “6”; |
| chr6 | transcript | 23400822 | 23408352 | + | gene_id “aRMS_NG13”; transcript_id |
| | | | | | “aRMS_NG13.1”; RPKM “1.9057”; cov |
| | | | | | “26.2803”; |
| chr6 | exon | 23400822 | 23403351 | + | gene_id “aRMS_NG13”; transcript_id |
| | | | | | “aRMS_NG13.1”; exon “1”; |
| chr6 | exon | 23404092 | 23408352 | + | gene_id “aRMS_NG13”; transcript_id |
| | | | | | “aRMS_NG13.1”; exon “2”; |
| chr6 | transcript | 44141763 | 44149316 | − | gene_id “aRMS_NG14”; transcript_id |
| | | | | | “aRMS_NG14.1”; RPKM “5.8352”; cov |
| | | | | | “80.4675”; |
| chr6 | exon | 44141763 | 44149316 | − | gene_id “aRMS_NG14”; transcript_id |
| | | | | | “aRMS_NG14.1”; exon “1”; |
| chr6 | transcript | 58137660 | 58147788 | − | gene_id “aRMS_NG15”; transcript_id |
| | | | | | “aRMS_NG15.1”; RPKM “1.6815”; cov |
| | | | | | “23.1882”; |
| chr6 | exon | 58137660 | 58141849 | − | gene_id “aRMS_NG15”; transcript_id |
| | | | | | “aRMS_NG15.1”; exon “1”; |
| chr6 | exon | 58142270 | 58142394 | − | gene_id “aRMS_NG15”; transcript_id |
| | | | | | “aRMS_NG15.1”; exon “2”; |
| chr6 | exon | 58142768 | 58142858 | − | gene_id “aRMS_NG15”; transcript_id |
| | | | | | “aRMS_NG15.1”; exon “3”; |
| chr6 | exon | 58147164 | 58147788 | − | gene_id “aRMS_NG15”; transcript_id |
| | | | | | “aRMS_NG15.1”; exon “4”; |
| chr6 | transcript | 58137660 | 58147788 | − | gene_id “aRMS_NG15”; transcript_id |
| | | | | | “aRMS_NG15.2”; RPKM “13.3887”; cov |
| | | | | | “184.6310”; |
| chr6 | exon | 58137660 | 58141849 | − | gene_id “aRMS_NG15”; transcript_id |
| | | | | | “aRMS_NG15.2”; exon “1”; |
| chr6 | exon | 58142270 | 58142858 | − | gene_id “aRMS_NG15”; transcript_id |
| | | | | | “aRMS_NG15.2”; exon “2”; |
| chr6 | exon | 58147164 | 58147788 | − | gene_id “aRMS_NG15”; transcript_id |
| | | | | | “aRMS_NG15.2”; exon “3”; |
| chr6 | transcript | 58137660 | 58147788 | − | gene_id “aRMS_NG15”; transcript_id |
| | | | | | “aRMS_NG15.3”; RPKM “10.3603”; cov |
| | | | | | “142.8692”; |
| chr6 | exon | 58137660 | 58141849 | − | gene_id “aRMS_NG15”; transcript_id |
| | | | | | “aRMS_NG15.3”; exon “1”; |
| chr6 | exon | 58142270 | 58142394 | − | gene_id “aRMS_NG15”; transcript_id |
| | | | | | “aRMS_NG15.3”; exon “2”; |
| chr6 | exon | 58142768 | 58146783 | − | gene_id “aRMS_NG15”; transcript_id |
| | | | | | “aRMS_NG15.3”; exon “3”; |
| chr6 | exon | 58147164 | 58147788 | − | gene_id “aRMS_NG15”; transcript_id |
| | | | | | “aRMS_NG15.3”; exon “4”; |
| chr6 | transcript | 58137660 | 58147788 | − | gene_id “aRMS_NG15”; transcript_id |
| | | | | | “aRMS_NG15.4”; RPKM “9.9952”; cov |
| | | | | | “137.8339”; |
| chr6 | exon | 58137660 | 58141849 | − | gene_id “aRMS_NG15”; transcript_id |
| | | | | | “aRMS_NG15.4”; exon “1”; |
| chr6 | exon | 58142270 | 58142394 | − | gene_id “aRMS_NG15”; transcript_id |
| | | | | | “aRMS_NG15.4”; exon “2”; |
| chr6 | exon | 58142768 | 58147788 | − | gene_id “aRMS_NG15”; transcript_id |
| | | | | | “aRMS_NG15.4”; exon “3”; |
| chr7 | transcript | 21181330 | 21196346 | + | gene_id “aRMS_NG16”; transcript_id |
| | | | | | “aRMS_NG16.1”; RPKM “1.2801”; cov |
| | | | | | “17.6532”; |
| chr7 | exon | 21181330 | 21181466 | + | gene_id “aRMS_NG16”; transcript_id |
| | | | | | “aRMS_NG16.1”; exon “1”; |
| chr7 | exon | 21183109 | 21183255 | + | gene_id “aRMS_NG16”; transcript_id |
| | | | | | “aRMS_NG16.1”; exon “2”; |
| chr7 | exon | 21196000 | 21196346 | + | gene_id “aRMS_NG16”; transcript_id |
| | | | | | “aRMS_NG16.1”; exon “3”; |
| chr7 | transcript | 21181330 | 21226342 | + | gene_id “aRMS_NG16”; transcript_id |
| | | | | | “aRMS_NG16.4”; RPKM “4.8782”; cov |
| | | | | | “67.2706”; |
| chr7 | exon | 21181330 | 21181466 | + | gene_id “aRMS_NG16”; transcript_id |
| | | | | | “aRMS_NG16.4”; exon “1”; |
| chr7 | exon | 21183109 | 21183255 | + | gene_id “aRMS_NG16”; transcript_id |
| | | | | | “aRMS_NG16.4”; exon “2”; |
| chr7 | exon | 21213219 | 21213297 | + | gene_id “aRMS_NG16”; transcript_id |
| | | | | | “aRMS_NG16.4”; exon “3”; |
| chr7 | exon | 21224748 | 21225268 | + | gene_id “aRMS_NG16”; transcript_id |
| | | | | | “aRMS_NG16.4”; exon “4”; |
| chr7 | exon | 21225368 | 21226342 | + | gene_id “aRMS_NG16”; transcript_id |
| | | | | | “aRMS_NG16.4”; exon “5”; |
| chr7 | transcript | 21181330 | 21213989 | + | gene_id “aRMS_NG16”; transcript_id |
| | | | | | “aRMS_NG16.3”; RPKM “0.7341”; cov |
| | | | | | “10.1231”; |
| chr7 | exon | 21181330 | 21181466 | + | gene_id “aRMS_NG16”; transcript_id |
| | | | | | “aRMS_NG16.3”; exon “1”; |
| chr7 | exon | 21183109 | 21183255 | + | gene_id “aRMS_NG16”; transcript_id |
| | | | | | “aRMS_NG16.3”; exon “2”; |
| chr7 | exon | 21213219 | 21213989 | + | gene_id “aRMS_NG16”; transcript_id |
| | | | | | “aRMS_NG16.3”; exon “3”; |
| chr7 | transcript | 21221439 | 21226342 | + | gene_id “aRMS_NG16”; transcript_id |
| | | | | | “aRMS_NG16.2”; RPKM “3.2121”; cov |
| | | | | | “44.2951”; |
| chr7 | exon | 21221439 | 21225268 | + | gene_id “aRMS_NG16”; transcript_id |
| | | | | | “aRMS_NG16.2”; exon “1”; |
| chr7 | exon | 21225368 | 21226342 | + | gene_id “aRMS_NG16”; transcript_id |
| | | | | | “aRMS_NG16.2”; exon “2”; |
| chr7 | transcript | 21243598 | 21253288 | + | gene_id “aRMS_NG16”; transcript_id |
| | | | | | “aRMS_NG16.5”; RPKM “2.4381”; cov |
| | | | | | “33.6209”; |
| chr7 | exon | 21243598 | 21246606 | + | gene_id “aRMS_NG16”; transcript_id |
| | | | | | “aRMS_NG16.5”; exon “1”; |
| chr7 | exon | 21247751 | 21247959 | + | gene_id “aRMS_NG16”; transcript_id |
| | | | | | “aRMS_NG16.5”; exon “2”; |
| chr7 | exon | 21249037 | 21249190 | + | gene_id “aRMS_NG16”; transcript_id |
| | | | | | “aRMS_NG16.5”; exon “3”; |
| chr7 | exon | 21252579 | 21253288 | + | gene_id “aRMS_NG16”; transcript_id |
| | | | | | “aRMS_NG16.5”; exon “4”; |
| chr7 | transcript | 21243598 | 21253288 | + | gene_id “aRMS_NG16”; transcript_id |
| | | | | | “aRMS_NG16.6”; RPKM “1.0126”; cov |
| | | | | | “13.9639”; |
| chr7 | exon | 21243598 | 21246869 | + | gene_id “aRMS_NG16”; transcript_id |
| | | | | | “aRMS_NG16.6”; exon “1”; |
| chr7 | exon | 21247751 | 21247959 | + | gene_id “aRMS_NG16”; transcript_id |
| | | | | | “aRMS_NG16.6”; exon “2”; |
| chr7 | exon | 21249037 | 21249190 | + | gene_id “aRMS_NG16”; transcript_id |
| | | | | | “aRMS_NG16.6”; exon “3”; |
| chr7 | exon | 21252579 | 21253288 | + | gene_id “aRMS_NG16”; transcript_id |
| | | | | | “aRMS_NG16.6”; exon “4”; |
| chr7 | transcript | 21243598 | 21253288 | + | gene_id “aRMS_NG16”; transcript_id |
| | | | | | “aRMS_NG16.7”; RPKM “15.4019”; cov |
| | | | | | “212.3930”; |
| chr7 | exon | 21243598 | 21247959 | + | gene_id “aRMS_NG16”; transcript_id |
| | | | | | “aRMS_NG16.7”; exon “1”; |
| chr7 | exon | 21249037 | 21249190 | + | gene_id “aRMS_NG16”; transcript_id |
| | | | | | “aRMS_NG16.7”; exon “2”; |
| chr7 | exon | 21252579 | 21253288 | + | gene_id “aRMS_NG16”; transcript_id |
| | | | | | “aRMS_NG16.7”; exon “3”; |
| chr8 | transcript | 41297207 | 41305208 | − | gene_id “aRMS_NG17”; transcript_id |
| | | | | | “aRMS_NG17.1”; RPKM “1.6833”; cov |
| | | | | | “23.2131”; |
| chr8 | exon | 41297207 | 41297935 | − | gene_id “aRMS_NG17”; transcript_id |
| | | | | | “aRMS_NG17.1”; exon “1”; |
| chr8 | exon | 41300116 | 41300269 | − | gene_id “aRMS_NG17”; transcript_id |
| | | | | | “aRMS_NG17.1”; exon “2”; |
| chr8 | exon | 41304960 | 41305208 | − | gene_id “aRMS_NG17”; transcript_id |
| | | | | | “aRMS_NG17.1”; exon “3”; |
| chr9 | transcript | 38674710 | 38677849 | − | gene_id “aRMS_NG18”; transcript_id |
| | | | | | “aRMS_NG18.1”; RPKM “1.4443”; cov |
| | | | | | “19.9172”; |
| chr9 | exon | 38674710 | 38676393 | − | gene_id “aRMS_NG18”; transcript_id |
| | | | | | “aRMS_NG18.1”; exon “1”; |
| chr9 | exon | 38677121 | 38677849 | − | gene_id “aRMS_NG18”; transcript_id |
| | | | | | “aRMS_NG18.1”; exon “2”; |
| chr9 | transcript | 70914397 | 70918487 | − | gene_id “aRMS_NG19”; transcript_id |
| | | | | | “aRMS_NG19.1”; RPKM “5.8290”; cov |
| | | | | | “80.3818”; |
| chr9 | exon | 70914397 | 70918487 | − | gene_id “aRMS_NG19”; transcript_id |
| | | | | | “aRMS_NG19.1”; exon “1”; |
| chr9 | transcript | 139595470 | 139596560 | + | gene_id “aRMS_NG20”; transcript_id |
| | | | | | “aRMS_NG20.2”; RPKM “1.6449”; cov |
| | | | | | “22.6837”; |
| chr9 | exon | 139595470 | 139596560 | + | gene_id “aRMS_NG20”; transcript_id |
| | | | | | “aRMS_NG20.2”; exon “1”; |
| chr9 | transcript | 139599667 | 139603134 | + | gene_id “aRMS_NG20”; transcript_id |
| | | | | | “aRMS_NG20.1”; RPKM “2.7191”; cov |
| | | | | | “37.4965”; |
| chr9 | exon | 139599667 | 139603134 | + | gene_id “aRMS_NG20”; transcript_id |
| | | | | | “aRMS_NG20.1”; exon “1”; |
| chrX | transcript | 47658608 | 47663864 | − | gene_id “aRMS_NG35”; transcript_id |
| | | | | | “aRMS_NG35.1”; RPKM “5.1432”; cov |
| | | | | | “70.9254”; |
| chrX | exon | 47658608 | 47663553 | − | gene_id “aRMS_NG35”; transcript_id |
| | | | | | “aRMS_NG35.1”; exon “1”; |
| chrX | exon | 47663769 | 47663864 | − | gene_id “aRMS_NG35”; transcript_id |
| | | | | | “aRMS_NG35.1”; exon “2”; |
| chrX | transcript | 47658608 | 47663864 | − | gene_id “aRMS_NG35”; transcript_id |
| | | | | | “aRMS_NG35.2”; RPKM “1.5505”; cov |
| | | | | | “21.3817”; |
| chrX | exon | 47658608 | 47663186 | − | gene_id “aRMS_NG35”; transcript_id |
| | | | | | “aRMS_NG35.2”; exon “1”; |
| chrX | exon | 47663375 | 47663553 | − | gene_id “aRMS_NG35”; transcript_id |
| | | | | | “aRMS_NG35.2”; exon “2”; |
| chrX | exon | 47663769 | 47663864 | − | gene_id “aRMS_NG35”; transcript_id |
| | | | | | “aRMS_NG35.2”; exon “3”; |
| chrX | transcript | 100849711 | 100852909 | − | gene_id “aRMS_NG36”; transcript_id |
| | | | | | “aRMS_NG36.1”; RPKM “0.7543”; cov |
| | | | | | “10.4015”; |
| chrX | exon | 100849711 | 100851871 | − | gene_id “aRMS_NG36”; transcript_id |
| | | | | | “aRMS_NG36.1”; exon “1”; |
| chrX | exon | 100851972 | 100852909 | − | gene_id “aRMS_NG36”; transcript_id |
| | | | | | “aRMS_NG36.1”; exon “2”; |
| chrX | transcript | 100849711 | 100852909 | − | gene_id “aRMS_NG36”; transcript_id |
| | | | | | “aRMS_NG36.2”; RPKM “3.1388”; cov |
| | | | | | “43.2842”; |
| chrX | exon | 100849711 | 100852909 | − | gene_id “aRMS_NG36”; transcript_id |
| | | | | | “aRMS_NG36.2”; exon “1”; |
| chr17 | transcript | 8029992 | 8030281 | − | gene_id “BCOR_NG6”; transcript_id |
| | | | | | “BOOR NG6.1”; RPKM “1.1532”; cov |
| | | | | | “20.9793”; |
| chr17 | exon | 8029992 | 8030281 | − | gene_id “BCOR_NG6”; transcript_id |
| | | | | | “BCOR_NG6.1”; exon “1”; |
| chr5 | transcript | 2707873 | 2714605 | − | gene_id “BCOR_NG1”; transcript_id |
| | | | | | “BCOR_NG1.1”; RPKM “1.5621”; cov |
| | | | | | “28.4169”; |
| chr5 | exon | 2707873 | 2714605 | − | gene_id “BCOR_NG1”; transcript_id |
| | | | | | “BCOR_NG1.1”; exon “1”; |
| chr6 | transcript | 137239596 | 137243510 | − | gene_id “BCOR_NG2”; transcript_id |
| | | | | | “BCOR_NG2.1”; RPKM “4.8026”; cov |
| | | | | | “87.3659”; |
| chr6 | exon | 137239596 | 137243510 | − | gene_id “BCOR_NG2”; transcript_id |
| | | | | | “BCOR_NG2.1”; exon “1”; |
| chr6 | transcript | 137243518 | 137244385 | − | gene_id “BCOR_NG3”; transcript_id |
| | | | | | “BOOR NG3.1”; RPKM “6.2187”; cov |
| | | | | | “113.1279”; |
| chr6 | exon | 137243518 | 137244385 | − | gene_id “BCOR_NG3”; transcript_id |
| | | | | | “BCOR_NG3.1”; exon “1”; |
| chr7 | transcript | 1709521 | 1709794 | + | gene_id “BCOR_NG4”; transcript_id |
| | | | | | “BCOR_NG4.1”; RPKM “9.1474”; cov |
| | | | | | “166.4059”; |
| chr7 | exon | 1709521 | 1709794 | + | gene_id “BCOR_NG4”; transcript_id |
| | | | | | “BCOR_NG4.1”; exon “1”; |
| chr9 | transcript | 99896877 | 99897463 | − | gene_id “BCOR_NG5”; transcript_id |
| | | | | | “BCOR_NG5.1”; RPKM “9.3141”; cov |
| | | | | | “169.4378”; |
| chr9 | exon | 99896877 | 99897463 | − | gene_id “BCOR_NG5”; transcript_id |
| | | | | | “BCOR_NG5.1”; exon “1”; |
| chr11 | transcript | 134451325 | 134454292 | − | gene_id “CCS_NG8”; transcript_id |
| | | | | | “CCS_NG8.1”; RPKM “1.4735”; cov |
| | | | | | “23.4078”; |
| chr11 | exon | 134451325 | 134451453 | − | gene_id “CCS_NG8”; transcript_id |
| | | | | | “CCS_NG8.1”; exon “1”; |
| chr11 | exon | 134453612 | 134454292 | − | gene_id “CCS_NG8”; transcript_id |
| | | | | | “CCS_NG8.1”; exon “2”; |
| chr13 | transcript | 24042286 | 24053871 | − | gene_id “CCS_NG9”; transcript_id |
| | | | | | “CCS_NG9.1”; RPKM “1.2721”; cov |
| | | | | | “20.2076”; |
| chr13 | exon | 24042286 | 24053871 | − | gene_id “CCS_NG9”; transcript_id |
| | | | | | “CCS_NG9.1”; exon “1”; |
| chr13 | transcript | 24077827 | 24119254 | − | gene_id “CCS_NG10”; transcript_id |
| | | | | | “CCS_NG10.1”; RPKM “0.6573”; cov |
| | | | | | “10.4417”; |
| chr13 | exon | 24077827 | 24079692 | − | gene_id “CCS_NG10”; transcript_id |
| | | | | | “CCS_NG10.1”; exon “1”; |
| chr13 | exon | 24106933 | 24107104 | − | gene_id “CCS_NG10”; transcript_id |
| | | | | | “CCS_NG10.1”; exon “2”; |
| chr13 | exon | 24108270 | 24108606 | − | gene_id “CCS_NG10”; transcript_id |
| | | | | | “CCS_NG10.1”; exon “3”; |
| chr13 | exon | 24119056 | 24119254 | − | gene_id “CCS_NG10”; transcript_id |
| | | | | | “CCS_NG10.1”; exon “4”; |
| chr15 | transcript | 101661721 | 101662920 | + | gene_id “CCS_NG11”; transcript_id |
| | | | | | “CCS_NG11.1”; RPKM “1.8440”; cov |
| | | | | | “29.2938”; |
| chr15 | exon | 101661721 | 101662920 | + | gene_id “CCS_NG11”; transcript_id |
| | | | | | “CCS_NG11.1”; exon “1”; |
| chr17 | transcript | 75878857 | 75881424 | + | gene_id “CCS_NG12”; transcript_id |
| | | | | | “CCS_NG12.1”; RPKM “11.6927”; cov |
| | | | | | “185.7470”; |
| chr17 | exon | 75878857 | 75881424 | + | gene_id “CCS_NG12”; transcript_id |
| | | | | | “CCS_NG12.1”; exon “1”; |
| chr17 | transcript | 75881718 | 75884774 | + | gene_id “CCS_NG13”; transcript_id |
| | | | | | “CCS_NG13.1”; RPKM “6.6437”; cov |
| | | | | | “105.5400”; |
| chr17 | exon | 75881718 | 75884774 | + | gene_id “CCS_NG13”; transcript_id |
| | | | | | “CCS_NG13.1”; exon “1”; |
| chr20 | transcript | 60496906 | 60500873 | − | gene_id “CCS_NG14”; transcript_id |
| | | | | | “CCS_NG14.1”; RPKM “1.1475”; cov |
| | | | | | “18.2292”; |
| chr20 | exon | 60496906 | 60499589 | − | gene_id “CCS_NG14”; transcript_id |
| | | | | | “CCS_NG14.1”; exon “1”; |
| chr20 | exon | 60499834 | 60499897 | − | gene_id “CCS_NG14”; transcript_id |
| | | | | | “COS NG14.1”; exon “2”; |
| chr20 | exon | 60500429 | 60500873 | − | gene_id “CCS_NG14”; transcript_id |
| | | | | | “CCS_NG14.1”; exon “3”; |
| chr20 | transcript | 60496906 | 60500873 | − | gene_id “CCS_NG14”; transcript_id |
| | | | | | “CCS_NG14.2”; RPKM “2.7225”; cov |
| | | | | | “43.2492”; |
| chr20 | exon | 60496906 | 60500873 | − | gene_id “CCS_NG14”; transcript_id |
| | | | | | “CCS_NG14.2”; exon “1”; |
| chr20 | transcript | 60496906 | 60500873 | − | gene_id “CCS_NG14”; transcript_id |
| | | | | | “CCS_NG14.3”; RPKM “4.9048”; cov |
| | | | | | “77.9159”; |
| chr20 | exon | 60496906 | 60499897 | − | gene_id “CCS_NG14”; transcript_id |
| | | | | | “CCS_NG14.3”; exon “1”; |
| chr20 | exon | 60500429 | 60500873 | − | gene_id “CCS_NG14”; transcript_id |
| | | | | | “CCS_NG14.3”; exon “2”; |
| chr5 | transcript | 6773174 | 6773937 | + | gene_id “CCS_NG1”; transcript_id |
| | | | | | “CCS_NG1.1”; RPKM “0.9169”; cov |
| | | | | | “14.5652”; |
| chr5 | exon | 6773174 | 6773334 | + | gene_id “CCS_NG1”; transcript_id |
| | | | | | “CCS_NG1.1”; exon “1”; |
| chr5 | exon | 6773568 | 6773937 | + | gene_id “CCS_NG1”; transcript_id |
| | | | | | “CCS_NG1.1”; exon “2”; |
| chr5 | transcript | 15066053 | 15072044 | − | gene_id “CCS_NG2”; transcript_id |
| | | | | | “CCS_NG2.1”; RPKM “0.6364”; cov |
| | | | | | “10.1093”; |
| chr5 | exon | 15066053 | 15066397 | − | gene_id “CCS_NG2”; transcript_id |
| | | | | | “CCS_NG2.1”; exon “1”; |
| chr5 | exon | 15068595 | 15072044 | − | gene_id “CCS_NG2”; transcript_id |
| | | | | | “CCS_NG2.1”; exon “2”; |
| chr5 | transcript | 15081079 | 15097665 | − | gene_id “CCS_NG2”; transcript_id |
| | | | | | “CCS_NG2.2”; RPKM “0.9396”; cov |
| | | | | | “14.9259”; |
| chr5 | exon | 15081079 | 15081814 | − | gene_id “CCS_NG2”; transcript_id |
| | | | | | “CCS_NG2.2”; exon “1”; |
| chr5 | exon | 15082978 | 15086448 | − | gene_id “CCS_NG2”; transcript_id |
| | | | | | “CCS_NG2.2”; exon “2”; |
| chr5 | exon | 15097230 | 15097665 | − | gene_id “CCS_NG2”; transcript_id |
| | | | | | “CCS_NG2.2”; exon “3”; |
| chr5 | transcript | 15081079 | 15097665 | − | gene_id “CCS_NG2”; transcript_id |
| | | | | | “CCS_NG2.3”; RPKM “0.6475”; cov |
| | | | | | “10.2855”; |
| chr5 | exon | 15081079 | 15086448 | − | gene_id “CCS_NG2”; transcript_id |
| | | | | | “CCS_NG2.3”; exon “1”; |
| chr5 | exon | 15097230 | 15097665 | − | gene_id “CCS_NG2”; transcript_id |
| | | | | | “CCS_NG2.3”; exon “2”; |
| chr6 | transcript | 369156 | 387345 | − | gene_id “CCS_NG3”; transcript_id |
| | | | | | “CCS_NG3.1”; RPKM “2.2894”; cov |
| | | | | | “36.3695”; |
| chr6 | exon | 369156 | 385963 | − | gene_id “CCS_NG3”; transcript_id |
| | | | | | “CCS_NG3.1”; exon “1”; |
| chr6 | exon | 386981 | 387345 | − | gene_id “CCS_NG3”; transcript_id |
| | | | | | “CCS_NG3.1”; exon “2”; |
| chr6 | transcript | 369156 | 387345 | − | gene_id “CCS_NG3”; transcript_id |
| | | | | | “CCS_NG3.2”; RPKM “1.4191”; cov |
| | | | | | “22.5438”; |
| chr6 | exon | 369156 | 386277 | − | gene_id “CCS_NG3”; transcript_id |
| | | | | | “CCS_NG3.2”; exon “1”; |
| chr6 | exon | 386981 | 387345 | − | gene_id “CCS_NG3”; transcript_id |
| | | | | | “CCS_NG3.2”; exon “2”; |
| chr6 | transcript | 369156 | 387345 | − | gene_id “CCS_NG3”; transcript_id |
| | | | | | “CCS_NG3.3”; RPKM “5.4322”; cov |
| | | | | | “86.2941”; |
| chr6 | exon | 369156 | 387345 | − | gene_id “CCS_NG3”; transcript_id |
| | | | | | “CCS_NG3.3”; exon “1”; |
| chr6 | transcript | 170336545 | 170346969 | − | gene_id “CCS_NG4”; transcript_id |
| | | | | | “CCS_NG4.1”; RPKM “2.1722”; cov |
| | | | | | “34.5074”; |
| chr6 | exon | 170336545 | 170346969 | − | gene_id “CCS_NG4”; transcript_id |
| | | | | | “CCS_NG4.1”; exon “1”; |
| chr6 | transcript | 170336545 | 170346969 | − | gene_id “CCS_NG4”; transcript_id |
| | | | | | “CCS_NG4.2”; RPKM “2.8185”; cov |
| | | | | | “44.7739”; |
| chr6 | exon | 170336545 | 170345549 | − | gene_id “CCS_NG4”; transcript_id |
| | | | | | “CCS_NG4.2”; exon “1”; |
| chr6 | exon | 170346508 | 170346969 | − | gene_id “CCS_NG4”; transcript_id |
| | | | | | “CCS_NG4.2”; exon “2”; |
| chr6 | transcript | 170336545 | 170346969 | − | gene_id “CCS_NG4”; transcript_id |
| | | | | | “CCS_NG4.3”; RPKM “0.8487”; cov |
| | | | | | “13.4816”; |
| chr6 | exon | 170336545 | 170345113 | − | gene_id “CCS_NG4”; transcript_id |
| | | | | | “CCS_NG4.3”; exon “1”; |
| chr6 | exon | 170345325 | 170346969 | − | gene_id “CCS_NG4”; transcript_id |
| | | | | | “CCS_NG4.3”; exon “2”; |
| chr6 | transcript | 170336545 | 170346969 | − | gene_id “CCS_NG4”; transcript_id |
| | | | | | “CCS_NG4.4”; RPKM “0.8487”; cov |
| | | | | | “13.4816”; |
| chr6 | exon | 170336545 | 170345113 | − | gene_id “CCS_NG4”; transcript_id |
| | | | | | “CCS_NG4.4”; exon “1”; |
| chr6 | exon | 170345325 | 170345549 | − | gene_id “CCS_NG4”; transcript_id |
| | | | | | “CCS_NG4.4”; exon “2”; |
| chr6 | exon | 170346508 | 170346969 | − | gene_id “CCS_NG4”; transcript_id |
| | | | | | “CCS_NG4.4”; exon “3”; |
| chr6_mcf_hap5 | transcript | 4605092 | 4606016 | − | gene_id “CCS_NG15”; transcript_id |
| | | | | | “CCS_NG15.1”; RPKM “2.9833”; cov |
| | | | | | “47.3916”; |
| chr6_mcf_hap5 | exon | 4605092 | 4606016 | − | gene_id “CCS_NG15”; transcript_id |
| | | | | | “CCS_NG15.1”; exon “1”; |
| chr8 | transcript | 123350339 | 123398217 | − | gene_id “CCS_NG5”; transcript_id |
| | | | | | “CCS_NG5.1”; RPKM “1.3597”; cov |
| | | | | | “21.5996”; |
| chr8 | exon | 123350339 | 123351435 | − | gene_id “CCS_NG5”; transcript_id |
| | | | | | “CCS_NG5.1”; exon “1”; |
| chr8 | exon | 123391653 | 123398217 | − | gene_id “CCS_NG5”; transcript_id |
| | | | | | “CCS_NG5.1”; exon “2”; |
| chr8 | transcript | 123485277 | 123489187 | − | gene_id “CCS_NG7”; transcript_id |
| | | | | | “CCS_NG7.1”; RPKM “2.6632”; cov |
| | | | | | “42.3076”; |
| chr8 | exon | 123485277 | 123489187 | − | gene_id “CCS_NG7”; transcript_id |
| | | | | | “CCS_NG7.1”; exon “1”; |
| chr8 | transcript | 123662431 | 123666910 | − | gene_id “CCS_NG6”; transcript_id |
| | | | | | “CCS_NG6.1”; RPKM “1.6155”; cov |
| | | | | | “25.6629”; |
| chr8 | exon | 123662431 | 123666910 | − | gene_id “CCS_NG6”; transcript_id |
| | | | | | “CCS_NG6.1”; exon “1”; |
| chr10 | transcript | 1585047 | 1587025 | + | gene_id “CIC_NG3”; transcript_id |
| | | | | | “CIC_NG3.1”; RPKM “2.4429”; cov |
| | | | | | “33.7457”; |
| chr10 | exon | 1585047 | 1587025 | + | gene_id “CIC_NG3”; transcript_id |
| | | | | | “CIC_NG3.1”; exon “1”; |
| chr11 | transcript | 127957209 | 128107409 | − | gene_id “CIC_NG4”; transcript_id |
| | | | | | “CIC_NG4.2”; RPKM “3.2069”; cov |
| | | | | | “44.3001”; |
| chr11 | exon | 127957209 | 127957760 | − | gene_id “CIC_NG4”; transcript_id |
| | | | | | “CIC_NG4.2”; exon “1”; |
| chr11 | exon | 128106139 | 128106205 | − | gene_id “CIC_NG4”; transcript_id |
| | | | | | “CIC_NG4.2”; exon “2”; |
| chr11 | exon | 128106796 | 128107409 | − | gene_id “CIC_NG4”; transcript_id |
| | | | | | “CIC_NG4.2”; exon “3”; |
| chr11 | transcript | 127957209 | 128063759 | − | gene_id “CIC_NG4”; transcript_id |
| | | | | | “CIC_NG4.1”; RPKM “5.7983”; cov |
| | | | | | “80.0970”; |
| chr11 | exon | 127957209 | 127957760 | − | gene_id “CIC_NG4”; transcript_id |
| | | | | | “CIC_NG4.1”; exon “1”; |
| chr11 | exon | 128062757 | 128063759 | − | gene_id “CIC_NG4”; transcript_id |
| | | | | | “CIC_NG4.1”; exon “2”; |
| chr11 | transcript | 127957209 | 128063759 | − | gene_id “CIC_NG4”; transcript_id |
| | | | | | “CIC_NG4.4”; RPKM “1.4642”; cov |
| | | | | | “20.2259”; |
| chr11 | exon | 127957209 | 127957760 | − | gene_id “CIC_NG4”; transcript_id |
| | | | | | “CIC_NG4.4”; exon “1”; |
| chr11 | exon | 128053701 | 128053841 | − | gene_id “CIC_NG4”; transcript_id |
| | | | | | “CIC_NG4.4”; exon “2”; |
| chr11 | exon | 128062757 | 128063759 | − | gene_id “CIC_NG4”; transcript_id |
| | | | | | “CIC_NG4.4”; exon “3”; |
| chr11 | transcript | 127957209 | 128107409 | − | gene_id “CIC_NG4”; transcript_id |
| | | | | | “CIC_NG4.3”; RPKM “1.2670”; cov |
| | | | | | “17.5016”; |
| chr11 | exon | 127957209 | 127957760 | − | gene_id “CIC_NG4”; transcript_id |
| | | | | | “CIC_NG4.3”; exon “1”; |
| chr11 | exon | 128053701 | 128053841 | − | gene_id “CIC_NG4”; transcript_id |
| | | | | | “CIC_NG4.3”; exon “2”; |
| chr11 | exon | 128106139 | 128106205 | − | gene_id “CIC_NG4”; transcript_id |
| | | | | | “CIC_NG4.3”; exon “3”; |
| chr11 | exon | 128106796 | 128107409 | − | gene_id “CIC_NG4”; transcript_id |
| | | | | | “CIC_NG4.3”; exon “4”; |
| chr11 | transcript | 128160177 | 128166003 | + | gene_id “CIC_NG5”; transcript_id |
| | | | | | “CIC_NG5.1”; RPKM “3.4212”; cov |
| | | | | | “47.2600”; |
| chr11 | exon | 128160177 | 128166003 | + | gene_id “CIC_NG5”; transcript_id |
| | | | | | “CIC_NG5.1”; exon “1”; |
| chr12 | transcript | 4136824 | 4233631 | + | gene_id “CIC_NG6”; transcript_id |
| | | | | | “CIC_NG6.3”; RPKM “1.8071”; cov |
| | | | | | “24.9635”; |
| chr12 | exon | 4136824 | 4137037 | + | gene_id “CIC_NG6”; transcript_id |
| | | | | | “CIC_NG6.3”; exon “1”; |
| chr12 | exon | 4145480 | 4145574 | + | gene_id “CIC_NG6”; transcript_id |
| | | | | | “CIC_NG6.3”; exon “2”; |
| chr12 | exon | 4232458 | 4233631 | + | gene_id “CIC_NG6”; transcript_id |
| | | | | | “CIC_NG6.3”; exon “3”; |
| chr12 | transcript | 4136824 | 4158306 | + | gene_id “CIC_NG6”; transcript_id |
| | | | | | “CIC_NG6.1”; RPKM “2.4306”; cov |
| | | | | | “33.5761”; |
| chr12 | exon | 4136824 | 4137037 | + | gene_id “CIC_NG6”; transcript_id |
| | | | | | “CIC_NG6.1”; exon “1”; |
| chr12 | exon | 4145480 | 4145574 | + | gene_id “CIC_NG6”; transcript_id |
| | | | | | “CIC_NG6.1”; exon “2”; |
| chr12 | exon | 4156756 | 4158306 | + | gene_id “CIC_NG6”; transcript_id |
| | | | | | “CIC_NG6.1”; exon “3”; |
| chr12 | transcript | 4136824 | 4184020 | + | gene_id “CIC_NG6”; transcript_id |
| | | | | | “CIC_NG6.2”; RPKM “4.3612”; cov |
| | | | | | “60.2453”; |
| chr12 | exon | 4136824 | 4137037 | + | gene_id “CIC_NG6”; transcript_id |
| | | | | | “CIC_NG6.2”; exon “1”; |
| chr12 | exon | 4145480 | 4145574 | + | gene_id “CIC_NG6”; transcript_id |
| | | | | | “CIC_NG6.2”; exon “2”; |
| chr12 | exon | 4183752 | 4184020 | + | gene_id “CIC_NG6”; transcript_id |
| | | | | | “CIC_NG6.2”; exon “3”; |
| chr12 | transcript | 4233680 | 4248549 | + | gene_id “CIC_NG6”; transcript_id |
| | | | | | “CIC_NG6.4”; RPKM “1.8038”; cov |
| | | | | | “24.9169”; |
| chr12 | exon | 4233680 | 4248549 | + | gene_id “CIC_NG6”; transcript_id |
| | | | | | “CIC_NG6.4”; exon “1”; |
| chr12 | transcript | 129193358 | 129227492 | + | gene_id “CIC_NG7”; transcript_id |
| | | | | | “CIC_NG7.1”; RPKM “2.5434”; cov |
| | | | | | “35.1344”; |
| chr12 | exon | 129193358 | 129193654 | + | gene_id “CIC_NG7”; transcript_id |
| | | | | | “CIC_NG7.1”; exon “1”; |
| chr12 | exon | 129206945 | 129207255 | + | gene_id “CIC_NG7”; transcript_id |
| | | | | | “CIC_NG7.1”; exon “2”; |
| chr12 | exon | 129207622 | 129227492 | + | gene_id “CIC_NG7”; transcript_id |
| | | | | | “CIC_NG7.1”; exon “3”; |
| chr12 | transcript | 129193358 | 129227492 | + | gene_id “CIC_NG7”; transcript_id |
| | | | | | “CIC_NG7.2”; RPKM “2.4590”; cov |
| | | | | | “33.9676”; |
| chr12 | exon | 129193358 | 129193654 | + | gene_id “CIC_NG7”; transcript_id |
| | | | | | “CIC_NG7.2”; exon “1”; |
| chr12 | exon | 129206945 | 129227492 | + | gene_id “CIC_NG7”; transcript_id |
| | | | | | “CIC_NG7.2”; exon “2”; |
| chr12 | transcript | 129193358 | 129227492 | + | gene_id “CIC_NG7”; transcript_id |
| | | | | | “CIC_NG7.3”; RPKM “3.7403”; cov |
| | | | | | “51.6674”; |
| chr12 | exon | 129193358 | 129193654 | + | gene_id “CIC_NG7”; transcript_id |
| | | | | | “CIC_NG7.3”; exon “1”; |
| chr12 | exon | 129198470 | 129227492 | + | gene_id “CIC_NG7”; transcript_id |
| | | | | | “CIC_NG7.3”; exon “2”; |
| chr21 | transcript | 45609540 | 45624622 | − | gene_id “CIC_NG8”; transcript_id |
| | | | | | “CIC_NG8.1”; RPKM “0.9020”; cov |
| | | | | | “12.4603”; |
| chr21 | exon | 45609540 | 45609637 | − | gene_id “CIC_NG8”; transcript_id |
| | | | | | “CIC_NG8.1”; exon “1”; |
| chr21 | exon | 45620722 | 45624622 | − | gene_id “CIC_NG8”; transcript_id |
| | | | | | “CIC_NG8.1”; exon “2”; |
| chr6 | transcript | 147139695 | 147155171 | − | gene_id “CIC_NG1”; transcript_id |
| | | | | | “CIC_NG1.1”; RPKM “9.6448”; cov |
| | | | | | “133.2322”; |
| chr6 | exon | 147139695 | 147155171 | − | gene_id “CIC_NG1”; transcript_id |
| | | | | | “CIC_NG1.1”; exon “1”; |
| chr8 | transcript | 67090474 | 67090871 | + | gene_id “CIC_NG2”; transcript_id |
| | | | | | “CIC_NG2.1”; RPKM “1.7209”; cov |
| | | | | | “23.7723”; |
| chr8 | exon | 67090474 | 67090871 | + | gene_id “CIC_NG2”; transcript_id |
| | | | | | “CIC_NG2.1”; exon “1”; |
| chr1 | transcript | 1027186 | 1037920 | + | gene_id “DSRCT_NG1”; transcript_id |
| | | | | | “DSRCT_NG1.1”; RPKM “3.8908”; cov |
| | | | | | “53.2383”; |
| chr1 | exon | 1027186 | 1037920 | + | gene_id “DSRCT_NG1”; transcript_id |
| | | | | | “DSRCT_NG1.1”; exon “1”; |
| chr1 | transcript | 189902140 | 189989055 | − | gene_id “DSRCT_NG2”; transcript_id |
| | | | | | “DSRCT_NG2.1”; RPKM “1.7504”; cov |
| | | | | | “23.9510”; |
| chr1 | exon | 189902140 | 189903220 | − | gene_id “DSRCT_NG2”; transcript_id |
| | | | | | “DSRCT_NG2.1”; exon “1”; |
| chr1 | exon | 189904368 | 189904735 | − | gene_id “DSRCT_NG2”; transcript_id |
| | | | | | “DSRCT_NG2.1”; exon “2”; |
| chr1 | exon | 189988920 | 189989055 | − | gene_id “DSRCT_NG2”; transcript_id |
| | | | | | “DSRCT_NG2.1”; exon “3”; |
| chr1 | transcript | 189902140 | 189989055 | − | gene_id “DSRCT_NG2”; transcript_id |
| | | | | | “DSRCT_NG2.2”; RPKM “0.9166”; cov |
| | | | | | “12.5414”; |
| chr1 | exon | 189902140 | 189904735 | − | gene_id “DSRCT_NG2”; transcript_id |
| | | | | | “DSRCT_NG2.2”; exon “1”; |
| chr1 | exon | 189909284 | 189909394 | − | gene_id “DSRCT_NG2”; transcript_id |
| | | | | | “DSRCT_NG2.2”; exon “2”; |
| chr1 | exon | 189910092 | 189910243 | − | gene_id “DSRCT_NG2”; transcript_id |
| | | | | | “DSRCT_NG2.2”; exon “3”; |
| chr1 | exon | 189975471 | 189975575 | − | gene_id “DSRCT_NG2”; transcript_id |
| | | | | | “DSRCT_NG2.2”; exon “4”; |
| chr1 | exon | 189988920 | 189989055 | − | gene_id “DSRCT_NG2”; transcript_id |
| | | | | | “DSRCT_NG2.2”; exon “5”; |
| chr1 | transcript | 189902140 | 189989055 | − | gene_id “DSRCT_NG2”; transcript_id |
| | | | | | “DSRCT_NG2.4”; RPKM “9.5213”; cov |
| | | | | | “130.2815”; |
| chr1 | exon | 189902140 | 189904735 | − | gene_id “DSRCT_NG2”; transcript_id |
| | | | | | “DSRCT_NG2.4”; exon “1”; |
| chr1 | exon | 189909284 | 189909394 | − | gene_id “DSRCT_NG2”; transcript_id |
| | | | | | “DSRCT_NG2.4”; exon “2”; |
| chr1 | exon | 189910092 | 189910243 | − | gene_id “DSRCT_NG2”; transcript_id |
| | | | | | “DSRCT_NG2.4”; exon “3”; |
| chr1 | exon | 189988920 | 189989055 | − | gene_id “DSRCT_NG2”; transcript_id |
| | | | | | “DSRCT_NG2.4”; exon “4”; |
| chr1 | transcript | 189905062 | 189989055 | − | gene_id “DSRCT_NG2”; transcript_id |
| | | | | | “DSRCT_NG2.3”; RPKM “1.2552”; cov |
| | | | | | “17.1751”; |
| chr1 | exon | 189905062 | 189909394 | − | gene_id “DSRCT_NG2”; transcript_id |
| | | | | | “DSRCT_NG2.3”; exon “1”; |
| chr1 | exon | 189910092 | 189910243 | − | gene_id “DSRCT_NG2”; transcript_id |
| | | | | | “DSRCT_NG2.3”; exon “2”; |
| chr1 | exon | 189988920 | 189989055 | − | gene_id “DSRCT_NG2”; transcript_id |
| | | | | | “DSRCT_NG2.3”; exon “3”; |
| chr1 | transcript | 221356206 | 221396611 | + | gene_id “DSRCT_NG3”; transcript_id |
| | | | | | “DSRCT_NG3.1”; RPKM “28.9172”; cov |
| | | | | | “395.6792”; |
| chr1 | exon | 221356206 | 221356337 | + | gene_id “DSRCT_NG3”; transcript_id |
| | | | | | “DSRCT_NG3.1”; exon “1”; |
| chr1 | exon | 221359585 | 221364769 | + | gene_id “DSRCT_NG3”; transcript_id |
| | | | | | “DSRCT_NG3.1”; exon “2”; |
| chr1 | exon | 221365457 | 221365737 | + | gene_id “DSRCT_NG3”; transcript_id |
| | | | | | “DSRCT_NG3.1”; exon “3”; |
| chr1 | exon | 221389466 | 221389738 | + | gene_id “DSRCT_NG3”; transcript_id |
| | | | | | “DSRCT_NG3.1”; exon “4”; |
| chr1 | exon | 221391639 | 221396611 | + | gene_id “DSRCT_NG3”; transcript_id |
| | | | | | “DSRCT_NG3.1”; exon “5”; |
| chr1 | transcript | 221356206 | 221396611 | + | gene_id “DSRCT_NG3”; transcript_id |
| | | | | | “DSRCT_NG3.2”; RPKM “16.0576”; cov |
| | | | | | “219.7189”; |
| chr1 | exon | 221356206 | 221356337 | + | gene_id “DSRCT_NG3”; transcript_id |
| | | | | | “DSRCT_NG3.2”; exon “1”; |
| chr1 | exon | 221359585 | 221364769 | + | gene_id “DSRCT_NG3”; transcript_id |
| | | | | | “DSRCT_NG3.2”; exon “2”; |
| chr1 | exon | 221365457 | 221365737 | + | gene_id “DSRCT_NG3”; transcript_id |
| | | | | | “DSRCT_NG3.2”; exon “3”; |
| chr1 | exon | 221389466 | 221396611 | + | gene_id “DSRCT_NG3”; transcript_id |
| | | | | | “DSRCT_NG3.2”; exon “4”; |
| chr1 | transcript | 231296969 | 231297603 | + | gene_id “DSRCT_NG4”; transcript_id |
| | | | | | “DSRCT_NG4.1”; RPKM “5.4594”; cov |
| | | | | | “74.7023”; |
| chr1 | exon | 231296969 | 231297603 | + | gene_id “DSRCT_NG4”; transcript_id |
| | | | | | “DSRCT_NG4.1”; exon “1”; |
| chr10 | transcript | 3318786 | 3320209 | − | gene_id “DSRCT_NG23”; transcript_id |
| | | | | | “DSRCT_NG23.1”; RPKM “1.6156”; cov |
| | | | | | “22.1066”; |
| chr10 | exon | 3318786 | 3320209 | − | gene_id “DSRCT_NG23”; transcript_id |
| | | | | | “DSRCT_NG23.1”; exon “1”; |
| chr10 | transcript | 3328726 | 3338019 | − | gene_id “DSRCT_NG24”; transcript_id |
| | | | | | “DSRCT_NG24.1”; RPKM “7.3306”; cov |
| | | | | | “100.3057”; |
| chr10 | exon | 3328726 | 3329780 | − | gene_id “DSRCT_NG24”; transcript_id |
| | | | | | “DSRCT_NG24.1”; exon “1”; |
| chr10 | exon | 3337014 | 3338019 | − | gene_id “DSRCT_NG24”; transcript_id |
| | | | | | “DSRCT_NG24.1”; exon “2”; |
| chr10 | transcript | 3328726 | 3338019 | − | gene_id “DSRCT_NG24”; transcript_id |
| | | | | | “DSRCT_NG24.2”; RPKM “18.6396”; |
| | | | | | cov “255.0492”; |
| chr10 | exon | 3328726 | 3338019 | − | gene_id “DSRCT_NG24”; transcript_id |
| | | | | | “DSRCT_NG24.2”; exon “1”; |
| chr10 | transcript | 3328726 | 3338019 | − | gene_id “DSRCT_NG24”; transcript_id |
| | | | | | “DSRCT_NG24.3”; RPKM “4.4747”; cov |
| | | | | | “61.2285”; |
| chr10 | exon | 3328726 | 3329780 | − | gene_id “DSRCT_NG24”; transcript_id |
| | | | | | “DSRCT_NG24.3”; exon “1”; |
| chr10 | exon | 3330392 | 3338019 | − | gene_id “DSRCT_NG24”; transcript_id |
| | | | | | “DSRCT_NG24.3”; exon “2”; |
| chr11 | transcript | 1371528 | 1388488 | + | gene_id “DSRCT_NG25”; transcript_id |
| | | | | | “DSRCT_NG25.1”; RPKM “2.5051”; cov |
| | | | | | “34.2780”; |
| chr11 | exon | 1371528 | 1371992 | + | gene_id “DSRCT_NG25”; transcript_id |
| | | | | | “DSRCT_NG25.1”; exon “1”; |
| chr11 | exon | 1381945 | 1387003 | + | gene_id “DSRCT_NG25”; transcript_id |
| | | | | | “DSRCT_NG25.1”; exon “2”; |
| chr11 | exon | 1387332 | 1388488 | + | gene_id “DSRCT_NG25”; transcript_id |
| | | | | | “DSRCT_NG25.1”; exon “3”; |
| chr11 | transcript | 1371528 | 1388488 | + | gene_id “DSRCT_NG25”; transcript_id |
| | | | | | “DSRCT_NG25.2”; RPKM “2.6761”; cov |
| | | | | | “36.6182”; |
| chr11 | exon | 1371528 | 1371992 | + | gene_id “DSRCT_NG25”; transcript_id |
| | | | | | “DSRCT_NG25.2”; exon “1”; |
| chr11 | exon | 1381945 | 1388488 | + | gene_id “DSRCT_NG25”; transcript_id |
| | | | | | “DSRCT_NG25.2”; exon “2”; |
| chr11 | transcript | 68435596 | 68438308 | − | gene_id “DSRCT_NG26”; transcript_id |
| | | | | | “DSRCT_NG26.1”; RPKM “0.7566”; cov |
| | | | | | “10.3533”; |
| chr11 | exon | 68435596 | 68436309 | − | gene_id “DSRCT_NG26”; transcript_id |
| | | | | | “DSRCTJMG26.1”; exon “1”; |
| chr11 | exon | 68436644 | 68436839 | − | gene_id “DSRCT_NG26”; transcript_id |
| | | | | | “DSRCT_NG26.1”; exon “2”; |
| chr11 | exon | 68437321 | 68437374 | − | gene_id “DSRCT_NG26”; transcript_id |
| | | | | | “DSRCT_NG26.1”; exon “3”; |
| chr11 | exon | 68437481 | 68438308 | − | gene_id “DSRCT_NG26”; transcript_id |
| | | | | | “DSRCT_NG26.1”; exon “4”; |
| chr11 | transcript | 68435596 | 68438308 | − | gene_id “DSRCT_NG26”; transcript_id |
| | | | | | “DSRCT_NG26.2”; RPKM “2.0863”; cov |
| | | | | | “28.5468”; |
| chr11 | exon | 68435596 | 68436309 | − | gene_id “DSRCT_NG26”; transcript_id |
| | | | | | “DSRCT_NG26.2”; exon “1”; |
| chr11 | exon | 68436644 | 68436839 | − | gene_id “DSRCT_NG26”; transcript_id |
| | | | | | “DSRCT_NG26.2”; exon “2”; |
| chr11 | exon | 68437321 | 68438308 | − | gene_id “DSRCT_NG26”; transcript_id |
| | | | | | “DSRCT_NG26.2”; exon “3”; |
| chr11 | transcript | 68435596 | 68438308 | − | gene_id “DSRCT_NG26”; transcript_id |
| | | | | | “DSRCT_NG26.3”; RPKM “2.7082”; cov |
| | | | | | “37.0574”; |
| chr11 | exon | 68435596 | 68436309 | − | gene_id “DSRCT_NG26”; transcript_id |
| | | | | | “DSRCT_NG26.3”; exon “1”; |
| chr11 | exon | 68436644 | 68438308 | − | gene_id “DSRCT_NG26”; transcript_id |
| | | | | | “DSRCT_NG26.3”; exon “2”; |
| chr11 | transcript | 68435596 | 68438308 | − | gene_id “DSRCT_NG26”; transcript_id |
| | | | | | “DSRCT_NG26.4”; RPKM “2.6560”; cov |
| | | | | | “36.3418”; |
| chr11 | exon | 68435596 | 68438308 | − | gene_id “DSRCT_NG26”; transcript_id |
| | | | | | “DSRCT_NG26.4”; exon “1”; |
| chr15 | transcript | 98227047 | 98233708 | + | gene_id “DSRCT_NG27”; transcript_id |
| | | | | | “DSRCT_NG27.1”; RPKM “0.8123”; cov |
| | | | | | “11.1145”; |
| chr15 | exon | 98227047 | 98227171 | + | gene_id “DSRCT_NG27”; transcript_id |
| | | | | | “DSRCT_NG27.1”; exon “1”; |
| chr15 | exon | 98233077 | 98233708 | + | gene_id “DSRCT_NG27”; transcript_id |
| | | | | | “DSRCT_NG27.1”; exon “2”; |
| chr15 | transcript | 98227047 | 98233708 | + | gene_id “DSRCT_NG27”; transcript_id |
| | | | | | “DSRCT_NG27.2”; RPKM “0.7749”; cov |
| | | | | | “10.6032”; |
| chr15 | exon | 98227047 | 98227171 | + | gene_id “DSRCT_NG27”; transcript_id |
| | | | | | “DSRCT_NG27.2”; exon “1”; |
| chr15 | exon | 98233072 | 98233708 | + | gene_id “DSRCT_NG27”; transcript_id |
| | | | | | “DSRCT_NG27.2”; exon “2”; |
| chr15 | transcript | 98227047 | 98233708 | + | gene_id “DSRCT_NG27”; transcript_id |
| | | | | | “DSRCT_NG27.3”; RPKM “24.7998”; |
| | | | | | cov “339.3390”; |
| chr15 | exon | 98227047 | 98227171 | + | gene_id “DSRCT_NG27”; transcript_id |
| | | | | | “DSRCT_NG27.3”; exon “1”; |
| chr15 | exon | 98232944 | 98233708 | + | gene_id “DSRCT_NG27”; transcript_id |
| | | | | | “DSRCT_NG27.3”; exon “2”; |
| chr16 | transcript | 86149446 | 86154409 | − | gene_id “DSRCT_NG28”; transcript_id |
| | | | | | “DSRCT_NG28.1”; RPKM “1.9972”; cov |
| | | | | | “27.3286”; |
| chr16 | exon | 86149446 | 86154409 | − | gene_id “DSRCT_NG28”; transcript_id |
| | | | | | “DSRCT_NG28.1”; exon “1”; |
| chr16 | transcript | 86149446 | 86156729 | − | gene_id “DSRCT_NG28”; transcript_id |
| | | | | | “DSRCT_NG28.2”; RPKM “1.5067”; cov |
| | | | | | “20.6168”; |
| chr16 | exon | 86149446 | 86151884 | − | gene_id “DSRCT_NG28”; transcript_id |
| | | | | | “DSRCT_NG28.2”; exon “1”; |
| chr16 | exon | 86155828 | 86156729 | − | gene_id “DSRCT_NG28”; transcript_id |
| | | | | | “DSRCT_NG28.2”; exon “2”; |
| chr16 | transcript | 86151184 | 86163138 | + | gene_id “DSRCT_NG29”; transcript_id |
| | | | | | “DSRCT_NG29.1”; RPKM “0.7455”; cov |
| | | | | | “10.2011”; |
| chr16 | exon | 86151184 | 86151602 | + | gene_id “DSRCT_NG29”; transcript_id |
| | | | | | “DSRCT_NG29.1”; exon “1”; |
| chr16 | exon | 86155402 | 86155431 | + | gene_id “DSRCT_NG29”; transcript_id |
| | | | | | “DSRCT_NG29.1”; exon “2”; |
| chr16 | exon | 86155547 | 86163138 | + | gene_id “DSRCT_NG29”; transcript_id |
| | | | | | “DSRCT_NG29.1”; exon “3”; |
| chr19 | transcript | 1697219 | 1744600 | − | gene_id “DSRCT_NG30”; transcript_id |
| | | | | | “DSRCT_NG30.15”; RPKM “3.5576”; |
| | | | | | cov “48.6786”; |
| chr19 | exon | 1697219 | 1706398 | − | gene_id “DSRCT_NG30”; transcript_id |
| | | | | | “DSRCT_NG30.15”; exon “1”; |
| chr19 | exon | 1707363 | 1707460 | − | gene_id “DSRCT_NG30”; transcript_id |
| | | | | | “DSRCT_NG30.15”; exon “2”; |
| chr19 | exon | 1708336 | 1708505 | − | gene_id “DSRCT_NG30”; transcript_id |
| | | | | | “DSRCT_NG30.15”; exon “3”; |
| chr19 | exon | 1738954 | 1739048 | − | gene_id “DSRCT_NG30”; transcript_id |
| | | | | | “DSRCT_NG30.15”; exon “4”; |
| chr19 | exon | 1744334 | 1744600 | − | gene_id “DSRCT_NG30”; transcript_id |
| | | | | | “DSRCT_NG30.15”; exon “5”; |
| chr19 | transcript | 1697219 | 1744600 | − | gene_id “DSRCT_NG30”; transcript_id |
| | | | | | “DSRCT_NG30.14”; RPKM “4.6634”; |
| | | | | | cov “63.8098”; |
| chr19 | exon | 1697219 | 1697386 | − | gene_id “DSRCT_NG30”; transcript_id |
| | | | | | “DSRCT_NG30.14”; exon “1”; |
| chr19 | exon | 1698169 | 1706398 | − | gene_id “DSRCT_NG30”; transcript_id |
| | | | | | “DSRCT_NG30.14”; exon “2”; |
| chr19 | exon | 1707363 | 1707460 | − | gene_id “DSRCT_NG30”; transcript_id |
| | | | | | “DSRCT_NG30.14”; exon “3”; |
| chr19 | exon | 1708336 | 1708505 | − | gene_id “DSRCT_NG30”; transcript_id |
| | | | | | “DSRCT_NG30.14”; exon “4”; |
| chr19 | exon | 1738954 | 1739048 | − | gene_id “DSRCT_NG30”; transcript_id |
| | | | | | “DSRCT_NG30.14”; exon “5”; |
| chr19 | exon | 1744334 | 1744600 | − | gene_id “DSRCT_NG30”; transcript_id |
| | | | | | “DSRCT_NG30.14”; exon “6”; |
| chr19 | transcript | 1725870 | 1727366 | − | gene_id “DSRCT_NG30”; transcript_id |
| | | | | | “DSRCT_NG30.1”; RPKM “2.7999”; cov |
| | | | | | “38.3120”; |
| chr19 | exon | 1725870 | 1727366 | − | gene_id “DSRCT_NG30”; transcript_id |
| | | | | | “DSRCT_NG30.1”; exon “1”; |
| chr19 | transcript | 1725870 | 1744600 | − | gene_id “DSRCT_NG30”; transcript_id |
| | | | | | “DSRCT_NG30.10”; RPKM “1.0972”; |
| | | | | | cov “15.0133”; |
| chr19 | exon | 1725870 | 1726170 | − | gene_id “DSRCT_NG30”; transcript_id |
| | | | | | “DSRCT_NG30.10”; exon “1”; |
| chr19 | exon | 1738954 | 1739048 | − | gene_id “DSRCT_NG30”; transcript_id |
| | | | | | “DSRCT_NG30.10”; exon “2”; |
| chr19 | exon | 1740185 | 1740327 | − | gene_id “DSRCT_NG30”; transcript_id |
| | | | | | “DSRCT_NG30.10”; exon “3”; |
| chr19 | exon | 1744334 | 1744600 | − | gene_id “DSRCT_NG30”; transcript_id |
| | | | | | “DSRCT_NG30.10”; exon “4”; |
| chr19 | transcript | 1733413 | 1744600 | − | gene_id “DSRCT_NG30”; transcript_id |
| | | | | | “DSRCT_NG30.4”; RPKM “1.7305”; cov |
| | | | | | “23.6791”; |
| chr19 | exon | 1733413 | 1737515 | − | gene_id “DSRCT_NG30”; transcript_id |
| | | | | | “DSRCT_NG30.4”; exon “1”; |
| chr19 | exon | 1738954 | 1739048 | − | gene_id “DSRCT_NG30”; transcript_id |
| | | | | | “DSRCT_NG30.4”; exon “2”; |
| chr19 | exon | 1744334 | 1744600 | − | gene_id “DSRCT_NG30”; transcript_id |
| | | | | | “DSRCT_NG30.4”; exon “3”; |
| chr19 | transcript | 1733413 | 1744600 | − | gene_id “DSRCT_NG30”; transcript_id |
| | | | | | “DSRCT_NG30.5”; RPKM “1.4668”; cov |
| | | | | | “20.0707”; |
| chr19 | exon | 1733413 | 1739048 | − | gene_id “DSRCT_NG30”; transcript_id |
| | | | | | “DSRCT_NG30.5”; exon “1”; |
| chr19 | exon | 1740177 | 1740327 | − | gene_id “DSRCT_NG30”; transcript_id |
| | | | | | “DSRCT_NG30.5”; exon “2”; |
| chr19 | exon | 1744334 | 1744600 | − | gene_id “DSRCT_NG30”; transcript_id |
| | | | | | “DSRCT_NG30.5”; exon “3”; |
| chr19 | transcript | 1733413 | 1744600 | − | gene_id “DSRCT_NG30”; transcript_id |
| | | | | | “DSRCT_NG30.6”; RPKM “1.2835”; cov |
| | | | | | “17.5619”; |
| chr19 | exon | 1733413 | 1736229 | − | gene_id “DSRCT_NG30”; transcript_id |
| | | | | | “DSRCT_NG30.6”; exon “1”; |
| chr19 | exon | 1738954 | 1739048 | − | gene_id “DSRCT_NG30”; transcript_id |
| | | | | | “DSRCT_NG30.6”; exon “2”; |
| chr19 | exon | 1740177 | 1740327 | − | gene_id “DSRCT_NG30”; transcript_id |
| | | | | | “DSRCT_NG30.6”; exon “3”; |
| chr19 | exon | 1744334 | 1744600 | − | gene_id “DSRCT_NG30”; transcript_id |
| | | | | | “DSRCT_NG30.6”; exon “4”; |
| chr19 | transcript | 1733413 | 1744600 | − | gene_id “DSRCT_NG30”; transcript_id |
| | | | | | “DSRCT_NG30.7”; RPKM “1.7106”; cov |
| | | | | | “23.4064”; |
| chr19 | exon | 1733413 | 1737515 | − | gene_id “DSRCT_NG30”; transcript_id |
| | | | | | “DSRCT_NG30.7”; exon “1”; |
| chr19 | exon | 1738954 | 1739048 | − | gene_id “DSRCT_NG30”; transcript_id |
| | | | | | “DSRCT_NG30.7”; exon “2”; |
| chr19 | exon | 1740177 | 1740327 | − | gene_id “DSRCT_NG30”; transcript_id |
| | | | | | “DSRCT_NG30.7”; exon “3”; |
| chr19 | exon | 1744334 | 1744600 | − | gene_id “DSRCT_NG30”; transcript_id |
| | | | | | “DSRCT_NG30.7”; exon “4”; |
| chr19 | transcript | 1733413 | 1744600 | − | gene_id “DSRCT_NG30”; transcript_id |
| | | | | | “DSRCT_NG30.13”; RPKM “7.0674”; |
| | | | | | cov “96.7040”; |
| chr19 | exon | 1733413 | 1737515 | − | gene_id “DSRCT_NG30”; transcript_id |
| | | | | | “DSRCT_NG30.13”; exon “1”; |
| chr19 | exon | 1738954 | 1739048 | − | gene_id “DSRCT_NG30”; transcript_id |
| | | | | | “DSRCT_NG30.13”; exon “2”; |
| chr19 | exon | 1740185 | 1740327 | − | gene_id “DSRCT_NG30”; transcript_id |
| | | | | | “DSRCT_NG30.13”; exon “3”; |
| chr19 | exon | 1744334 | 1744600 | − | gene_id “DSRCT_NG30”; transcript_id |
| | | | | | “DSRCT_NG30.13”; exon “4”; |
| chr19 | transcript | 1733413 | 1744600 | − | gene_id “DSRCT_NG30”; transcript_id |
| | | | | | “DSRCT_NG30.12”; RPKM “1.0329”; |
| | | | | | cov “14.1336”; |
| chr19 | exon | 1733413 | 1737515 | − | gene_id “DSRCT_NG30”; transcript_id |
| | | | | | “DSRCT_NG30.12”; exon “1”; |
| chr19 | exon | 1738954 | 1739048 | − | gene_id “DSRCT_NG30”; transcript_id |
| | | | | | “DSRCT_NG30.12”; exon “2”; |
| chr19 | exon | 1740185 | 1740327 | − | gene_id “DSRCT_NG30”; transcript_id |
| | | | | | “DSRCT_NG30.12”; exon “3”; |
| chr19 | exon | 1743263 | 1744600 | − | gene_id “DSRCT_NG30”; transcript_id |
| | | | | | “DSRCT_NG30.12”; exon “4”; |
| chr19 | transcript | 1733413 | 1744600 | − | gene_id “DSRCT_NG30”; transcript_id |
| | | | | | “DSRCT_NG30.11”; RPKM “3.1202”; |
| | | | | | cov “42.6940”; |
| chr19 | exon | 1733413 | 1739048 | − | gene_id “DSRCT_NG30”; transcript_id |
| | | | | | “DSRCT_NG30.11”; exon “1”; |
| chr19 | exon | 1740185 | 1740327 | − | gene_id “DSRCT_NG30”; transcript_id |
| | | | | | “DSRCT_NG30.11”; exon “2”; |
| chr19 | exon | 1744334 | 1744600 | − | gene_id “DSRCT_NG30”; transcript_id |
| | | | | | “DSRCT_NG30.11”; exon “3”; |
| chr19 | transcript | 1733413 | 1744600 | − | gene_id “DSRCT_NG30”; transcript_id |
| | | | | | “DSRCT_NG30.3”; RPKM “1.9927”; cov |
| | | | | | “27.2660”; |
| chr19 | exon | 1733413 | 1736229 | − | gene_id “DSRCT_NG30”; transcript_id |
| | | | | | “DSRCT_NG30.3”; exon “1”; |
| chr19 | exon | 1738954 | 1739048 | − | gene_id “DSRCT_NG30”; transcript_id |
| | | | | | “DSRCT_NG30.3”; exon “2”; |
| chr19 | exon | 1744334 | 1744600 | − | gene_id “DSRCT_NG30”; transcript_id |
| | | | | | “DSRCT_NG30.3”; exon “3”; |
| chr19 | transcript | 1733413 | 1744600 | − | gene_id “DSRCT_NG30”; transcript_id |
| | | | | | “DSRCT_NG30.2”; RPKM “1.2488”; cov |
| | | | | | “17.0879”; |
| chr19 | exon | 1733413 | 1739048 | − | gene_id “DSRCT_NG30”; transcript_id |
| | | | | | “DSRCT_NG30.2”; exon “1”; |
| chr19 | exon | 1744334 | 1744600 | − | gene_id “DSRCT_NG30”; transcript_id |
| | | | | | “DSRCT_NG30.2”; exon “2”; |
| chr19 | transcript | 1733413 | 1744600 | − | gene_id “DSRCT_NG30”; transcript_id |
| | | | | | “DSRCT_NG30.9”; RPKM “3.4540”; cov |
| | | | | | “47.2619”; |
| chr19 | exon | 1733413 | 1736229 | − | gene_id “DSRCT_NG30”; transcript_id |
| | | | | | “DSRCT_NG30.9”; exon “1”; |
| chr19 | exon | 1738954 | 1739048 | − | gene_id “DSRCT_NG30”; transcript_id |
| | | | | | “DSRCT_NG30.9”; exon “2”; |
| chr19 | exon | 1740185 | 1740327 | − | gene_id “DSRCT_NG30”; transcript_id |
| | | | | | “DSRCT_NG30.9”; exon “3”; |
| chr19 | exon | 1744334 | 1744600 | − | gene_id “DSRCT_NG30”; transcript_id |
| | | | | | “DSRCT_NG30.9”; exon “4”; |
| chr19 | transcript | 1744745 | 1745892 | − | gene_id “DSRCT_NG30”; transcript_id |
| | | | | | “DSRCT_NG30.8”; RPKM “0.8496”; cov |
| | | | | | “11.6254”; |
| chr19 | exon | 1744745 | 1744941 | − | gene_id “DSRCT_NG30”; transcript_id |
| | | | | | “DSRCT_NG30.8”; exon “1”; |
| chr19 | exon | 1745206 | 1745309 | − | gene_id “DSRCT_NG30”; transcript_id |
| | | | | | “DSRCT_NG30.8”; exon “2”; |
| chr19 | exon | 1745707 | 1745892 | − | gene_id “DSRCT_NG30”; transcript_id |
| | | | | | “DSRCT_NG30.8”; exon “3”; |
| chr19 | transcript | 1524034 | 1528426 | − | gene_id “DSRCT_NG31”; transcript_id |
| | | | | | “DSRCT_NG31.1”; RPKM “25.2127”; |
| | | | | | cov “344.9894”; |
| chr19 | exon | 1524034 | 1528426 | − | gene_id “DSRCT_NG31”; transcript_id |
| | | | | | “DSRCT_NG31.1”; exon “1”; |
| chr2 | transcript | 74791316 | 74837588 | + | gene_id “DSRCT_NG5”; transcript_id |
| | | | | | “DSRCT_NG5.1”; RPKM “0.7311”; cov |
| | | | | | “10.0042”; |
| chr2 | exon | 74791316 | 74791490 | + | gene_id “DSRCT_NG5”; transcript_id |
| | | | | | “DSRCT_NG5.1”; exon “1”; |
| chr2 | exon | 74837320 | 74837588 | + | gene_id “DSRCT_NG5”; transcript_id |
| | | | | | “DSRCT_NG5.1”; exon “2”; |
| chr2 | transcript | 103978490 | 104042949 | + | gene_id “DSRCT_NG6”; transcript_id |
| | | | | | “DSRCT_NG6.7”; RPKM “0.7836”; cov |
| | | | | | “10.7224”; |
| chr2 | exon | 103978490 | 103979070 | + | gene_id “DSRCT_NG6”; transcript_id |
| | | | | | “DSRCT_NG6.7”; exon “1”; |
| chr2 | exon | 104019436 | 104019546 | + | gene_id “DSRCT_NG6”; transcript_id |
| | | | | | “DSRCT_NG6.7”; exon “2”; |
| chr2 | exon | 104021412 | 104021484 | + | gene_id “DSRCT_NG6”; transcript_id |
| | | | | | “DSRCT_NG6.7”; exon “3”; |
| chr2 | exon | 104026652 | 104042949 | + | gene_id “DSRCT_NG6”; transcript_id |
| | | | | | “DSRCT_NG6.7”; exon “4”; |
| chr2 | transcript | 103978490 | 104042949 | + | gene_id “DSRCT_NG6”; transcript_id |
| | | | | | “DSRCT_NG6.6”; RPKM “0.8644”; cov |
| | | | | | “11.8277”; |
| chr2 | exon | 103978490 | 103979070 | + | gene_id “DSRCT_NG6”; transcript_id |
| | | | | | “DSRCT_NG6.6”; exon “1”; |
| chr2 | exon | 104019431 | 104019546 | + | gene_id “DSRCT_NG6”; transcript_id |
| | | | | | “DSRCT_NG6.6”; exon “2”; |
| chr2 | exon | 104021412 | 104021484 | + | gene_id “DSRCT_NG6”; transcript_id |
| | | | | | “DSRCT_NG6.6”; exon “3”; |
| chr2 | exon | 104027078 | 104042949 | + | gene_id “DSRCT_NG6”; transcript_id |
| | | | | | “DSRCT_NG6.6”; exon “4”; |
| chr2 | transcript | 103978490 | 104042949 | + | gene_id “DSRCT_NG6”; transcript_id |
| | | | | | “DSRCT_NG6.5”; RPKM “8.5436”; cov |
| | | | | | “116.9036”; |
| chr2 | exon | 103978490 | 103979070 | + | gene_id “DSRCT_NG6”; transcript_id |
| | | | | | “DSRCT_NG6.5”; exon “1”; |
| chr2 | exon | 103995841 | 103995896 | + | gene_id “DSRCT_NG6”; transcript_id |
| | | | | | “DSRCT_NG6.5”; exon “2”; |
| chr2 | exon | 104019436 | 104019546 | + | gene_id “DSRCT_NG6”; transcript_id |
| | | | | | “DSRCT_NG6.5”; exon “3”; |
| chr2 | exon | 104021412 | 104042949 | + | gene_id “DSRCT_NG6”; transcript_id |
| | | | | | “DSRCT_NG6.5”; exon “4”; |
| chr2 | transcript | 103978490 | 104042949 | + | gene_id “DSRCT_NG6”; transcript_id |
| | | | | | “DSRCT_NG6.4”; RPKM “9.5843”; cov |
| | | | | | “131.1431”; |
| chr2 | exon | 103978490 | 103979070 | + | gene_id “DSRCT_NG6”; transcript_id |
| | | | | | “DSRCT_NG6.4”; exon “1”; |
| chr2 | exon | 103995841 | 103995896 | + | gene_id “DSRCT_NG6”; transcript_id |
| | | | | | “DSRCT_NG6.4”; exon “2”; |
| chr2 | exon | 104019436 | 104019546 | + | gene_id “DSRCT_NG6”; transcript_id |
| | | | | | “DSRCT_NG6.4”; exon “3”; |
| chr2 | exon | 104021412 | 104021484 | + | gene_id “DSRCT_NG6”; transcript_id |
| | | | | | “DSRCT_NG6.4”; exon “4”; |
| chr2 | exon | 104027078 | 104042949 | + | gene_id “DSRCT_NG6”; transcript_id |
| | | | | | “DSRCT_NG6.4”; exon “5”; |
| chr2 | transcript | 103978490 | 104042949 | + | gene_id “DSRCT_NG6”; transcript_id |
| | | | | | “DSRCT_NG6.3”; RPKM “3.0324”; cov |
| | | | | | “41.4926”; |
| chr2 | exon | 103978490 | 103979070 | + | gene_id “DSRCT_NG6”; transcript_id |
| | | | | | “DSRCT_NG6.3”; exon “1”; |
| chr2 | exon | 104019436 | 104019546 | + | gene_id “DSRCT_NG6”; transcript_id |
| | | | | | “DSRCT_NG6.3”; exon “2”; |
| chr2 | exon | 104021412 | 104021484 | + | gene_id “DSRCT_NG6”; transcript_id |
| | | | | | “DSRCT_NG6.3”; exon “3”; |
| chr2 | exon | 104027078 | 104042949 | + | gene_id “DSRCT_NG6”; transcript_id |
| | | | | | “DSRCT_NG6.3”; exon “4”; |
| chr2 | transcript | 103978490 | 104042949 | + | gene_id “DSRCT_NG6”; transcript_id |
| | | | | | “DSRCT_NG6.2”; RPKM “1.2526”; cov |
| | | | | | “17.1401”; |
| chr2 | exon | 103978490 | 103979070 | + | gene_id “DSRCT_NG6”; transcript_id |
| | | | | | “DSRCT_NG6.2”; exon “1”; |
| chr2 | exon | 104019436 | 104019546 | + | gene_id “DSRCT_NG6”; transcript_id |
| | | | | | “DSRCT_NG6.2”; exon “2”; |
| chr2 | exon | 104021412 | 104021484 | + | gene_id “DSRCT_NG6”; transcript_id |
| | | | | | “DSRCT_NG6.2”; exon “3”; |
| chr2 | exon | 104026297 | 104042949 | + | gene_id “DSRCT_NG6”; transcript_id |
| | | | | | “DSRCT_NG6.2”; exon “4”; |
| chr2 | transcript | 103978490 | 104042949 | + | gene_id “DSRCT_NG6”; transcript_id |
| | | | | | “DSRCT_NG6.1”; RPKM “0.8806”; cov |
| | | | | | “12.0494”; |
| chr2 | exon | 103978490 | 103979070 | + | gene_id “DSRCT_NG6”; transcript_id |
| | | | | | “DSRCT_NG6.1”; exon “1”; |
| chr2 | exon | 103995841 | 103995896 | + | gene_id “DSRCT_NG6”; transcript_id |
| | | | | | “DSRCT_NG6.1”; exon “2”; |
| chr2 | exon | 104019436 | 104042949 | + | gene_id “DSRCT_NG6”; transcript_id |
| | | | | | “DSRCT_NG6.1”; exon “3”; |
| chr2 | transcript | 119808116 | 119810916 | + | gene_id “DSRCT_NG7”; transcript_id |
| | | | | | “DSRCT_NG7.1”; RPKM “1.7067”; cov |
| | | | | | “23.3530”; |
| chr2 | exon | 119808116 | 119808455 | + | gene_id “DSRCT_NG7”; transcript_id |
| | | | | | “DSRCT_NG7.1”; exon “1”; |
| chr2 | exon | 119808654 | 119810916 | + | gene_id “DSRCT_NG7”; transcript_id |
| | | | | | “DSRCT_NG7.1”; exon “2”; |
| chr2 | transcript | 119808116 | 119810916 | + | gene_id “DSRCT_NG7”; transcript_id |
| | | | | | “DSRCT_NG7.2”; RPKM “1.6632”; cov |
| | | | | | “22.7584”; |
| chr2 | exon | 119808116 | 119808455 | + | gene_id “DSRCT_NG7”; transcript_id |
| | | | | | “DSRCT_NG7.2”; exon “1”; |
| chr2 | exon | 119808591 | 119810916 | + | gene_id “DSRCT_NG7”; transcript_id |
| | | | | | “DSRCT_NG7.2”; exon “2”; |
| chr2 | transcript | 119808116 | 119819342 | + | gene_id “DSRCT_NG7”; transcript_id |
| | | | | | “DSRCT_NG7.3”; RPKM “0.7416”; cov |
| | | | | | “10.1472”; |
| chr2 | exon | 119808116 | 119808455 | + | gene_id “DSRCT_NG7”; transcript_id |
| | | | | | “DSRCT_NG7.3”; exon “1”; |
| chr2 | exon | 119818971 | 119819342 | + | gene_id “DSRCT_NG7”; transcript_id |
| | | | | | “DSRCT_NG7.3”; exon “2”; |
| chr2 | transcript | 119808116 | 119821530 | + | gene_id “DSRCT_NG7”; transcript_id |
| | | | | | “DSRCT_NG7.4”; RPKM “1.0583”; cov |
| | | | | | “14.4808”; |
| chr2 | exon | 119808116 | 119808455 | + | gene_id “DSRCT_NG7”; transcript_id |
| | | | | | “DSRCT_NG7.4”; exon “1”; |
| chr2 | exon | 119818971 | 119819088 | + | gene_id “DSRCT_NG7”; transcript_id |
| | | | | | “DSRCT_NG7.4”; exon “2”; |
| chr2 | exon | 119821417 | 119821530 | + | gene_id “DSRCT_NG7”; transcript_id |
| | | | | | “DSRCT_NG7.4”; exon “3”; |
| chr2 | transcript | 233225399 | 233232735 | − | gene_id “DSRCT_NG8”; transcript_id |
| | | | | | “DSRCT_NG8.2”; RPKM “0.7340”; cov |
| | | | | | “10.0430”; |
| chr2 | exon | 233225399 | 233229389 | − | gene_id “DSRCT_NG8”; transcript_id |
| | | | | | “DSRCT_NG8.2”; exon “1”; |
| chr2 | exon | 233230020 | 233232735 | − | gene_id “DSRCT_NG8”; transcript_id |
| | | | | | “DSRCT_NG8.2”; exon “2”; |
| chr2 | transcript | 233225399 | 233232735 | − | gene_id “DSRCT_NG8”; transcript_id |
| | | | | | “DSRCT_NG8.3”; RPKM “4.9522”; cov |
| | | | | | “67.7611”; |
| chr2 | exon | 233225399 | 233227395 | − | gene_id “DSRCT_NG8”; transcript_id |
| | | | | | “DSRCT_NG8.3”; exon “1”; |
| chr2 | exon | 233229150 | 233229389 | − | gene_id “DSRCT_NG8”; transcript_id |
| | | | | | “DSRCT_NG8.3”; exon “2”; |
| chr2 | exon | 233230831 | 233232735 | − | gene_id “DSRCT_NG8”; transcript_id |
| | | | | | “DSRCT_NG8.3”; exon “3”; |
| chr2 | transcript | 233225399 | 233232735 | − | gene_id “DSRCT_NG8”; transcript_id |
| | | | | | “DSRCT_NG8.4”; RPKM “1.6400”; cov |
| | | | | | “22.4397”; |
| chr2 | exon | 233225399 | 233229389 | − | gene_id “DSRCT_NG8”; transcript_id |
| | | | | | “DSRCT_NG8.4”; exon “1”; |
| chr2 | exon | 233230831 | 233232735 | − | gene_id “DSRCT_NG8”; transcript_id |
| | | | | | “DSRCT_NG8.4”; exon “2”; |
| chr2 | transcript | 233225399 | 233232735 | − | gene_id “DSRCT_NG8”; transcript_id |
| | | | | | “DSRCT_NG8.1”; RPKM “1.7049”; cov |
| | | | | | “23.3277”; |
| chr2 | exon | 233225399 | 233232735 | − | gene_id “DSRCT_NG8”; transcript_id |
| | | | | | “DSRCT_NG8.1”; exon “1”; |
| chr21 | transcript | 46468246 | 46478349 | − | gene_id “DSRCT_NG32”; transcript_id |
| | | | | | “DSRCT_NG32.1”; RPKM “1.7334”; cov |
| | | | | | “23.7189”; |
| chr21 | exon | 46468246 | 46469914 | − | gene_id “DSRCT_NG32”; transcript_id |
| | | | | | “DSRCT_NG32.1”; exon “1”; |
| chr21 | exon | 46478093 | 46478349 | − | gene_id “DSRCT_NG32”; transcript_id |
| | | | | | “DSRCT_NG32.1”; exon “2”; |
| chr21 | transcript | 46468246 | 46478349 | − | gene_id “DSRCT_NG32”; transcript_id |
| | | | | | “DSRCT_NG32.2”; RPKM “0.9838”; cov |
| | | | | | “13.4609”; |
| chr21 | exon | 46468246 | 46473906 | − | gene_id “DSRCT_NG32”; transcript_id |
| | | | | | “DSRCT_NG32.2”; exon “1”; |
| chr21 | exon | 46473932 | 46478349 | − | gene_id “DSRCT_NG32”; transcript_id |
| | | | | | “DSRCT_NG32.2”; exon “2”; |
| chr21 | transcript | 46468246 | 46478349 | − | gene_id “DSRCT_NG32”; transcript_id |
| | | | | | “DSRCT_NG32.3”; RPKM “1.6527”; cov |
| | | | | | “22.6142”; |
| chr21 | exon | 46468246 | 46478349 | − | gene_id “DSRCT_NG32”; transcript_id |
| | | | | | “DSRCT_NG32.3”; exon “1”; |
| chr22 | transcript | 34996069 | 35029990 | − | gene_id “DSRCT_NG33”; transcript_id |
| | | | | | “DSRCT_NG33.3”; RPKM “10.2084”; |
| | | | | | cov “139.6836”; |
| chr22 | exon | 34996069 | 35009698 | − | gene_id “DSRCT_NG33”; transcript_id |
| | | | | | “DSRCT_NG33.3”; exon “1”; |
| chr22 | exon | 35022242 | 35025264 | − | gene_id “DSRCT_NG33”; transcript_id |
| | | | | | “DSRCT_NG33.3”; exon “2”; |
| chr22 | exon | 35029343 | 35029990 | − | gene_id “DSRCT_NG33”; transcript_id |
| | | | | | “DSRCT_NG33.3”; exon “3”; |
| chr22 | transcript | 34996069 | 35029990 | − | gene_id “DSRCT_NG33”; transcript_id |
| | | | | | “DSRCT_NG33.4”; RPKM “4.8531”; cov |
| | | | | | “66.4057”; |
| chr22 | exon | 34996069 | 35009698 | − | gene_id “DSRCT_NG33”; transcript_id |
| | | | | | “DSRCT_NG33.4”; exon “1”; |
| chr22 | exon | 35017288 | 35017570 | − | gene_id “DSRCT_NG33”; transcript_id |
| | | | | | “DSRCT_NG33.4”; exon “2”; |
| chr22 | exon | 35022242 | 35025264 | − | gene_id “DSRCT_NG33”; transcript_id |
| | | | | | “DSRCT_NG33.4”; exon “3”; |
| chr22 | exon | 35029343 | 35029990 | − | gene_id “DSRCT_NG33”; transcript_id |
| | | | | | “DSRCT_NG33.4”; exon “4”; |
| chr22 | transcript | 34996069 | 35018039 | − | gene_id “DSRCT_NG33”; transcript_id |
| | | | | | “DSRCT_NG33.1”; RPKM “1.6945”; cov |
| | | | | | “23.1865”; |
| chr22 | exon | 34996069 | 35018039 | − | gene_id “DSRCT_NG33”; transcript_id |
| | | | | | “DSRCT_NG33.1”; exon “1”; |
| chr22 | transcript | 35018113 | 35029990 | − | gene_id “DSRCT_NG33”; transcript_id |
| | | | | | “DSRCT_NG33.2”; RPKM “0.8066”; cov |
| | | | | | “11.0366”; |
| chr22 | exon | 35018113 | 35022935 | − | gene_id “DSRCT_NG33”; transcript_id |
| | | | | | “DSRCT_NG33.2”; exon “1”; |
| chr22 | exon | 35029343 | 35029990 | − | gene_id “DSRCT_NG33”; transcript_id |
| | | | | | “DSRCT_NG33.2”; exon “2”; |
| chr22 | transcript | 35018113 | 35029990 | − | gene_id “DSRCT_NG33”; transcript_id |
| | | | | | “DSRCT_NG33.5”; RPKM “2.4835”; cov |
| | | | | | “33.9821”; |
| chr22 | exon | 35018113 | 35025264 | − | gene_id “DSRCT_NG33”; transcript_id |
| | | | | | “DSRCT_NG33.5”; exon “1”; |
| chr22 | exon | 35029343 | 35029990 | − | gene_id “DSRCT_NG33”; transcript_id |
| | | | | | “DSRCT_NG33.5”; exon “2”; |
| chr22 | transcript | 49208801 | 49212288 | + | gene_id “DSRCT_NG34”; transcript_id |
| | | | | | “DSRCT_NG34.1”; RPKM “1.3134”; cov |
| | | | | | “17.9714”; |
| chr22 | exon | 49208801 | 49210781 | + | gene_id “DSRCT_NG34”; transcript_id |
| | | | | | “DSRCT_NG34.1”; exon “1”; |
| chr22 | exon | 49210822 | 49212288 | + | gene_id “DSRCT_NG34”; transcript_id |
| | | | | | “DSRCT_NG34.1”; exon “2”; |
| chr3 | transcript | 30029973 | 30123668 | − | gene_id “DSRCT_NG9”; transcript_id |
| | | | | | “DSRCT_NG9.1”; RPKM “0.9252”; cov |
| | | | | | “12.6592”; |
| chr3 | exon | 30029973 | 30030248 | − | gene_id “DSRCT_NG9”; transcript_id |
| | | | | | “DSRCT_NG9.1”; exon “1”; |
| chr3 | exon | 30031870 | 30031940 | − | gene_id “DSRCT_NG9”; transcript_id |
| | | | | | “DSRCT_NG9.1”; exon “2”; |
| chr3 | exon | 30122117 | 30122232 | − | gene_id “DSRCT_NG9”; transcript_id |
| | | | | | “DSRCT_NG9.1”; exon “3”; |
| chr3 | exon | 30123561 | 30123668 | − | gene_id “DSRCT_NG9”; transcript_id |
| | | | | | “DSRCT_NG9.1”; exon “4”; |
| chr5 | transcript | 1193219 | 1194599 | + | gene_id “DSRCT_NG10”; transcript_id |
| | | | | | “DSRCT_NG10.1”; RPKM “15.9992”; |
| | | | | | cov “218.9200”; |
| chr5 | exon | 1193219 | 1194599 | + | gene_id “DSRCT_NG10”; transcript_id |
| | | | | | “DSRCT_NG10.1”; exon “1”; |
| chr5 | transcript | 1867478 | 1878173 | + | gene_id “DSRCT_NG11”; transcript_id |
| | | | | | “DSRCT_NG11.1”; RPKM “9.1007”; cov |
| | | | | | “124.5265”; |
| chr5 | exon | 1867478 | 1878173 | + | gene_id “DSRCT_NG11”; transcript_id |
| | | | | | “DSRCT_NG11.1”; exon “1”; |
| chr6 | transcript | 15736183 | 15752850 | + | gene_id “DSRCT_NG12”; transcript_id |
| | | | | | “DSRCT_NG12.1”; RPKM “6.0068”; cov |
| | | | | | “82.1916”; |
| chr6 | exon | 15736183 | 15736264 | + | gene_id “DSRCT_NG12”; transcript_id |
| | | | | | “DSRCT_NG12.1”; exon “1”; |
| chr6 | exon | 15743656 | 15743700 | + | gene_id “DSRCT_NG12”; transcript_id |
| | | | | | “DSRCT_NG12.1”; exon “2”; |
| chr6 | exon | 15752293 | 15752850 | + | gene_id “DSRCT_NG12”; transcript_id |
| | | | | | “DSRCT_NG12.1”; exon “3”; |
| chr7 | transcript | 1377404 | 1404247 | − | gene_id “DSRCT_NG13”; transcript_id |
| | | | | | “DSRCT_NG13.2”; RPKM “0.7915”; cov |
| | | | | | “10.8306”; |
| chr7 | exon | 1377404 | 1388668 | − | gene_id “DSRCT_NG13”; transcript_id |
| | | | | | “DSRCT_NG13.2”; exon “1”; |
| chr7 | exon | 1403678 | 1404247 | − | gene_id “DSRCT_NG13”; transcript_id |
| | | | | | “DSRCT_NG13.2”; exon “2”; |
| chr7 | transcript | 1377404 | 1404247 | − | gene_id “DSRCT_NG13”; transcript_id |
| | | | | | “DSRCT_NG13.3”; RPKM “0.9096”; cov |
| | | | | | “12.4460”; |
| chr7 | exon | 1377404 | 1378670 | − | gene_id “DSRCT_NG13”; transcript_id |
| | | | | | “DSRCT_NG13.3”; exon “1”; |
| chr7 | exon | 1384724 | 1385133 | − | gene_id “DSRCT_NG13”; transcript_id |
| | | | | | “DSRCT_NG13.3”; exon “2”; |
| chr7 | exon | 1388467 | 1388668 | − | gene_id “DSRCT_NG13”; transcript_id |
| | | | | | “DSRCT_NG13.3”; exon “3”; |
| chr7 | exon | 1402665 | 1402827 | − | gene_id “DSRCT_NG13”; transcript_id |
| | | | | | “DSRCT_NG13.3”; exon “4”; |
| chr7 | exon | 1403678 | 1404247 | − | gene_id “DSRCT_NG13”; transcript_id |
| | | | | | “DSRCT_NG13.3”; exon “5”; |
| chr7 | transcript | 1398106 | 1404247 | − | gene_id “DSRCT_NG13”; transcript_id |
| | | | | | “DSRCT_NG13.1”; RPKM “2.2803”; cov |
| | | | | | “31.2013”; |
| chr7 | exon | 1398106 | 1402827 | − | gene_id “DSRCT_NG13”; transcript_id |
| | | | | | “DSRCT_NG13.1”; exon “1”; |
| chr7 | exon | 1403678 | 1404247 | − | gene_id “DSRCT_NG13”; transcript_id |
| | | | | | “DSRCT_NG13.1”; exon “2”; |
| chr7 | transcript | 1407280 | 1419292 | + | gene_id “DSRCT_NG14”; transcript_id |
| | | | | | “DSRCT_NG14.1”; RPKM “0.8518”; cov |
| | | | | | “11.6555”; |
| chr7 | exon | 1407280 | 1408593 | + | gene_id “DSRCT_NG14”; transcript_id |
| | | | | | “DSRCT_NG14.1”; exon “1”; |
| chr7 | exon | 1417089 | 1419292 | + | gene_id “DSRCT_NG14”; transcript_id |
| | | | | | “DSRCT_NG14.1”; exon “2”; |
| chr7 | transcript | 1407280 | 1413820 | + | gene_id “DSRCT_NG14”; transcript_id |
| | | | | | “DSRCT_NG14.3”; RPKM “0.7694”; cov |
| | | | | | “10.5282”; |
| chr7 | exon | 1407280 | 1408593 | + | gene_id “DSRCT_NG14”; transcript_id |
| | | | | | “DSRCT_NG14.3”; exon “1”; |
| chr7 | exon | 1410383 | 1413820 | + | gene_id “DSRCT_NG14”; transcript_id |
| | | | | | “DSRCT_NG14.3”; exon “2”; |
| chr7 | transcript | 1409045 | 1413820 | + | gene_id “DSRCT_NG14”; transcript_id |
| | | | | | “DSRCT_NG14.2”; RPKM “3.2007”; cov |
| | | | | | “43.7953”; |
| chr7 | exon | 1409045 | 1413820 | + | gene_id “DSRCT_NG14”; transcript_id |
| | | | | | “DSRCT_NG14.2”; exon “1”; |
| chr7 | transcript | 5878487 | 5887078 | − | gene_id “DSRCT_NG15”; transcript_id |
| | | | | | “DSRCT_NG15.1”; RPKM “1.2742”; cov |
| | | | | | “17.4355”; |
| chr7 | exon | 5878487 | 5880534 | − | gene_id “DSRCT_NG15”; transcript_id |
| | | | | | “DSRCT_NG15.1”; exon “1”; |
| chr7 | exon | 5886636 | 5887078 | − | gene_id “DSRCT_NG15”; transcript_id |
| | | | | | “DSRCT_NG15.1”; exon “2”; |
| chr7 | transcript | 131758168 | 131777037 | − | gene_id “DSRCT_NG16”; transcript_id |
| | | | | | “DSRCT_NG16.1”; RPKM “1.8583”; cov |
| | | | | | “25.4270”; |
| chr7 | exon | 131758168 | 131759334 | − | gene_id “DSRCT_NG16”; transcript_id |
| | | | | | “DSRCT_NG16.1”; exon “1”; |
| chr7 | exon | 131761158 | 131765750 | − | gene_id “DSRCT_NG16”; transcript_id |
| | | | | | “DSRCT_NG16.1”; exon “2”; |
| chr7 | exon | 131776730 | 131777037 | − | gene_id “DSRCT_NG16”; transcript_id |
| | | | | | “DSRCT_NG16.1”; exon “3”; |
| chr7 | transcript | 131758168 | 131777037 | − | gene_id “DSRCT_NG16”; transcript_id |
| | | | | | “DSRCT_NG16.2”; RPKM “2.1962”; cov |
| | | | | | “30.0511”; |
| chr7 | exon | 131758168 | 131765750 | − | gene_id “DSRCT_NG16”; transcript_id |
| | | | | | “DSRCT_NG16.2”; exon “1”; |
| chr7 | exon | 131776730 | 131777037 | − | gene_id “DSRCT_NG16”; transcript_id |
| | | | | | “DSRCT_NG16.2”; exon “2”; |
| chr7 | transcript | 152986290 | 152987100 | − | gene_id “DSRCT_NG17”; transcript_id |
| | | | | | “DSRCT_NG17.1”; RPKM “5.8472”; cov |
| | | | | | “80.0080”; |
| chr7 | exon | 152986290 | 152986596 | − | gene_id “DSRCT_NG17”; transcript_id |
| | | | | | “DSRCT_NG17.1”; exon “1”; |
| chr7 | exon | 152986682 | 152987100 | − | gene_id “DSRCT_NG17”; transcript_id |
| | | | | | “DSRCT_NG17.1”; exon “2”; |
| chr7 | transcript | 152986290 | 152987100 | − | gene_id “DSRCT_NG17”; transcript_id |
| | | | | | “DSRCT_NG17.2”; RPKM “1.6965”; cov |
| | | | | | “23.2136”; |
| chr7 | exon | 152986290 | 152987100 | − | gene_id “DSRCT_NG17”; transcript_id |
| | | | | | “DSRCT_NG17.2”; exon “1”; |
| chr8 | transcript | 25979059 | 25994825 | − | gene_id “DSRCT_NG18”; transcript_id |
| | | | | | “DSRCT_NG18.3”; RPKM “0.9659”; cov |
| | | | | | “13.2161”; |
| chr8 | exon | 25979059 | 25979858 | − | gene_id “DSRCT_NG18”; transcript_id |
| | | | | | “DSRCT_NG18.3”; exon “1”; |
| chr8 | exon | 25983085 | 25983243 | − | gene_id “DSRCT_NG18”; transcript_id |
| | | | | | “DSRCT_NG18.3”; exon “2”; |
| chr8 | exon | 25983746 | 25983916 | − | gene_id “DSRCT_NG18”; transcript_id |
| | | | | | “DSRCT_NG18.3”; exon “3”; |
| chr8 | exon | 25984629 | 25994825 | − | gene_id “DSRCT_NG18”; transcript_id |
| | | | | | “DSRCT_NG18.3”; exon “4”; |
| chr8 | transcript | 25979059 | 26055947 | − | gene_id “DSRCT_NG18”; transcript_id |
| | | | | | “DSRCT_NG18.4”; RPKM “0.9707”; cov |
| | | | | | “13.2816”; |
| chr8 | exon | 25979059 | 25979858 | − | gene_id “DSRCT_NG18”; transcript_id |
| | | | | | “DSRCT_NG18.4”; exon “1”; |
| chr8 | exon | 25983085 | 25983243 | − | gene_id “DSRCT_NG18”; transcript_id |
| | | | | | “DSRCT_NG18.4”; exon “2”; |
| chr8 | exon | 25983746 | 25983916 | − | gene_id “DSRCT_NG18”; transcript_id |
| | | | | | “DSRCT_NG18.4”; exon “3”; |
| chr8 | exon | 25984629 | 25994512 | − | gene_id “DSRCT_NG18”; transcript_id |
| | | | | | “DSRCT_NG18.4”; exon “4”; |
| chr8 | exon | 25999885 | 26000057 | − | gene_id “DSRCT_NG18”; transcript_id |
| | | | | | “DSRCT_NG18.4”; exon “5”; |
| chr8 | exon | 26048270 | 26048392 | − | gene_id “DSRCT_NG18”; transcript_id |
| | | | | | “DSRCT_NG18.4”; exon “6”; |
| chr8 | exon | 26055614 | 26055947 | − | gene_id “DSRCT_NG18”; transcript_id |
| | | | | | “DSRCT_NG18.4”; exon “7”; |
| chr8 | transcript | 26046318 | 26055947 | − | gene_id “DSRCT_NG18”; transcript_id |
| | | | | | “DSRCT_NG18.2”; RPKM “2.1028”; cov |
| | | | | | “28.7736”; |
| chr8 | exon | 26046318 | 26048392 | − | gene_id “DSRCT_NG18”; transcript_id |
| | | | | | “DSRCT_NG18.2”; exon “1”; |
| chr8 | exon | 26055614 | 26055947 | − | gene_id “DSRCT_NG18”; transcript_id |
| | | | | | “DSRCT_NG18.2”; exon “2”; |
| chr8 | transcript | 26054041 | 26055947 | − | gene_id “DSRCT_NG18”; transcript_id |
| | | | | | “DSRCT_NG18.1”; RPKM “4.3022”; cov |
| | | | | | “58.8673”; |
| chr8 | exon | 26054041 | 26055947 | − | gene_id “DSRCT_NG18”; transcript_id |
| | | | | | “DSRCT_NG18.1”; exon “1”; |
| chr8 | transcript | 116047600 | 116055631 | − | gene_id “DSRCT_NG19”; transcript_id |
| | | | | | “DSRCT_NG19.1”; RPKM “1.1066”; cov |
| | | | | | “15.1411”; |
| chr8 | exon | 116047600 | 116048248 | − | gene_id “DSRCT_NG19”; transcript_id |
| | | | | | “DSRCT_NG19.1”; exon “1”; |
| chr8 | exon | 116048445 | 116048588 | − | gene_id “DSRCT_NG19”; transcript_id |
| | | | | | “DSRCT_NG19.1”; exon “2”; |
| chr8 | exon | 116049816 | 116050126 | − | gene_id “DSRCT_NG19”; transcript_id |
| | | | | | “DSRCT_NG19.1”; exon “3”; |
| chr8 | exon | 116055449 | 116055631 | − | gene_id “DSRCT_NG19”; transcript_id |
| | | | | | “DSRCT_NG19.1”; exon “4”; |
| chr9 | transcript | 136356276 | 136371612 | + | gene_id “DSRCT_NG20”; transcript_id |
| | | | | | “DSRCT_NG20.1”; RPKM “9.6178”; cov |
| | | | | | “131.6026”; |
| chr9 | exon | 136356276 | 136356452 | + | gene_id “DSRCT_NG20”; transcript_id |
| | | | | | “DSRCT_NG20.1”; exon “1”; |
| chr9 | exon | 136356833 | 136360492 | + | gene_id “DSRCT_NG20”; transcript_id |
| | | | | | “DSRCT_NG20.1”; exon “2”; |
| chr9 | exon | 136364876 | 136364997 | + | gene_id “DSRCT_NG20”; transcript_id |
| | | | | | “DSRCT_NG20.1”; exon “3”; |
| chr9 | exon | 136365707 | 136371612 | + | gene_id “DSRCT_NG20”; transcript_id |
| | | | | | “DSRCT_NG20.1”; exon “4”; |
| chr9 | transcript | 136356276 | 136371612 | + | gene_id “DSRCT_NG20”; transcript_id |
| | | | | | “DSRCT_NG20.2”; RPKM “3.5503”; cov |
| | | | | | “48.5787”; |
| chr9 | exon | 136356276 | 136356610 | + | gene_id “DSRCT_NG20”; transcript_id |
| | | | | | “DSRCT_NG20.2”; exon “1”; |
| chr9 | exon | 136356833 | 136360492 | + | gene_id “DSRCT_NG20”; transcript_id |
| | | | | | “DSRCT_NG20.2”; exon “2”; |
| chr9 | exon | 136364876 | 136364997 | + | gene_id “DSRCT_NG20”; transcript_id |
| | | | | | “DSRCT_NG20.2”; exon “3”; |
| chr9 | exon | 136365707 | 136371612 | + | gene_id “DSRCT_NG20”; transcript_id |
| | | | | | “DSRCT_NG20.2”; exon “4”; |
| chr9 | transcript | 136356276 | 136371612 | + | gene_id “DSRCT_NG20”; transcript_id |
| | | | | | “DSRCT_NG20.3”; RPKM “10.4007”; |
| | | | | | cov “142.3147”; |
| chr9 | exon | 136356276 | 136356498 | + | gene_id “DSRCT_NG20”; transcript_id |
| | | | | | “DSRCT_NG20.3”; exon “1”; |
| chr9 | exon | 136356833 | 136360492 | + | gene_id “DSRCT_NG20”; transcript_id |
| | | | | | “DSRCT_NG20.3”; exon “2”; |
| chr9 | exon | 136364876 | 136364997 | + | gene_id “DSRCT_NG20”; transcript_id |
| | | | | | “DSRCT_NG20.3”; exon “3”; |
| chr9 | exon | 136365707 | 136371612 | + | gene_id “DSRCT_NG20”; transcript_id |
| | | | | | “DSRCT_NG20.3”; exon “4”; |
| chr9 | transcript | 136356276 | 136371612 | + | gene_id “DSRCT_NG20”; transcript_id |
| | | | | | “DSRCT_NG20.4”; RPKM “0.8778”; cov |
| | | | | | “12.0113”; |
| chr9 | exon | 136356276 | 136356498 | + | gene_id “DSRCT_NG20”; transcript_id |
| | | | | | “DSRCT_NG20.4”; exon “1”; |
| chr9 | exon | 136356833 | 136356948 | + | gene_id “DSRCT_NG20”; transcript_id |
| | | | | | “DSRCT_NG20.4”; exon “2”; |
| chr9 | exon | 136358438 | 136360492 | + | gene_id “DSRCT_NG20”; transcript_id |
| | | | | | “DSRCT_NG20.4”; exon “3”; |
| chr9 | exon | 136364876 | 136364997 | + | gene_id “DSRCT_NG20”; transcript_id |
| | | | | | “DSRCT_NG20.4”; exon “4”; |
| chr9 | exon | 136365707 | 136371612 | + | gene_id “DSRCT_NG20”; transcript_id |
| | | | | | “DSRCT_NG20.4”; exon “5”; |
| chr9 | transcript | 136356276 | 136371612 | + | gene_id “DSRCT_NG20”; transcript_id |
| | | | | | “DSRCT_NG20.5”; RPKM “1.0534”; cov |
| | | | | | “14.4136”; |
| chr9 | exon | 136356276 | 136356452 | + | gene_id “DSRCT_NG20”; transcript_id |
| | | | | | “DSRCT_NG20.5”; exon “1”; |
| chr9 | exon | 136356833 | 136356948 | + | gene_id “DSRCT_NG20”; transcript_id |
| | | | | | “DSRCT_NG20.5”; exon “2”; |
| chr9 | exon | 136358438 | 136360492 | + | gene_id “DSRCT_NG20”; transcript_id |
| | | | | | “DSRCT_NG20.5”; exon “3”; |
| chr9 | exon | 136364876 | 136364997 | + | gene_id “DSRCT_NG20”; transcript_id |
| | | | | | “DSRCT_NG20.5”; exon “4”; |
| chr9 | exon | 136365707 | 136371612 | + | gene_id “DSRCT_NG20”; transcript_id |
| | | | | | “DSRCT_NG20.5”; exon “5”; |
| chr9 | transcript | 136356276 | 136371612 | + | gene_id “DSRCT_NG20”; transcript_id |
| | | | | | “DSRCT_NG20.6”; RPKM “1.6491”; cov |
| | | | | | “22.5646”; |
| chr9 | exon | 136356276 | 136356498 | + | gene_id “DSRCT_NG20”; transcript_id |
| | | | | | “DSRCT_NG20.6”; exon “1”; |
| chr9 | exon | 136356833 | 136356948 | + | gene_id “DSRCT_NG20”; transcript_id |
| | | | | | “DSRCT_NG20.6”; exon “2”; |
| chr9 | exon | 136358438 | 136358534 | + | gene_id “DSRCT_NG20”; transcript_id |
| | | | | | “DSRCT_NG20.6”; exon “3”; |
| chr9 | exon | 136360173 | 136360492 | + | gene_id “DSRCT_NG20”; transcript_id |
| | | | | | “DSRCT_NG20.6”; exon “4”; |
| chr9 | exon | 136364876 | 136364997 | + | gene_id “DSRCT_NG20”; transcript_id |
| | | | | | “DSRCT_NG20.6”; exon “5”; |
| chr9 | exon | 136365707 | 136371612 | + | gene_id “DSRCT_NG20”; transcript_id |
| | | | | | “DSRCT_NG20.6”; exon “6”; |
| chr9 | transcript | 136356276 | 136371612 | + | gene_id “DSRCT_NG20”; transcript_id |
| | | | | | “DSRCT_NG20.7”; RPKM “3.1882”; cov |
| | | | | | “43.6246”; |
| chr9 | exon | 136356276 | 136356452 | + | gene_id “DSRCT_NG20”; transcript_id |
| | | | | | “DSRCT_NG20.7”; exon “1”; |
| chr9 | exon | 136356833 | 136356948 | + | gene_id “DSRCT_NG20”; transcript_id |
| | | | | | “DSRCT_NG20.7”; exon “2”; |
| chr9 | exon | 136358438 | 136358534 | + | gene_id “DSRCT_NG20”; transcript_id |
| | | | | | “DSRCT_NG20.7”; exon “3”; |
| chr9 | exon | 136360173 | 136360492 | + | gene_id “DSRCT_NG20”; transcript_id |
| | | | | | “DSRCT_NG20.7”; exon “4”; |
| chr9 | exon | 136364876 | 136364997 | + | gene_id “DSRCT_NG20”; transcript_id |
| | | | | | “DSRCT_NG20.7”; exon “5”; |
| chr9 | exon | 136365707 | 136371612 | + | gene_id “DSRCT_NG20”; transcript_id |
| | | | | | “DSRCT_NG20.7”; exon “6”; |
| chr9 | transcript | 136356276 | 136371612 | + | gene_id “DSRCT_NG20”; transcript_id |
| | | | | | “DSRCT_NG20.8”; RPKM “1.6490”; cov |
| | | | | | “22.5641”; |
| chr9 | exon | 136356276 | 136356610 | + | gene_id “DSRCT_NG20”; transcript_id |
| | | | | | “DSRCT_NG20.8”; exon “1”; |
| chr9 | exon | 136356833 | 136356948 | + | gene_id “DSRCT_NG20”; transcript_id |
| | | | | | “DSRCT_NG20.8”; exon “2”; |
| chr9 | exon | 136358438 | 136358534 | + | gene_id “DSRCT_NG20”; transcript_id |
| | | | | | “DSRCT_NG20.8”; exon “3”; |
| chr9 | exon | 136360173 | 136360492 | + | gene_id “DSRCT_NG20”; transcript_id |
| | | | | | “DSRCT_NG20.8”; exon “4”; |
| chr9 | exon | 136364876 | 136364997 | + | gene_id “DSRCT_NG20”; transcript_id |
| | | | | | “DSRCT_NG20.8”; exon “5”; |
| chr9 | exon | 136365707 | 136371612 | + | gene_id “DSRCT_NG20”; transcript_id |
| | | | | | “DSRCT_NG20.8”; exon “6”; |
| chr9 | transcript | 138495151 | 138500931 | − | gene_id “DSRCT_NG21”; transcript_id |
| | | | | | “DSRCT_NG21.1”; RPKM “0.9881”; cov |
| | | | | | “13.5208”; |
| chr9 | exon | 138495151 | 138495696 | − | gene_id “DSRCT_NG21”; transcript_id |
| | | | | | “DSRCT_NG21.1”; exon “1”; |
| chr9 | exon | 138495870 | 138495952 | − | gene_id “DSRCT_NG21”; transcript_id |
| | | | | | “DSRCT_NG21.1”; exon “2”; |
| chr9 | exon | 138500647 | 138500931 | − | gene_id “DSRCT_NG21”; transcript_id |
| | | | | | “DSRCT_NG21.1”; exon “3”; |
| chr9 | transcript | 138495151 | 138500931 | − | gene_id “DSRCT_NG21”; transcript_id |
| | | | | | “DSRCT_NG21.2”; RPKM “3.7878”; cov |
| | | | | | “51.8293”; |
| chr9 | exon | 138495151 | 138495696 | − | gene_id “DSRCT_NG21”; transcript_id |
| | | | | | “DSRCT_NG21.2”; exon “1”; |
| chr9 | exon | 138495870 | 138495952 | − | gene_id “DSRCT_NG21”; transcript_id |
| | | | | | “DSRCT_NG21.2”; exon “2”; |
| chr9 | exon | 138496856 | 138498324 | − | gene_id “DSRCT_NG21”; transcript_id |
| | | | | | “DSRCT_NG21.2”; exon “3”; |
| chr9 | exon | 138498650 | 138500931 | − | gene_id “DSRCT_NG21”; transcript_id |
| | | | | | “DSRCT_NG21.2”; exon “4”; |
| chr9 | transcript | 138495151 | 138500931 | − | gene_id “DSRCT_NG21”; transcript_id |
| | | | | | “DSRCT_NG21.3”; RPKM “18.2064”; |
| | | | | | cov “249.1209”; |
| chr9 | exon | 138495151 | 138495696 | − | gene_id “DSRCT_NG21”; transcript_id |
| | | | | | “DSRCT_NG21.3”; exon “1”; |
| chr9 | exon | 138495870 | 138495952 | − | gene_id “DSRCT_NG21”; transcript_id |
| | | | | | “DSRCT_NG21.3”; exon “2”; |
| chr9 | exon | 138496856 | 138500931 | − | gene_id “DSRCT_NG21”; transcript_id |
| | | | | | “DSRCT_NG21.3”; exon “3”; |
| chr9 | transcript | 138500941 | 138511703 | − | gene_id “DSRCT_NG21”; transcript_id |
| | | | | | “DSRCT_NG21.4”; RPKM “5.6489”; cov |
| | | | | | “77.2948”; |
| chr9 | exon | 138500941 | 138509226 | − | gene_id “DSRCT_NG21”; transcript_id |
| | | | | | “DSRCT_NG21.4”; exon “1”; |
| chr9 | exon | 138511488 | 138511703 | − | gene_id “DSRCT_NG21”; transcript_id |
| | | | | | “DSRCT_NG21.4”; exon “2”; |
| chr9 | transcript | 138500941 | 138511703 | − | gene_id “DSRCT_NG21”; transcript_id |
| | | | | | “DSRCT_NG21.5”; RPKM “2.8431”; cov |
| | | | | | “38.9020”; |
| chr9 | exon | 138500941 | 138508699 | − | gene_id “DSRCT_NG21”; transcript_id |
| | | | | | “DSRCT_NG21.5”; exon “1”; |
| chr9 | exon | 138508847 | 138511703 | − | gene_id “DSRCT_NG21”; transcript_id |
| | | | | | “DSRCT_NG21.5”; exon “2”; |
| chr9 | transcript | 141094206 | 141096020 | + | gene_id “DSRCT_NG22”; transcript_id |
| | | | | | “DSRCT_NG22.2”; RPKM “1.0558”; cov |
| | | | | | “14.4472”; |
| chr9 | exon | 141094206 | 141095399 | + | gene_id “DSRCT_NG22”; transcript_id |
| | | | | | “DSRCT_NG22.2”; exon “1”; |
| chr9 | exon | 141095572 | 141096020 | + | gene_id “DSRCT_NG22”; transcript_id |
| | | | | | “DSRCT_NG22.2”; exon “2”; |
| chr9 | transcript | 141097131 | 141105737 | + | gene_id “DSRCT_NG22”; transcript_id |
| | | | | | “DSRCT_NG22.1”; RPKM “2.1738”; cov |
| | | | | | “29.7445”; |
| chr9 | exon | 141097131 | 141105737 | + | gene_id “DSRCT_NG22”; transcript_id |
| | | | | | “DSRCT_NG22.1”; exon “1”; |
| chrX | transcript | 46062674 | 46067276 | + | gene_id “DSRCT_NG35”; transcript_id |
| | | | | | “DSRCT_NG35.1”; RPKM “4.4527”; cov |
| | | | | | “60.9276”; |
| chrX | exon | 46062674 | 46063184 | + | gene_id “DSRCT_NG35”; transcript_id |
| | | | | | “DSRCT_NG35.1”; exon “1”; |
| chrX | exon | 46067111 | 46067276 | + | gene_id “DSRCT_NG35”; transcript_id |
| | | | | | “DSRCT_NG35.1”; exon “2”; |
| chrX | transcript | 72094599 | 72151083 | + | gene_id “DSRCT_NG36”; transcript_id |
| | | | | | “DSRCT_NG36.1”; RPKM “4.4849”; cov |
| | | | | | “61.3680”; |
| chrX | exon | 72094599 | 72095190 | + | gene_id “DSRCT_NG36”; transcript_id |
| | | | | | “DSRCT_NG36.1”; exon “1”; |
| chrX | exon | 72150325 | 72151083 | + | gene_id “DSRCT_NG36”; transcript_id |
| | | | | | “DSRCT_NG36.1”; exon “2”; |
| chrX | transcript | 72094599 | 72135474 | + | gene_id “DSRCT_NG36”; transcript_id |
| | | | | | “DSRCT_NG36.2”; RPKM “9.9352”; cov |
| | | | | | “135.9456”; |
| chrX | exon | 72094599 | 72095190 | + | gene_id “DSRCT_NG36”; transcript_id |
| | | | | | “DSRCT_NG36.2”; exon “1”; |
| chrX | exon | 72131963 | 72135474 | + | gene_id “DSRCT_NG36”; transcript_id |
| | | | | | “DSRCT_NG36.2”; exon “2”; |
| chrX | transcript | 72154383 | 72160192 | − | gene_id “DSRCT_NG37”; transcript_id |
| | | | | | “DSRCT_NG37.2”; RPKM “2.4319”; cov |
| | | | | | “33.2755”; |
| chrX | exon | 72154383 | 72155867 | − | gene_id “DSRCT_NG37”; transcript_id |
| | | | | | “DSRCT_NG37.2”; exon “1”; |
| chrX | exon | 72156441 | 72156527 | − | gene_id “DSRCT_NG37”; transcript_id |
| | | | | | “DSRCT_NG37.2”; exon “2”; |
| chrX | exon | 72157185 | 72160192 | − | gene_id “DSRCT_NG37”; transcript_id |
| | | | | | “DSRCT_NG37.2”; exon “3”; |
| chrX | transcript | 72154383 | 72160192 | − | gene_id “DSRCT_NG37”; transcript_id |
| | | | | | “DSRCT_NG37.1”; RPKM “3.1563”; cov |
| | | | | | “43.1876”; |
| chrX | exon | 72154383 | 72155867 | − | gene_id “DSRCT_NG37”; transcript_id |
| | | | | | “DSRCT_NG37.1”; exon “1”; |
| chrX | exon | 72156441 | 72156527 | − | gene_id “DSRCT_NG37”; transcript_id |
| | | | | | “DSRCT_NG37.1”; exon “2”; |
| chrX | exon | 72157185 | 72157326 | − | gene_id “DSRCT_NG37”; transcript_id |
| | | | | | “DSRCT_NG37.1”; exon “3”; |
| chrX | exon | 72157425 | 72157531 | − | gene_id “DSRCT_NG37”; transcript_id |
| | | | | | “DSRCT_NG37.1”; exon “4”; |
| chrX | exon | 72157738 | 72160192 | − | gene_id “DSRCT_NG37”; transcript_id |
| | | | | | “DSRCT_NG37.1”; exon “5”; |
| chr1 | transcript | 48072017 | 48122983 | − | gene_id “NFAT_NG1”; transcript_id |
| | | | | | “NFAT_NG1.2”; RPKM “8.0423”; cov |
| | | | | | “111.3846”; |
| chr1 | exon | 48072017 | 48073614 | − | gene_id “NFAT_NG1”; transcript_id |
| | | | | | “NFAT_NG1.2”; exon “1”; |
| chr1 | exon | 48122775 | 48122983 | − | gene_id “NFAT_NG1”; transcript_id |
| | | | | | “NFAT_NG1.2”; exon “2”; |
| chr1 | transcript | 48119261 | 48122983 | − | gene_id “NFAT_NG1”; transcript_id |
| | | | | | “NFAT_NG1.1”; RPKM “3.1750”; cov |
| | | | | | “43.9732”; |
| chr1 | exon | 48119261 | 48122983 | − | gene_id “NFAT_NG1”; transcript_id |
| | | | | | “NFAT_NG1.1”; exon “1”; |
| chr1 | transcript | 105112322 | 105116825 | − | gene_id “NFAT_NG2”; transcript_id |
| | | | | | “NFAT_NG2.1”; RPKM “4.9522”; cov |
| | | | | | “68.5868”; |
| chr1 | exon | 105112322 | 105116825 | − | gene_id “NFAT_NG2”; transcript_id |
| | | | | | “NFAT_NG2.1”; exon “1”; |
| chr1 | transcript | 157350793 | 157360394 | + | gene_id “NFAT_NG3”; transcript_id |
| | | | | | “NFAT_NG3.1”; RPKM “2.7227”; cov |
| | | | | | “37.7090”; |
| chr1 | exon | 157350793 | 157351005 | + | gene_id “NFAT_NG3”; transcript_id |
| | | | | | “NFAT_NG3.1”; exon “1”; |
| chr1 | exon | 157353091 | 157353222 | + | gene_id “NFAT_NG3”; transcript_id |
| | | | | | “NFAT_NG3.1”; exon “2”; |
| chr1 | exon | 157360083 | 157360394 | + | gene_id “NFAT_NG3”; transcript_id |
| | | | | | “NFAT_NG3.1”; exon “3”; |
| chr1 | transcript | 157381478 | 157389278 | + | gene_id “NFAT_NG4”; transcript_id |
| | | | | | “NFAT_NG4.1”; RPKM “1.0039”; cov |
| | | | | | “13.9041”; |
| chr1 | exon | 157381478 | 157384579 | + | gene_id “NFAT_NG4”; transcript_id |
| | | | | | “NFAT_NG4.1”; exon “1”; |
| chr1 | exon | 157386528 | 157386619 | + | gene_id “NFAT_NG4”; transcript_id |
| | | | | | “NFAT_NG4.1”; exon “2”; |
| chr1 | exon | 157386907 | 157389278 | + | gene_id “NFAT_NG4”; transcript_id |
| | | | | | “NFAT_NG4.1”; exon “3”; |
| chr1 | transcript | 157381478 | 157389278 | + | gene_id “NFAT_NG4”; transcript_id |
| | | | | | “NFAT_NG4.2”; RPKM “1.1455”; cov |
| | | | | | “15.8646”; |
| chr1 | exon | 157381478 | 157386619 | + | gene_id “NFAT_NG4”; transcript_id |
| | | | | | “NFAT_NG4.2”; exon “1”; |
| chr1 | exon | 157386907 | 157389278 | + | gene_id “NFAT_NG4”; transcript_id |
| | | | | | “NFAT_NG4.2”; exon “2”; |
| chr1 | transcript | 247451836 | 247454103 | + | gene_id “NFAT_NG5”; transcript_id |
| | | | | | “NFAT_NG5.1”; RPKM “2.2625”; cov |
| | | | | | “31.3354”; |
| chr1 | exon | 247451836 | 247452084 | + | gene_id “NFAT_NG5”; transcript_id |
| | | | | | “NFAT_NG5.1”; exon “1”; |
| chr1 | exon | 247453742 | 247454103 | + | gene_id “NFAT_NG5”; transcript_id |
| | | | | | “NFAT_NG5.1”; exon “2”; |
| chr1 | transcript | 247451836 | 247454103 | + | gene_id “NFAT_NG5”; transcript_id |
| | | | | | “NFAT_NG5.2”; RPKM “3.1260”; cov |
| | | | | | “43.2954”; |
| chr1 | exon | 247451836 | 247452084 | + | gene_id “NFAT_NG5”; transcript_id |
| | | | | | “NFAT_NG5.2”; exon “1”; |
| chr1 | exon | 247453496 | 247454103 | + | gene_id “NFAT_NG5”; transcript_id |
| | | | | | “NFAT_NG5.2”; exon “2”; |
| chr10 | transcript | 123371298 | 123371778 | + | gene_id “NFAT_NG31”; transcript_id |
| | | | | | “NFAT_NG31.1”; RPKM “14.4823”; cov |
| | | | | | “200.5780”; |
| chr10 | exon | 123371298 | 123371778 | + | gene_id “NFAT_NG31”; transcript_id |
| | | | | | “NFAT_NG31.1”; exon “1”; |
| chr10 | transcript | 123395103 | 123399638 | − | gene_id “NFAT_NG32”; transcript_id |
| | | | | | “NFAT_NG32.1”; RPKM “6.4070”; cov |
| | | | | | “88.7368”; |
| chr10 | exon | 123395103 | 123399638 | − | gene_id “NFAT_NG32”; transcript_id |
| | | | | | “NFAT_NG32.1”; exon “1”; |
| chr11 | transcript | 30811427 | 30812101 | − | gene_id “NFAT_NG33”; transcript_id |
| | | | | | “NFAT_NG33.1”; RPKM “18.1084”; cov |
| | | | | | “250.8000”; |
| chr11 | exon | 30811427 | 30812101 | − | gene_id “NFAT_NG33”; transcript_id |
| | | | | | “NFAT_NG33.1”; exon “1”; |
| chr11 | transcript | 30839762 | 30840096 | − | gene_id “NFAT_NG34”; transcript_id |
| | | | | | “NFAT_NG34.1”; RPKM “4.1968”; cov |
| | | | | | “58.1254”; |
| chr11 | exon | 30839762 | 30840096 | − | gene_id “NFAT_NG34”; transcript_id |
| | | | | | “NFAT_NG34.1”; exon “1”; |
| chr11 | transcript | 119846297 | 119894280 | + | gene_id “NFAT_NG35”; transcript_id |
| | | | | | “NFAT_NG35.1”; RPKM “1.9097”; cov |
| | | | | | “26.4496”; |
| chr11 | exon | 119846297 | 119846861 | + | gene_id “NFAT_NG35”; transcript_id |
| | | | | | “NFAT_NG35.1”; exon “1”; |
| chr11 | exon | 119881564 | 119894280 | + | gene_id “NFAT_NG35”; transcript_id |
| | | | | | “NFAT_NG35.1”; exon “2”; |
| chr12 | transcript | 67840399 | 67900475 | − | gene_id “NFAT_NG36”; transcript_id |
| | | | | | “NFAT_NG36.1”; RPKM “3.0848”; cov |
| | | | | | “42.7241”; |
| chr12 | exon | 67840399 | 67840630 | − | gene_id “NFAT_NG36”; transcript_id |
| | | | | | “NFAT_NG36.1”; exon “1”; |
| chr12 | exon | 67900251 | 67900475 | − | gene_id “NFAT_NG36”; transcript_id |
| | | | | | “NFAT_NG36.1”; exon “2”; |
| chr12 | transcript | 97370395 | 97372279 | + | gene_id “NFAT_NG37”; transcript_id |
| | | | | | “NFAT_NG37.1”; RPKM “7.7168”; cov |
| | | | | | “106.8767”; |
| chr12 | exon | 97370395 | 97370521 | + | gene_id “NFAT_NG37”; transcript_id |
| | | | | | “NFAT_NG37.1”; exon “1”; |
| chr12 | exon | 97372118 | 97372279 | + | gene_id “NFAT_NG37”; transcript_id |
| | | | | | “NFAT_NG37.1”; exon “2”; |
| chr12 | transcript | 97370395 | 97375253 | + | gene_id “NFAT_NG37”; transcript_id |
| | | | | | “NFAT_NG37.2”; RPKM “1.9197”; cov |
| | | | | | “26.5872”; |
| chr12 | exon | 97370395 | 97370521 | + | gene_id “NFAT_NG37”; transcript_id |
| | | | | | “NFAT_NG37.2”; exon “1”; |
| chr12 | exon | 97373311 | 97375253 | + | gene_id “NFAT_NG37”; transcript_id |
| | | | | | “NFAT_NG37.2”; exon “2”; |
| chr13 | transcript | 98714143 | 98716660 | − | gene_id “NFAT_NG38”; transcript_id |
| | | | | | “NFAT_NG38.1”; RPKM “41.0034”; cov |
| | | | | | “567.8922”; |
| chr13 | exon | 98714143 | 98714574 | − | gene_id “NFAT_NG38”; transcript_id |
| | | | | | “NFAT_NG38.1”; exon “1”; |
| chr13 | exon | 98716286 | 98716660 | − | gene_id “NFAT_NG38”; transcript_id |
| | | | | | “NFAT_NG38.1”; exon “2”; |
| chr17 | transcript | 40322903 | 40324207 | + | gene_id “NFAT_NG39”; transcript_id |
| | | | | | “NFAT_NG39.1”; RPKM “1.4773”; cov |
| | | | | | “20.4599”; |
| chr17 | exon | 40322903 | 40323018 | + | gene_id “NFAT_NG39”; transcript_id |
| | | | | | “NFAT_NG39.1”; exon “1”; |
| chr17 | exon | 40323865 | 40324207 | + | gene_id “NFAT_NG39”; transcript_id |
| | | | | | “NFAT_NG39.1”; exon “2”; |
| chr17 | transcript | 40322903 | 40324207 | + | gene_id “NFAT_NG39”; transcript_id |
| | | | | | “NFAT_NG39.2”; RPKM “9.6034”; cov |
| | | | | | “133.0066”; |
| chr17 | exon | 40322903 | 40323160 | + | gene_id “NFAT_NG39”; transcript_id |
| | | | | | “NFAT_NG39.2”; exon “1”; |
| chr17 | exon | 40323865 | 40324207 | + | gene_id “NFAT_NG39”; transcript_id |
| | | | | | “NFAT_NG39.2”; exon “2”; |
| chr17 | transcript | 67140805 | 67141049 | + | gene_id “NFAT_NG40”; transcript_id |
| | | | | | “NFAT_NG40.1”; RPKM “6.3512”; cov |
| | | | | | “87.9633”; |
| chr17 | exon | 67140805 | 67141049 | + | gene_id “NFAT_NG40”; transcript_id |
| | | | | | “NFAT_NG40.1”; exon “1”; |
| chr19 | transcript | 13779661 | 13782996 | − | gene_id “NFAT_NG41”; transcript_id |
| | | | | | “NFAT_NG41.1”; RPKM “1.6627”; cov |
| | | | | | “23.0275”; |
| chr19 | exon | 13779661 | 13782996 | − | gene_id “NFAT_NG41”; transcript_id |
| | | | | | “NFAT_NG41.1”; exon “1”; |
| chr19 | transcript | 13781526 | 13794556 | − | gene_id “NFAT_NG41”; transcript_id |
| | | | | | “NFAT_NG41.2”; RPKM “2.7593”; cov |
| | | | | | “38.2165”; |
| chr19 | exon | 13781526 | 13782513 | − | gene_id “NFAT_NG41”; transcript_id |
| | | | | | “NFAT_NG41.2”; exon “1”; |
| chr19 | exon | 13789861 | 13790010 | − | gene_id “NFAT_NG41”; transcript_id |
| | | | | | “NFAT_NG41.2”; exon “2”; |
| chr19 | exon | 13792124 | 13794556 | − | gene_id “NFAT_NG41”; transcript_id |
| | | | | | “NFAT_NG41.2”; exon “3”; |
| chr19 | transcript | 13794639 | 13805316 | − | gene_id “NFAT_NG41”; transcript_id |
| | | | | | “NFAT_NG41.13”; RPKM “2.5583”; cov |
| | | | | | “35.4322”; |
| chr19 | exon | 13794639 | 13796795 | − | gene_id “NFAT_NG41”; transcript_id |
| | | | | | “NFAT_NG41.13”; exon “1”; |
| chr19 | exon | 13798951 | 13805316 | − | gene_id “NFAT_NG41”; transcript_id |
| | | | | | “NFAT_NG41.13”; exon “2”; |
| chr19 | transcript | 13794639 | 13805316 | − | gene_id “NFAT_NG41”; transcript_id |
| | | | | | “NFAT_NG41.9”; RPKM “1.1001”; cov |
| | | | | | “15.2365”; |
| chr19 | exon | 13794639 | 13795080 | − | gene_id “NFAT_NG41”; transcript_id |
| | | | | | “NFAT_NG41.9”; exon “1”; |
| chr19 | exon | 13796644 | 13796795 | − | gene_id “NFAT_NG41”; transcript_id |
| | | | | | “NFAT_NG41.9”; exon “2”; |
| chr19 | exon | 13798951 | 13805316 | − | gene_id “NFAT_NG41”; transcript_id |
| | | | | | “NFAT_NG41.9”; exon “3”; |
| chr19 | transcript | 13805638 | 13813987 | − | gene_id “NFAT_NG41”; transcript_id |
| | | | | | “NFAT_NG41.11”; RPKM “3.8037”; cov |
| | | | | | “52.6811”; |
| chr19 | exon | 13805638 | 13813987 | − | gene_id “NFAT_NG41”; transcript_id |
| | | | | | “NFAT_NG41.11”; exon “1”; |
| chr19 | transcript | 13805638 | 13818383 | − | gene_id “NFAT_NG41”; transcript_id |
| | | | | | “NFAT_NG41.12”; RPKM “1.6994”; cov |
| | | | | | “23.5360”; |
| chr19 | exon | 13805638 | 13808777 | − | gene_id “NFAT_NG41”; transcript_id |
| | | | | | “NFAT_NG41.12”; exon “1”; |
| chr19 | exon | 13815689 | 13815806 | − | gene_id “NFAT_NG41”; transcript_id |
| | | | | | “NFAT_NG41.12”; exon “2”; |
| chr19 | exon | 13816006 | 13818383 | − | gene_id “NFAT_NG41”; transcript_id |
| | | | | | “NFAT_NG41.12”; exon “3”; |
| chr19 | transcript | 13815247 | 13835941 | − | gene_id “NFAT_NG41”; transcript_id |
| | | | | | “NFAT_NG41.6”; RPKM “1.3817”; cov |
| | | | | | “19.1371”; |
| chr19 | exon | 13815247 | 13815806 | − | gene_id “NFAT_NG41”; transcript_id |
| | | | | | “NFAT_NG41.6”; exon “1”; |
| chr19 | exon | 13823325 | 13823486 | − | gene_id “NFAT_NG41”; transcript_id |
| | | | | | “NFAT_NG41.6”; exon “2”; |
| chr19 | exon | 13835733 | 13835941 | − | gene_id “NFAT_NG41”; transcript_id |
| | | | | | “NFAT_NG41.6”; exon “3”; |
| chr19 | transcript | 13815247 | 13824618 | − | gene_id “NFAT_NG41”; transcript_id |
| | | | | | “NFAT_NG41.8”; RPKM “1.1501”; cov |
| | | | | | “15.9294”; |
| chr19 | exon | 13815247 | 13815806 | − | gene_id “NFAT_NG41”; transcript_id |
| | | | | | “NFAT_NG41.8”; exon “1”; |
| chr19 | exon | 13823325 | 13824618 | − | gene_id “NFAT_NG41”; transcript_id |
| | | | | | “NFAT_NG41.8”; exon “2”; |
| chr19 | transcript | 13815247 | 13835941 | − | gene_id “NFAT_NG41”; transcript_id |
| | | | | | “NFAT_NG41.14”; RPKM “1.1135”; cov |
| | | | | | “15.4213”; |
| chr19 | exon | 13815247 | 13823229 | − | gene_id “NFAT_NG41”; transcript_id |
| | | | | | “NFAT_NG41.14”; exon “1”; |
| chr19 | exon | 13823325 | 13823486 | − | gene_id “NFAT_NG41”; transcript_id |
| | | | | | “NFAT_NG41.14”; exon “2”; |
| chr19 | exon | 13835098 | 13835219 | − | gene_id “NFAT_NG41”; transcript_id |
| | | | | | “NFAT_NG41.14”; exon “3”; |
| chr19 | exon | 13835733 | 13835941 | − | gene_id “NFAT_NG41”; transcript_id |
| | | | | | “NFAT_NG41.14”; exon “4”; |
| chr19 | transcript | 13818392 | 13835941 | − | gene_id “NFAT_NG41”; transcript_id |
| | | | | | “NFAT_NG41.15”; RPKM “0.9362”; cov |
| | | | | | “12.9659”; |
| chr19 | exon | 13818392 | 13823229 | − | gene_id “NFAT_NG41”; transcript_id |
| | | | | | “NFAT_NG41.15”; exon “1”; |
| chr1 9 | exon | 13823325 | 13823486 | − | gene id “NFAT_NG41”; transcript_id |
| | | | | | “NFAT_NG41.15”; exon “2”; |
| chr19 | exon | 13825184 | 13825247 | − | gene_id “NFAT_NG41”; transcript_id |
| | | | | | “NFAT_NG41.15”; exon “3”; |
| chr19 | exon | 13827990 | 13828053 | − | gene_id “NFAT_NG41”; transcript_id |
| | | | | | “NFAT_NG41.15”; exon “4”; |
| chr19 | exon | 13835098 | 13835219 | − | gene_id “NFAT_NG41”; transcript_id |
| | | | | | “NFAT_NG41.15”; exon “5”; |
| chr19 | exon | 13835733 | 13835941 | − | gene_id “NFAT_NG41”; transcript_id |
| | | | | | “NFAT_NG41.15”; exon “6”; |
| chr19 | transcript | 13818392 | 13825176 | − | gene_id “NFAT_NG41”; transcript_id |
| | | | | | “NFAT_NG41.7”; RPKM “2.3998”; cov |
| | | | | | “33.2371”; |
| chr19 | exon | 13818392 | 13823229 | − | gene_id “NFAT_NG41”; transcript_id |
| | | | | | “NFAT_NG41.7”; exon “1”; |
| chr19 | exon | 13823325 | 13823486 | − | gene_id “NFAT_NG41”; transcript_id |
| | | | | | “NFAT_NG41.7”; exon “2”; |
| chr19 | exon | 13823805 | 13825176 | − | gene_id “NFAT_NG41”; transcript_id |
| | | | | | “NFAT_NG41.7”; exon “3”; |
| chr19 | transcript | 13818392 | 13835941 | − | gene_id “NFAT_NG41”; transcript_id |
| | | | | | “NFAT_NG41.10”; RPKM “2.4982”; cov |
| | | | | | “34.5998”; |
| chr19 | exon | 13818392 | 13824781 | − | gene_id “NFAT_NG41”; transcript_id |
| | | | | | “NFAT_NG41.10”; exon “1”; |
| chr19 | exon | 13825184 | 13825247 | − | gene_id “NFAT_NG41”; transcript_id |
| | | | | | “NFAT_NG41.10”; exon “2”; |
| chr19 | exon | 13827990 | 13828053 | − | gene_id “NFAT_NG41”; transcript_id |
| | | | | | “NFAT_NG41.10”; exon “3”; |
| chr19 | exon | 13835098 | 13835219 | − | gene_id “NFAT_NG41”; transcript_id |
| | | | | | “NFAT_NG41.10”; exon “4”; |
| chr19 | exon | 13835733 | 13835941 | − | gene_id “NFAT_NG41”; transcript_id |
| | | | | | “NFAT_NG41.10”; exon “5”; |
| chr19 | transcript | 13824639 | 13835941 | − | gene_id “NFAT_NG41”; transcript_id |
| | | | | | “NFAT_NG41.4”; RPKM “1.1554”; cov |
| | | | | | “16.0026”; |
| chr19 | exon | 13824639 | 13824781 | − | gene_id “NFAT_NG41”; transcript_id |
| | | | | | “NFAT_NG41.4”; exon “1”; |
| chr19 | exon | 13825184 | 13825247 | − | gene_id “NFAT_NG41”; transcript_id |
| | | | | | “NFAT_NG41.4”; exon “2”; |
| chr19 | exon | 13835098 | 13835219 | − | gene_id “NFAT_NG41”; transcript_id |
| | | | | | “NFAT_NG41.4”; exon “3”; |
| chr19 | exon | 13835733 | 13835941 | − | gene_id “NFAT_NG41”; transcript_id |
| | | | | | “NFAT_NG41.4”; exon “4”; |
| chr19 | transcript | 13826505 | 13828691 | − | gene_id “NFAT_NG41”; transcript_id |
| | | | | | “NFAT_NG41.3”; RPKM “2.2391”; cov |
| | | | | | “31.0114”; |
| chr19 | exon | 13826505 | 13828691 | − | gene_id “NFAT_NG41”; transcript_id |
| | | | | | “NFAT_NG41.3”; exon “1”; |
| chr19 | transcript | 13834709 | 13835941 | − | gene_id “NFAT_NG41”; transcript_id |
| | | | | | “NFAT_NG41.5”; RPKM “1.8692”; cov |
| | | | | | “25.8888”; |
| chr19 | exon | 13834709 | 13835219 | − | gene_id “NFAT_NG41”; transcript_id |
| | | | | | “NFAT_NG41.5”; exon “1”; |
| chr19 | exon | 13835733 | 13835941 | − | gene_id “NFAT_NG41”; transcript_id |
| | | | | | “NFAT_NG41.5”; exon “2”; |
| chr2 | transcript | 45683440 | 45730602 | + | gene_id “NFAT_NG6”; transcript_id |
| | | | | | “NFAT_NG6.1”; RPKM “0.9316”; cov |
| | | | | | “12.9030”; |
| chr2 | exon | 45683440 | 45683520 | + | gene_id “NFAT_NG6”; transcript_id |
| | | | | | “NFAT_NG6.1”; exon “1”; |
| chr2 | exon | 45730420 | 45730602 | + | gene_id “NFAT_NG6”; transcript_id |
| | | | | | “NFAT_NG6.1”; exon “2”; |
| chr2 | transcript | 45683440 | 45730602 | + | gene_id “NFAT_NG6”; transcript_id |
| | | | | | “NFAT_NG6.2”; RPKM “1.2417”; cov |
| | | | | | “17.1967”; |
| chr2 | exon | 45683440 | 45683520 | + | gene_id “NFAT_NG6”; transcript_id |
| | | | | | “NFAT_NG6.2”; exon “1”; |
| chr2 | exon | 45712323 | 45712399 | + | gene_id “NFAT_NG6”; transcript_id |
| | | | | | “NFAT_NG6.2”; exon “2”; |
| chr2 | exon | 45730420 | 45730602 | + | gene_id “NFAT_NG6”; transcript_id |
| | | | | | “NFAT_NG6.2”; exon “3”; |
| chr2 | transcript | 101255231 | 101270778 | − | gene_id “NFAT_NG7”; transcript_id |
| | | | | | “NFAT_NG7.1”; RPKM “1.7283”; cov |
| | | | | | “23.9365”; |
| chr2 | exon | 101255231 | 101258063 | − | gene_id “NFAT_NG7”; transcript_id |
| | | | | | “NFAT_NG7.1”; exon “1”; |
| chr2 | exon | 101262704 | 101262761 | − | gene_id “NFAT_NG7”; transcript_id |
| | | | | | “NFAT_NG7.1”; exon “2”; |
| chr2 | exon | 101268370 | 101268451 | − | gene_id “NFAT_NG7”; transcript_id |
| | | | | | “NFAT_NG7.1”; exon “3”; |
| chr2 | exon | 101268970 | 101269081 | − | gene_id “NFAT_NG7”; transcript_id |
| | | | | | “NFAT_NG7.1”; exon “4”; |
| chr2 | exon | 101269594 | 101270778 | − | gene_id “NFAT_NG7”; transcript_id |
| | | | | | “NFAT_NG7.1”; exon “5”; |
| chr2 | transcript | 101255231 | 101359466 | − | gene_id “NFAT_NG7”; transcript_id |
| | | | | | “NFAT_NG7.2”; RPKM “1.3501”; cov |
| | | | | | “18.6990”; |
| chr2 | exon | 101255231 | 101258063 | − | gene_id “NFAT_NG7”; transcript_id |
| | | | | | “NFAT_NG7.2”; exon “1”; |
| chr2 | exon | 101262704 | 101262761 | − | gene_id “NFAT_NG7”; transcript_id |
| | | | | | “NFAT_NG7.2”; exon “2”; |
| chr2 | exon | 101268370 | 101269081 | − | gene_id “NFAT_NG7”; transcript_id |
| | | | | | “NFAT_NG7.2”; exon “3”; |
| chr2 | exon | 101269594 | 101269721 | − | gene_id “NFAT_NG7”; transcript_id |
| | | | | | “NFAT_NG7.2”; exon “4”; |
| chr2 | exon | 101359253 | 101359466 | − | gene_id “NFAT_NG7”; transcript_id |
| | | | | | “NFAT_NG7.2”; exon “5”; |
| chr2 | transcript | 198028367 | 198055139 | + | gene_id “NFAT_NG8”; transcript_id |
| | | | | | “NFAT_NG8.1”; RPKM “2.0569”; cov |
| | | | | | “28.4884”; |
| chr2 | exon | 198028367 | 198029989 | + | gene_id “NFAT_NG8”; transcript_id |
| | | | | | “NFAT_NG8.1”; exon “1”; |
| chr2 | exon | 198051611 | 198051897 | + | gene_id “NFAT_NG8”; transcript_id |
| | | | | | “NFAT_NG8.1”; exon “2”; |
| chr2 | exon | 198052444 | 198055139 | + | gene_id “NFAT_NG8”; transcript_id |
| | | | | | “NFAT_NG8.1”; exon “3”; |
| chr2 | transcript | 198028367 | 198055139 | + | gene_id “NFAT_NG8”; transcript_id |
| | | | | | “NFAT_NG8.2”; RPKM “1.2755”; cov |
| | | | | | “17.6651”; |
| chr2 | exon | 198028367 | 198028669 | + | gene_id “NFAT_NG8”; transcript_id |
| | | | | | “NFAT_NG8.2”; exon “1”; |
| chr2 | exon | 198029918 | 198029989 | + | gene_id “NFAT_NG8”; transcript_id |
| | | | | | “NFAT_NG8.2”; exon “2”; |
| chr2 | exon | 198051611 | 198051671 | + | gene_id “NFAT_NG8”; transcript_id |
| | | | | | “NFAT_NG8.2”; exon “3”; |
| chr2 | exon | 198051767 | 198051897 | + | gene_id “NFAT_NG8”; transcript_id |
| | | | | | “NFAT_NG8.2”; exon “4”; |
| chr2 | exon | 198052444 | 198055139 | + | gene_id “NFAT_NG8”; transcript_id |
| | | | | | “NFAT_NG8.2”; exon “5”; |
| chr2 | transcript | 198028367 | 198055139 | + | gene_id “NFAT_NG8”; transcript_id |
| | | | | | “NFAT_NG8.3”; RPKM “1.4057”; cov |
| | | | | | “19.4689”; |
| chr2 | exon | 198028367 | 198042181 | + | gene_id “NFAT_NG8”; transcript_id |
| | | | | | “NFAT_NG8.3”; exon “1”; |
| chr2 | exon | 198043262 | 198043392 | + | gene_id “NFAT_NG8”; transcript_id |
| | | | | | “NFAT_NG8.3”; exon “2”; |
| chr2 | exon | 198051611 | 198051897 | + | gene_id “NFAT_NG8”; transcript_id |
| | | | | | “NFAT_NG8.3”; exon “3”; |
| chr2 | exon | 198052444 | 198055139 | + | gene_id “NFAT_NG8”; transcript_id |
| | | | | | “NFAT_NG8.3”; exon “4”; |
| chr2 | transcript | 198028367 | 198055139 | + | gene_id “NFAT_NG8”; transcript_id |
| | | | | | “NFAT_NG8.4”; RPKM “0.7892”; cov |
| | | | | | “10.9298”; |
| chr2 | exon | 198028367 | 198042181 | + | gene_id “NFAT_NG8”; transcript_id |
| | | | | | “NFAT_NG8.4”; exon “1”; |
| chr2 | exon | 198051611 | 198051897 | + | gene_id “NFAT_NG8”; transcript_id |
| | | | | | “NFAT_NG8.4”; exon “2”; |
| chr2 | exon | 198052444 | 198055139 | + | gene_id “NFAT_NG8”; transcript_id |
| | | | | | “NFAT_NG8.4”; exon “3”; |
| chr2 | transcript | 198028367 | 198055139 | + | gene_id “NFAT_NG8”; transcript_id |
| | | | | | “NFAT_NG8.5”; RPKM “1.0391”; cov |
| | | | | | “14.3920”; |
| chr2 | exon | 198028367 | 198042181 | + | gene_id “NFAT_NG8”; transcript_id |
| | | | | | “NFAT_NG8.5”; exon “1”; |
| chr2 | exon | 198051611 | 198051671 | + | gene_id “NFAT_NG8”; transcript_id |
| | | | | | “NFAT_NG8.5”; exon “2”; |
| chr2 | exon | 198051767 | 198051897 | + | gene_id “NFAT_NG8”; transcript_id |
| | | | | | “NFAT_NG8.5”; exon “3”; |
| chr2 | exon | 198052444 | 198055139 | + | gene_id “NFAT_NG8”; transcript_id |
| | | | | | “NFAT_NG8.5”; exon “4”; |
| chr2 | transcript | 238353802 | 238357577 | − | gene_id “NFAT_NG9”; transcript_id |
| | | | | | “NFAT_NG9.1”; RPKM “2.7658”; cov |
| | | | | | “38.3058”; |
| chr2 | exon | 238353802 | 238357577 | − | gene_id “NFAT_NG9”; transcript_id |
| | | | | | “NFAT_NG9.1”; exon “1”; |
| chr20 | transcript | 37227074 | 37230330 | − | gene_id “NFAT_NG42”; transcript_id |
| | | | | | “NFAT_NG42.1”; RPKM “0.7901”; cov |
| | | | | | “10.9434”; |
| chr20 | exon | 37227074 | 37227273 | − | gene_id “NFAT_NG42”; transcript_id |
| | | | | | “NFAT_NG42.1”; exon “1”; |
| chr20 | exon | 37228492 | 37230330 | − | gene_id “NFAT_NG42”; transcript_id |
| | | | | | “NFAT_NG42.1”; exon “2”; |
| chr20 | transcript | 37227074 | 37230330 | − | gene_id “NFAT_NG42”; transcript_id |
| | | | | | “NFAT_NG42.2”; RPKM “1.6120”; cov |
| | | | | | “22.3256”; |
| chr20 | exon | 37227074 | 37230330 | − | gene_id “NFAT_NG42”; transcript_id |
| | | | | | “NFAT_NG42.2”; exon “1”; |
| chr20 | transcript | 44546832 | 44557544 | + | gene_id “NFAT_NG43”; transcript_id |
| | | | | | “NFAT_NG43.1”; RPKM “14.7633”; cov |
| | | | | | “204.4700”; |
| chr20 | exon | 44546832 | 44547056 | + | gene_id “NFAT_NG43”; transcript_id |
| | | | | | “NFAT_NG43.1”; exon “1”; |
| chr20 | exon | 44548365 | 44557544 | + | gene_id “NFAT_NG43”; transcript_id |
| | | | | | “NFAT_NG43.1”; exon “2”; |
| chr20 | transcript | 47523284 | 47523755 | + | gene_id “NFAT_NG44”; transcript_id |
| | | | | | “NFAT_NG44.1”; RPKM “12.4721”; cov |
| | | | | | “172.7373”; |
| chr20 | exon | 47523284 | 47523755 | + | gene_id “NFAT_NG44”; transcript_id |
| | | | | | “NFAT_NG44.1”; exon “1”; |
| chr20 | transcript | 47957753 | 47960887 | + | gene_id “NFAT_NG45”; transcript_id |
| | | | | | “NFAT_NG45.1”; RPKM “5.1391”; cov |
| | | | | | “71.1756”; |
| chr20 | exon | 47957753 | 47960887 | + | gene_id “NFAT_NG45”; transcript_id |
| | | | | | “NFAT_NG45.1”; exon “1”; |
| chr21 | transcript | 43476107 | 43477132 | − | gene_id “NFAT_NG46”; transcript_id |
| | | | | | “NFAT_NG46.1”; RPKM “4.7532”; cov |
| | | | | | “65.8307”; |
| chr21 | exon | 43476107 | 43477132 | − | gene_id “NFAT_NG46”; transcript_id |
| | | | | | “NFAT_NG46.1”; exon “1”; |
| chr3 | transcript | 134098263 | 134106446 | − | gene_id “NFAT_NG10”; transcript_id |
| | | | | | “NFAT_NG10.1”; RPKM “1.9791”; cov |
| | | | | | “27.4104”; |
| chr3 | exon | 134098263 | 134106446 | − | gene_id “NFAT_NG10”; transcript_id |
| | | | | | “NFAT_NG10.1”; exon “1”; |
| chr3 | transcript | 134098263 | 134112901 | − | gene_id “NFAT_NG10”; transcript_id |
| | | | | | “NFAT_NG10.2”; RPKM “2.1210”; cov |
| | | | | | “29.3757”; |
| chr3 | exon | 134098263 | 134101497 | − | gene_id “NFAT_NG10”; transcript_id |
| | | | | | “NFAT_NG10.2”; exon “1”; |
| chr3 | exon | 134112482 | 134112901 | − | gene_id “NFAT_NG10”; transcript_id |
| | | | | | “NFAT_NG10.2”; exon “2”; |
| chr4 | transcript | 42153350 | 42241350 | + | gene_id “NFAT_NG11”; transcript_id |
| | | | | | “NFAT_NG11.1”; RPKM “1.1726”; cov |
| | | | | | “16.2401”; |
| chr4 | exon | 42153350 | 42153580 | + | gene_id “NFAT_NG11”; transcript_id |
| | | | | | “NFAT_NG11.1”; exon “1”; |
| chr4 | exon | 42240711 | 42241350 | + | gene_id “NFAT_NG11”; transcript_id |
| | | | | | “NFAT_NG11.1”; exon “2”; |
| chr4 | transcript | 42234509 | 42241350 | + | gene_id “NFAT_NG11”; transcript_id |
| | | | | | “NFAT_NG11.2”; RPKM “3.8366”; cov |
| | | | | | “53.1359”; |
| chr4 | exon | 42234509 | 42234675 | + | gene_id “NFAT_NG11”; transcript_id |
| | | | | | “NFAT_NG11.2”; exon “1”; |
| chr4 | exon | 42240711 | 42241350 | + | gene_id “NFAT_NG11”; transcript_id |
| | | | | | “NFAT_NG11.2”; exon “2”; |
| chr4 | transcript | 42234509 | 42241350 | + | gene_id “NFAT_NG11”; transcript_id |
| | | | | | “NFAT_NG11.3”; RPKM “0.7633”; cov |
| | | | | | “10.5714”; |
| chr4 | exon | 42234509 | 42234675 | + | gene_id “NFAT_NG11”; transcript_id |
| | | | | | “NFAT_NG11.3”; exon “1”; |
| chr4 | exon | 42236179 | 42236293 | + | gene_id “NFAT_NG11”; transcript_id |
| | | | | | “NFAT_NG11.3”; exon “2”; |
| chr4 | exon | 42240711 | 42241350 | + | gene_id “NFAT_NG11”; transcript_id |
| | | | | | “NFAT_NG11.3”; exon “3”; |
| chr4 | transcript | 150065027 | 150065608 | + | gene_id “NFAT_NG12”; transcript_id |
| | | | | | “NFAT_NG12.1”; RPKM “6.0835”; cov |
| | | | | | “84.2560”; |
| chr4 | exon | 150065027 | 150065608 | + | gene_id “NFAT_NG12”; transcript_id |
| | | | | | “NFAT_NG12.1”; exon “1”; |
| chr4 | transcript | 184483153 | 184483751 | + | gene_id “NFAT_NG13”; transcript_id |
| | | | | | “NFAT_NG13.1”; RPKM “2.0545”; cov |
| | | | | | “28.4545”; |
| chr4 | exon | 184483153 | 184483751 | + | gene_id “NFAT_NG13”; transcript_id |
| | | | | | “NFAT_NG13.1”; exon “1”; |
| chr4 | transcript | 184483153 | 184490910 | + | gene_id “NFAT_NG13”; transcript_id |
| | | | | | “NFAT_NG13.2”; RPKM “1.0838”; cov |
| | | | | | “15.0099”; |
| chr4 | exon | 184483153 | 184483416 | + | gene_id “NFAT_NG13”; transcript_id |
| | | | | | “NFAT_NG13.2”; exon “1”; |
| chr4 | exon | 184485161 | 184490910 | + | gene_id “NFAT_NG13”; transcript_id |
| | | | | | “NFAT_NG13.2”; exon “2”; |
| chr5 | transcript | 127530275 | 127547812 | − | gene_id “NFAT_NG14”; transcript_id |
| | | | | | “NFAT_NG14.2”; RPKM “7.6426”; cov |
| | | | | | “105.8495”; |
| chr5 | exon | 127530275 | 127537771 | − | gene_id “NFAT_NG14”; transcript_id |
| | | | | | “NFAT_NG14.2”; exon “1”; |
| chr5 | exon | 127543631 | 127543759 | − | gene_id “NFAT_NG14”; transcript_id |
| | | | | | “NFAT_NG14.2”; exon “2”; |
| chr5 | exon | 127547699 | 127547812 | − | gene_id “NFAT_NG14”; transcript_id |
| | | | | | “NFAT_NG14.2”; exon “3”; |
| chr5 | transcript | 127530275 | 127547812 | − | gene_id “NFAT_NG14”; transcript_id |
| | | | | | “NFAT_NG14.3”; RPKM “21.1724”; cov |
| | | | | | “293.2362”; |
| chr5 | exon | 127530275 | 127537414 | − | gene_id “NFAT_NG14”; transcript_id |
| | | | | | “NFAT_NG14.3”; exon “1”; |
| chr5 | exon | 127537619 | 127537771 | − | gene_id “NFAT_NG14”; transcript_id |
| | | | | | “NFAT_NG14.3”; exon “2”; |
| chr5 | exon | 127543631 | 127543759 | − | gene_id “NFAT_NG14”; transcript_id |
| | | | | | “NFAT_NG14.3”; exon “3”; |
| chr5 | exon | 127547699 | 127547812 | − | gene_id “NFAT_NG14”; transcript_id |
| | | | | | “NFAT_NG14.3”; exon “4”; |
| chr5 | transcript | 127530275 | 127547812 | − | gene_id “NFAT_NG14”; transcript_id |
| | | | | | “NFAT_NG14.4”; RPKM “2.7836”; cov |
| | | | | | “38.5520”; |
| chr5 | exon | 127530275 | 127537467 | − | gene_id “NFAT_NG14”; transcript_id |
| | | | | | “NFAT_NG14.4”; exon “1”; |
| chr5 | exon | 127537619 | 127537771 | − | gene_id “NFAT_NG14”; transcript_id |
| | | | | | “NFAT_NG14.4”; exon “2”; |
| chr5 | exon | 127543631 | 127543759 | − | gene_id “NFAT_NG14”; transcript_id |
| | | | | | “NFAT_NG14.4”; exon “3”; |
| chr5 | exon | 127547699 | 127547812 | − | gene_id “NFAT_NG14”; transcript_id |
| | | | | | “NFAT_NG14.4”; exon “4”; |
| chr5 | transcript | 127530275 | 127547812 | − | gene_id “NFAT_NG14”; transcript_id |
| | | | | | “NFAT_NG14.1”; RPKM “0.8567”; cov |
| | | | | | “11.8649”; |
| chr5 | exon | 127530275 | 127537414 | − | gene_id “NFAT_NG14”; transcript_id |
| | | | | | “NFAT_NG14.1”; exon “1”; |
| chr5 | exon | 127537619 | 127537786 | − | gene_id “NFAT_NG14”; transcript_id |
| | | | | | “NFAT_NG14.1”; exon “2”; |
| chr5 | exon | 127543631 | 127543759 | − | gene_id “NFAT_NG14”; transcript_id |
| | | | | | “NFAT_NG14.1”; exon “3”; |
| chr5 | exon | 127547699 | 127547812 | − | gene_id “NFAT_NG14”; transcript_id |
| | | | | | “NFAT_NG14.1”; exon “4”; |
| chr5 | transcript | 127527055 | 127530260 | − | gene_id “NFAT_NG15”; transcript_id |
| | | | | | “NFAT_NG15.1”; RPKM “4.3381”; cov |
| | | | | | “60.0823”; |
| chr5 | exon | 127527055 | 127530260 | − | gene_id “NFAT_NG15”; transcript_id |
| | | | | | “NFAT_NG15.1”; exon “1”; |
| chr5 | transcript | 173943485 | 174000176 | − | gene_id “NFAT_NG16”; transcript_id |
| | | | | | “NFAT_NG16.1”; RPKM “0.9706”; cov |
| | | | | | “13.4421”; |
| chr5 | exon | 173943485 | 173944076 | − | gene_id “NFAT_NG16”; transcript_id |
| | | | | | “NFAT_NG16.1”; exon “1”; |
| chr5 | exon | 174000005 | 174000176 | − | gene_id “NFAT_NG16”; transcript_id |
| | | | | | “NFAT_NG16.1”; exon “2”; |
| chr6 | transcript | 95830544 | 95833565 | + | gene_id “NFAT_NG17”; transcript_id |
| | | | | | “NFAT_NG17.1”; RPKM “4.7975”; cov |
| | | | | | “66.4447”; |
| chr6 | exon | 95830544 | 95833565 | + | gene_id “NFAT_NG17”; transcript_id |
| | | | | | “NFAT_NG17.1”; exon “1”; |
| chr6 | transcript | 95892635 | 95896748 | + | gene_id “NFAT_NG18”; transcript_id |
| | | | | | “NFAT_NG18.2”; RPKM “1.1184”; cov |
| | | | | | “15.4898”; |
| chr6 | exon | 95892635 | 95893251 | + | gene_id “NFAT_NG18”; transcript_id |
| | | | | | “NFAT_NG18.2”; exon “1”; |
| chr6 | exon | 95893369 | 95893551 | + | gene_id “NFAT_NG18”; transcript_id |
| | | | | | “NFAT_NG18.2”; exon “2”; |
| chr6 | exon | 95896077 | 95896748 | + | gene_id “NFAT_NG18”; transcript_id |
| | | | | | “NFAT_NG18.2”; exon “3”; |
| chr6 | transcript | 95912884 | 95915515 | + | gene_id “NFAT_NG18”; transcript_id |
| | | | | | “NFAT_NG18.1”; RPKM “1.8439”; cov |
| | | | | | “25.5380”; |
| chr6 | exon | 95912884 | 95915515 | + | gene_id “NFAT_NG18”; transcript_id |
| | | | | | “NFAT_NG18.1”; exon “1”; |
| chr6 | transcript | 107389053 | 107394010 | + | gene_id “NFAT_NG19”; transcript_id |
| | | | | | “NFAT_NG19.1”; RPKM “1.9609”; cov |
| | | | | | “27.1578”; |
| chr6 | exon | 107389053 | 107389369 | + | gene_id “NFAT_NG19”; transcript_id |
| | | | | | “NFAT_NG19.1”; exon “1”; |
| chr6 | exon | 107389938 | 107394010 | + | gene_id “NFAT_NG19”; transcript_id |
| | | | | | “NFAT_NG19.1”; exon “2”; |
| chr6 | transcript | 107389053 | 107394010 | + | gene_id “NFAT_NG19”; transcript_id |
| | | | | | “NFAT_NG19.2”; RPKM “1.8672”; cov |
| | | | | | “25.8601”; |
| chr6 | exon | 107389053 | 107394010 | + | gene_id “NFAT_NG19”; transcript_id |
| | | | | | “NFAT_NG19.2”; exon “1”; |
| chr6 | transcript | 148520686 | 148521133 | + | gene_id “NFAT_NG20”; transcript_id |
| | | | | | “NFAT_NG20.1”; RPKM “1.7504”; cov |
| | | | | | “24.2433”; |
| chr6 | exon | 148520686 | 148521133 | + | gene_id “NFAT_NG20”; transcript_id |
| | | | | | “NFAT_NG20.1”; exon “1”; |
| chr7 | transcript | 101241162 | 101248766 | − | gene_id “NFAT_NG21”; transcript_id |
| | | | | | “NFAT_NG21.2”; RPKM “1.3389”; cov |
| | | | | | “18.5436”; |
| chr7 | exon | 101241162 | 101245578 | − | gene_id “NFAT_NG21”; transcript_id |
| | | | | | “NFAT_NG21.2”; exon “1”; |
| chr7 | exon | 101248543 | 101248766 | − | gene_id “NFAT_NG21”; transcript_id |
| | | | | | “NFAT_NG21.2”; exon “2”; |
| chr7 | transcript | 101248290 | 101248766 | − | gene_id “NFAT_NG21”; transcript_id |
| | | | | | “NFAT_NG21.1”; RPKM “2.1601”; cov |
| | | | | | “29.9176”; |
| chr7 | exon | 101248290 | 101248766 | − | gene_id “NFAT_NG21”; transcript_id |
| | | | | | “NFAT_NG21.1”; exon “1”; |
| chr7 | transcript | 134053526 | 134059044 | − | gene_id “NFAT_NG22”; transcript_id |
| | | | | | “NFAT_NG22.1”; RPKM “0.8111”; cov |
| | | | | | “11.2330”; |
| chr7 | exon | 134053526 | 134054296 | − | gene_id “NFAT_NG22”; transcript_id |
| | | | | | “NFAT_NG22.1”; exon “1”; |
| chr7 | exon | 134055467 | 134055670 | − | gene_id “NFAT_NG22”; transcript_id |
| | | | | | “NFAT_NG22.1”; exon “2”; |
| chr7 | exon | 134056097 | 134056272 | − | gene_id “NFAT_NG22”; transcript_id |
| | | | | | “NFAT_NG22.1”; exon “3”; |
| chr7 | exon | 134057814 | 134059044 | − | gene_id “NFAT_NG22”; transcript_id |
| | | | | | “NFAT_NG22.1”; exon “4”; |
| chr7 | transcript | 134301798 | 134322444 | − | gene_id “NFAT_NG23”; transcript_id |
| | | | | | “NFAT_NG23.2”; RPKM “4.1677”; cov |
| | | | | | “57.7223”; |
| chr7 | exon | 134301798 | 134304700 | − | gene_id “NFAT_NG23”; transcript_id |
| | | | | | “NFAT_NG23.2”; exon “1”; |
| chr7 | exon | 134321927 | 134322444 | − | gene_id “NFAT_NG23”; transcript_id |
| | | | | | “NFAT_NG23.2”; exon “2”; |
| chr7 | transcript | 134320932 | 134322444 | − | gene_id “NFAT_NG23”; transcript_id |
| | | | | | “NFAT_NG23.1”; RPKM “3.2531”; cov |
| | | | | | “45.0554”; |
| chr7 | exon | 134320932 | 134322444 | − | gene_id “NFAT_NG23”; transcript_id |
| | | | | | “NFAT_NG23.1”; exon “1”; |
| chr8 | transcript | 6663895 | 6670084 | + | gene_id “NFAT_NG24”; transcript_id |
| | | | | | “NFAT_NG24.1”; RPKM “1.7246”; cov |
| | | | | | “23.8854”; |
| chr8 | exon | 6663895 | 6664534 | + | gene_id “NFAT_NG24”; transcript_id |
| | | | | | “NFAT_NG24.1”; exon “1”; |
| chr8 | exon | 6664559 | 6666952 | + | gene_id “NFAT_NG24”; transcript_id |
| | | | | | “NFAT_NG24.1”; exon “2”; |
| chr8 | exon | 6668584 | 6670084 | + | gene_id “NFAT_NG24”; transcript_id |
| | | | | | “NFAT_NG24.1”; exon “3”; |
| chr8 | transcript | 6663895 | 6670084 | + | gene_id “NFAT_NG24”; transcript_id |
| | | | | | “NFAT_NG24.2”; RPKM “1.3166”; cov |
| | | | | | “18.2351”; |
| chr8 | exon | 6663895 | 6666952 | + | gene_id “NFAT_NG24”; transcript_id |
| | | | | | “NFAT_NG24.2”; exon “1”; |
| chr8 | exon | 6668584 | 6670084 | + | gene_id “NFAT_NG24”; transcript_id |
| | | | | | “NFAT_NG24.2”; exon “2”; |
| chr8 | transcript | 23450381 | 23528450 | + | gene_id “NFAT_NG25”; transcript_id |
| | | | | | “NFAT_NG25.2”; RPKM “2.3581”; cov |
| | | | | | “32.6593”; |
| chr8 | exon | 23450381 | 23450576 | + | gene_id “NFAT_NG25”; transcript_id |
| | | | | | “NFAT_NG25.2”; exon “1”; |
| chr8 | exon | 23523018 | 23523115 | + | gene_id “NFAT_NG25”; transcript_id |
| | | | | | “NFAT_NG25.2”; exon “2”; |
| chr8 | exon | 23526156 | 23528450 | + | gene_id “NFAT_NG25”; transcript_id |
| | | | | | “NFAT_NG25.2”; exon “3”; |
| chr8 | transcript | 23731395 | 23735163 | + | gene_id “NFAT_NG25”; transcript_id |
| | | | | | “NFAT_NG25.1”; RPKM “3.9569”; cov |
| | | | | | “54.8028”; |
| chr8 | exon | 23731395 | 23735163 | + | gene_id “NFAT_NG25”; transcript_id |
| | | | | | “NFAT_NG25.1”; exon “1”; |
| chr8 | transcript | 23657162 | 23663146 | + | gene_id “NFAT_NG26”; transcript_id |
| | | | | | “NFAT_NG26.1”; RPKM “5.4070”; cov |
| | | | | | “74.8857”; |
| chr8 | exon | 23657162 | 23663146 | + | gene_id “NFAT_NG26”; transcript_id |
| | | | | | “NFAT_NG26.1”; exon “1”; |
| chr9 | transcript | 18413705 | 18415691 | − | gene_id “NFAT_NG27”; transcript_id |
| | | | | | “NFAT_NG27.2”; RPKM “2.4870”; cov |
| | | | | | “34.4442”; |
| chr9 | exon | 18413705 | 18415691 | − | gene_id “NFAT_NG27”; transcript_id |
| | | | | | “NFAT_NG27.2”; exon “1”; |
| chr9 | transcript | 18413705 | 18422989 | − | gene_id “NFAT_NG27”; transcript_id |
| | | | | | “NFAT_NG27.3”; RPKM “1.0119”; cov |
| | | | | | “14.0148”; |
| chr9 | exon | 18413705 | 18413849 | − | gene_id “NFAT_NG27”; transcript_id |
| | | | | | “NFAT_NG27.3”; exon “1”; |
| chr9 | exon | 18419419 | 18419454 | − | gene_id “NFAT_NG27”; transcript_id |
| | | | | | “NFAT_NG27.3”; exon “2”; |
| chr9 | exon | 18420593 | 18421081 | − | gene_id “NFAT_NG27”; transcript_id |
| | | | | | “NFAT_NG27.3”; exon “3”; |
| chr9 | exon | 18422664 | 18422989 | − | gene_id “NFAT_NG27”; transcript_id |
| | | | | | “NFAT_NG27.3”; exon “4”; |
| chr9 | transcript | 18419752 | 18422989 | − | gene_id “NFAT_NG27”; transcript_id |
| | | | | | “NFAT_NG27.1”; RPKM “7.6140”; cov |
| | | | | | “105.4533”; |
| chr9 | exon | 18419752 | 18421081 | − | gene_id “NFAT_NG27”; transcript_id |
| | | | | | “NFAT_NG27.1”; exon “1”; |
| chr9 | exon | 18422664 | 18422989 | − | gene_id “NFAT_NG27”; transcript_id |
| | | | | | “NFAT_NG27.1”; exon “2”; |
| chr9 | transcript | 25756141 | 25757060 | − | gene_id “NFAT_NG28”; transcript_id |
| | | | | | “NFAT_NG28.1”; RPKM “9.2744”; cov |
| | | | | | “128.4489”; |
| chr9 | exon | 25756141 | 25757060 | − | gene_id “NFAT_NG28”; transcript_id |
| | | | | | “NFAT_NG28.1”; exon “1”; |
| chr9 | transcript | 104114272 | 104114549 | − | gene_id “NFAT_NG29”; transcript_id |
| | | | | | “NFAT_NG29.1”; RPKM “6.9270”; cov |
| | | | | | “95.9388”; |
| chr9 | exon | 104114272 | 104114549 | − | gene_id “NFAT_NG29”; transcript_id |
| | | | | | “NFAT_NG29.1”; exon “1”; |
| chr9 | transcript | 116980060 | 116996390 | − | gene_id “NFAT_NG30”; transcript_id |
| | | | | | “NFAT_NG30.1”; RPKM “1.0994”; cov |
| | | | | | “15.2270”; |
| chr9 | exon | 116980060 | 116996045 | − | gene_id “NFAT_NG30”; transcript_id |
| | | | | | “NFAT_NG30.1”; exon “1”; |
| chr9 | exon | 116996321 | 116996390 | − | gene_id “NFAT_NG30”; transcript_id |
| | | | | | “NFAT_NG30.1”; exon “2”; |
| chrY | transcript | 26535432 | 26541102 | + | gene_id “NFAT_NG47”; transcript_id |
| | | | | | “NFAT_NG47.1”; RPKM “4.3719”; cov |
| | | | | | “60.5497”; |
| chrY | exon | 26535432 | 26535553 | + | gene_id “NFAT_NG47”; transcript_id |
| | | | | | “NFAT_NG47.1”; exon “1”; |
| chrY | exon | 26540808 | 26541102 | + | gene_id “NFAT_NG47”; transcript_id |
| | | | | | “NFAT_NG47.1”; exon “2”; |
| chr15 | transcript | 70421911 | 70434789 | − | gene_id “SS_NG3”; transcript_id |
| | | | | | “SS_NG3.6”; RPKM “0.7613”; cov |
| | | | | | “12.0951”; |
| chr15 | exon | 70421911 | 70422897 | − | gene_id “SS_NG3”; transcript_id |
| | | | | | “SS_NG3.6”; exon “1”; |
| chr15 | exon | 70423121 | 70423230 | − | gene_id “SS_NG3”; transcript_id |
| | | | | | “SS_NG3.6”; exon “2”; |
| chr15 | exon | 70427598 | 70434789 | − | gene_id “SS_NG3”; transcript_id |
| | | | | | “SS_NG3.6”; exon “3”; |
| chr15 | transcript | 70421911 | 70434789 | − | gene_id “SS_NG3”; transcript_id |
| | | | | | “SS_NG3.5”; RPKM “1.1654”; cov |
| | | | | | “18.5167”; |
| chr15 | exon | 70421911 | 70422897 | − | gene_id “SS_NG3”; transcript_id |
| | | | | | “SS_NG3.5”; exon “1”; |
| chr15 | exon | 70423121 | 70423230 | − | gene_id “SS_NG3”; transcript_id |
| | | | | | “SS_NG3.5”; exon “2”; |
| chr15 | exon | 70430217 | 70434789 | − | gene_id “SS_NG3”; transcript_id |
| | | | | | “SS_NG3.5”; exon “3”; |
| chr15 | transcript | 70421911 | 70878937 | − | gene_id “SS_NG3”; transcript_id |
| | | | | | “SS_NG3.4”; RPKM “1.2170”; cov |
| | | | | | “19.3367”; |
| chr15 | exon | 70421911 | 70422897 | − | gene_id “SS_NG3”; transcript_id |
| | | | | | “SS_NG3.4”; exon “1”; |
| chr15 | exon | 70423121 | 70423230 | − | gene_id “SS_NG3”; transcript_id |
| | | | | | “SS_NG3.4”; exon “2”; |
| chr15 | exon | 70640197 | 70640258 | − | gene_id “SS_NG3”; transcript_id |
| | | | | | “SS_NG3.4”; exon “3”; |
| chr15 | exon | 70878214 | 70878937 | − | gene_id “SS_NG3”; transcript_id |
| | | | | | “SS_NG3.4”; exon “4”; |
| chr15 | transcript | 70421911 | 70878937 | − | gene_id “SS_NG3”; transcript_id |
| | | | | | “SS_NG3.3”; RPKM “1.7942”; cov |
| | | | | | “28.5071”; |
| chr15 | exon | 70421911 | 70422897 | − | gene_id “SS_NG3”; transcript_id |
| | | | | | “SS_NG3.3”; exon “1”; |
| chr15 | exon | 70423121 | 70423230 | − | gene_id “SS_NG3”; transcript_id |
| | | | | | “SS_NG3.3”; exon “2”; |
| chr15 | exon | 70878214 | 70878937 | − | gene_id “SS_NG3”; transcript_id |
| | | | | | “SS_NG3.3”; exon “3”; |
| chr15 | transcript | 70585466 | 70878937 | − | gene_id “SS_NG3”; transcript_id |
| | | | | | “SS_NG3.2”; RPKM “0.9038”; cov |
| | | | | | “14.3600”; |
| chr15 | exon | 70585466 | 70588662 | − | gene_id “SS_NG3”; transcript_id |
| | | | | | “SS_NG3.2”; exon “1”; |
| chr15 | exon | 70640197 | 70640258 | − | gene_id “SS_NG3”; transcript_id |
| | | | | | “SS_NG3.2”; exon “2”; |
| chr15 | exon | 70878214 | 70878937 | − | gene_id “SS_NG3”; transcript_id |
| | | | | | “SS_NG3.2”; exon “3”; |
| chr15 | transcript | 70709440 | 70878937 | − | gene_id “SS_NG3”; transcript_id |
| | | | | | “SS_NG3.1”; RPKM “4.1940”; cov |
| | | | | | “66.6363”; |
| chr15 | exon | 70709440 | 70710841 | − | gene_id “SS_NG3”; transcript_id |
| | | | | | “SS_NG3.1”; exon “1”; |
| chr15 | exon | 70878214 | 70878937 | − | gene_id “SS_NG3”; transcript_id |
| | | | | | “SS_NG3.1”; exon “2”; |
| chr5 | transcript | 124228949 | 124296969 | + | gene_id “SS_NG1”; transcript_id |
| | | | | | “SS_NG1.1”; RPKM “0.9631”; cov |
| | | | | | “15.3022”; |
| chr5 | exon | 124228949 | 124229256 | + | gene_id “SS_NG1”; transcript_id |
| | | | | | “SS_NG1.1”; exon “1”; |
| chr5 | exon | 124286633 | 124286884 | + | gene_id “SS_NG1”; transcript_id |
| | | | | | “SS_NG1.1”; exon “2”; |
| chr5 | exon | 124286977 | 124288649 | + | gene_id “SS_NG1”; transcript_id |
| | | | | | “SS_NG1.1”; exon “3”; |
| chr5 | exon | 124294320 | 124296969 | + | gene_id “SS_NG1”; transcript_id |
| | | | | | “SS_NG1.1”; exon “4”; |
| chr5 | transcript | 124280949 | 124296969 | + | gene_id “SS_NG1”; transcript_id |
| | | | | | “SS_NG1.2”; RPKM “1.0913”; cov |
| | | | | | “17.3396”; |
| chr5 | exon | 124280949 | 124286884 | + | gene_id “SS_NG1”; transcript_id |
| | | | | | “SS_NG1.2”; exon “1”; |
| chr5 | exon | 124286977 | 124288649 | + | gene_id “SS_NG1”; transcript_id |
| | | | | | “SS_NG1.2”; exon “2”; |
| chr5 | exon | 124294320 | 124296969 | + | gene_id “SS_NG1”; transcript_id |
| | | | | | “SS_NG1.2”; exon “3”; |
| chr8 | transcript | 78055063 | 78153568 | + | gene_id “SS_NG2”; transcript_id |
| | | | | | “SS_NG2.8”; RPKM “1.3989”; cov |
| | | | | | “22.2262”; |
| chr8 | exon | 78055063 | 78055362 | + | gene_id “SS_NG2”; transcript_id |
| | | | | | “SS_NG2.8”; exon “1”; |
| chr8 | exon | 78117473 | 78117569 | + | gene_id “SS_NG2”; transcript_id |
| | | | | | “SS_NG2.8”; exon “2”; |
| chr8 | exon | 78120033 | 78120105 | + | gene_id “SS_NG2”; transcript_id |
| | | | | | “SS_NG2.8”; exon “3”; |
| chr8 | exon | 78120459 | 78120563 | + | gene_id “SS_NG2”; transcript_id |
| | | | | | “SS_NG2.8”; exon “4”; |
| chr8 | exon | 78121543 | 78121597 | + | gene_id “SS_NG2”; transcript_id |
| | | | | | “SS_NG2.8”; exon “5”; |
| chr8 | exon | 78124941 | 78127498 | + | gene_id “SS_NG2”; transcript_id |
| | | | | | “SS_NG2.8”; exon “6”; |
| chr8 | exon | 78130820 | 78130871 | + | gene_id “SS_NG2”; transcript_id |
| | | | | | “SS_NG2.8”; exon “7”; |
| chr8 | exon | 78141177 | 78141667 | + | gene_id “SS_NG2”; transcript_id |
| | | | | | “SS_NG2.8”; exon “8”; |
| chr8 | exon | 78147134 | 78148348 | + | gene_id “SS_NG2”; transcript_id |
| | | | | | “SS_NG2.8”; exon “9”; |
| chr8 | exon | 78148841 | 78153568 | + | gene_id “SS_NG2”; transcript_id |
| | | | | | “SS_NG2.8”; exon “10”; |
| chr8 | transcript | 78055063 | 78153568 | + | gene_id “SS_NG2”; transcript_id |
| | | | | | “SS_NG2.7”; RPKM “1.0090”; cov |
| | | | | | “16.0320”; |
| chr8 | exon | 78055063 | 78055362 | + | gene_id “SS_NG2”; transcript_id |
| | | | | | “SS_NG2.7”; exon “1”; |
| chr8 | exon | 78117473 | 78117569 | + | gene_id “SS_NG2”; transcript_id |
| | | | | | “SS_NG2.7”; exon “2”; |
| chr8 | exon | 78120033 | 78120105 | + | gene_id “SS_NG2”; transcript_id |
| | | | | | “SS_NG2.7”; exon “3”; |
| chr8 | exon | 78120459 | 78120563 | + | gene_id “SS_NG2”; transcript_id |
| | | | | | “SS_NG2.7”; exon “4”; |
| chr8 | exon | 78121543 | 78121597 | + | gene_id “SS_NG2”; transcript_id |
| | | | | | “SS_NG2.7”; exon “5”; |
| chr8 | exon | 78124941 | 78127498 | + | gene_id “SS_NG2”; transcript_id |
| | | | | | “SS_NG2.7”; exon “6”; |
| chr8 | exon | 78130820 | 78130871 | + | gene_id “SS_NG2”; transcript_id |
| | | | | | “SS_NG2.7”; exon “7”; |
| chr8 | exon | 78141177 | 78141667 | + | gene_id “SS_NG2”; transcript_id |
| | | | | | “SS_NG2.7”; exon “8”; |
| chr8 | exon | 78147134 | 78149012 | + | gene_id “SS_NG2”; transcript_id |
| | | | | | “SS_NG2.7”; exon “9”; |
| chr8 | exon | 78150146 | 78153568 | + | gene_id “SS_NG2”; transcript_id |
| | | | | | “SS_NG2.7”; exon “10”; |
| chr8 | transcript | 78055063 | 78123090 | + | gene_id “SS_NG2”; transcript_id |
| | | | | | “SS_NG2.6”; RPKM “1.1318”; cov |
| | | | | | “17.9828”; |
| chr8 | exon | 78055063 | 78055362 | + | gene_id “SS_NG2”; transcript_id |
| | | | | | “SS_NG2.6”; exon “1”; |
| chr8 | exon | 78117473 | 78117569 | + | gene_id “SS_NG2”; transcript_id |
| | | | | | “SS_NG2.6”; exon “2”; |
| chr8 | exon | 78120033 | 78120105 | + | gene_id “SS_NG2”; transcript_id |
| | | | | | “SS_NG2.6”; exon “3”; |
| chr8 | exon | 78120459 | 78120563 | + | gene_id “SS_NG2”; transcript_id |
| | | | | | “SS_NG2.6”; exon “4”; |
| chr8 | exon | 78121365 | 78121412 | + | gene_id “SS_NG2”; transcript_id |
| | | | | | “SS_NG2.6”; exon “5”; |
| chr8 | exon | 78121543 | 78123090 | + | gene_id “SS_NG2”; transcript_id |
| | | | | | “SS_NG2.6”; exon “6”; |
| chr8 | transcript | 78055063 | 78100889 | + | gene_id “SS_NG2”; transcript_id |
| | | | | | “SS_NG2.1”; RPKM “1.0720”; cov |
| | | | | | “17.0326”; |
| chr8 | exon | 78055063 | 78055362 | + | gene_id “SS_NG2”; transcript_id |
| | | | | | “SS_NG2.1”; exon “1”; |
| chr8 | exon | 78100600 | 78100889 | + | gene_id “SS_NG2”; transcript_id |
| | | | | | “SS_NG2.1”; exon “2”; |
| chr8 | transcript | 78171057 | 78182416 | + | gene_id “SS_NG2”; transcript_id |
| | | | | | “SS_NG2.2”; RPKM “0.9917”; cov |
| | | | | | “15.7564”; |
| chr8 | exon | 78171057 | 78172951 | + | gene_id “SS_NG2”; transcript_id |
| | | | | | “SS_NG2.2”; exon “1”; |
| chr8 | exon | 78175327 | 78175472 | + | gene_id “SS_NG2”; transcript_id |
| | | | | | “SS_NG2.2”; exon “2”; |
| chr8 | exon | 78176363 | 78176499 | + | gene_id “SS_NG2”; transcript_id |
| | | | | | “SS_NG2.2”; exon “3”; |
| chr8 | exon | 78177038 | 78182416 | + | gene_id “SS_NG2”; transcript_id |
| | | | | | “SS_NG2.2”; exon “4”; |
| chr8 | transcript | 78171057 | 78182416 | + | gene_id “SS_NG2”; transcript_id |
| | | | | | “SS_NG2.3”; RPKM “3.6477”; cov |
| | | | | | “57.9561”; |
| chr8 | exon | 78171057 | 78176499 | + | gene_id “SS_NG2”; transcript_id |
| | | | | | “SS_NG2.3”; exon “1”; |
| chr8 | exon | 78177038 | 78182416 | + | gene_id “SS_NG2”; transcript_id |
| | | | | | “SS_NG2.3”; exon “2”; |
| chr8 | transcript | 78171057 | 78182416 | + | gene_id “SS_NG2”; transcript_id |
| | | | | | “SS_NG2.4”; RPKM “1.5006”; cov |
| | | | | | “23.8413”; |
| chr8 | exon | 78171057 | 78172951 | + | gene_id “SS_NG2”; transcript_id |
| | | | | | “SS_NG2.4”; exon “1”; |
| chr8 | exon | 78175411 | 78175472 | + | gene_id “SS_NG2”; transcript_id |
| | | | | | “SS_NG2.4”; exon “2”; |
| chr8 | exon | 78176363 | 78176499 | + | gene_id “SS_NG2”; transcript_id |
| | | | | | “SS_NG2.4”; exon “3”; |
| chr8 | exon | 78177038 | 78182416 | + | gene_id “SS_NG2”; transcript_id |
| | | | | | “SS_NG2.4”; exon “4”; |
| chr8 | transcript | 78171057 | 78182416 | + | gene_id “SS_NG2”; transcript_id |
| | | | | | “SS_NG2.5”; RPKM “1.4691”; cov |
| | | | | | “23.3421”; |
| chr8 | exon | 78171057 | 78172951 | + | gene_id “SS_NG2”; transcript_id |
| | | | | | “SS_NG2.5”; exon “1”; |
| chr8 | exon | 78175411 | 78176499 | + | gene_id “SS_NG2”; transcript_id |
| | | | | | “SS_NG2.5”; exon “2”; |
| chr8 | exon | 78177038 | 78182416 | + | gene_id “SS_NG2”; transcript_id |
| | | | | | “SS_NG2.5”; exon “3”; |
| chr10 | transcript | 19012211 | 19206483 | − | gene_id “Ew_NG10”; transcript_id |
| | | | | | “Ew_NG10.5”; RPKM “0.7306”; cov |
| | | | | | “11.2000”; |
| chr10 | exon | 19012211 | 19018797 | − | gene_id “Ew_NG10”; transcript_id |
| | | | | | “Ew_NG10.5”; exon “1”; |
| chr10 | exon | 19130037 | 19130094 | − | gene_id “Ew_NG10”; transcript_id |
| | | | | | “Ew_NG10.5”; exon “2”; |
| chr10 | exon | 19149907 | 19149966 | − | gene_id “Ew_NG10”; transcript_id |
| | | | | | “Ew_NG10.5”; exon “3”; |
| chr10 | exon | 19206021 | 19206483 | − | gene_id “Ew_NG10”; transcript_id |
| | | | | | “Ew_NG10.5”; exon “4”; |
| chr10 | transcript | 19064680 | 19206483 | − | gene_id “Ew_NG10”; transcript_id |
| | | | | | “Ew_NG10.2”; RPKM “1.6734”; cov |
| | | | | | “25.6509”; |
| chr10 | exon | 19064680 | 19067502 | − | gene_id “Ew_NG10”; transcript_id |
| | | | | | “Ew_NG10.2”; exon “1”; |
| chr10 | exon | 19130037 | 19130094 | − | gene_id “Ew_NG10”; transcript_id |
| | | | | | “Ew_NG10.2”; exon “2”; |
| chr10 | exon | 19149907 | 19149966 | − | gene_id “Ew_NG10”; transcript_id |
| | | | | | “Ew_NG10.2”; exon “3”; |
| chr10 | exon | 19206021 | 19206483 | − | gene_id “Ew_NG10”; transcript_id |
| | | | | | “Ew_NG10.2”; exon “4”; |
| chr10 | transcript | 19064680 | 19206483 | − | gene_id “Ew_NG10”; transcript_id |
| | | | | | “Ew_NG10.3”; RPKM “0.7329”; cov |
| | | | | | “11.2348”; |
| chr10 | exon | 19064680 | 19067502 | − | gene_id “Ew_NG10”; transcript_id |
| | | | | | “Ew_NG10.3”; exon “1”; |
| chr10 | exon | 19130037 | 19130273 | − | gene_id “Ew_NG10”; transcript_id |
| | | | | | “Ew_NG10.3”; exon “2”; |
| chr10 | exon | 19149907 | 19149966 | − | gene_id “Ew_NG10”; transcript_id |
| | | | | | “Ew_NG10.3”; exon “3”; |
| chr10 | exon | 19206021 | 19206483 | − | gene_id “Ew_NG10”; transcript_id |
| | | | | | “Ew_NG10.3”; exon “4”; |
| chr10 | transcript | 19064680 | 19206483 | − | gene_id “Ew_NG10”; transcript_id |
| | | | | | “Ew_NG10.1”; RPKM “1.7240”; cov |
| | | | | | “26.4277”; |
| chr10 | exon | 19064680 | 19067502 | − | gene_id “Ew_NG10”; transcript_id |
| | | | | | “Ew_NG10.1”; exon “1”; |
| chr10 | exon | 19130037 | 19130094 | − | gene_id “Ew_NG10”; transcript_id |
| | | | | | “Ew_NG10.1”; exon “2”; |
| chr10 | exon | 19206021 | 19206483 | − | gene_id “Ew_NG10”; transcript_id |
| | | | | | “Ew_NG10.1”; exon “3”; |
| chr10 | transcript | 19142228 | 19206483 | − | gene_id “Ew_NG10”; transcript_id |
| | | | | | “Ew_NG10.4”; RPKM “1.0386”; cov |
| | | | | | “15.9205”; |
| chr10 | exon | 19142228 | 19149966 | − | gene_id “Ew_NG10”; transcript_id |
| | | | | | “Ew_NG10.4”; exon “1”; |
| chr10 | exon | 19206021 | 19206483 | − | gene_id “Ew_NG10”; transcript_id |
| | | | | | “Ew_NG10.4”; exon “2”; |
| chr10 | transcript | 26931917 | 26942059 | − | gene_id “Ew_NG11”; transcript_id |
| | | | | | “Ew_NG11.1”; RPKM “3.7572”; cov |
| | | | | | “57.5939”; |
| chr10 | exon | 26931917 | 26942059 | − | gene_id “Ew_NG11”; transcript_id |
| | | | | | “Ew_NG11.1”; exon “1”; |
| chr12 | transcript | 17234067 | 17238065 | − | gene_id “Ew_NG14”; transcript_id |
| | | | | | “Ew_NG14.1”; RPKM “1.4597”; cov |
| | | | | | “22.3758”; |
| chr12 | exon | 17234067 | 17238065 | − | gene_id “Ew_NG14”; transcript_id |
| | | | | | “Ew_NG14.1”; exon “1”; |
| chr12 | transcript | 17297942 | 17303506 | − | gene_id “Ew_NG14”; transcript_id |
| | | | | | “Ew_NG14.2”; RPKM “2.0704”; cov |
| | | | | | “31.7370”; |
| chr12 | exon | 17297942 | 17303506 | − | gene_id “Ew_NG14”; transcript_id |
| | | | | | “Ew_NG14.2”; exon “1”; |
| chr12 | transcript | 17297942 | 17315613 | − | gene_id “Ew_NG14”; transcript_id |
| | | | | | “Ew_NG14.3”; RPKM “0.7621”; cov |
| | | | | | “11.6819”; |
| chr12 | exon | 17297942 | 17299924 | − | gene_id “Ew_NG14”; transcript_id |
| | | | | | “Ew_NG14.3”; exon “1”; |
| chr12 | exon | 17309657 | 17315613 | − | gene_id “Ew_NG14”; transcript_id |
| | | | | | “Ew_NG14.3”; exon “2”; |
| chr12 | transcript | 17297942 | 17320826 | − | gene_id “Ew_NG14”; transcript_id |
| | | | | | “Ew_NG14.4”; RPKM “1.1479”; cov |
| | | | | | “17.5959”; |
| chr12 | exon | 17297942 | 17299924 | − | gene_id “Ew_NG14”; transcript_id |
| | | | | | “Ew_NG14.4”; exon “1”; |
| chr12 | exon | 17309657 | 17309808 | − | gene_id “Ew_NG14”; transcript_id |
| | | | | | “Ew_NG14.4”; exon “2”; |
| chr12 | exon | 17317578 | 17318077 | − | gene_id “Ew_NG14”; transcript_id |
| | | | | | “Ew_NG14.4”; exon “3”; |
| chr12 | exon | 17320594 | 17320826 | − | gene_id “Ew_NG14”; transcript_id |
| | | | | | “Ew_NG14.4”; exon “4”; |
| chr12 | transcript | 17297942 | 17320826 | − | gene_id “Ew_NG14”; transcript_id |
| | | | | | “Ew_NG14.5”; RPKM “1.4469”; cov |
| | | | | | “22.1798”; |
| chr12 | exon | 17297942 | 17299924 | − | gene_id “Ew_NG14”; transcript_id |
| | | | | | “Ew_NG14.5”; exon “1”; |
| chr12 | exon | 17309657 | 17309808 | − | gene_id “Ew_NG14”; transcript_id |
| | | | | | “Ew_NG14.5”; exon “2”; |
| chr12 | exon | 17317578 | 17318077 | − | gene_id “Ew_NG14”; transcript_id |
| | | | | | “Ew_NG14.5”; exon “3”; |
| chr12 | exon | 17319393 | 17319438 | − | gene_id “Ew_NG14”; transcript_id |
| | | | | | “Ew_NG14.5”; exon “4”; |
| chr12 | exon | 17320594 | 17320826 | − | gene_id “Ew_NG14”; transcript_id |
| | | | | | “Ew_NG14.5”; exon “5”; |
| chr12 | transcript | 100749011 | 100750710 | + | gene_id “Ew_NG15”; transcript_id |
| | | | | | “Ew_NG15.1”; RPKM “0.8672”; cov |
| | | | | | “13.2930”; |
| chr12 | exon | 100749011 | 100749133 | + | gene_id “Ew_NG15”; transcript_id |
| | | | | | “Ew_NG15.1”; exon “1”; |
| chr12 | exon | 100749361 | 100750710 | + | gene_id “Ew_NG15”; transcript_id |
| | | | | | “Ew_NG15.1”; exon “2”; |
| chr13 | transcript | 21852260 | 21871372 | − | gene_id “Ew_NG17”; transcript_id |
| | | | | | “Ew_NG17.1”; RPKM “1.3061”; cov |
| | | | | | “20.0207”; |
| chr13 | exon | 21852260 | 21861661 | − | gene_id “Ew_NG17”; transcript_id |
| | | | | | “Ew_NG17.1”; exon “1”; |
| chr13 | exon | 21861774 | 21861983 | − | gene_id “Ew_NG17”; transcript_id |
| | | | | | “Ew_NG17.1”; exon “2”; |
| chr13 | exon | 21866799 | 21866930 | − | gene_id “Ew_NG17”; transcript_id |
| | | | | | “Ew_NG17.1”; exon “3”; |
| chr13 | exon | 21867718 | 21867794 | − | gene_id “Ew_NG17”; transcript_id |
| | | | | | “Ew_NG17.1”; exon “4”; |
| chr13 | exon | 21871214 | 21871372 | − | gene_id “Ew_NG17”; transcript_id |
| | | | | | “Ew_NG17.1”; exon “5”; |
| chr13 | transcript | 21852260 | 21871372 | − | gene_id “Ew_NG17”; transcript_id |
| | | | | | “Ew_NG17.2”; RPKM “0.8464”; cov |
| | | | | | “12.9741”; |
| chr13 | exon | 21852260 | 21861661 | − | gene_id “Ew_NG17”; transcript_id |
| | | | | | “Ew_NG17.2”; exon “1”; |
| chr13 | exon | 21861774 | 21861965 | − | gene_id “Ew_NG17”; transcript_id |
| | | | | | “Ew_NG17.2”; exon “2”; |
| chr13 | exon | 21866799 | 21866930 | − | gene_id “Ew_NG17”; transcript_id |
| | | | | | “Ew_NG17.2”; exon “3”; |
| chr13 | exon | 21867718 | 21867794 | − | gene_id “Ew_NG17”; transcript_id |
| | | | | | “Ew_NG17.2”; exon “4”; |
| chr13 | exon | 21871214 | 21871372 | − | gene_id “Ew_NG17”; transcript_id |
| | | | | | “Ew_NG17.2”; exon “5”; |
| chr13 | transcript | 21852260 | 21871372 | − | gene_id “Ew_NG17”; transcript_id |
| | | | | | “Ew_NG17.3”; RPKM “0.8866”; cov |
| | | | | | “13.5901”; |
| chr13 | exon | 21852260 | 21861661 | − | gene_id “Ew_NG17”; transcript_id |
| | | | | | “Ew_NG17.3”; exon “1”; |
| chr13 | exon | 21861774 | 21861983 | − | gene_id “Ew_NG17”; transcript_id |
| | | | | | “Ew_NG17.3”; exon “2”; |
| chr13 | exon | 21866799 | 21871372 | − | gene_id “Ew_NG17”; transcript_id |
| | | | | | “Ew_NG17.3”; exon “3”; |
| chr13 | transcript | 21852260 | 21871372 | − | gene_id “Ew_NG17”; transcript_id |
| | | | | | “Ew_NG17.4”; RPKM “1.4717”; cov |
| | | | | | “22.5601”; |
| chr13 | exon | 21852260 | 21861661 | − | gene_id “Ew_NG17”; transcript_id |
| | | | | | “Ew_NG17.4”; exon “1”; |
| chr13 | exon | 21861774 | 21861965 | − | gene_id “Ew_NG17”; transcript_id |
| | | | | | “Ew_NG17.4”; exon “2”; |
| chr13 | exon | 21866799 | 21871372 | − | gene_id “Ew_NG17”; transcript_id |
| | | | | | “Ew_NG17.4”; exon “3”; |
| chr13 | transcript | 21852260 | 21871372 | − | gene_id “Ew_NG17”; transcript_id |
| | | | | | “Ew_NG17.5”; RPKM “1.3476”; cov |
| | | | | | “20.6569”; |
| chr13 | exon | 21852260 | 21861661 | − | gene_id “Ew_NG17”; transcript_id |
| | | | | | “Ew_NG17.5”; exon “1”; |
| chr13 | exon | 21861774 | 21861983 | − | gene_id “Ew_NG17”; transcript_id |
| | | | | | “Ew_NG17.5”; exon “2”; |
| chr13 | exon | 21866799 | 21866930 | − | gene_id “Ew_NG17”; transcript_id |
| | | | | | “Ew_NG17.5”; exon “3”; |
| chr13 | exon | 21867642 | 21867794 | − | gene_id “Ew_NG17”; transcript_id |
| | | | | | “Ew_NG17.5”; exon “4”; |
| chr13 | exon | 21871214 | 21871372 | − | gene_id “Ew_NG17”; transcript_id |
| | | | | | “Ew_NG17.5”; exon “5”; |
| chr13 | transcript | 21852260 | 21871372 | − | gene_id “Ew_NG17”; transcript_id |
| | | | | | “Ew_NG17.6”; RPKM “3.3424”; cov |
| | | | | | “51.2347”; |
| chr13 | exon | 21852260 | 21861661 | − | gene_id “Ew_NG17”; transcript_id |
| | | | | | “Ew_NG17.6”; exon “1”; |
| chr13 | exon | 21861774 | 21861965 | − | gene_id “Ew_NG17”; transcript_id |
| | | | | | “Ew_NG17.6”; exon “2”; |
| chr13 | exon | 21866799 | 21866930 | − | gene_id “Ew_NG17”; transcript_id |
| | | | | | “Ew_NG17.6”; exon “3”; |
| chr13 | exon | 21867642 | 21871372 | − | gene_id “Ew_NG17”; transcript_id |
| | | | | | “Ew_NG17.6”; exon “4”; |
| chr13 | transcript | 21852260 | 21871372 | − | gene_id “Ew_NG17”; transcript_id |
| | | | | | “Ew_NG17.7”; RPKM “1.1223”; cov |
| | | | | | “17.2034”; |
| chr13 | exon | 21852260 | 21861661 | − | gene_id “Ew_NG17”; transcript_id |
| | | | | | “Ew_NG17.7”; exon “1”; |
| chr13 | exon | 21861774 | 21861965 | − | gene_id “Ew_NG17”; transcript_id |
| | | | | | “Ew_NG17.7”; exon “2”; |
| chr13 | exon | 21866799 | 21866930 | − | gene_id “Ew_NG17”; transcript_id |
| | | | | | “Ew_NG17.7”; exon “3”; |
| chr13 | exon | 21867642 | 21867794 | − | gene_id “Ew_NG17”; transcript_id |
| | | | | | “Ew_NG17.7”; exon “4”; |
| chr13 | exon | 21871214 | 21871372 | − | gene_id “Ew_NG17”; transcript_id |
| | | | | | “Ew_NG17.7”; exon “5”; |
| chr13 | transcript | 31611186 | 31616347 | − | gene_id “Ew_NG18”; transcript_id |
| | | | | | “Ew_NG18.1”; RPKM “1.2160”; cov |
| | | | | | “18.6401”; |
| chr13 | exon | 31611186 | 31611750 | − | gene_id “Ew_NG18”; transcript_id |
| | | | | | “Ew_NG18.1”; exon “1”; |
| chr13 | exon | 31612458 | 31612628 | − | gene_id “Ew_NG18”; transcript_id |
| | | | | | “Ew_NG18.1”; exon “2”; |
| chr13 | exon | 31616031 | 31616347 | − | gene_id “Ew_NG18”; transcript_id |
| | | | | | “Ew_NG18.1”; exon “3”; |
| chr13 | transcript | 31611186 | 31616347 | − | gene_id “Ew_NG18”; transcript_id |
| | | | | | “Ew_NG18.2”; RPKM “1.7333”; cov |
| | | | | | “26.5694”; |
| chr13 | exon | 31611186 | 31612130 | − | gene_id “Ew_NG18”; transcript_id |
| | | | | | “Ew_NG18.2”; exon “1”; |
| chr13 | exon | 31612458 | 31612628 | − | gene_id “Ew_NG18”; transcript_id |
| | | | | | “Ew_NG18.2”; exon “2”; |
| chr13 | exon | 31616031 | 31616347 | − | gene_id “Ew_NG18”; transcript_id |
| | | | | | “Ew_NG18.2”; exon “3”; |
| chr13 | transcript | 31611186 | 31616347 | − | gene_id “Ew_NG18”; transcript_id |
| | | | | | “Ew_NG18.3”; RPKM “2.2950”; cov |
| | | | | | “35.1794”; |
| chr13 | exon | 31611186 | 31612628 | − | gene_id “Ew_NG18”; transcript_id |
| | | | | | “Ew_NG18.3”; exon “1”; |
| chr13 | exon | 31616031 | 31616347 | − | gene_id “Ew_NG18”; transcript_id |
| | | | | | “Ew_NG18.3”; exon “2”; |
| chr13 | transcript | 39626480 | 39629861 | + | gene_id “Ew_NG19”; transcript_id |
| | | | | | “Ew_NG19.1”; RPKM “2.2687”; cov |
| | | | | | “34.7764”; |
| chr13 | exon | 39626480 | 39629861 | + | gene_id “Ew_NG19”; transcript_id |
| | | | | | “Ew_NG19.1”; exon “1”; |
| chr13 | transcript | 39636412 | 39677230 | + | gene_id “Ew_NG19”; transcript_id |
| | | | | | “Ew_NG19.2”; RPKM “3.3646”; cov |
| | | | | | “51.5758”; |
| chr13 | exon | 39636412 | 39656665 | + | gene_id “Ew_NG19”; transcript_id |
| | | | | | “Ew_NG19.2”; exon “1”; |
| chr13 | exon | 39659264 | 39659391 | + | gene_id “Ew_NG19”; transcript_id |
| | | | | | “Ew_NG19.2”; exon “2”; |
| chr13 | exon | 39662919 | 39677230 | + | gene_id “Ew_NG19”; transcript_id |
| | | | | | “Ew_NG19.2”; exon “3”; |
| chr13 | transcript | 39636412 | 39677230 | + | gene_id “Ew_NG19”; transcript_id |
| | | | | | “Ew_NG19.3”; RPKM “1.0481”; cov |
| | | | | | “16.0663”; |
| chr13 | exon | 39636412 | 39655500 | + | gene_id “Ew_NG19”; transcript_id |
| | | | | | “Ew_NG19.3”; exon “1”; |
| chr13 | exon | 39656578 | 39656665 | + | gene_id “Ew_NG19”; transcript_id |
| | | | | | “Ew_NG19.3”; exon “2”; |
| chr13 | exon | 39659264 | 39659391 | + | gene_id “Ew_NG19”; transcript_id |
| | | | | | “Ew_NG19.3”; exon “3”; |
| chr13 | exon | 39662919 | 39677230 | + | gene_id “Ew_NG19”; transcript_id |
| | | | | | “Ew_NG19.3”; exon “4”; |
| chr19 | transcript | 40653693 | 40656644 | + | gene_id “Ew_NG21”; transcript_id |
| | | | | | “Ew_NG21.1”; RPKM “2.1567”; cov |
| | | | | | “33.0593”; |
| chr19 | exon | 40653693 | 40656644 | + | gene_id “Ew_NG21”; transcript_id |
| | | | | | “Ew_NG21.1”; exon “1”; |
| chr19 | transcript | 40615687 | 40649633 | − | gene_id “Ew_NG22”; transcript_id |
| | | | | | “Ew_NG22.1”; RPKM “1.5547”; cov |
| | | | | | “23.8321”; |
| chr19 | exon | 40615687 | 40617835 | − | gene_id “Ew_NG22”; transcript_id |
| | | | | | “Ew_NG22.1”; exon “1”; |
| chr19 | exon | 40649500 | 40649633 | − | gene_id “Ew_NG22”; transcript_id |
| | | | | | “Ew_NG22.1”; exon “2”; |
| chr19 | transcript | 40615687 | 40649633 | − | gene_id “Ew_NG22”; transcript_id |
| | | | | | “Ew_NG22.2”; RPKM “0.7890”; cov |
| | | | | | “12.0942”; |
| chr19 | exon | 40615687 | 40617835 | − | gene_id “Ew_NG22”; transcript_id |
| | | | | | “Ew_NG22.2”; exon “1”; |
| chr19 | exon | 40639615 | 40639707 | − | gene_id “Ew_NG22”; transcript_id |
| | | | | | “Ew_NG22.2”; exon “2”; |
| chr19 | exon | 40649500 | 40649633 | − | gene_id “Ew_NG22”; transcript_id |
| | | | | | “Ew_NG22.2”; exon “3”; |
| chr19 | transcript | 40615687 | 40649633 | − | gene_id “Ew_NG22”; transcript_id |
| | | | | | “Ew_NG22.4”; RPKM “1.7938”; cov |
| | | | | | “27.4973”; |
| chr19 | exon | 40615687 | 40617835 | − | gene_id “Ew_NG22”; transcript_id |
| | | | | | “Ew_NG22.4”; exon “1”; |
| chr19 | exon | 40619688 | 40620669 | − | gene_id “Ew_NG22”; transcript_id |
| | | | | | “Ew_NG22.4”; exon “2”; |
| chr19 | exon | 40649500 | 40649633 | − | gene_id “Ew_NG22”; transcript_id |
| | | | | | “Ew_NG22.4”; exon “3”; |
| chr19 | transcript | 40615687 | 40649633 | − | gene_id “Ew_NG22”; transcript_id |
| | | | | | “Ew_NG22.3”; RPKM “1.4628”; cov |
| | | | | | “22.4224”; |
| chr19 | exon | 40615687 | 40620669 | − | gene_id “Ew_NG22”; transcript_id |
| | | | | | “Ew_NG22.3”; exon “1”; |
| chr19 | exon | 40649500 | 40649633 | − | gene_id “Ew_NG22”; transcript_id |
| | | | | | “Ew_NG22.3”; exon “2”; |
| chr19 | transcript | 49754681 | 49756653 | + | gene_id “Ew_NG23”; transcript_id |
| | | | | | “Ew_NG23.1”; RPKM “1.3830”; cov |
| | | | | | “21.1997”; |
| chr19 | exon | 49754681 | 49756653 | + | gene_id “Ew_NG23”; transcript_id |
| | | | | | “Ew_NG23.1”; exon “1”; |
| chr19 | transcript | 49757333 | 49765359 | + | gene_id “Ew_NG23”; transcript_id |
| | | | | | “Ew_NG23.2”; RPKM “1.5690”; cov |
| | | | | | “24.0516”; |
| chr19 | exon | 49757333 | 49765359 | + | gene_id “Ew_NG23”; transcript_id |
| | | | | | “Ew_NG23.2”; exon “1”; |
| chr2 | transcript | 4633146 | 4637198 | + | gene_id “Ew_NG2”; transcript_id |
| | | | | | “Ew_NG2.2”; RPKM “0.7645”; cov |
| | | | | | “11.7183”; |
| chr2 | exon | 4633146 | 4633417 | + | gene_id “Ew_NG2”; transcript_id |
| | | | | | “Ew_NG2.2”; exon “1”; |
| chr2 | exon | 4634455 | 4634523 | + | gene_id “Ew_NG2”; transcript_id |
| | | | | | “Ew_NG2.2”; exon “2”; |
| chr2 | exon | 4636162 | 4637198 | + | gene_id “Ew_NG2”; transcript_id |
| | | | | | “Ew_NG2.2”; exon “3”; |
| chr2 | transcript | 4634024 | 4708904 | + | gene_id “Ew_NG2”; transcript_id |
| | | | | | “Ew_NG2.3”; RPKM “3.2015”; cov |
| | | | | | “49.0761”; |
| chr2 | exon | 4634024 | 4634523 | + | gene_id “Ew_NG2”; transcript_id |
| | | | | | “Ew_NG2.3”; exon “1”; |
| chr2 | exon | 4674955 | 4675043 | + | gene_id “Ew_NG2”; transcript_id |
| | | | | | “Ew_NG2.3”; exon “2”; |
| chr2 | exon | 4698519 | 4708904 | + | gene_id “Ew_NG2”; transcript_id |
| | | | | | “Ew_NG2.3”; exon “3”; |
| chr2 | transcript | 4795659 | 4801746 | + | gene_id “Ew_NG2”; transcript_id |
| | | | | | “Ew_NG2.1”; RPKM “1.6250”; cov |
| | | | | | “24.9093”; |
| chr2 | exon | 4795659 | 4801746 | + | gene_id “Ew_NG2”; transcript_id |
| | | | | | “Ew_NG2.1”; exon “1”; |
| chr2 | transcript | 133033581 | 133035142 | − | gene_id “Ew_NG3”; transcript_id |
| | | | | | “Ew_NG3.1”; RPKM “0.7274”; cov |
| | | | | | “11.1495”; |
| chr2 | exon | 133033581 | 133034074 | − | gene_id “Ew_NG3”; transcript_id |
| | | | | | “Ew_NG3.1”; exon “1”; |
| chr2 | exon | 133034603 | 133034739 | − | gene_id “Ew_NG3”; transcript_id |
| | | | | | “Ew_NG3.1”; exon “2”; |
| chr2 | exon | 133035011 | 133035142 | − | gene_id “Ew_NG3”; transcript_id |
| | | | | | “Ew_NG3.1”; exon “3”; |
| chr20 | transcript | 21537669 | 21543139 | + | gene_id “Ew_NG24”; transcript_id |
| | | | | | “Ew_NG24.1”; RPKM “2.8304”; cov |
| | | | | | “43.3865”; |
| chr20 | exon | 21537669 | 21538578 | + | gene_id “Ew_NG24”; transcript_id |
| | | | | | “Ew_NG24.1”; exon “1”; |
| chr20 | exon | 21540530 | 21543139 | + | gene_id “Ew_NG24”; transcript_id |
| | | | | | “Ew_NG24.1”; exon “2”; |
| chr20 | transcript | 21537669 | 21598613 | + | gene_id “Ew_NG24”; transcript_id |
| | | | | | “Ew_NG24.3”; RPKM “1.8896”; cov |
| | | | | | “28.9655”; |
| chr20 | exon | 21537669 | 21538578 | + | gene_id “Ew_NG24”; transcript_id |
| | | | | | “Ew_NG24.3”; exon “1”; |
| chr20 | exon | 21593186 | 21593321 | + | gene_id “Ew_NG24”; transcript_id |
| | | | | | “Ew_NG24.3”; exon “2”; |
| chr20 | exon | 21596021 | 21596165 | + | gene_id “Ew_NG24”; transcript_id |
| | | | | | “Ew_NG24.3”; exon “3”; |
| chr20 | exon | 21596251 | 21598613 | + | gene_id “Ew_NG24”; transcript_id |
| | | | | | “Ew_NG24.3”; exon “4”; |
| chr20 | transcript | 21593088 | 21598613 | + | gene_id “Ew_NG24”; transcript_id |
| | | | | | “Ew_NG24.2”; RPKM “1.1532”; cov |
| | | | | | “17.6775”; |
| chr20 | exon | 21593088 | 21593321 | + | gene_id “Ew_NG24”; transcript_id |
| | | | | | “Ew_NG24.2”; exon “1”; |
| chr20 | exon | 21596021 | 21596165 | + | gene_id “Ew_NG24”; transcript_id |
| | | | | | “Ew_NG24.2”; exon “2”; |
| chr20 | exon | 21596251 | 21598613 | + | gene_id “Ew_NG24”; transcript_id |
| | | | | | “Ew_NG24.2”; exon “3”; |
| chr20 | transcript | 21604484 | 21612837 | + | gene_id “Ew_NG25”; transcript_id |
| | | | | | “Ew_NG25.1”; RPKM “2.7790”; cov |
| | | | | | “42.5995”; |
| chr20 | exon | 21604484 | 21612837 | + | gene_id “Ew_NG25”; transcript_id |
| | | | | | “Ew_NG25.1”; exon “1”; |
| chr4 | transcript | 8078741 | 8087977 | + | gene_id “Ew_NG5”; transcript_id |
| | | | | | “Ew_NG5.1”; RPKM “12.6756”; cov |
| | | | | | “194.3033”; |
| chr4 | exon | 8078741 | 8087977 | + | gene_id “Ew_NG5”; transcript_id |
| | | | | | “Ew_NG5.1”; exon “1”; |
| chr5 | transcript | 157372872 | 157410161 | + | gene_id “Ew_NG6”; transcript_id |
| | | | | | “Ew_NG6.1”; RPKM “1.0807”; cov |
| | | | | | “16.5652”; |
| chr5 | exon | 157372872 | 157373508 | + | gene_id “Ew_NG6”; transcript_id |
| | | | | | “Ew_NG6.1”; exon “1”; |
| chr5 | exon | 157409256 | 157410161 | + | gene_id “Ew_NG6”; transcript_id |
| | | | | | “Ew_NG6.1”; exon “2”; |
| chr5 | transcript | 157372872 | 157410161 | + | gene_id “Ew_NG6”; transcript_id |
| | | | | | “Ew_NG6.2”; RPKM “0.7152”; cov |
| | | | | | “10.9632”; |
| chr5 | exon | 157372872 | 157372974 | + | gene_id “Ew_NG6”; transcript_id |
| | | | | | “Ew_NG6.2”; exon “1”; |
| chr5 | exon | 157403125 | 157403337 | + | gene_id “Ew_NG6”; transcript_id |
| | | | | | “Ew_NG6.2”; exon “2”; |
| chr5 | exon | 157409256 | 157410161 | + | gene_id “Ew_NG6”; transcript_id |
| | | | | | “Ew_NG6.2”; exon “3”; |
| chr5 | transcript | 157400974 | 157410161 | + | gene_id “Ew_NG6”; transcript_id |
| | | | | | “Ew_NG6.3”; RPKM “0.9828”; cov |
| | | | | | “15.0657”; |
| chr5 | exon | 157400974 | 157403337 | + | gene_id “Ew_NG6”; transcript_id |
| | | | | | “Ew_NG6.3”; exon “1”; |
| chr5 | exon | 157409256 | 157410161 | + | gene_id “Ew_NG6”; transcript_id |
| | | | | | “Ew_NG6.3”; exon “2”; |
| chr8 | transcript | 6183756 | 6189727 | + | gene_id “Ew_NG7”; transcript_id |
| | | | | | “Ew_NG7.1”; RPKM “1.1857”; cov |
| | | | | | “18.1754”; |
| chr8 | exon | 6183756 | 6183900 | + | gene_id “Ew_NG7”; transcript_id |
| | | | | | “Ew_NG7.1”; exon “1”; |
| chr8 | exon | 6189197 | 6189727 | + | gene_id “Ew_NG7”; transcript_id |
| | | | | | “Ew_NG7.1”; exon “2”; |
| chr8 | transcript | 27005650 | 27047229 | − | gene_id “Ew_NG8”; transcript_id |
| | | | | | “Ew_NG8.1”; RPKM “112.8233”; cov |
| | | | | | “1729.4563”; |
| chr8 | exon | 27005650 | 27009409 | − | gene_id “Ew_NG8”; transcript_id |
| | | | | | “Ew_NG8.1”; exon “1”; |
| chr8 | exon | 27046675 | 27047229 | − | gene_id “Ew_NG8”; transcript_id |
| | | | | | “Ew_NG8.1”; exon “2”; |
| chr8 | transcript | 27005650 | 27047229 | − | gene_id “Ew_NG8”; transcript_id |
| | | | | | “Ew_NG8.2”; RPKM “1.0177”; cov |
| | | | | | “15.6000”; |
| chr8 | exon | 27005650 | 27009409 | − | gene_id “Ew_NG8”; transcript_id |
| | | | | | “Ew_NG8.2”; exon “1”; |
| chr8 | exon | 27027995 | 27028040 | − | gene_id “Ew_NG8”; transcript_id |
| | | | | | “Ew_NG8.2”; exon “2”; |
| chr8 | exon | 27046675 | 27047229 | − | gene_id “Ew_NG8”; transcript_id |
| | | | | | “Ew_NG8.2”; exon “3”; |
| chr8 | transcript | 27005650 | 27047229 | − | gene_id “Ew_NG8”; transcript_id |
| | | | | | “Ew_NG8.7”; RPKM “0.9321”; cov |
| | | | | | “14.2882”; |
| chr8 | exon | 27005650 | 27016399 | − | gene_id “Ew_NG8”; transcript_id |
| | | | | | “Ew_NG8.7”; exon “1”; |
| chr8 | exon | 27021071 | 27021129 | − | gene_id “Ew_NG8”; transcript_id |
| | | | | | “Ew_NG8.7”; exon “2”; |
| chr8 | exon | 27046675 | 27047229 | − | gene_id “Ew_NG8”; transcript_id |
| | | | | | “Ew_NG8.7”; exon “3”; |
| chr8 | transcript | 27005650 | 27047229 | − | gene_id “Ew_NG8”; transcript_id |
| | | | | | “Ew_NG8.6”; RPKM “0.8994”; cov |
| | | | | | “13.7873”; |
| chr8 | exon | 27005650 | 27009409 | − | gene_id “Ew_NG8”; transcript_id |
| | | | | | “Ew_NG8.6”; exon “1”; |
| chr8 | exon | 27013135 | 27013241 | − | gene_id “Ew_NG8”; transcript_id |
| | | | | | “Ew_NG8.6”; exon “2”; |
| chr8 | exon | 27046675 | 27047229 | − | gene_id “Ew_NG8”; transcript_id |
| | | | | | “Ew_NG8.6”; exon “3”; |
| chr8 | transcript | 27005650 | 27047229 | − | gene_id “Ew_NG8”; transcript_id |
| | | | | | “Ew_NG8.5”; RPKM “20.5120”; cov |
| | | | | | “314.4266”; |
| chr8 | exon | 27005650 | 27009409 | − | gene_id “Ew_NG8”; transcript_id |
| | | | | | “Ew_NG8.5”; exon “1”; |
| chr8 | exon | 27021071 | 27021129 | − | gene_id “Ew_NG8”; transcript_id |
| | | | | | “Ew_NG8.5”; exon “2”; |
| chr8 | exon | 27046675 | 27047229 | − | gene_id “Ew_NG8”; transcript_id |
| | | | | | “Ew_NG8.5”; exon “3”; |
| chr8 | transcript | 27026051 | 27047229 | − | gene_id “Ew_NG8”; transcript_id |
| | | | | | “Ew_NG8.4”; RPKM “6.8060”; cov |
| | | | | | “104.3279”; |
| chr8 | exon | 27026051 | 27042494 | − | gene_id “Ew_NG8”; transcript_id |
| | | | | | “Ew_NG8.4”; exon “1”; |
| chr8 | exon | 27046675 | 27047229 | − | gene_id “Ew_NG8”; transcript_id |
| | | | | | “Ew_NG8.4”; exon “2”; |
| chr8 | transcript | 27033709 | 27047229 | − | gene_id “Ew_NG8”; transcript_id |
| | | | | | “Ew_NG8.3”; RPKM “6.4353”; cov |
| | | | | | “98.6458”; |
| chr8 | exon | 27033709 | 27042018 | − | gene_id “Ew_NG8”; transcript_id |
| | | | | | “Ew_NG8.3”; exon “1”; |
| chr8 | exon | 27046675 | 27047229 | − | gene_id “Ew_NG8”; transcript_id |
| | | | | | “Ew_NG8.3”; exon “2”; |
| chr9 | transcript | 16084058 | 16095297 | + | gene_id “Ew_NG9”; transcript_id |
| | | | | | “Ew_NG9.1”; RPKM “1.5231”; cov |
| | | | | | “23.3469”; |
| chr9 | exon | 16084058 | 16095297 | + | gene_id “Ew_NG9”; transcript_id |
| | | | | | “Ew_NG9.1”; exon “1”; |
| chr1 | transcript | 73171083 | 73221254 | + | gene_id “Ew_NG1”; transcript_id |
| | | | | | “Ew_NG1.1”; RPKM “1.7032”; cov |
| | | | | | “30.4365”; |
| chr1 | exon | 73171083 | 73171273 | + | gene_id “Ew_NG1”; transcript_id |
| | | | | | “Ew_NG1.1”; exon “1”; |
| chr1 | exon | 73174742 | 73174919 | + | gene_id “Ew_NG1”; transcript_id |
| | | | | | “Ew_NG1.1”; exon “2”; |
| chr1 | exon | 73219351 | 73221254 | + | gene_id “Ew_NG1”; transcript_id |
| | | | | | “Ew_NG1.1”; exon “3”; |
| chr13 | transcript | 31571787 | 31590888 | − | gene_id “Ew_NG12”; transcript_id |
| | | | | | “Ew_NG12.1”; RPKM “1.8525”; cov |
| | | | | | “33.1034”; |
| chr13 | exon | 31571787 | 31573006 | − | gene_id “Ew_NG12”; transcript_id |
| | | | | | “Ew_NG12.1”; exon “1”; |
| chr13 | exon | 31575483 | 31575583 | − | gene_id “Ew_NG12”; transcript_id |
| | | | | | “Ew_NG12.1”; exon “2”; |
| chr13 | exon | 31583450 | 31583627 | − | gene_id “Ew_NG12”; transcript_id |
| | | | | | “Ew_NG12.1”; exon “3”; |
| chr13 | exon | 31590612 | 31590888 | − | gene_id “Ew_NG12”; transcript_id |
| | | | | | “Ew_NG12.1”; exon “4”; |
| chr13 | transcript | 31577913 | 31590888 | − | gene_id “Ew_NG12”; transcript_id |
| | | | | | “Ew_NG12.2”; RPKM “0.8159”; cov |
| | | | | | “14.5800”; |
| chr13 | exon | 31577913 | 31579070 | − | gene_id “Ew_NG12”; transcript_id |
| | | | | | “Ew_NG12.2”; exon “1”; |
| chr13 | exon | 31583450 | 31583627 | − | gene_id “Ew_NG12”; transcript_id |
| | | | | | “Ew_NG12.2”; exon “2”; |
| chr13 | exon | 31590612 | 31590888 | − | gene_id “Ew_NG12”; transcript_id |
| | | | | | “Ew_NG12.2”; exon “3”; |
| chr14 | transcript | 62935997 | 63066538 | − | gene_id “Ew_NG13”; transcript_id |
| | | | | | “Ew_NG13.1”; RPKM “0.6622”; cov |
| | | | | | “11.8340”; |
| chr14 | exon | 62935997 | 62938240 | − | gene_id “Ew_NG13”; transcript_id |
| | | | | | “Ew_NG13.1”; exon “1”; |
| chr14 | exon | 62952321 | 62952545 | − | gene_id “Ew_NG13”; transcript_id |
| | | | | | “Ew_NG13.1”; exon “2”; |
| chr14 | exon | 62954188 | 62954496 | − | gene_id “Ew_NG13”; transcript_id |
| | | | | | “Ew_NG13.1”; exon “3”; |
| chr14 | exon | 62954981 | 62955105 | − | gene_id “Ew_NG13”; transcript_id |
| | | | | | “Ew_NG13.1”; exon “4”; |
| chr14 | exon | 62959391 | 62959490 | − | gene_id “Ew_NG13”; transcript_id |
| | | | | | “Ew_NG13.1”; exon “5”; |
| chr14 | exon | 63066366 | 63066538 | − | gene_id “Ew_NG13”; transcript_id |
| | | | | | “Ew_NG13.1”; exon “6”; |
| chr16 | transcript | 8686922 | 8687845 | + | gene_id “Ew_NG16”; transcript_id |
| | | | | | “Ew_NG16.1”; RPKM “4.7102”; cov |
| | | | | | “84.1710”; |
| chr16 | exon | 8686922 | 8687845 | + | gene_id “Ew_NG16”; transcript_id |
| | | | | | “Ew_NG16.1”; exon “1”; |
| chr4 | transcript | 118075235 | 118081651 | + | gene_id “Ew_NG4”; transcript_id |
| | | | | | “Ew_NG4.1”; RPKM “1.7974”; cov |
| | | | | | “32.1197”; |
| chr4 | exon | 118075235 | 118075393 | + | gene_id “Ew_NG4”; transcript_id |
| | | | | | “Ew_NG4.1”; exon “1”; |
| chr4 | exon | 118080857 | 118081651 | + | gene_id “Ew_NG4”; transcript_id |
| | | | | | “Ew_NG4.1”; exon “2”; |
| chrX | transcript | 52799508 | 52810286 | − | gene_id “Ew_NG20”; transcript_id |
| | | | | | “Ew_NG20.1”; RPKM “0.6979”; cov |
| | | | | | “12.4714”; |
| chrX | exon | 52799508 | 52799624 | − | gene_id “Ew_NG20”; transcript_id |
| | | | | | “Ew_NG20.1”; exon “1”; |
| chrX | exon | 52809582 | 52809671 | − | gene_id “Ew_NG20”; transcript_id |
| | | | | | “Ew_NG20.1”; exon “2”; |
| chrX | exon | 52809783 | 52810286 | − | gene_id “Ew_NG20”; transcript_id |
| | | | | | “Ew_NG20.1”; exon “3”; |
| chrX | transcript | 52808948 | 52810286 | − | gene_id “Ew_NG20”; transcript_id |
| | | | | | “Ew_NG20.2”; RPKM “8.6550”; cov |
| | | | | | “154.6633”; |
| chrX | exon | 52808948 | 52809671 | − | gene_id “Ew_NG20”; transcript_id |
| | | | | | “Ew_NG20.2”; exon “1”; |
| chrX | exon | 52809783 | 52810286 | − | gene_id “Ew_NG20”; transcript_id |
| | | | | | “Ew_NG20.2”; exon “2”; |
| chr10 | transcript | 1778736 | 1780373 | + | gene_id “AFH_NG9”; transcript_id |
| | | | | | “AFH_NG9.1”; RPKM “4.2559”; cov |
| | | | | | “47.3341”; |
| chr10 | exon | 1778736 | 1780373 | + | gene_id “AFH_NG9”; transcript_id |
| | | | | | “AFH_NG9.1”; exon “1”; |
| chr10 | transcript | 1595092 | 1600429 | − | gene_id “AFH_NG10”; transcript_id |
| | | | | | “AFH_NG10.1”; RPKM “5.0731”; cov |
| | | | | | “56.4228”; |
| chr10 | exon | 1595092 | 1600429 | − | gene_id “AFH_NG10”; transcript_id |
| | | | | | “AFH_NG10.1”; exon “1”; |
| chr11 | transcript | 134450851 | 134454089 | − | gene_id “AFH_NG11”; transcript_id |
| | | | | | “AFH_NG11.1”; RPKM “2.3309”; cov |
| | | | | | “25.9249”; |
| chr11 | exon | 134450851 | 134451453 | − | gene_id “AFH_NG11”; transcript_id |
| | | | | | “AFH_NG11.1”; exon “1”; |
| chr11 | exon | 134453612 | 134454089 | − | gene_id “AFH_NG11”; transcript_id |
| | | | | | “AFH_NG11.1”; exon “2”; |
| chr12 | transcript | 5531617 | 5543161 | − | gene_id “AFH_NG12”; transcript_id |
| | | | | | “AFH_NG12.1”; RPKM “10.4014”; cov |
| | | | | | “115.6853”; |
| chr12 | exon | 5531617 | 5538474 | − | gene_id “AFH_NG12”; transcript_id |
| | | | | | “AFH_NG12.1”; exon “1”; |
| chr12 | exon | 5539507 | 5540451 | − | gene_id “AFH_NG12”; transcript_id |
| | | | | | “AFH_NG12.1”; exon “2”; |
| chr12 | exon | 5540611 | 5541467 | − | gene_id “AFH_NG12”; transcript_id |
| | | | | | “AFH_NG12.1”; exon “3”; |
| chr12 | exon | 5542222 | 5543161 | − | gene_id “AFH_NG12”; transcript_id |
| | | | | | “AFH_NG12.1”; exon “4”; |
| chr12 | transcript | 5531617 | 5543161 | − | gene_id “AFH_NG12”; transcript_id |
| | | | | | “AFH_NG12.2”; RPKM “11.1707”; cov |
| | | | | | “124.2416”; |
| chr12 | exon | 5531617 | 5538474 | − | gene_id “AFH_NG12”; transcript_id |
| | | | | | “AFH_NG12.2”; exon “1”; |
| chr12 | exon | 5539507 | 5540451 | − | gene_id “AFH_NG12”; transcript_id |
| | | | | | “AFH_NG12.2”; exon “2”; |
| chr12 | exon | 5540611 | 5543161 | − | gene_id “AFH_NG12”; transcript_id |
| | | | | | “AFH_NG12.2”; exon “3”; |
| chr12 | transcript | 5637918 | 5641928 | + | gene_id “AFH_NG13”; transcript_id |
| | | | | | “AFH_NG13.1”; RPKM “5.5218”; cov |
| | | | | | “61.4136”; |
| chr1 2 | exon | 5637918 | 5641928 | + | gene_id “AFH_NG13”; transcript_id |
| | | | | | “AFH_NG13.1”; exon “1”; |
| chr12 | transcript | 129537390 | 129539654 | − | gene_id “AFH_NG14”; transcript_id |
| | | | | | “AFH_NG14.1”; RPKM “2.1949”; cov |
| | | | | | “24.4115”; |
| chr12 | exon | 129537390 | 129539654 | − | gene_id “AFH_NG14”; transcript_id |
| | | | | | “AFH_NG14.1”; exon “1”; |
| chr1 3 | transcript | 29295709 | 29305491 | + | gene_id “AFH_NG15”; transcript_id |
| | | | | | “AFH_NG15.1”; RPKM “2.6231”; cov |
| | | | | | “29.1742”; |
| chr13 | exon | 29295709 | 29305491 | + | gene_id “AFH_NG15”; transcript_id |
| | | | | | “AFH_NG15.1”; exon “1”; |
| chr16 | transcript | 86663670 | 86666438 | + | gene_id “AFH_NG16”; transcript_id |
| | | | | | “AFH_NG16.1”; RPKM “5.3721”; cov |
| | | | | | “59.7488”; |
| chr16 | exon | 86663670 | 86666438 | + | gene_id “AFH_NG16”; transcript_id |
| | | | | | “AFH_NG16.1”; exon “1”; |
| chr17 | transcript | 6245849 | 6268793 | − | gene_id “AFH_NG17”; transcript_id |
| | | | | | “AFH_NG17.2”; RPKM “1.0621”; cov |
| | | | | | “11.8122”; |
| chr17 | exon | 6245849 | 6246191 | − | gene_id “AFH_NG17”; transcript_id |
| | | | | | “AFH_NG17.2”; exon “1”; |
| chr17 | exon | 6258068 | 6258152 | − | gene_id “AFH_NG17”; transcript_id |
| | | | | | “AFH_NG17.2”; exon “2”; |
| chr17 | exon | 6268472 | 6268793 | − | gene_id “AFH_NG17”; transcript_id |
| | | | | | “AFH_NG17.2”; exon “3”; |
| chr17 | transcript | 6255407 | 6268793 | − | gene_id “AFH_NG17”; transcript_id |
| | | | | | “AFH_NG17.1”; RPKM “1.4945”; cov |
| | | | | | “16.6225”; |
| chr17 | exon | 6255407 | 6258152 | − | gene_id “AFH_NG17”; transcript_id |
| | | | | | “AFH_NG17.1”; exon “1”; |
| chr17 | exon | 6268472 | 6268793 | − | gene_id “AFH_NG17”; transcript_id |
| | | | | | “AFH_NG17.1”; exon “2”; |
| chr1 7 | transcript | 77813452 | 77816523 | + | gene_id “AFH_NG18”; transcript_id |
| | | | | | “AFH_NG18.1”; RPKM “0.9375”; cov |
| | | | | | “10.4269”; |
| chr1 7 | exon | 77813452 | 77815330 | + | gene_id “AFH_NG18”; transcript_id |
| | | | | | “AFH_NG18.1”; exon “1”; |
| chr1 7 | exon | 77815460 | 77816523 | + | gene_id “AFH_NG18”; transcript_id |
| | | | | | “AFH_NG18.1”; exon “2”; |
| chr1 7 | transcript | 77813452 | 77816523 | + | gene_id “AFH_NG18”; transcript_id |
| | | | | | “AFH_NG18.2”; RPKM “8.0905”; cov |
| | | | | | “89.9834”; |
| chr17 | exon | 77813452 | 77816523 | + | gene_id “AFH_NG18”; transcript_id |
| | | | | | “AFH_NG18.2”; exon “1”; |
| chr19 | transcript | 9089290 | 9114010 | + | gene_id “AFH_NG19”; transcript_id |
| | | | | | “AFH_NG19.2”; RPKM “1.2534”; cov |
| | | | | | “13.9400”; |
| chr19 | exon | 9089290 | 9089931 | + | gene_id “AFH_NG19”; transcript_id |
| | | | | | “AFH_NG19.2”; exon “1”; |
| chr19 | exon | 9113063 | 9114010 | + | gene_id “AFH_NG19”; transcript_id |
| | | | | | “AFH_NG19.2”; exon “2”; |
| chr19 | transcript | 9114907 | 9120441 | + | gene_id “AFH_NG19”; transcript_id |
| | | | | | “AFH_NG19.1”; RPKM “1.8572”; cov |
| | | | | | “20.6563”; |
| chr1 9 | exon | 9114907 | 9120441 | + | gene_id “AFH_NG19”; transcript_id |
| | | | | | “AFH_NG19.1”; exon “1”; |
| chr22 | transcript | 35918439 | 35921521 | + | gene_id “AFH_NG20”; transcript_id |
| | | | | | “AFH_NG20.1”; RPKM “1.0768”; cov |
| | | | | | “11.9760”; |
| chr22 | exon | 35918439 | 35919124 | + | gene_id “AFH_NG20”; transcript_id |
| | | | | | “AFH_NG20.1”; exon “1”; |
| chr22 | exon | 35919943 | 35921521 | + | gene_id “AFH_NG20”; transcript_id |
| | | | | | “AFH_NG20.1”; exon “2”; |
| chr3 | transcript | 67141203 | 67229790 | + | gene_id “AFH_NG1”; transcript_id |
| | | | | | “AFH_NG1.2”; RPKM “1.4165”; cov |
| | | | | | “15.7549”; |
| chr3 | exon | 67141203 | 67141315 | + | gene_id “AFH_NG1”; transcript_id |
| | | | | | “AFH_NG1.2”; exon “1”; |
| chr3 | exon | 67224541 | 67224668 | + | gene_id “AFH_NG1”; transcript_id |
| | | | | | “AFH_NG1.2”; exon “2”; |
| chr3 | exon | 67226608 | 67227350 | + | gene_id “AFH_NG1”; transcript_id |
| | | | | | “AFH_NG1.2”; exon “3”; |
| chr3 | exon | 67227862 | 67229790 | + | gene_id “AFH_NG1”; transcript_id |
| | | | | | “AFH_NG1.2”; exon “4”; |
| chr3 | transcript | 67221043 | 67229790 | + | gene_id “AFH_NG1”; transcript_id |
| | | | | | “AFH_NG1.3”; RPKM “1.4170”; cov |
| | | | | | “15.7603”; |
| chr3 | exon | 67221043 | 67224335 | + | gene_id “AFH_NG1”; transcript_id |
| | | | | | “AFH_NG1.3”; exon “1”; |
| chr3 | exon | 67224541 | 67224668 | + | gene_id “AFH_NG1”; transcript_id |
| | | | | | “AFH_NG1.3”; exon “2”; |
| chr3 | exon | 67226608 | 67227350 | + | gene_id “AFH_NG1”; transcript_id |
| | | | | | “AFH_NG1.3”; exon “3”; |
| chr3 | exon | 67227862 | 67229790 | + | gene_id “AFH_NG1”; transcript_id |
| | | | | | “AFH_NG1.3”; exon “4”; |
| chr3 | transcript | 67240721 | 67244755 | + | gene_id “AFH_NG1”; transcript_id |
| | | | | | “AFH_NG1.1”; RPKM “3.2035”; cov |
| | | | | | “35.6300”; |
| chr3 | exon | 67240721 | 67244755 | + | gene_id “AFH_NG1”; transcript_id |
| | | | | | “AFH_NG1.1”; exon “1”; |
| chr5 | transcript | 15063076 | 15097733 | − | gene_id “AFH_NG2”; transcript_id |
| | | | | | “AFH_NG2.1”; RPKM “19.1635”; cov |
| | | | | | “213.1369”; |
| chr5 | exon | 15063076 | 15066397 | − | gene_id “AFH_NG2”; transcript_id |
| | | | | | “AFH_NG2.1”; exon “1”; |
| chr5 | exon | 15068595 | 15081814 | − | gene_id “AFH_NG2”; transcript_id |
| | | | | | “AFH_NG2.1”; exon “2”; |
| chr5 | exon | 15082978 | 15086448 | − | gene_id “AFH_NG2”; transcript_id |
| | | | | | “AFH_NG2.1”; exon “3”; |
| chr5 | exon | 15097230 | 15097733 | − | gene_id “AFH_NG2”; transcript_id |
| | | | | | “AFH_NG2.1”; exon “4”; |
| chr5 | transcript | 15063076 | 15097733 | − | gene_id “AFH_NG2”; transcript_id |
| | | | | | “AFH_NG2.2”; RPKM “25.0145”; cov |
| | | | | | “278.2129”; |
| chr5 | exon | 15063076 | 15066397 | − | gene_id “AFH_NG2”; transcript_id |
| | | | | | “AFH_NG2.2”; exon “1”; |
| chr5 | exon | 15068595 | 15086448 | − | gene_id “AFH_NG2”; transcript_id |
| | | | | | “AFH_NG2.2”; exon “2”; |
| chr5 | exon | 15097230 | 15097733 | − | gene_id “AFH_NG2”; transcript_id |
| | | | | | “AFH_NG2.2”; exon “3”; |
| chr5 | transcript | 15091700 | 15096714 | − | gene_id “AFH_NG3”; transcript_id |
| | | | | | “AFH_NG3.1”; RPKM “8.6436”; cov |
| | | | | | “96.1346”; |
| chr5 | exon | 15091700 | 15096714 | − | gene_id “AFH_NG3”; transcript_id |
| | | | | | “AFH_NG3.1”; exon “1”; |
| chr5 | transcript | 170062653 | 170070157 | + | gene_id “AFH_NG4”; transcript_id |
| | | | | | “AFH_NG4.1”; RPKM “2.8235”; cov |
| | | | | | “31.4032”; |
| chr5 | exon | 170062653 | 170070157 | + | gene_id “AFH_NG4”; transcript_id |
| | | | | | “AFH_NG4.1”; exon “1”; |
| chr8 | transcript | 123485229 | 123488871 | − | gene_id “AFH_NG7”; transcript_id |
| | | | | | “AFH_NG7.1”; RPKM “7.7204”; cov |
| | | | | | “85.8669”; |
| chr8 | exon | 123485229 | 123488871 | − | gene_id “AFH_NG7”; transcript_id |
| | | | | | “AFH_NG7.1”; exon “1”; |
| chr8 | transcript | 123639003 | 123640851 | − | gene_id “AFH_NG5”; transcript_id |
| | | | | | “AFH_NG5.1”; RPKM “3.2829”; cov |
| | | | | | “36.5122”; |
| chr8 | exon | 123639003 | 123640851 | − | gene_id “AFH_NG5”; transcript_id |
| | | | | | “AFH_NG5.1”; exon “1”; |
| chr8 | transcript | 123663969 | 123666843 | − | gene_id “AFH_NG6”; transcript_id |
| | | | | | “AFH_NG6.1”; RPKM “3.5461”; cov |
| | | | | | “39.4397”; |
| chr8 | exon | 123663969 | 123666843 | − | gene_id “AFH_NG6”; transcript_id |
| | | | | | “AFH_NG6.1”; exon “1”; |
| chr9 | transcript | 117706247 | 117762704 | − | gene_id “AFH_NG8”; transcript_id |
| | | | | | “AFH_NG8.1”; RPKM “3.6544”; cov |
| | | | | | “40.6447”; |
| chr9 | exon | 117706247 | 117706470 | − | gene_id “AFH_NG8”; transcript_id |
| | | | | | “AFH_NG8.1”; exon “1”; |
| chr9 | exon | 117715682 | 117715903 | − | gene_id “AFH_NG8”; transcript_id |
| | | | | | “AFH_NG8.1”; exon “2”; |
| chr9 | exon | 117762333 | 117762704 | − | gene_id “AFH_NG8”; transcript_id |
| | | | | | “AFH_NG8.1”; exon “3”; |
| chr9 | transcript | 117712105 | 117762704 | − | gene_id “AFH_NG8”; transcript_id |
| | | | | | “AFH_NG8.2”; RPKM “2.8967”; cov |
| | | | | | “32.2169”; |
| chr9 | exon | 117712105 | 117715903 | − | gene_id “AFH_NG8”; transcript_id |
| | | | | | “AFH_NG8.2”; exon “1”; |
| chr9 | exon | 117762333 | 117762704 | − | gene_id “AFH_NG8”; transcript_id |
| | | | | | “AFH_NG8.2”; exon “2”; |
| chr1 | transcript | 201147343 | 201157055 | − | gene_id “ASPS_NG1”; transcript_id |
| | | | | | “ASPS_NG1.2”; RPKM “3.3560”; cov |
| | | | | | “44.4954”; |
| chr1 | exon | 201147343 | 201150774 | − | gene_id “ASPS_NG1”; transcript_id |
| | | | | | “ASPS_NG1.2”; exon “1”; |
| chr1 | exon | 201155753 | 201155909 | − | gene_id “ASPS_NG1”; transcript_id |
| | | | | | “ASPS_NG1.2”; exon “2”; |
| chr1 | exon | 201157033 | 201157055 | − | gene_id “ASPS_NG1”; transcript_id |
| | | | | | “ASPS_NG1.2”; exon “3”; |
| chr1 | transcript | 201155105 | 201157055 | − | gene_id “ASPS_NG1”; transcript_id |
| | | | | | “ASPS_NG1.1”; RPKM “0.7948”; cov |
| | | | | | “10.5373”; |
| chr1 | exon | 201155105 | 201155909 | − | gene_id “ASPS_NG1”; transcript_id |
| | | | | | “ASPS_NG1.1”; exon “1”; |
| chr1 | exon | 201157033 | 201157055 | − | gene_id “ASPS_NG1”; transcript_id |
| | | | | | “ASPS_NG1.1”; exon “2”; |
| chr1 | transcript | 222181182 | 222182054 | − | gene_id “ASPS_NG2”; transcript_id |
| | | | | | “ASPS_NG2.1”; RPKM “5.6644”; cov |
| | | | | | “75.1008”; |
| chr1 | exon | 222181182 | 222182054 | − | gene_id “ASPS_NG2”; transcript_id |
| | | | | | “ASPS_NG2.1”; exon “1”; |
| chr10 | transcript | 47657110 | 47671291 | − | gene_id “ASPS_NG23”; transcript_id |
| | | | | | “ASPS_NG23.1”; RPKM “12.5628”; cov |
| | | | | | “166.5635”; |
| chr10 | exon | 47657110 | 47667516 | − | gene_id “ASPS_NG23”; transcript_id |
| | | | | | “ASPS_NG23.1”; exon “1”; |
| chr10 | exon | 47670549 | 47671291 | − | gene_id “ASPS_NG23”; transcript_id |
| | | | | | “ASPS_NG23.1”; exon “2”; |
| chr10 | transcript | 47657110 | 47671291 | − | gene_id “ASPS_NG23”; transcript_id |
| | | | | | “ASPS_NG23.2”; RPKM “1.5928”; cov |
| | | | | | “21.1180”; |
| chr10 | exon | 47657110 | 47671291 | − | gene_id “ASPS_NG23”; transcript_id |
| | | | | | “ASPS_NG23.2”; exon “1”; |
| chr10 | transcript | 73632708 | 73633978 | + | gene_id “ASPS_NG24”; transcript_id |
| | | | | | “ASPS_NG24.1”; RPKM “1.1933”; cov |
| | | | | | “15.8214”; |
| chr10 | exon | 73632708 | 73632841 | + | gene_id “ASPS_NG24”; transcript_id |
| | | | | | “ASPS_NG24.1”; exon “1”; |
| chr10 | exon | 73633342 | 73633978 | + | gene_id “ASPS_NG24”; transcript_id |
| | | | | | “ASPS_NG24.1”; exon “2”; |
| chr10 | transcript | 134197448 | 134200655 | − | gene_id “ASPS_NG25”; transcript_id |
| | | | | | “ASPS_NG25.1”; RPKM “3.4364”; cov |
| | | | | | “45.5608”; |
| chr10 | exon | 134197448 | 134200655 | − | gene_id “ASPS_NG25”; transcript_id |
| | | | | | “ASPS_NG25.1”; exon “1”; |
| chr10 | transcript | 134202353 | 134205682 | − | gene_id “ASPS_NG26”; transcript_id |
| | | | | | “ASPS_NG26.1”; RPKM “2.5685”; cov |
| | | | | | “34.0538”; |
| chr10 | exon | 134202353 | 134205682 | − | gene_id “ASPS_NG26”; transcript_id |
| | | | | | “ASPS_NG26.1”; exon “1”; |
| chr10 | transcript | 134206139 | 134210244 | − | gene_id “ASPS_NG27”; transcript_id |
| | | | | | “ASPS_NG27.1”; RPKM “2.0637”; cov |
| | | | | | “27.3609”; |
| chr10 | exon | 134206139 | 134207470 | − | gene_id “ASPS_NG27”; transcript_id |
| | | | | | “ASPS_NG27.1”; exon “1”; |
| chr10 | exon | 134207774 | 134207834 | − | gene_id “ASPS_NG27”; transcript_id |
| | | | | | “ASPS_NG27.1”; exon “2”; |
| chr10 | exon | 134208165 | 134208375 | − | gene_id “ASPS_NG27”; transcript_id |
| | | | | | “ASPS_NG27.1”; exon “3”; |
| chr10 | exon | 134209384 | 134210244 | − | gene_id “ASPS_NG27”; transcript_id |
| | | | | | “ASPS_NG27.1”; exon “4”; |
| chr10 | transcript | 134206139 | 134210244 | − | gene_id “ASPS_NG27”; transcript_id |
| | | | | | “ASPS_NG27.2”; RPKM “2.2388”; cov |
| | | | | | “29.6833”; |
| chr10 | exon | 134206139 | 134207470 | − | gene_id “ASPS_NG27”; transcript_id |
| | | | | | “ASPS_NG27.2”; exon “1”; |
| chr10 | exon | 134207774 | 134207834 | − | gene_id “ASPS_NG27”; transcript_id |
| | | | | | “ASPS_NG27.2”; exon “2”; |
| chr10 | exon | 134208165 | 134210244 | − | gene_id “ASPS_NG27”; transcript_id |
| | | | | | “ASPS_NG27.2”; exon “3”; |
| chr10 | transcript | 134206139 | 134210244 | − | gene_id “ASPS_NG27”; transcript_id |
| | | | | | “ASPS_NG27.3”; RPKM “0.9150”; cov |
| | | | | | “12.1308”; |
| chr10 | exon | 134206139 | 134207470 | − | gene_id “ASPS_NG27”; transcript_id |
| | | | | | “ASPS_NG27.3”; exon “1”; |
| chr10 | exon | 134208165 | 134210244 | − | gene_id “ASPS_NG27”; transcript_id |
| | | | | | “ASPS_NG27.3”; exon “2”; |
| chr10 | transcript | 134206139 | 134210244 | − | gene_id “ASPS_NG27”; transcript_id |
| | | | | | “ASPS_NG27.4”; RPKM “3.6605”; cov |
| | | | | | “48.5324”; |
| chr10 | exon | 134206139 | 134210244 | − | gene_id “ASPS_NG27”; transcript_id |
| | | | | | “ASPS_NG27.4”; exon “1”; |
| chr10 | transcript | 134206139 | 134210244 | − | gene_id “ASPS_NG27”; transcript_id |
| | | | | | “ASPS_NG27.5”; RPKM “5.1391”; cov |
| | | | | | “68.1367”; |
| chr10 | exon | 134206139 | 134207470 | − | gene_id “ASPS_NG27”; transcript_id |
| | | | | | “ASPS_NG27.5”; exon “1”; |
| chr10 | exon | 134207774 | 134210244 | − | gene_id “ASPS_NG27”; transcript_id |
| | | | | | “ASPS_NG27.5”; exon “2”; |
| chr10 | transcript | 134210711 | 134211528 | − | gene_id “ASPS_NG28”; transcript_id |
| | | | | | “ASPS_NG28.1”; RPKM “3.9826”; cov |
| | | | | | “52.8029”; |
| chr10 | exon | 134210711 | 134211528 | − | gene_id “ASPS_NG28”; transcript_id |
| | | | | | “ASPS_NG28.1”; exon “1”; |
| chr11 | transcript | 99924000 | 100217666 | − | gene_id “ASPS_NG29”; transcript_id |
| | | | | | “ASPS_NG29.5”; RPKM “0.7870”; cov |
| | | | | | “10.4344”; |
| chr11 | exon | 99924000 | 99925375 | − | gene_id “ASPS_NG29”; transcript_id |
| | | | | | “ASPS_NG29.5”; exon “1”; |
| chr11 | exon | 99925648 | 99925758 | − | gene_id “ASPS_NG29”; transcript_id |
| | | | | | “ASPS_NG29.5”; exon “2”; |
| chr11 | exon | 99926128 | 99926262 | − | gene_id “ASPS_NG29”; transcript_id |
| | | | | | “ASPS_NG29.5”; exon “3”; |
| chr11 | exon | 99976767 | 99976891 | − | gene_id “ASPS_NG29”; transcript_id |
| | | | | | “ASPS_NG29.5”; exon “4”; |
| chr11 | exon | 99984667 | 99984894 | − | gene_id “ASPS_NG29”; transcript_id |
| | | | | | “ASPS_NG29.5”; exon “5”; |
| chr11 | exon | 100000960 | 100001126 | − | gene_id “ASPS_NG29”; transcript_id |
| | | | | | “ASPS_NG29.5”; exon “6”; |
| chr11 | exon | 100007599 | 100007654 | − | gene_id “ASPS_NG29”; transcript_id |
| | | | | | “ASPS_NG29.5”; exon “7”; |
| chr11 | exon | 100010471 | 100010574 | − | gene_id “ASPS_NG29”; transcript_id |
| | | | | | “ASPS_NG29.5”; exon “8”; |
| chr11 | exon | 100174163 | 100174228 | − | gene_id “ASPS_NG29”; transcript_id |
| | | | | | “ASPS_NG29.5”; exon “9”; |
| chr11 | exon | 100217587 | 100217666 | − | gene_id “ASPS_NG29”; transcript_id |
| | | | | | “ASPS_NG29.5”; exon “10”; |
| chr11 | transcript | 99978045 | 100217666 | − | gene_id “ASPS_NG29”; transcript_id |
| | | | | | “ASPS_NG29.6”; RPKM “2.8095”; cov |
| | | | | | “37.2493”; |
| chr11 | exon | 99978045 | 99980154 | − | gene_id “ASPS_NG29”; transcript_id |
| | | | | | “ASPS_NG29.6”; exon “1”; |
| chr11 | exon | 99984667 | 99984894 | − | gene_id “ASPS_NG29”; transcript_id |
| | | | | | “ASPS_NG29.6”; exon “2”; |
| chr11 | exon | 100000960 | 100001126 | − | gene_id “ASPS_NG29”; transcript_id |
| | | | | | “ASPS_NG29.6”; exon “3”; |
| chr11 | exon | 100007599 | 100007654 | − | gene_id “ASPS_NG29”; transcript_id |
| | | | | | “ASPS_NG29.6”; exon “4”; |
| chr11 | exon | 100010471 | 100010574 | − | gene_id “ASPS_NG29”; transcript_id |
| | | | | | “ASPS_NG29.6”; exon “5”; |
| chr11 | exon | 100174163 | 100174228 | − | gene_id “ASPS_NG29”; transcript_id |
| | | | | | “ASPS_NG29.6”; exon “6”; |
| chr11 | exon | 100217587 | 100217666 | − | gene_id “ASPS_NG29”; transcript_id |
| | | | | | “ASPS_NG29.6”; exon “7”; |
| chr11 | transcript | 99978045 | 100217666 | − | gene_id “ASPS_NG29”; transcript_id |
| | | | | | “ASPS_NG29.4”; RPKM “5.2036”; cov |
| | | | | | “68.9913”; |
| chr11 | exon | 99978045 | 99980154 | − | gene_id “ASPS_NG29”; transcript_id |
| | | | | | “ASPS_NG29.4”; exon “1”; |
| chr11 | exon | 99984667 | 99984894 | − | gene_id “ASPS_NG29”; transcript_id |
| | | | | | “ASPS_NG29.4”; exon “2”; |
| chr11 | exon | 99985662 | 99985832 | − | gene_id “ASPS_NG29”; transcript_id |
| | | | | | “ASPS_NG29.4”; exon “3”; |
| chr11 | exon | 100000960 | 100001126 | − | gene_id “ASPS_NG29”; transcript_id |
| | | | | | “ASPS_NG29.4”; exon “4”; |
| chr11 | exon | 100007599 | 100007654 | − | gene_id “ASPS_NG29”; transcript_id |
| | | | | | “ASPS_NG29.4”; exon “5”; |
| chr11 | exon | 100010471 | 100010574 | − | gene_id “ASPS_NG29”; transcript_id |
| | | | | | “ASPS_NG29.4”; exon “6”; |
| chr11 | exon | 100174163 | 100174228 | − | gene_id “ASPS_NG29”; transcript_id |
| | | | | | “ASPS_NG29.4”; exon “7”; |
| chr11 | exon | 100217587 | 100217666 | − | gene_id “ASPS_NG29”; transcript_id |
| | | | | | “ASPS_NG29.4”; exon “8”; |
| chr11 | transcript | 99978045 | 100216028 | − | gene_id “ASPS_NG29”; transcript_id |
| | | | | | “ASPS_NG29.3”; RPKM “1.8790”; cov |
| | | | | | “24.9125”; |
| chr11 | exon | 99978045 | 99980154 | − | gene_id “ASPS_NG29”; transcript_id |
| | | | | | “ASPS_NG29.3”; exon “1”; |
| chr11 | exon | 99984667 | 99984894 | − | gene_id “ASPS_NG29”; transcript_id |
| | | | | | “ASPS_NG29.3”; exon “2”; |
| chr11 | exon | 99985662 | 99985832 | − | gene_id “ASPS_NG29”; transcript_id |
| | | | | | “ASPS_NG29.3”; exon “3”; |
| chr11 | exon | 100007599 | 100007654 | − | gene_id “ASPS_NG29”; transcript_id |
| | | | | | “ASPS_NG29.3”; exon “4”; |
| chr11 | exon | 100010471 | 100010574 | − | gene_id “ASPS_NG29”; transcript_id |
| | | | | | “ASPS_NG29.3”; exon “5”; |
| chr11 | exon | 100174163 | 100174228 | − | gene_id “ASPS_NG29”; transcript_id |
| | | | | | “ASPS_NG29.3”; exon “6”; |
| chr11 | exon | 100215879 | 100216028 | − | gene_id “ASPS_NG29”; transcript_id |
| | | | | | “ASPS_NG29.3”; exon “7”; |
| chr11 | transcript | 100020238 | 100217666 | − | gene_id “ASPS_NG29”; transcript_id |
| | | | | | “ASPS_NG29.2”; RPKM “5.5298”; cov |
| | | | | | “73.3161”; |
| chr11 | exon | 100020238 | 100020906 | − | gene_id “ASPS_NG29”; transcript_id |
| | | | | | “ASPS_NG29.2”; exon “1”; |
| chr11 | exon | 100174163 | 100174228 | − | gene_id “ASPS_NG29”; transcript_id |
| | | | | | “ASPS_NG29.2”; exon “2”; |
| chr11 | exon | 100217587 | 100217666 | − | gene_id “ASPS_NG29”; transcript_id |
| | | | | | “ASPS_NG29.2”; exon “3”; |
| chr11 | transcript | 100020238 | 100216028 | − | gene_id “ASPS_NG29”; transcript_id |
| | | | | | “ASPS_NG29.1”; RPKM “2.0420”; cov |
| | | | | | “27.0740”; |
| chr11 | exon | 100020238 | 100020906 | − | gene_id “ASPS_NG29”; transcript_id |
| | | | | | “ASPS_NG29.1”; exon “1”; |
| chr11 | exon | 100174163 | 100174228 | − | gene_id “ASPS_NG29”; transcript_id |
| | | | | | “ASPS_NG29.1”; exon “2”; |
| chr11 | exon | 100215879 | 100216028 | − | gene_id “ASPS_NG29”; transcript_id |
| | | | | | “ASPS_NG29.1”; exon “3”; |
| chr13 | transcript | 90809686 | 90858367 | − | gene_id “ASPS_NG30”; transcript_id |
| | | | | | “ASPS_NG30.4”; RPKM “1.1466”; cov |
| | | | | | “15.2025”; |
| chr13 | exon | 90809686 | 90810047 | − | gene_id “ASPS_NG30”; transcript_id |
| | | | | | “ASPS_NG30.4”; exon “1”; |
| chr13 | exon | 90835751 | 90835923 | − | gene_id “ASPS_NG30”; transcript_id |
| | | | | | “ASPS_NG30.4”; exon “2”; |
| chr13 | exon | 90836930 | 90837000 | − | gene_id “ASPS_NG30”; transcript_id |
| | | | | | “ASPS_NG30.4”; exon “3”; |
| chr13 | exon | 90839026 | 90839162 | − | gene_id “ASPS_NG30”; transcript_id |
| | | | | | “ASPS_NG30.4”; exon “4”; |
| chr13 | exon | 90858239 | 90858367 | − | gene_id “ASPS_NG30”; transcript_id |
| | | | | | “ASPS_NG30.4”; exon “5”; |
| chr13 | transcript | 90825311 | 90858367 | − | gene_id “ASPS_NG30”; transcript_id |
| | | | | | “ASPS_NG30.3”; RPKM “0.7944”; cov |
| | | | | | “10.5322”; |
| chr13 | exon | 90825311 | 90827199 | − | gene_id “ASPS_NG30”; transcript_id |
| | | | | | “ASPS_NG30.3”; exon “1”; |
| chr13 | exon | 90835751 | 90835923 | − | gene_id “ASPS_NG30”; transcript_id |
| | | | | | “ASPS_NG30.3”; exon “2”; |
| chr13 | exon | 90836930 | 90837000 | − | gene_id “ASPS_NG30”; transcript_id |
| | | | | | “ASPS_NG30.3”; exon “3”; |
| chr13 | exon | 90839026 | 90839162 | − | gene_id “ASPS_NG30”; transcript_id |
| | | | | | “ASPS_NG30.3”; exon “4”; |
| chr13 | exon | 90858239 | 90858367 | − | gene_id “ASPS_NG30”; transcript_id |
| | | | | | “ASPS_NG30.3”; exon “5”; |
| chr13 | transcript | 90829372 | 90858367 | − | gene_id “ASPS_NG30”; transcript_id |
| | | | | | “ASPS_NG30.1”; RPKM “1.3676”; cov |
| | | | | | “18.1325”; |
| chr13 | exon | 90829372 | 90829985 | − | gene_id “ASPS_NG30”; transcript_id |
| | | | | | “ASPS_NG30.1”; exon “1”; |
| chr13 | exon | 90835751 | 90835923 | − | gene_id “ASPS_NG30”; transcript_id |
| | | | | | “ASPS_NG30.1”; exon “2”; |
| chr13 | exon | 90836930 | 90837000 | − | gene_id “ASPS_NG30”; transcript_id |
| | | | | | “ASPS_NG30.1”; exon “3”; |
| chr13 | exon | 90858239 | 90858367 | − | gene_id “ASPS_NG30”; transcript_id |
| | | | | | “ASPS_NG30.1”; exon “4”; |
| chr13 | transcript | 90829372 | 90858367 | − | gene_id “ASPS_NG30”; transcript_id |
| | | | | | “ASPS_NG30.5”; RPKM “5.5499”; cov |
| | | | | | “73.5832”; |
| chr13 | exon | 90829372 | 90829985 | − | gene_id “ASPS_NG30”; transcript_id |
| | | | | | “ASPS_NG30.5”; exon “1”; |
| chr13 | exon | 90835751 | 90835923 | − | gene_id “ASPS_NG30”; transcript_id |
| | | | | | “ASPS_NG30.5”; exon “2”; |
| chr13 | exon | 90836930 | 90837000 | − | gene_id “ASPS_NG30”; transcript_id |
| | | | | | “ASPS_NG30.5”; exon “3”; |
| chr13 | exon | 90839026 | 90839162 | − | gene_id “ASPS_NG30”; transcript_id |
| | | | | | “ASPS_NG30.5”; exon “4”; |
| chr13 | exon | 90858239 | 90858367 | − | gene_id “ASPS_NG30”; transcript_id |
| | | | | | “ASPS_NG30.5”; exon “5”; |
| chr13 | transcript | 90834817 | 90858367 | − | gene_id “ASPS_NG30”; transcript_id |
| | | | | | “ASPS_NG30.2”; RPKM “1.3777”; cov |
| | | | | | “18.2667”; |
| chr13 | exon | 90834817 | 90835923 | − | gene_id “ASPS_NG30”; transcript_id |
| | | | | | “ASPS_NG30.2”; exon “1”; |
| chr13 | exon | 90836930 | 90837000 | − | gene_id “ASPS_NG30”; transcript_id |
| | | | | | “ASPS_NG30.2”; exon “2”; |
| chr13 | exon | 90839026 | 90839162 | − | gene_id “ASPS_NG30”; transcript_id |
| | | | | | “ASPS_NG30.2”; exon “3”; |
| chr13 | exon | 90858239 | 90858367 | − | gene_id “ASPS_NG30”; transcript_id |
| | | | | | “ASPS_NG30.2”; exon “4”; |
| chr17 | transcript | 884526 | 886818 | − | gene_id “ASPS_NG31”; transcript_id |
| | | | | | “ASPS_NG31.1”; RPKM “9.1903”; cov |
| | | | | | “121.8487”; |
| chr17 | exon | 884526 | 886818 | − | gene_id “ASPS_NG31”; transcript_id |
| | | | | | “ASPS_NG31.1”; exon “1”; |
| chr17 | transcript | 886855 | 892420 | − | gene_id “ASPS_NG32”; transcript_id |
| | | | | | “ASPS_NG32.1”; RPKM “4.5389”; cov |
| | | | | | “60.1788”; |
| chr17 | exon | 886855 | 892420 | − | gene_id “ASPS_NG32”; transcript_id |
| | | | | | “ASPS_NG32.1”; exon “1”; |
| chr2 | transcript | 180266 | 186655 | + | gene_id “ASPS_NG3”; transcript_id |
| | | | | | “ASPS_NG3.2”; RPKM “2.1870”; cov |
| | | | | | “28.9955”; |
| chr2 | exon | 180266 | 180670 | + | gene_id “ASPS_NG3”; transcript_id |
| | | | | | “ASPS_NG3.2”; exon “1”; |
| chr2 | exon | 184874 | 184969 | + | gene_id “ASPS_NG3”; transcript_id |
| | | | | | “ASPS_NG3.2”; exon “2”; |
| chr2 | exon | 186314 | 186655 | + | gene_id “ASPS_NG3”; transcript_id |
| | | | | | “ASPS_NG3.2”; exon “3”; |
| chr2 | transcript | 204337 | 211384 | + | gene_id “ASPS_NG3”; transcript_id |
| | | | | | “ASPS_NG3.1”; RPKM “3.1220”; cov |
| | | | | | “41.3928”; |
| chr2 | exon | 204337 | 204388 | + | gene_id “ASPS_NG3”; transcript_id |
| | | | | | “ASPS_NG3.1”; exon “1”; |
| chr2 | exon | 209485 | 211384 | + | gene_id “ASPS_NG3”; transcript_id |
| | | | | | “ASPS_NG3.1”; exon “2”; |
| chr2 | transcript | 21116965 | 21117794 | + | gene_id “ASPS_NG4”; transcript_id |
| | | | | | “ASPS_NG4.1”; RPKM “1.6680”; cov |
| | | | | | “22.1144”; |
| chr2 | exon | 21116965 | 21117097 | + | gene_id “ASPS_NG4”; transcript_id |
| | | | | | “ASPS_NG4.1”; exon “1”; |
| chr2 | exon | 21117283 | 21117794 | + | gene_id “ASPS_NG4”; transcript_id |
| | | | | | “ASPS_NG4.1”; exon “2”; |
| chr2 | transcript | 23941529 | 23944173 | − | gene_id “ASPS_NG5”; transcript_id |
| | | | | | “ASPS_NG5.1”; RPKM “3.8692”; cov |
| | | | | | “51.3000”; |
| chr2 | exon | 23941529 | 23944173 | − | gene_id “ASPS_NG5”; transcript_id |
| | | | | | “ASPS_NG5.1”; exon “1”; |
| chr2 | transcript | 151555817 | 151669720 | − | gene_id “ASPS_NG6”; transcript_id |
| | | | | | “ASPS_NG6.3”; RPKM “0.9263”; cov |
| | | | | | “12.2815”; |
| chr2 | exon | 151555817 | 151555980 | − | gene_id “ASPS_NG6”; transcript_id |
| | | | | | “ASPS_NG6.3”; exon “1”; |
| chr2 | exon | 151564915 | 151565082 | − | gene_id “ASPS_NG6”; transcript_id |
| | | | | | “ASPS_NG6.3”; exon “2”; |
| chr2 | exon | 151669618 | 151669720 | − | gene_id “ASPS_NG6”; transcript_id |
| | | | | | “ASPS_NG6.3”; exon “3”; |
| chr2 | transcript | 151555817 | 151669720 | − | gene_id “ASPS_NG6”; transcript_id |
| | | | | | “ASPS_NG6.4”; RPKM “1.7786”; cov |
| | | | | | “23.5810”; |
| chr2 | exon | 151555817 | 151555980 | − | gene_id “ASPS_NG6”; transcript_id |
| | | | | | “ASPS_NG6.4”; exon “1”; |
| chr2 | exon | 151564915 | 151565082 | − | gene_id “ASPS_NG6”; transcript_id |
| | | | | | “ASPS_NG6.4”; exon “2”; |
| chr2 | exon | 151668506 | 151668771 | − | gene_id “ASPS_NG6”; transcript_id |
| | | | | | “ASPS_NG6.4”; exon “3”; |
| chr2 | exon | 151669618 | 151669720 | − | gene_id “ASPS_NG6”; transcript_id |
| | | | | | “ASPS_NG6.4”; exon “4”; |
| chr2 | transcript | 151555817 | 151669720 | − | gene_id “ASPS_NG6”; transcript_id |
| | | | | | “ASPS_NG6.5”; RPKM “2.2600”; cov |
| | | | | | “29.9639”; |
| chr2 | exon | 151555817 | 151555980 | − | gene_id “ASPS_NG6”; transcript_id |
| | | | | | “ASPS_NG6.5”; exon “1”; |
| chr2 | exon | 151564915 | 151565082 | − | gene_id “ASPS_NG6”; transcript_id |
| | | | | | “ASPS_NG6.5”; exon “2”; |
| chr2 | exon | 151668506 | 151668719 | − | gene_id “ASPS_NG6”; transcript_id |
| | | | | | “ASPS_NG6.5”; exon “3”; |
| chr2 | exon | 151669618 | 151669720 | − | gene_id “ASPS_NG6”; transcript_id |
| | | | | | “ASPS_NG6.5”; exon “4”; |
| chr2 | transcript | 151664180 | 151669720 | − | gene_id “ASPS_NG6”; transcript_id |
| | | | | | “ASPS_NG6.1”; RPKM “1.5241”; cov |
| | | | | | “20.2072”; |
| chr2 | exon | 151664180 | 151668635 | − | gene_id “ASPS_NG6”; transcript_id |
| | | | | | “ASPS_NG6.1”; exon “1”; |
| chr2 | exon | 151669618 | 151669720 | − | gene_id “ASPS_NG6”; transcript_id |
| | | | | | “ASPS_NG6.1”; exon “2”; |
| chr2 | transcript | 151664180 | 151669720 | − | gene_id “ASPS_NG6”; transcript_id |
| | | | | | “ASPS_NG6.2”; RPKM “1.9095”; cov |
| | | | | | “25.3172”; |
| chr2 | exon | 151664180 | 151667376 | − | gene_id “ASPS_NG6”; transcript_id |
| | | | | | “ASPS_NG6.2”; exon “1”; |
| chr2 | exon | 151668506 | 151668719 | − | gene_id “ASPS_NG6”; transcript_id |
| | | | | | “ASPS_NG6.2”; exon “2”; |
| chr2 | exon | 151669618 | 151669720 | − | gene_id “ASPS_NG6”; transcript_id |
| | | | | | “ASPS_NG6.2”; exon “3”; |
| chr20 | transcript | 3061971 | 3069430 | + | gene_id “ASPS_NG33”; transcript_id |
| | | | | | “ASPS_NG33.1”; RPKM “1.4885”; cov |
| | | | | | “19.7356”; |
| chr20 | exon | 3061971 | 3062371 | + | gene_id “ASPS_NG33”; transcript_id |
| | | | | | “ASPS_NG33.1”; exon “1”; |
| chr20 | exon | 3068860 | 3069430 | + | gene_id “ASPS_NG33”; transcript_id |
| | | | | | “ASPS_NG33.1”; exon “2”; |
| chr3 | transcript | 12925178 | 12984474 | + | gene_id “ASPS_NG7”; transcript_id |
| | | | | | “ASPS_NG7.1”; RPKM “1.4348”; cov |
| | | | | | “19.0230”; |
| chr3 | exon | 12925178 | 12925504 | + | gene_id “ASPS_NG7”; transcript_id |
| | | | | | “ASPS_NG7.1”; exon “1”; |
| chr3 | exon | 12984324 | 12984474 | + | gene_id “ASPS_NG7”; transcript_id |
| | | | | | “ASPS_NG7.1”; exon “2”; |
| chr3 | transcript | 15383020 | 15412040 | + | gene_id “ASPS_NG8”; transcript_id |
| | | | | | “ASPS_NG8.1”; RPKM “14.2407”; cov |
| | | | | | “188.8098”; |
| chr3 | exon | 15383020 | 15383151 | + | gene_id “ASPS_NG8”; transcript_id |
| | | | | | “ASPS_NG8.1”; exon “1”; |
| chr3 | exon | 15400598 | 15400730 | + | gene_id “ASPS_NG8”; transcript_id |
| | | | | | “ASPS_NG8.1”; exon “2”; |
| chr3 | exon | 15403312 | 15412040 | + | gene_id “ASPS_NG8”; transcript_id |
| | | | | | “ASPS_NG8.1”; exon “3”; |
| chr3 | transcript | 15383020 | 15387151 | + | gene_id “ASPS_NG8”; transcript_id |
| | | | | | “ASPS_NG8.2”; RPKM “86.3023”; cov |
| | | | | | “1144.2316”; |
| chr3 | exon | 15383020 | 15383151 | + | gene_id “ASPS_NG8”; transcript_id |
| | | | | | “ASPS_NG8.2”; exon “1”; |
| chr3 | exon | 15385004 | 15387151 | + | gene_id “ASPS_NG8”; transcript_id |
| | | | | | “ASPS_NG8.2”; exon “2”; |
| chr3 | transcript | 32122887 | 32130810 | − | gene_id “ASPS_NG9”; transcript_id |
| | | | | | “ASPS_NG9.1”; RPKM “1.0641”; cov |
| | | | | | “14.1087”; |
| chr3 | exon | 32122887 | 32123487 | − | gene_id “ASPS_NG9”; transcript_id |
| | | | | | “ASPS_NG9.1”; exon “1”; |
| chr3 | exon | 32130480 | 32130810 | − | gene_id “ASPS_NG9”; transcript_id |
| | | | | | “ASPS_NG9.1”; exon “2”; |
| chr3 | transcript | 32122887 | 32130810 | − | gene_id “ASPS_NG9”; transcript_id |
| | | | | | “ASPS_NG9.2”; RPKM “3.8198”; cov |
| | | | | | “50.6445”; |
| chr3 | exon | 32122887 | 32130810 | − | gene_id “ASPS_NG9”; transcript_id |
| | | | | | “ASPS_NG9.2”; exon “1”; |
| chr3 | transcript | 42487320 | 42520982 | + | gene_id “ASPS_NG10”; transcript_id |
| | | | | | “ASPS_NG10.1”; RPKM “1.2629”; cov |
| | | | | | “16.7434”; |
| chr3 | exon | 42487320 | 42487400 | + | gene_id “ASPS_NG10”; transcript_id |
| | | | | | “ASPS_NG10.1”; exon “1”; |
| chr3 | exon | 42495101 | 42495193 | + | gene_id “ASPS_NG10”; transcript_id |
| | | | | | “ASPS_NG10.1”; exon “2”; |
| chr3 | exon | 42514879 | 42515017 | + | gene_id “ASPS_NG10”; transcript_id |
| | | | | | “ASPS_NG10.1”; exon “3”; |
| chr3 | exon | 42515110 | 42520982 | + | gene_id “ASPS_NG10”; transcript_id |
| | | | | | “ASPS_NG10.1”; exon “4”; |
| chr3 | transcript | 42487320 | 42520982 | + | gene_id “ASPS_NG10”; transcript_id |
| | | | | | “ASPS_NG10.2”; RPKM “1.8681”; cov |
| | | | | | “24.7680”; |
| chr3 | exon | 42487320 | 42487400 | + | gene_id “ASPS_NG10”; transcript_id |
| | | | | | “ASPS_NG10.2”; exon “1”; |
| chr3 | exon | 42495101 | 42495193 | + | gene_id “ASPS_NG10”; transcript_id |
| | | | | | “ASPS_NG10.2”; exon “2”; |
| chr3 | exon | 42514879 | 42515017 | + | gene_id “ASPS_NG10”; transcript_id |
| | | | | | “ASPS_NG10.2”; exon “3”; |
| chr3 | exon | 42516185 | 42520982 | + | gene_id “ASPS_NG10”; transcript_id |
| | | | | | “ASPS_NG10.2”; exon “4”; |
| chr3 | transcript | 42487320 | 42520982 | + | gene_id “ASPS_NG10”; transcript_id |
| | | | | | “ASPS_NG10.3”; RPKM “3.6655”; cov |
| | | | | | “48.5982”; |
| chr3 | exon | 42487320 | 42487400 | + | gene_id “ASPS_NG10”; transcript_id |
| | | | | | “ASPS_NG10.3”; exon “1”; |
| chr3 | exon | 42495101 | 42495193 | + | gene_id “ASPS_NG10”; transcript_id |
| | | | | | “ASPS_NG10.3”; exon “2”; |
| chr3 | exon | 42514879 | 42520982 | + | gene_id “ASPS_NG10”; transcript_id |
| | | | | | “ASPS_NG10.3”; exon “3”; |
| chr3 | transcript | 42487320 | 42520982 | + | gene_id “ASPS_NG10”; transcript_id |
| | | | | | “ASPS_NG10.4”; RPKM “1.0101”; cov |
| | | | | | “13.3928”; |
| chr3 | exon | 42487320 | 42487400 | + | gene_id “ASPS_NG10”; transcript_id |
| | | | | | “ASPS_NG10.4”; exon “1”; |
| chr3 | exon | 42495101 | 42495193 | + | gene_id “ASPS_NG10”; transcript_id |
| | | | | | “ASPS_NG10.4”; exon “2”; |
| chr3 | exon | 42513841 | 42520982 | + | gene_id “ASPS_NG10”; transcript_id |
| | | | | | “ASPS_NG10.4”; exon “3”; |
| chr3 | transcript | 46095612 | 46127414 | − | gene_id “ASPS_NG11”; transcript_id |
| | | | | | “ASPS_NG11.3”; RPKM “0.8976”; cov |
| | | | | | “11.9009”; |
| chr3 | exon | 46095612 | 46095875 | − | gene_id “ASPS_NG11”; transcript_id |
| | | | | | “ASPS_NG11.3”; exon “1”; |
| chr3 | exon | 46108391 | 46108470 | − | gene_id “ASPS_NG11”; transcript_id |
| | | | | | “ASPS_NG11.3”; exon “2”; |
| chr3 | exon | 46127286 | 46127414 | − | gene_id “ASPS_NG11”; transcript_id |
| | | | | | “ASPS_NG11.3”; exon “3”; |
| chr3 | transcript | 46102539 | 46127414 | − | gene_id “ASPS_NG11”; transcript_id |
| | | | | | “ASPS_NG11.9”; RPKM “2.7665”; cov |
| | | | | | “36.6800”; |
| chr3 | exon | 46102539 | 46103187 | − | gene_id “ASPS_NG11”; transcript_id |
| | | | | | “ASPS_NG11.9”; exon “1”; |
| chr3 | exon | 46108391 | 46108470 | − | gene_id “ASPS_NG11”; transcript_id |
| | | | | | “ASPS_NG11.9”; exon “2”; |
| chr3 | exon | 46116143 | 46116207 | − | gene_id “ASPS_NG11”; transcript_id |
| | | | | | “ASPS_NG11.9”; exon “3”; |
| chr3 | exon | 46127286 | 46127414 | − | gene_id “ASPS_NG11”; transcript_id |
| | | | | | “ASPS_NG11.9”; exon “4”; |
| chr3 | transcript | 46102539 | 46127414 | − | gene_id “ASPS_NG11”; transcript_id |
| | | | | | “ASPS_NG11.8”; RPKM “1.0028”; cov |
| | | | | | “13.2957”; |
| chr3 | exon | 46102539 | 46103187 | − | gene_id “ASPS_NG11”; transcript_id |
| | | | | | “ASPS_NG11.8”; exon “1”; |
| chr3 | exon | 46108391 | 46108470 | − | gene_id “ASPS_NG11”; transcript_id |
| | | | | | “ASPS_NG11.8”; exon “2”; |
| chr3 | exon | 46116143 | 46116207 | − | gene_id “ASPS_NG11”; transcript_id |
| | | | | | “ASPS_NG11.8”; exon “3”; |
| chr3 | exon | 46118232 | 46118418 | − | gene_id “ASPS_NG11”; transcript_id |
| | | | | | “ASPS_NG11.8”; exon “4”; |
| chr3 | exon | 46127286 | 46127414 | − | gene_id “ASPS_NG11”; transcript_id |
| | | | | | “ASPS_NG11.8”; exon “5”; |
| chr3 | transcript | 46102539 | 46127414 | − | gene_id “ASPS_NG11”; transcript_id |
| | | | | | “ASPS_NG11.7”; RPKM “0.9001”; cov |
| | | | | | “11.9334”; |
| chr3 | exon | 46102539 | 46103187 | − | gene_id “ASPS_NG11”; transcript_id |
| | | | | | “ASPS_NG11.7”; exon “1”; |
| chr3 | exon | 46108391 | 46108470 | − | gene_id “ASPS_NG11”; transcript_id |
| | | | | | “ASPS_NG11.7”; exon “2”; |
| chr3 | exon | 46114955 | 46115053 | − | gene_id “ASPS_NG11”; transcript_id |
| | | | | | “ASPS_NG11.7”; exon “3”; |
| chr3 | exon | 46116143 | 46116207 | − | gene_id “ASPS_NG11”; transcript_id |
| | | | | | “ASPS_NG11.7”; exon “4”; |
| chr3 | exon | 46127286 | 46127414 | − | gene_id “ASPS_NG11”; transcript_id |
| | | | | | “ASPS_NG11.7”; exon “5”; |
| chr3 | transcript | 46102539 | 46127414 | − | gene_id “ASPS_NG11”; transcript_id |
| | | | | | “ASPS_NG11.6”; RPKM “2.5335”; cov |
| | | | | | “33.5906”; |
| chr3 | exon | 46102539 | 46103187 | − | gene_id “ASPS_NG11”; transcript_id |
| | | | | | “ASPS_NG11.6”; exon “1”; |
| chr3 | exon | 46108391 | 46108470 | − | gene_id “ASPS_NG11”; transcript_id |
| | | | | | “ASPS_NG11.6”; exon “2”; |
| chr3 | exon | 46114955 | 46115053 | − | gene_id “ASPS_NG11”; transcript_id |
| | | | | | “ASPS_NG11.6”; exon “3”; |
| chr3 | exon | 46127286 | 46127414 | − | gene_id “ASPS_NG11”; transcript_id |
| | | | | | “ASPS_NG11.6”; exon “4”; |
| chr3 | transcript | 46102539 | 46106555 | − | gene_id “ASPS_NG11”; transcript_id |
| | | | | | “ASPS_NG11.1”; RPKM “1.8421”; cov |
| | | | | | “24.4230”; |
| chr3 | exon | 46102539 | 46106555 | − | gene_id “ASPS_NG11”; transcript_id |
| | | | | | “ASPS_NG11.1”; exon “1”; |
| chr3 | transcript | 46102539 | 46127414 | − | gene_id “ASPS_NG11”; transcript_id |
| | | | | | “ASPS_NG11.5”; RPKM “25.0939”; cov |
| | | | | | “332.7060”; |
| chr3 | exon | 46102539 | 46103187 | − | gene_id “ASPS_NG11”; transcript_id |
| | | | | | “ASPS_NG11.5”; exon “1”; |
| chr3 | exon | 46108391 | 46108470 | − | gene_id “ASPS_NG11”; transcript_id |
| | | | | | “ASPS_NG11.5”; exon “2”; |
| chr3 | exon | 46127286 | 46127414 | − | gene_id “ASPS_NG11”; transcript_id |
| | | | | | “ASPS_NG11.5”; exon “3”; |
| chr3 | transcript | 46102539 | 46127414 | − | gene_id “ASPS_NG11”; transcript_id |
| | | | | | “ASPS_NG11.4”; RPKM “1.0967”; cov |
| | | | | | “14.5411”; |
| chr3 | exon | 46102539 | 46102906 | − | gene_id “ASPS_NG11”; transcript_id |
| | | | | | “ASPS_NG11.4”; exon “1”; |
| chr3 | exon | 46108391 | 46108470 | − | gene_id “ASPS_NG11”; transcript_id |
| | | | | | “ASPS_NG11.4”; exon “2”; |
| chr3 | exon | 46127286 | 46127414 | − | gene_id “ASPS_NG11”; transcript_id |
| | | | | | “ASPS_NG11.4”; exon “3”; |
| chr3 | transcript | 46102539 | 46127414 | − | gene_id “ASPS_NG11”; transcript_id |
| | | | | | “ASPS_NG11.2”; RPKM “1.9235”; cov |
| | | | | | “25.5028”; |
| chr3 | exon | 46102539 | 46103187 | − | gene_id “ASPS_NG11”; transcript_id |
| | | | | | “ASPS_NG11.2”; exon “1”; |
| chr3 | exon | 46127286 | 46127414 | − | gene_id “ASPS_NG11”; transcript_id |
| | | | | | “ASPS_NG11.2”; exon “2”; |
| chr4 | transcript | 65721483 | 65756904 | + | gene_id “ASPS_NG12”; transcript_id |
| | | | | | “ASPS_NG12.2”; RPKM “13.3595”; cov |
| | | | | | “177.1252”; |
| chr4 | exon | 65721483 | 65721563 | + | gene_id “ASPS_NG12”; transcript_id |
| | | | | | “ASPS_NG12.2”; exon “1”; |
| chr4 | exon | 65723424 | 65723576 | + | gene_id “ASPS_NG12”; transcript_id |
| | | | | | “ASPS_NG12.2”; exon “2”; |
| chr4 | exon | 65754191 | 65756904 | + | gene_id “ASPS_NG12”; transcript_id |
| | | | | | “ASPS_NG12.2”; exon “3”; |
| chr4 | transcript | 65721483 | 65756904 | + | gene_id “ASPS_NG12”; transcript_id |
| | | | | | “ASPS_NG12.3”; RPKM “3.5416”; cov |
| | | | | | “46.9560”; |
| chr4 | exon | 65721483 | 65721527 | + | gene_id “ASPS_NG12”; transcript_id |
| | | | | | “ASPS_NG12.3”; exon “1”; |
| chr4 | exon | 65723424 | 65723576 | + | gene_id “ASPS_NG12”; transcript_id |
| | | | | | “ASPS_NG12.3”; exon “2”; |
| chr4 | exon | 65754191 | 65756904 | + | gene_id “ASPS_NG12”; transcript_id |
| | | | | | “ASPS_NG12.3”; exon “3”; |
| chr4 | transcript | 65721483 | 65756904 | + | gene_id “ASPS_NG12”; transcript_id |
| | | | | | “ASPS_NG12.4”; RPKM “1.1409”; cov |
| | | | | | “15.1264”; |
| chr4 | exon | 65721483 | 65721563 | + | gene_id “ASPS_NG12”; transcript_id |
| | | | | | “ASPS_NG12.4”; exon “1”; |
| chr4 | exon | 65723424 | 65723777 | + | gene_id “ASPS_NG12”; transcript_id |
| | | | | | “ASPS_NG12.4”; exon “2”; |
| chr4 | exon | 65754191 | 65756904 | + | gene_id “ASPS_NG12”; transcript_id |
| | | | | | “ASPS_NG12.4”; exon “3”; |
| chr4 | transcript | 65721483 | 65724294 | + | gene_id “ASPS_NG12”; transcript_id |
| | | | | | “ASPS_NG12.5”; RPKM “1.5355”; cov |
| | | | | | “20.3582”; |
| chr4 | exon | 65721483 | 65721527 | + | gene_id “ASPS_NG12”; transcript_id |
| | | | | | “ASPS_NG12.5”; exon “1”; |
| chr4 | exon | 65723424 | 65724294 | + | gene_id “ASPS_NG12”; transcript_id |
| | | | | | “ASPS_NG12.5”; exon “2”; |
| chr4 | transcript | 65751008 | 65756904 | + | gene_id “ASPS_NG12”; transcript_id |
| | | | | | “ASPS_NG12.1”; RPKM “1.8233”; cov |
| | | | | | “24.1747”; |
| chr4 | exon | 65751008 | 65756904 | + | gene_id “ASPS_NG12”; transcript_id |
| | | | | | “ASPS_NG12.1”; exon “1”; |
| chr5 | transcript | 112949280 | 113334096 | + | gene_id “ASPS_NG13”; transcript_id |
| | | | | | “ASPS_NG13.16”; RPKM “2.7699”; cov |
| | | | | | “36.7245”; |
| chr5 | exon | 112949280 | 112949426 | + | gene_id “ASPS_NG13”; transcript_id |
| | | | | | “ASPS_NG13.16”; exon “1”; |
| chr5 | exon | 113026756 | 113026851 | + | gene_id “ASPS_NG13”; transcript_id |
| | | | | | “ASPS_NG13.16”; exon “2”; |
| chr5 | exon | 113333873 | 113334096 | + | gene_id “ASPS_NG13”; transcript_id |
| | | | | | “ASPS_NG13.16”; exon “3”; |
| chr5 | transcript | 112949280 | 113052437 | + | gene_id “ASPS_NG13”; transcript_id |
| | | | | | “ASPS_NG13.21”; RPKM “1.2884”; cov |
| | | | | | “17.0818”; |
| chr5 | exon | 112949280 | 112949426 | + | gene_id “ASPS_NG13”; transcript_id |
| | | | | | “ASPS_NG13.21”; exon “1”; |
| chr5 | exon | 112969011 | 112969061 | + | gene_id “ASPS_NG13”; transcript_id |
| | | | | | “ASPS_NG13.21”; exon “2”; |
| chr5 | exon | 113026756 | 113026851 | + | gene_id “ASPS_NG13”; transcript_id |
| | | | | | “ASPS_NG13.21”; exon “3”; |
| chr5 | exon | 113030776 | 113031030 | + | gene_id “ASPS_NG13”; transcript_id |
| | | | | | “ASPS_NG13.21”; exon “4”; |
| chr5 | exon | 113048088 | 113052437 | + | gene_id “ASPS_NG13”; transcript_id |
| | | | | | “ASPS_NG13.21”; exon “5”; |
| chr5 | transcript | 112949280 | 113052437 | + | gene_id “ASPS_NG13”; transcript_id |
| | | | | | “ASPS_NG13.20”; RPKM “1.6945”; cov |
| | | | | | “22.4666”; |
| chr5 | exon | 112949280 | 112949426 | + | gene_id “ASPS_NG13”; transcript_id |
| | | | | | “ASPS_NG13.20”; exon “1”; |
| chr5 | exon | 112988136 | 112988282 | + | gene_id “ASPS_NG13”; transcript_id |
| | | | | | “ASPS_NG13.20”; exon “2”; |
| chr5 | exon | 112989134 | 112989299 | + | gene_id “ASPS_NG13”; transcript_id |
| | | | | | “ASPS_NG13.20”; exon “3”; |
| chr5 | exon | 113026756 | 113026851 | + | gene_id “ASPS_NG13”; transcript_id |
| | | | | | “ASPS_NG13.20”; exon “4”; |
| chr5 | exon | 113030776 | 113031030 | + | gene_id “ASPS_NG13”; transcript_id |
| | | | | | “ASPS_NG13.20”; exon “5”; |
| chr5 | exon | 113048088 | 113048242 | + | gene_id “ASPS_NG13”; transcript_id |
| | | | | | “ASPS_NG13.20”; exon “6”; |
| chr5 | exon | 113050251 | 113050354 | + | gene_id “ASPS_NG13”; transcript_id |
| | | | | | “ASPS_NG13.20”; exon “7”; |
| chr5 | exon | 113051700 | 113052437 | + | gene_id “ASPS_NG13”; transcript_id |
| | | | | | “ASPS_NG13.20”; exon “8”; |
| chr5 | transcript | 112949280 | 113259872 | + | gene_id “ASPS_NG13”; transcript_id |
| | | | | | “ASPS_NG13.5”; RPKM “1.9936”; cov |
| | | | | | “26.4316”; |
| chr5 | exon | 112949280 | 112949426 | + | gene_id “ASPS_NG13”; transcript_id |
| | | | | | “ASPS_NG13.5”; exon “1”; |
| chr5 | exon | 113026756 | 113026851 | + | gene_id “ASPS_NG13”; transcript_id |
| | | | | | “ASPS_NG13.5”; exon “2”; |
| chr5 | exon | 113069258 | 113069343 | + | gene_id “ASPS_NG13”; transcript_id |
| | | | | | “ASPS_NG13.5”; exon “3”; |
| chr5 | exon | 113257231 | 113259872 | + | gene_id “ASPS_NG13”; transcript_id |
| | | | | | “ASPS_NG13.5”; exon “4”; |
| chr5 | transcript | 112949280 | 112967699 | + | gene_id “ASPS_NG13”; transcript_id |
| | | | | | “ASPS_NG13.4”; RPKM “1.6734”; cov |
| | | | | | “22.1861”; |
| chr5 | exon | 112949280 | 112949426 | + | gene_id “ASPS_NG13”; transcript_id |
| | | | | | “ASPS_NG13.4”; exon “1”; |
| chr5 | exon | 112966660 | 112967699 | + | gene_id “ASPS_NG13”; transcript_id |
| | | | | | “ASPS_NG13.4”; exon “2”; |
| chr5 | transcript | 112949280 | 113052437 | + | gene_id “ASPS_NG13”; transcript_id |
| | | | | | “ASPS_NG13.3”; RPKM “0.8982”; cov |
| | | | | | “11.9091”; |
| chr5 | exon | 112949280 | 112949426 | + | gene_id “ASPS_NG13”; transcript_id |
| | | | | | “ASPS_NG13.3”; exon “1”; |
| chr5 | exon | 113026756 | 113026851 | + | gene_id “ASPS_NG13”; transcript_id |
| | | | | | “ASPS_NG13.3”; exon “2”; |
| chr5 | exon | 113050251 | 113050354 | + | gene_id “ASPS_NG13”; transcript_id |
| | | | | | “ASPS_NG13.3”; exon “3”; |
| chr5 | exon | 113051707 | 113052437 | + | gene_id “ASPS_NG13”; transcript_id |
| | | | | | “ASPS_NG13.3”; exon “4”; |
| chr5 | transcript | 112949280 | 113659840 | + | gene_id “ASPS_NG13”; transcript_id |
| | | | | | “ASPS_NG13.18”; RPKM “2.3720”; cov |
| | | | | | “31.4491”; |
| chr5 | exon | 112949280 | 112949426 | + | gene_id “ASPS_NG13”; transcript_id |
| | | | | | “ASPS_NG13.18”; exon “1”; |
| chr5 | exon | 112966660 | 112966985 | + | gene_id “ASPS_NG13”; transcript_id |
| | | | | | “ASPS_NG13.18”; exon “2”; |
| chr5 | exon | 112969011 | 112969061 | + | gene_id “ASPS_NG13”; transcript_id |
| | | | | | “ASPS_NG13.18”; exon “3”; |
| chr5 | exon | 113026756 | 113026851 | + | gene_id “ASPS_NG13”; transcript_id |
| | | | | | “ASPS_NG13.18”; exon “4”; |
| chr5 | exon | 113069258 | 113069343 | + | gene_id “ASPS_NG13”; transcript_id |
| | | | | | “ASPS_NG13.18”; exon “5”; |
| chr5 | exon | 113333873 | 113334059 | + | gene_id “ASPS_NG13”; transcript_id |
| | | | | | “ASPS_NG13.18”; exon “6”; |
| chr5 | exon | 113557177 | 113557262 | + | gene_id “ASPS_NG13”; transcript_id |
| | | | | | “ASPS_NG13.18”; exon “7”; |
| chr5 | exon | 113659398 | 113659840 | + | gene_id “ASPS_NG13”; transcript_id |
| | | | | | “ASPS_NG13.18”; exon “8”; |
| chr5 | transcript | 112949280 | 113334096 | + | gene_id “ASPS_NG13”; transcript_id |
| | | | | | “ASPS_NG13.14”; RPKM “5.9752”; cov |
| | | | | | “79.2222”; |
| chr5 | exon | 112949280 | 112949426 | + | gene_id “ASPS_NG13”; transcript_id |
| | | | | | “ASPS_NG13.14”; exon “1”; |
| chr5 | exon | 112969011 | 112969061 | + | gene_id “ASPS_NG13”; transcript_id |
| | | | | | “ASPS_NG13.14”; exon “2”; |
| chr5 | exon | 113026756 | 113026851 | + | gene_id “ASPS_NG13”; transcript_id |
| | | | | | “ASPS_NG13.14”; exon “3”; |
| chr5 | exon | 113333873 | 113334096 | + | gene_id “ASPS_NG13”; transcript_id |
| | | | | | “ASPS_NG13.14”; exon “4”; |
| chr5 | transcript | 112949280 | 113031854 | + | gene_id “ASPS_NG13”; transcript_id |
| | | | | | “ASPS_NG13.15”; RPKM “5.7787”; cov |
| | | | | | “76.6166”; |
| chr5 | exon | 112949280 | 112949426 | + | gene_id “ASPS_NG13”; transcript_id |
| | | | | | “ASPS_NG13.15”; exon “1”; |
| chr5 | exon | 112969011 | 112969061 | + | gene_id “ASPS_NG13”; transcript_id |
| | | | | | “ASPS_NG13.15”; exon “2”; |
| chr5 | exon | 113026756 | 113026851 | + | gene_id “ASPS_NG13”; transcript_id |
| | | | | | “ASPS_NG13.15”; exon “3”; |
| chr5 | exon | 113030776 | 113031854 | + | gene_id “ASPS_NG13”; transcript_id |
| | | | | | “ASPS_NG13.15”; exon “4”; |
| chr5 | transcript | 112949280 | 113052437 | + | gene_id “ASPS_NG13”; transcript_id |
| | | | | | “ASPS_NG13.13”; RPKM “7.3834”; cov |
| | | | | | “97.8922”; |
| chr5 | exon | 112949280 | 112949426 | + | gene_id “ASPS_NG13”; transcript_id |
| | | | | | “ASPS_NG13.13”; exon “1”; |
| chr5 | exon | 112969011 | 112969061 | + | gene_id “ASPS_NG13”; transcript_id |
| | | | | | “ASPS_NG13.13”; exon “2”; |
| chr5 | exon | 112988136 | 112988282 | + | gene_id “ASPS_NG13”; transcript_id |
| | | | | | “ASPS_NG13.13”; exon “3”; |
| chr5 | exon | 112989134 | 112989299 | + | gene_id “ASPS_NG13”; transcript_id |
| | | | | | “ASPS_NG13.13”; exon “4”; |
| chr5 | exon | 113026756 | 113026851 | + | gene_id “ASPS_NG13”; transcript_id |
| | | | | | “ASPS_NG13.13”; exon “5”; |
| chr5 | exon | 113030776 | 113031030 | + | gene_id “ASPS_NG13”; transcript_id |
| | | | | | “ASPS_NG13.13”; exon “6”; |
| chr5 | exon | 113048088 | 113048242 | + | gene_id “ASPS_NG13”; transcript_id |
| | | | | | “ASPS_NG13.13”; exon “7”; |
| chr5 | exon | 113050251 | 113050354 | + | gene_id “ASPS_NG13”; transcript_id |
| | | | | | “ASPS_NG13.13”; exon “8”; |
| chr5 | exon | 113051707 | 113052437 | + | gene_id “ASPS_NG13”; transcript_id |
| | | | | | “ASPS_NG13.13”; exon “9”; |
| chr5 | transcript | 112949280 | 113052437 | + | gene_id “ASPS_NG13”; transcript_id |
| | | | | | “ASPS_NG13.11”; RPKM “9.3305”; cov |
| | | | | | “123.7077”; |
| chr5 | exon | 112949280 | 112949426 | + | gene_id “ASPS_NG13”; transcript_id |
| | | | | | “ASPS_NG13.11”; exon “1”; |
| chr5 | exon | 112966660 | 112966985 | + | gene_id “ASPS_NG13”; transcript_id |
| | | | | | “ASPS_NG13.11”; exon “2”; |
| chr5 | exon | 112969011 | 112969061 | + | gene_id “ASPS_NG13”; transcript_id |
| | | | | | “ASPS_NG13.11”; exon “3”; |
| chr5 | exon | 113026756 | 113026851 | + | gene_id “ASPS_NG13”; transcript_id |
| | | | | | “ASPS_NG13.11”; exon “4”; |
| chr5 | exon | 113030776 | 113031030 | + | gene_id “ASPS_NG13”; transcript_id |
| | | | | | “ASPS_NG13.11”; exon “5”; |
| chr5 | exon | 113048088 | 113052437 | + | gene_id “ASPS_NG13”; transcript_id |
| | | | | | “ASPS_NG13.11”; exon “6”; |
| chr5 | transcript | 112949280 | 112990349 | + | gene_id “ASPS_NG13”; transcript_id |
| | | | | | “ASPS_NG13.10”; RPKM “1.2471”; cov |
| | | | | | “16.5342”; |
| chr5 | exon | 112949280 | 112949426 | + | gene_id “ASPS_NG13”; transcript_id |
| | | | | | “ASPS_NG13.10”; exon “1”; |
| chr5 | exon | 112966660 | 112966985 | + | gene_id “ASPS_NG13”; transcript_id |
| | | | | | “ASPS_NG13.10”; exon “2”; |
| chr5 | exon | 112969011 | 112969061 | + | gene_id “ASPS_NG13”; transcript_id |
| | | | | | “ASPS_NG13.10”; exon “3”; |
| chr5 | exon | 112988136 | 112988282 | + | gene_id “ASPS_NG13”; transcript_id |
| | | | | | “ASPS_NG13.10”; exon “4”; |
| chr5 | exon | 112989134 | 112990349 | + | gene_id “ASPS_NG13”; transcript_id |
| | | | | | “ASPS_NG13.10”; exon “5”; |
| chr5 | transcript | 112949280 | 113259872 | + | gene_id “ASPS_NG13”; transcript_id |
| | | | | | “ASPS_NG13.8”; RPKM “3.1123”; cov |
| | | | | | “41.2645”; |
| chr5 | exon | 112949280 | 112949426 | + | gene_id “ASPS_NG13”; transcript_id |
| | | | | | “ASPS_NG13.8”; exon “1”; |
| chr5 | exon | 112969011 | 112969061 | + | gene_id “ASPS_NG13”; transcript_id |
| | | | | | “ASPS_NG13.8”; exon “2”; |
| chr5 | exon | 113026756 | 113026851 | + | gene_id “ASPS_NG13”; transcript_id |
| | | | | | “ASPS_NG13.8”; exon “3”; |
| chr5 | exon | 113069258 | 113069343 | + | gene_id “ASPS_NG13”; transcript_id |
| | | | | | “ASPS_NG13.8”; exon “4”; |
| chr5 | exon | 113257231 | 113259872 | + | gene_id “ASPS_NG13”; transcript_id |
| | | | | | “ASPS_NG13.8”; exon “5”; |
| chr5 | transcript | 112949280 | 113052437 | + | gene_id “ASPS_NG13”; transcript_id |
| | | | | | “ASPS_NG13.7”; RPKM “8.9994”; cov |
| | | | | | “119.3183”; |
| chr5 | exon | 112949280 | 112949426 | + | gene_id “ASPS_NG13”; transcript_id |
| | | | | | “ASPS_NG13.7”; exon “1”; |
| chr5 | exon | 112969011 | 112969061 | + | gene_id “ASPS_NG13”; transcript_id |
| | | | | | “ASPS_NG13.7”; exon “2”; |
| chr5 | exon | 113026756 | 113026851 | + | gene_id “ASPS_NG13”; transcript_id |
| | | | | | “ASPS_NG13.7”; exon “3”; |
| chr5 | exon | 113048088 | 113048242 | + | gene_id “ASPS_NG13”; transcript_id |
| | | | | | “ASPS_NG13.7”; exon “4”; |
| chr5 | exon | 113050251 | 113052437 | + | gene_id “ASPS_NG13”; transcript_id |
| | | | | | “ASPS_NG13.7”; exon “5”; |
| chr5 | transcript | 112949280 | 113052437 | + | gene_id “ASPS_NG13”; transcript_id |
| | | | | | “ASPS_NG13.6”; RPKM “18.2483”; cov |
| | | | | | “241.9431”; |
| chr5 | exon | 112949280 | 112949426 | + | gene_id “ASPS_NG13”; transcript_id |
| | | | | | “ASPS_NG13.6”; exon “1”; |
| chr5 | exon | 113026756 | 113026851 | + | gene_id “ASPS_NG13”; transcript_id |
| | | | | | “ASPS_NG13.6”; exon “2”; |
| chr5 | exon | 113048088 | 113048242 | + | gene_id “ASPS_NG13”; transcript_id |
| | | | | | “ASPS_NG13.6”; exon “3”; |
| chr5 | exon | 113050251 | 113052437 | + | gene_id “ASPS_NG13”; transcript_id |
| | | | | | “ASPS_NG13.6”; exon “4”; |
| chr5 | transcript | 112949280 | 113052437 | + | gene_id “ASPS_NG13”; transcript_id |
| | | | | | “ASPS_NG13.12”; RPKM “8.5899”; cov |
| | | | | | “113.8881”; |
| chr5 | exon | 112949280 | 112949426 | + | gene_id “ASPS_NG13”; transcript_id |
| | | | | | “ASPS_NG13.12”; exon “1”; |
| chr5 | exon | 113026756 | 113026851 | + | gene_id “ASPS_NG13”; transcript_id |
| | | | | | “ASPS_NG13.12”; exon “2”; |
| chr5 | exon | 113030776 | 113031030 | + | gene_id “ASPS_NG13”; transcript_id |
| | | | | | “ASPS_NG13.12”; exon “3”; |
| chr5 | exon | 113048088 | 113048242 | + | gene_id “ASPS_NG13”; transcript_id |
| | | | | | “ASPS_NG13.12”; exon “4”; |
| chr5 | exon | 113050251 | 113050354 | + | gene_id “ASPS_NG13”; transcript_id |
| | | | | | “ASPS_NG13.12”; exon “5”; |
| chr5 | exon | 113051707 | 113052437 | + | gene_id “ASPS_NG13”; transcript_id |
| | | | | | “ASPS_NG13.12”; exon “6”; |
| chr5 | transcript | 112949280 | 112993964 | + | gene_id “ASPS_NG13”; transcript_id |
| | | | | | “ASPS_NG13.9”; RPKM “6.8310”; cov |
| | | | | | “90.5684”; |
| chr5 | exon | 112949280 | 112949426 | + | gene_id “ASPS_NG13”; transcript_id |
| | | | | | “ASPS_NG13.9”; exon “1”; |
| chr5 | exon | 112966660 | 112966985 | + | gene_id “ASPS_NG13”; transcript_id |
| | | | | | “ASPS_NG13.9”; exon “2”; |
| chr5 | exon | 112969011 | 112969061 | + | gene_id “ASPS_NG13”; transcript_id |
| | | | | | “ASPS_NG13.9”; exon “3”; |
| chr5 | exon | 112988136 | 112988282 | + | gene_id “ASPS_NG13”; transcript_id |
| | | | | | “ASPS_NG13.9”; exon “4”; |
| chr5 | exon | 112989134 | 112989299 | + | gene_id “ASPS_NG13”; transcript_id |
| | | | | | “ASPS_NG13.9”; exon “5”; |
| chr5 | exon | 112992477 | 112993964 | + | gene_id “ASPS_NG13”; transcript_id |
| | | | | | “ASPS_NG13.9”; exon “6”; |
| chr5 | transcript | 112949280 | 113129757 | + | gene_id “ASPS_NG13”; transcript_id |
| | | | | | “ASPS_NG13.19”; RPKM “2.2739”; cov |
| | | | | | “30.1487”; |
| chr5 | exon | 112949280 | 112949426 | + | gene_id “ASPS_NG13”; transcript_id |
| | | | | | “ASPS_NG13.19”; exon “1”; |
| chr5 | exon | 112969011 | 112969061 | + | gene_id “ASPS_NG13”; transcript_id |
| | | | | | “ASPS_NG13.19”; exon “2”; |
| chr5 | exon | 113026756 | 113026851 | + | gene_id “ASPS_NG13”; transcript_id |
| | | | | | “ASPS_NG13.19”; exon “3”; |
| chr5 | exon | 113069258 | 113069343 | + | gene_id “ASPS_NG13”; transcript_id |
| | | | | | “ASPS_NG13.19”; exon “4”; |
| chr5 | exon | 113086902 | 113086943 | + | gene_id “ASPS_NG13”; transcript_id |
| | | | | | “ASPS_NG13.19”; exon “5”; |
| chr5 | exon | 113098038 | 113098138 | + | gene_id “ASPS_NG13”; transcript_id |
| | | | | | “ASPS_NG13.19”; exon “6”; |
| chr5 | exon | 113127131 | 113129757 | + | gene_id “ASPS_NG13”; transcript_id |
| | | | | | “ASPS_NG13.19”; exon “7”; |
| chr5 | transcript | 113008180 | 113011670 | + | gene_id “ASPS_NG13”; transcript_id |
| | | | | | “ASPS_NG13.2”; RPKM “3.9156”; cov |
| | | | | | “51.9142”; |
| chr5 | exon | 113008180 | 113011670 | + | gene_id “ASPS_NG13”; transcript_id |
| | | | | | “ASPS_NG13.2”; exon “1”; |
| chr5 | transcript | 113071583 | 113334096 | + | gene_id “ASPS_NG13”; transcript_id |
| | | | | | “ASPS_NG13.17”; RPKM “2.4834”; cov |
| | | | | | “32.9261”; |
| chr5 | exon | 113071583 | 113072029 | + | gene_id “ASPS_NG13”; transcript_id |
| | | | | | “ASPS_NG13.17”; exon “1”; |
| chr5 | exon | 113333873 | 113334096 | + | gene_id “ASPS_NG13”; transcript_id |
| | | | | | “ASPS_NG13.17”; exon “2”; |
| chr5 | transcript | 113086065 | 113157528 | + | gene_id “ASPS_NG13”; transcript_id |
| | | | | | “ASPS_NG13.22”; RPKM “0.9950”; cov |
| | | | | | “13.1927”; |
| chr5 | exon | 113086065 | 113086943 | + | gene_id “ASPS_NG13”; transcript_id |
| | | | | | “ASPS_NG13.22”; exon “1”; |
| chr5 | exon | 113098038 | 113098138 | + | gene_id “ASPS_NG13”; transcript_id |
| | | | | | “ASPS_NG13.22”; exon “2”; |
| chr5 | exon | 113154692 | 113157528 | + | gene_id “ASPS_NG13”; transcript_id |
| | | | | | “ASPS_NG13.22”; exon “3”; |
| chr5 | transcript | 113121979 | 113129757 | + | gene_id “ASPS_NG13”; transcript_id |
| | | | | | “ASPS_NG13.1”; RPKM “6.8772”; cov |
| | | | | | “91.1809”; |
| chr5 | exon | 113121979 | 113129757 | + | gene_id “ASPS_NG13”; transcript_id |
| | | | | | “ASPS_NG13.1”; exon “1”; |
| chr5 | transcript | 113000528 | 113006158 | + | gene_id “ASPS_NG15”; transcript_id |
| | | | | | “ASPS_NG15.1”; RPKM “13.6474”; cov |
| | | | | | “180.9435”; |
| chr5 | exon | 113000528 | 113006158 | + | gene_id “ASPS_NG15”; transcript_id |
| | | | | | “ASPS_NG15.1”; exon “1”; |
| chr5 | transcript | 113212587 | 113216859 | + | gene_id “ASPS_NG14”; transcript_id |
| | | | | | “ASPS_NG14.1”; RPKM “9.6733”; cov |
| | | | | | “128.2527”; |
| chr5 | exon | 113212587 | 113216859 | + | gene_id “ASPS_NG14”; transcript_id |
| | | | | | “ASPS_NG14.1”; exon “1”; |
| chr6 | transcript | 37741718 | 37745323 | − | gene_id “ASPS_NG16”; transcript_id |
| | | | | | “ASPS_NG16.1”; RPKM “1.7981”; cov |
| | | | | | “23.8405”; |
| chr6 | exon | 37741718 | 37742323 | − | gene_id “ASPS_NG16”; transcript_id |
| | | | | | “ASPS_NG16.1”; exon “1”; |
| chr6 | exon | 37744229 | 37744428 | − | gene_id “ASPS_NG16”; transcript_id |
| | | | | | “ASPS_NG16.1”; exon “2”; |
| chr6 | exon | 37745112 | 37745323 | − | gene_id “ASPS_NG16”; transcript_id |
| | | | | | “ASPS_NG16.1”; exon “3”; |
| chr6 | transcript | 37741718 | 37745323 | − | gene_id “ASPS_NG16”; transcript_id |
| | | | | | “ASPS_NG16.2”; RPKM “1.5995”; cov |
| | | | | | “21.2068”; |
| chr6 | exon | 37741718 | 37742323 | − | gene_id “ASPS_NG16”; transcript_id |
| | | | | | “ASPS_NG16.2”; exon “1”; |
| chr6 | exon | 37744229 | 37745323 | − | gene_id “ASPS_NG16”; transcript_id |
| | | | | | “ASPS_NG16.2”; exon “2”; |
| chr6 | transcript | 150621167 | 150627265 | + | gene_id “ASPS_NG17”; transcript_id |
| | | | | | “ASPS_NG17.1”; RPKM “7.9164”; cov |
| | | | | | “104.9589”; |
| chr6 | exon | 150621167 | 150621622 | + | gene_id “ASPS_NG17”; transcript_id |
| | | | | | “ASPS_NG17.1”; exon “1”; |
| chr6 | exon | 150626922 | 150627265 | + | gene_id “ASPS_NG17”; transcript_id |
| | | | | | “ASPS_NG17.1”; exon “2”; |
| chr8 | transcript | 118776780 | 118782163 | + | gene_id “ASPS_NG18”; transcript_id |
| | | | | | “ASPS_NG18.1”; RPKM “8.2527”; cov |
| | | | | | “109.4172”; |
| chr8 | exon | 118776780 | 118776876 | + | gene_id “ASPS_NG18”; transcript_id |
| | | | | | “ASPS_NG18.1”; exon “1”; |
| chr8 | exon | 118779033 | 118779103 | + | gene_id “ASPS_NG18”; transcript_id |
| | | | | | “ASPS_NG18.1”; exon “2”; |
| chr8 | exon | 118781925 | 118782163 | + | gene_id “ASPS_NG18”; transcript_id |
| | | | | | “ASPS_NG18.1”; exon “3”; |
| chr8 | transcript | 118776780 | 118782163 | + | gene_id “ASPS_NG18”; transcript_id |
| | | | | | “ASPS_NG18.2”; RPKM “3.9654”; cov |
| | | | | | “52.5752”; |
| chr8 | exon | 118776780 | 118776876 | + | gene_id “ASPS_NG18”; transcript_id |
| | | | | | “ASPS_NG18.2”; exon “1”; |
| chr8 | exon | 118779033 | 118779103 | + | gene_id “ASPS_NG18”; transcript_id |
| | | | | | “ASPS_NG18.2”; exon “2”; |
| chr8 | exon | 118781606 | 118782163 | + | gene_id “ASPS_NG18”; transcript_id |
| | | | | | “ASPS_NG18.2”; exon “3”; |
| chr8 | transcript | 118776780 | 118782163 | + | gene_id “ASPS_NG18”; transcript_id |
| | | | | | “ASPS_NG18.3”; RPKM “1.4728”; cov |
| | | | | | “19.5269”; |
| chr8 | exon | 118776780 | 118776876 | + | gene_id “ASPS_NG18”; transcript_id |
| | | | | | “ASPS_NG18.3”; exon “1”; |
| chr8 | exon | 118779033 | 118779103 | + | gene_id “ASPS_NG18”; transcript_id |
| | | | | | “ASPS_NG18.3”; exon “2”; |
| chr8 | exon | 118779974 | 118782163 | + | gene_id “ASPS_NG18”; transcript_id |
| | | | | | “ASPS_NG18.3”; exon “3”; |
| chr8 | transcript | 132845202 | 132864921 | − | gene_id “ASPS_NG19”; transcript_id |
| | | | | | “ASPS_NG19.2”; RPKM “2.7817”; cov |
| | | | | | “36.8812”; |
| chr8 | exon | 132845202 | 132854933 | − | gene_id “ASPS_NG19”; transcript_id |
| | | | | | “ASPS_NG19.2”; exon “1”; |
| chr8 | exon | 132855839 | 132855940 | − | gene_id “ASPS_NG19”; transcript_id |
| | | | | | “ASPS_NG19.2”; exon “2”; |
| chr8 | exon | 132864681 | 132864921 | − | gene_id “ASPS_NG19”; transcript_id |
| | | | | | “ASPS_NG19.2”; exon “3”; |
| chr8 | transcript | 132863046 | 132864921 | − | gene_id “ASPS_NG19”; transcript_id |
| | | | | | “ASPS_NG19.1”; RPKM “18.5467”; cov |
| | | | | | “245.8995”; |
| chr8 | exon | 132863046 | 132864921 | − | gene_id “ASPS_NG19”; transcript_id |
| | | | | | “ASPS_NG19.1”; exon “1”; |
| chr8 | transcript | 134939202 | 134948254 | + | gene_id “ASPS_NG20”; transcript_id |
| | | | | | “ASPS_NG20.3”; RPKM “1.2962”; cov |
| | | | | | “17.1850”; |
| chr8 | exon | 134939202 | 134939301 | + | gene_id “ASPS_NG20”; transcript_id |
| | | | | | “ASPS_NG20.3”; exon “1”; |
| chr8 | exon | 134940629 | 134940683 | + | gene_id “ASPS_NG20”; transcript_id |
| | | | | | “ASPS_NG20.3”; exon “2”; |
| chr8 | exon | 134945156 | 134948254 | + | gene_id “ASPS_NG20”; transcript_id |
| | | | | | “ASPS_NG20.3”; exon “3”; |
| chr8 | transcript | 134939202 | 134948254 | + | gene_id “ASPS_NG20”; transcript_id |
| | | | | | “ASPS_NG20.2”; RPKM “1.3587”; cov |
| | | | | | “18.0147”; |
| chr8 | exon | 134939202 | 134939301 | + | gene_id “ASPS_NG20”; transcript_id |
| | | | | | “ASPS_NG20.2”; exon “1”; |
| chr8 | exon | 134940629 | 134940683 | + | gene_id “ASPS_NG20”; transcript_id |
| | | | | | “ASPS_NG20.2”; exon “2”; |
| chr8 | exon | 134945156 | 134945173 | + | gene_id “ASPS_NG20”; transcript_id |
| | | | | | “ASPS_NG20.2”; exon “3”; |
| chr8 | exon | 134946835 | 134948254 | + | gene_id “ASPS_NG20”; transcript_id |
| | | | | | “ASPS_NG20.2”; exon “4”; |
| chr8 | transcript | 134939202 | 134948254 | + | gene_id “ASPS_NG20”; transcript_id |
| | | | | | “ASPS_NG20.1”; RPKM “1.0919”; cov |
| | | | | | “14.4768”; |
| chr8 | exon | 134939202 | 134939301 | + | gene_id “ASPS_NG20”; transcript_id |
| | | | | | “ASPS_NG20.1”; exon “1”; |
| chr8 | exon | 134940629 | 134940683 | + | gene_id “ASPS_NG20”; transcript_id |
| | | | | | “ASPS_NG20.1”; exon “2”; |
| chr8 | exon | 134946835 | 134948254 | + | gene_id “ASPS_NG20”; transcript_id |
| | | | | | “ASPS_NG20.1”; exon “3”; |
| chr8 | transcript | 134939202 | 134976707 | + | gene_id “ASPS_NG20”; transcript_id |
| | | | | | “ASPS_NG20.4”; RPKM “10.5807”; cov |
| | | | | | “140.2833”; |
| chr8 | exon | 134939202 | 134939301 | + | gene_id “ASPS_NG20”; transcript_id |
| | | | | | “ASPS_NG20.4”; exon “1”; |
| chr8 | exon | 134940629 | 134940683 | + | gene_id “ASPS_NG20”; transcript_id |
| | | | | | “ASPS_NG20.4”; exon “2”; |
| chr8 | exon | 134966933 | 134976707 | + | gene_id “ASPS_NG20”; transcript_id |
| | | | | | “ASPS_NG20.4”; exon “3”; |
| chr8 | transcript | 134939202 | 134976707 | + | gene_id “ASPS_NG20”; transcript_id |
| | | | | | “ASPS_NG20.5”; RPKM “5.2540”; cov |
| | | | | | “69.6596”; |
| chr8 | exon | 134939202 | 134939301 | + | gene_id “ASPS_NG20”; transcript_id |
| | | | | | “ASPS_NG20.5”; exon “1”; |
| chr8 | exon | 134940629 | 134940683 | + | gene_id “ASPS_NG20”; transcript_id |
| | | | | | “ASPS_NG20.5”; exon “2”; |
| chr8 | exon | 134975792 | 134976707 | + | gene_id “ASPS_NG20”; transcript_id |
| | | | | | “ASPS_NG20.5”; exon “3”; |
| chr8 | transcript | 134939202 | 134976707 | + | gene_id “ASPS_NG20”; transcript_id |
| | | | | | “ASPS_NG20.8”; RPKM “1.3655”; cov |
| | | | | | “18.1038”; |
| chr8 | exon | 134939202 | 134939301 | + | gene_id “ASPS_NG20”; transcript_id |
| | | | | | “ASPS_NG20.8”; exon “1”; |
| chr8 | exon | 134940629 | 134940683 | + | gene_id “ASPS_NG20”; transcript_id |
| | | | | | “ASPS_NG20.8”; exon “2”; |
| chr8 | exon | 134950565 | 134950673 | + | gene_id “ASPS_NG20”; transcript_id |
| | | | | | “ASPS_NG20.8”; exon “3”; |
| chr8 | exon | 134975792 | 134976707 | + | gene_id “ASPS_NG20”; transcript_id |
| | | | | | “ASPS_NG20.8”; exon “4”; |
| chr8 | transcript | 134939202 | 134976707 | + | gene_id “ASPS_NG20”; transcript_id |
| | | | | | “ASPS_NG20.7”; RPKM “5.0122”; cov |
| | | | | | “66.4538”; |
| chr8 | exon | 134939202 | 134939301 | + | gene_id “ASPS_NG20”; transcript_id |
| | | | | | “ASPS_NG20.7”; exon “1”; |
| chr8 | exon | 134940629 | 134940683 | + | gene_id “ASPS_NG20”; transcript_id |
| | | | | | “ASPS_NG20.7”; exon “2”; |
| chr8 | exon | 134950565 | 134950673 | + | gene_id “ASPS_NG20”; transcript_id |
| | | | | | “ASPS_NG20.7”; exon “3”; |
| chr8 | exon | 134966933 | 134976707 | + | gene_id “ASPS_NG20”; transcript_id |
| | | | | | “ASPS_NG20.7”; exon “4”; |
| chr8 | transcript | 134939202 | 135017839 | + | gene_id “ASPS_NG20”; transcript_id |
| | | | | | “ASPS_NG20.6”; RPKM “1.4324”; cov |
| | | | | | “18.9917”; |
| chr8 | exon | 134939202 | 134939301 | + | gene_id “ASPS_NG20”; transcript_id |
| | | | | | “ASPS_NG20.6”; exon “1”; |
| chr8 | exon | 134940629 | 134940683 | + | gene_id “ASPS_NG20”; transcript_id |
| | | | | | “ASPS_NG20.6”; exon “2”; |
| chr8 | exon | 134975792 | 134975934 | + | gene_id “ASPS_NG20”; transcript_id |
| | | | | | “ASPS_NG20.6”; exon “3”; |
| chr8 | exon | 135014343 | 135017839 | + | gene_id “ASPS_NG20”; transcript_id |
| | | | | | “ASPS_NG20.6”; exon “4”; |
| chr9 | transcript | 102121560 | 102134490 | + | gene_id “ASPS_NG21”; transcript_id |
| | | | | | “ASPS_NG21.2”; RPKM “0.9077”; cov |
| | | | | | “12.0343”; |
| chr9 | exon | 102121560 | 102121919 | + | gene_id “ASPS_NG21”; transcript_id |
| | | | | | “ASPS_NG21.2”; exon “1”; |
| chr9 | exon | 102123653 | 102123782 | + | gene_id “ASPS_NG21”; transcript_id |
| | | | | | “ASPS_NG21.2”; exon “2”; |
| chr9 | exon | 102133335 | 102134490 | + | gene_id “ASPS_NG21”; transcript_id |
| | | | | | “ASPS_NG21.2”; exon “3”; |
| chr9 | transcript | 102130287 | 102134490 | + | gene_id “ASPS_NG21”; transcript_id |
| | | | | | “ASPS_NG21.1”; RPKM “6.2035”; cov |
| | | | | | “82.2483”; |
| chr9 | exon | 102130287 | 102134490 | + | gene_id “ASPS_NG21”; transcript_id |
| | | | | | “ASPS_NG21.1”; exon “1”; |
| chr9 | transcript | 102125575 | 102130251 | + | gene_id “ASPS_NG22”; transcript_id |
| | | | | | “ASPS_NG22.1”; RPKM “2.3838”; cov |
| | | | | | “31.6060”; |
| chr9 | exon | 102125575 | 102130251 | + | gene_id “ASPS_NG22”; transcript_id |
| | | | | | “ASPS_NG22.1”; exon “1”; |
| chr4 | transcript | 117434454 | 117435921 | + | gene_id “emCS_NG1”; transcript_id |
| | | | | | “emCS_NG1.1”; RPKM “8.2663”; cov |
| | | | | | “20.7905”; |
| chr4 | exon | 117434454 | 117434782 | + | gene_id “emCS_NG1”; transcript_id |
| | | | | | “emCS_NG1.1”; exon “1”; |
| chr4 | exon | 117435427 | 117435921 | + | gene_id “emCS_NG1”; transcript_id |
| | | | | | “emCS_NG1.1”; exon “2”; |
| chr5 | transcript | 102073836 | 102079406 | − | gene_id “emCS_NG2”; transcript_id |
| | | | | | “emCS_NG2.1”; RPKM “10.4680”; cov |
| | | | | | “26.3281”; |
| chr5 | exon | 102073836 | 102079406 | − | gene_id “emCS_NG2”; transcript_id |
| | | | | | “emCS_NG2.1”; exon “1”; |
| chr10 | transcript | 2717538 | 2723495 | − | gene_id “LGFS_NG20”; transcript_id |
| | | | | | “LGFS_NG20.1”; RPKM “0.8914”; cov |
| | | | | | “11.5098”; |
| chr10 | exon | 2717538 | 2721708 | − | gene_id “LGFS_NG20”; transcript_id |
| | | | | | “LGFS_NG20.1”; exon “1”; |
| chr10 | exon | 2722106 | 2723495 | − | gene_id “LGFS_NG20”; transcript_id |
| | | | | | “LGFS_NG20.1”; exon “2”; |
| chr10 | transcript | 3057432 | 3065690 | + | gene_id “LGFS_NG21”; transcript_id |
| | | | | | “LGFS_NG21.1”; RPKM “2.3966”; cov |
| | | | | | “30.9453”; |
| chr10 | exon | 3057432 | 3057803 | + | gene_id “LGFS_NG21”; transcript_id |
| | | | | | “LGFS_NG21.1”; exon “1”; |
| chr10 | exon | 3064639 | 3065690 | + | gene_id “LGFS_NG21”; transcript_id |
| | | | | | “LGFS_NG21.1”; exon “2”; |
| chr10 | transcript | 133484169 | 133490738 | − | gene_id “LGFS_NG22”; transcript_id |
| | | | | | “LGFS_NG22.1”; RPKM “1.2974”; cov |
| | | | | | “16.7518”; |
| chr10 | exon | 133484169 | 133487846 | − | gene_id “LGFS_NG22”; transcript_id |
| | | | | | “LGFS_NG22.1”; exon “1”; |
| chr10 | exon | 133487991 | 133489239 | − | gene_id “LGFS_NG22”; transcript_id |
| | | | | | “LGFS_NG22.1”; exon “2”; |
| chr10 | exon | 133489871 | 133490738 | − | gene_id “LGFS_NG22”; transcript_id |
| | | | | | “LGFS_NG22.1”; exon “3”; |
| chr10 | transcript | 133484169 | 133508852 | − | gene_id “LGFS_NG22”; transcript_id |
| | | | | | “LGFS_NG22.2”; RPKM “1.3602”; cov |
| | | | | | “17.5633”; |
| chr10 | exon | 133484169 | 133487846 | − | gene_id “LGFS_NG22”; transcript_id |
| | | | | | “LGFS_NG22.2”; exon “1”; |
| chr10 | exon | 133487991 | 133489239 | − | gene_id “LGFS_NG22”; transcript_id |
| | | | | | “LGFS_NG22.2”; exon “2”; |
| chr10 | exon | 133489871 | 133490067 | − | gene_id “LGFS_NG22”; transcript_id |
| | | | | | “LGFS_NG22.2”; exon “3”; |
| chr10 | exon | 133503654 | 133503887 | − | gene_id “LGFS_NG22”; transcript_id |
| | | | | | “LGFS_NG22.2”; exon “4”; |
| chr10 | exon | 133507888 | 133508852 | − | gene_id “LGFS_NG22”; transcript_id |
| | | | | | “LGFS_NG22.2”; exon “5”; |
| chr10 | transcript | 133952078 | 133956557 | − | gene_id “LGFS_NG23”; transcript_id |
| | | | | | “LGFS_NG23.1”; RPKM “1.0552”; cov |
| | | | | | “13.6244”; |
| chr10 | exon | 133952078 | 133955051 | − | gene_id “LGFS_NG23”; transcript_id |
| | | | | | “LGFS_NG23.1”; exon “1”; |
| chr10 | exon | 133955384 | 133956557 | − | gene_id “LGFS_NG23”; transcript_id |
| | | | | | “LGFS_NG23.1”; exon “2”; |
| chr10 | transcript | 133952078 | 133956557 | − | gene_id “LGFS_NG23”; transcript_id |
| | | | | | “LGFS_NG23.2”; RPKM “3.8085”; cov |
| | | | | | “49.1765”; |
| chr10 | exon | 133952078 | 133956557 | − | gene_id “LGFS_NG23”; transcript_id |
| | | | | | “LGFS_NG23.2”; exon “1”; |
| chr11 | transcript | 744567 | 744849 | + | gene_id “LGFS_NG24”; transcript_id |
| | | | | | “LGFS_NG24.1”; RPKM “17.5981”; cov |
| | | | | | “227.2297”; |
| chr11 | exon | 744567 | 744849 | + | gene_id “LGFS_NG24”; transcript_id |
| | | | | | “LGFS_NG24.1”; exon “1”; |
| chr12 | transcript | 9479672 | 9508176 | + | gene_id “LGFS_NG25”; transcript_id |
| | | | | | “LGFS_NG25.1”; RPKM “0.8729”; cov |
| | | | | | “11.2711”; |
| chr12 | exon | 9479672 | 9480389 | + | gene_id “LGFS_NG25”; transcript_id |
| | | | | | “LGFS_NG25.1”; exon “1”; |
| chr12 | exon | 9493053 | 9493802 | + | gene_id “LGFS_NG25”; transcript_id |
| | | | | | “LGFS_NG25.1”; exon “2”; |
| chr12 | exon | 9494420 | 9494557 | + | gene_id “LGFS_NG25”; transcript_id |
| | | | | | “LGFS_NG25.1”; exon “3”; |
| chr12 | exon | 9498775 | 9498941 | + | gene_id “LGFS_NG25”; transcript_id |
| | | | | | “LGFS_NG25.1”; exon “4”; |
| chr12 | exon | 9499362 | 9499528 | + | gene_id “LGFS_NG25”; transcript_id |
| | | | | | “LGFS_NG25.1”; exon “5”; |
| chr12 | exon | 9500555 | 9500621 | + | gene_id “LGFS_NG25”; transcript_id |
| | | | | | “LGFS_NG25.1”; exon “6”; |
| chr12 | exon | 9502613 | 9504969 | + | gene_id “LGFS_NG25”; transcript_id |
| | | | | | “LGFS_NG25.1”; exon “7”; |
| chr12 | exon | 9505014 | 9508176 | + | gene_id “LGFS_NG25”; transcript_id |
| | | | | | “LGFS_NG25.1”; exon “8”; |
| chr12 | transcript | 9479672 | 9508176 | + | gene_id “LGFS_NG25”; transcript_id |
| | | | | | “LGFS_NG25.2”; RPKM “1.8003”; cov |
| | | | | | “23.2463”; |
| chr12 | exon | 9479672 | 9480389 | + | gene_id “LGFS_NG25”; transcript_id |
| | | | | | “LGFS_NG25.2”; exon “1”; |
| chr12 | exon | 9493053 | 9493802 | + | gene_id “LGFS_NG25”; transcript_id |
| | | | | | “LGFS_NG25.2”; exon “2”; |
| chr12 | exon | 9498775 | 9498941 | + | gene_id “LGFS_NG25”; transcript_id |
| | | | | | “LGFS_NG25.2”; exon “3”; |
| chr12 | exon | 9499362 | 9499528 | + | gene_id “LGFS_NG25”; transcript_id |
| | | | | | “LGFS_NG25.2”; exon “4”; |
| chr12 | exon | 9500555 | 9500621 | + | gene_id “LGFS_NG25”; transcript_id |
| | | | | | “LGFS_NG25.2”; exon “5”; |
| chr12 | exon | 9502613 | 9504969 | + | gene_id “LGFS_NG25”; transcript_id |
| | | | | | “LGFS_NG25.2”; exon “6”; |
| chr12 | exon | 9505014 | 9508176 | + | gene_id “LGFS_NG25”; transcript_id |
| | | | | | “LGFS_NG25.2”; exon “7”; |
| chr12 | transcript | 110525134 | 110528865 | − | gene_id “LGFS_NG26”; transcript_id |
| | | | | | “LGFS_NG26.1”; RPKM “1.4572”; cov |
| | | | | | “18.8160”; |
| chr12 | exon | 110525134 | 110527451 | − | gene_id “LGFS_NG26”; transcript_id |
| | | | | | “LGFS_NG26.1”; exon “1”; |
| chr12 | exon | 110528747 | 110528865 | − | gene_id “LGFS_NG26”; transcript_id |
| | | | | | “LGFS_NG26.1”; exon “2”; |
| chr12 | transcript | 131941007 | 131943876 | + | gene_id “LGFS_NG27”; transcript_id |
| | | | | | “LGFS_NG27.1”; RPKM “0.7775”; cov |
| | | | | | “10.0390”; |
| chr12 | exon | 131941007 | 131942198 | + | gene_id “LGFS_NG27”; transcript_id |
| | | | | | “LGFS_NG27.1”; exon “1”; |
| chr12 | exon | 131942548 | 131943876 | + | gene_id “LGFS_NG27”; transcript_id |
| | | | | | “LGFS_NG27.1”; exon “2”; |
| chr12 | transcript | 131941007 | 131943876 | + | gene_id “LGFS_NG27”; transcript_id |
| | | | | | “LGFS_NG27.2”; RPKM “4.6524”; cov |
| | | | | | “60.0726”; |
| chr12 | exon | 131941007 | 131943876 | + | gene_id “LGFS_NG27”; transcript_id |
| | | | | | “LGFS_NG27.2”; exon “1”; |
| chr13 | transcript | 114771820 | 114773546 | + | gene_id “LGFS_NG29”; transcript_id |
| | | | | | “LGFS_NG29.2”; RPKM “1.6462”; cov |
| | | | | | “21.2566”; |
| chr13 | exon | 114771820 | 114771869 | + | gene_id “LGFS_NG29”; transcript_id |
| | | | | | “LGFS_NG29.2”; exon “1”; |
| chr13 | exon | 114772887 | 114773546 | + | gene_id “LGFS_NG29”; transcript_id |
| | | | | | “LGFS_NG29.2”; exon “2”; |
| chr13 | transcript | 114772735 | 114773546 | + | gene_id “LGFS_NG29”; transcript_id |
| | | | | | “LGFS_NG29.1”; RPKM “4.3671”; cov |
| | | | | | “56.3894”; |
| chr13 | exon | 114772735 | 114773546 | + | gene_id “LGFS_NG29”; transcript_id |
| | | | | | “LGFS_NG29.1”; exon “1”; |
| chr13 | transcript | 114773614 | 114776189 | + | gene_id “LGFS_NG30”; transcript_id |
| | | | | | “LGFS_NG30.1”; RPKM “3.9277”; cov |
| | | | | | “50.7157”; |
| chr13 | exon | 114773614 | 114776189 | + | gene_id “LGFS_NG30”; transcript_id |
| | | | | | “LGFS_NG30.1”; exon “1”; |
| chr13 | transcript | 114779389 | 114792981 | + | gene_id “LGFS_NG30”; transcript_id |
| | | | | | “LGFS_NG30.2”; RPKM “3.7448”; cov |
| | | | | | “48.3539”; |
| chr13 | exon | 114779389 | 114785469 | + | gene_id “LGFS_NG30”; transcript_id |
| | | | | | “LGFS_NG30.2”; exon “1”; |
| chr13 | exon | 114785511 | 114792981 | + | gene_id “LGFS_NG30”; transcript_id |
| | | | | | “LGFS_NG30.2”; exon “2”; |
| chr13 | transcript | 114779389 | 114792981 | + | gene_id “LGFS_NG30”; transcript_id |
| | | | | | “LGFS_NG30.3”; RPKM “3.2017”; cov |
| | | | | | “41.3409”; |
| chr13 | exon | 114779389 | 114785469 | + | gene_id “LGFS_NG30”; transcript_id |
| | | | | | “LGFS_NG30.3”; exon “1”; |
| chr13 | exon | 114785550 | 114785588 | + | gene_id “LGFS_NG30”; transcript_id |
| | | | | | “LGFS_NG30.3”; exon “2”; |
| chr13 | exon | 114785669 | 114792981 | + | gene_id “LGFS_NG30”; transcript_id |
| | | | | | “LGFS_NG30.3”; exon “3”; |
| chr13 | transcript | 114779389 | 114792981 | + | gene_id “LGFS_NG30”; transcript_id |
| | | | | | “LGFS_NG30.4”; RPKM “1.1265”; cov |
| | | | | | “14.5458”; |
| chr13 | exon | 114779389 | 114785469 | + | gene_id “LGFS_NG30”; transcript_id |
| | | | | | “LGFS_NG30.4”; exon “1”; |
| chr13 | exon | 114785550 | 114792981 | + | gene_id “LGFS_NG30”; transcript_id |
| | | | | | “LGFS_NG30.4”; exon “2”; |
| chr13 | transcript | 114776253 | 114779358 | + | gene_id “LGFS_NG28”; transcript_id |
| | | | | | “LGFS_NG28.1”; RPKM “15.8583”; cov |
| | | | | | “204.7648”; |
| chr13 | exon | 114776253 | 114779358 | + | gene_id “LGFS_NG28”; transcript_id |
| | | | | | “LGFS_NG28.1”; exon “1”; |
| chr16 | transcript | 87174036 | 87178840 | + | gene_id “LGFS_NG31”; transcript_id |
| | | | | | “LGFS_NG31.1”; RPKM “8.7782”; cov |
| | | | | | “113.3456”; |
| chr16 | exon | 87174036 | 87178840 | + | gene_id “LGFS_NG31”; transcript_id |
| | | | | | “LGFS_NG31.1”; exon “1”; |
| chr17 | transcript | 78724631 | 78728879 | − | gene_id “LGFS_NG32”; transcript_id |
| | | | | | “LGFS_NG32.1”; RPKM “4.9448”; cov |
| | | | | | “63.8475”; |
| chr17 | exon | 78724631 | 78728324 | − | gene_id “LGFS_NG32”; transcript_id |
| | | | | | “LGFS_NG32.1”; exon “1”; |
| chr17 | exon | 78728674 | 78728879 | − | gene_id “LGFS_NG32”; transcript_id |
| | | | | | “LGFS_NG32.1”; exon “2”; |
| chr17 | transcript | 78724631 | 78728879 | − | gene_id “LGFS_NG32”; transcript_id |
| | | | | | “LGFS_NG32.2”; RPKM “22.3830”; cov |
| | | | | | “289.0130”; |
| chr17 | exon | 78724631 | 78728879 | − | gene_id “LGFS_NG32”; transcript_id |
| | | | | | “LGFS_NG32.2”; exon “1”; |
| chr18 | transcript | 6564865 | 6647080 | − | gene_id “LGFS_NG33”; transcript_id |
| | | | | | “LGFS_NG33.1”; RPKM “0.9888”; cov |
| | | | | | “12.7681”; |
| chr18 | exon | 6564865 | 6569057 | − | gene_id “LGFS_NG33”; transcript_id |
| | | | | | “LGFS_NG33.1”; exon “1”; |
| chr18 | exon | 6646484 | 6647080 | − | gene_id “LGFS_NG33”; transcript_id |
| | | | | | “LGFS_NG33.1”; exon “2”; |
| chr18 | transcript | 76568440 | 76569310 | − | gene_id “LGFS_NG34”; transcript_id |
| | | | | | “LGFS_NG34.1”; RPKM “3.6479”; cov |
| | | | | | “47.1021”; |
| chr18 | exon | 76568440 | 76569310 | − | gene_id “LGFS_NG34”; transcript_id |
| | | | | | “LGFS_NG34.1”; exon “1”; |
| chr18 | transcript | 76569320 | 76569806 | − | gene_id “LGFS_NG35”; transcript_id |
| | | | | | “LGFS_NG35.1”; RPKM “4.1581”; cov |
| | | | | | “53.6899”; |
| chr18 | exon | 76569320 | 76569806 | − | gene_id “LGFS_NG35”; transcript_id |
| | | | | | “LGFS_NG35.1”; exon “1”; |
| chr18 | transcript | 76570193 | 76574891 | − | gene_id “LGFS_NG36”; transcript_id |
| | | | | | “LGFS_NG36.1”; RPKM “3.3934”; cov |
| | | | | | “43.8161”; |
| chr18 | exon | 76570193 | 76574226 | − | gene_id “LGFS_NG36”; transcript_id |
| | | | | | “LGFS_NG36.1”; exon “1”; |
| chr18 | exon | 76574412 | 76574891 | − | gene_id “LGFS_NG36”; transcript_id |
| | | | | | “LGFS_NG36.1”; exon “2”; |
| chr19 | transcript | 21880472 | 21880758 | − | gene_id “LGFS_NG37”; transcript_id |
| | | | | | “LGFS_NG37.1”; RPKM “4.5531”; cov |
| | | | | | “58.7909”; |
| chr19 | exon | 21880472 | 21880758 | − | gene_id “LGFS_NG37”; transcript_id |
| | | | | | “LGFS_NG37.1”; exon “1”; |
| chr19 | transcript | 36062967 | 36078361 | − | gene_id “LGFS_NG38”; transcript_id |
| | | | | | “LGFS_NG38.1”; RPKM “0.9995”; cov |
| | | | | | “12.9055”; |
| chr19 | exon | 36062967 | 36063433 | − | gene_id “LGFS_NG38”; transcript_id |
| | | | | | “LGFS_NG38.1”; exon “1”; |
| chr19 | exon | 36065131 | 36065421 | − | gene_id “LGFS_NG38”; transcript_id |
| | | | | | “LGFS_NG38.1”; exon “2”; |
| chr19 | exon | 36078251 | 36078361 | − | gene_id “LGFS_NG38”; transcript_id |
| | | | | | “LGFS_NG38.1”; exon “3”; |
| chr19 | transcript | 36062967 | 36066499 | − | gene_id “LGFS_NG38”; transcript_id |
| | | | | | “LGFS_NG38.2”; RPKM “1.5987”; cov |
| | | | | | “20.6433”; |
| chr19 | exon | 36062967 | 36066499 | − | gene_id “LGFS_NG38”; transcript_id |
| | | | | | “LGFS_NG38.2”; exon “1”; |
| chr19 | transcript | 36062967 | 36078361 | − | gene_id “LGFS_NG38”; transcript_id |
| | | | | | “LGFS_NG38.3”; RPKM “1.6121”; cov |
| | | | | | “20.8160”; |
| chr19 | exon | 36062967 | 36065421 | − | gene_id “LGFS_NG38”; transcript_id |
| | | | | | “LGFS_NG38.3”; exon “1”; |
| chr19 | exon | 36078251 | 36078361 | − | gene_id “LGFS_NG38”; transcript_id |
| | | | | | “LGFS_NG38.3”; exon “2”; |
| chr2 | transcript | 918918 | 927590 | − | gene_id “LGFS_NG1”; transcript_id |
| | | | | | “LGFS_NG1.2”; RPKM “0.9455”; cov |
| | | | | | “12.2087”; |
| chr2 | exon | 918918 | 923733 | − | gene_id “LGFS_NG1”; transcript_id |
| | | | | | “LGFS_NG1.2”; exon “1”; |
| chr2 | exon | 924136 | 927590 | − | gene_id “LGFS_NG1”; transcript_id |
| | | | | | “LGFS_NG1.2”; exon “2”; |
| chr2 | transcript | 927932 | 929843 | − | gene_id “LGFS_NG1”; transcript_id |
| | | | | | “LGFS_NG1.1”; RPKM “0.8706”; cov |
| | | | | | “11.2410”; |
| chr2 | exon | 927932 | 928406 | − | gene_id “LGFS_NG1”; transcript_id |
| | | | | | “LGFS_NG1.1”; exon “1”; |
| chr2 | exon | 929520 | 929843 | − | gene_id “LGFS_NG1”; transcript_id |
| | | | | | “LGFS_NG1.1”; exon “2”; |
| chr2 | transcript | 52282569 | 52687122 | − | gene_id “LGFS_NG2”; transcript_id |
| | | | | | “LGFS_NG2.1”; RPKM “1.1419”; cov |
| | | | | | “14.7438”; |
| chr2 | exon | 52282569 | 52288567 | − | gene_id “LGFS_NG2”; transcript_id |
| | | | | | “LGFS_NG2.1”; exon “1”; |
| chr2 | exon | 52306971 | 52307069 | − | gene_id “LGFS_NG2”; transcript_id |
| | | | | | “LGFS_NG2.1”; exon “2”; |
| chr2 | exon | 52415416 | 52415490 | − | gene_id “LGFS_NG2”; transcript_id |
| | | | | | “LGFS_NG2.1”; exon “3”; |
| chr2 | exon | 52496887 | 52497096 | − | gene_id “LGFS_NG2”; transcript_id |
| | | | | | “LGFS_NG2.1”; exon “4”; |
| chr2 | exon | 52686843 | 52687122 | − | gene_id “LGFS_NG2”; transcript_id |
| | | | | | “LGFS_NG2.1”; exon “5”; |
| chr2 | transcript | 236014679 | 236046232 | + | gene_id “LGFS_NG3”; transcript_id |
| | | | | | “LGFS_NG3.1”; RPKM “2.5524”; cov |
| | | | | | “32.9572”; |
| chr2 | exon | 236014679 | 236014921 | + | gene_id “LGFS_NG3”; transcript_id |
| | | | | | “LGFS_NG3.1”; exon “1”; |
| chr2 | exon | 236043314 | 236046232 | + | gene_id “LGFS_NG3”; transcript_id |
| | | | | | “LGFS_NG3.1”; exon “2”; |
| chr20 | transcript | 16225622 | 16238481 | − | gene_id “LGFS_NG39”; transcript_id |
| | | | | | “LGFS_NG39.1”; RPKM “1.4572”; cov |
| | | | | | “18.8159”; |
| chr20 | exon | 16225622 | 16227234 | − | gene_id “LGFS_NG39”; transcript_id |
| | | | | | “LGFS_NG39.1”; exon “1”; |
| chr20 | exon | 16237581 | 16238481 | − | gene_id “LGFS_NG39”; transcript_id |
| | | | | | “LGFS_NG39.1”; exon “2”; |
| chr20 | transcript | 16225622 | 16238481 | − | gene_id “LGFS_NG39”; transcript_id |
| | | | | | “LGFS_NG39.2”; RPKM “4.5460”; cov |
| | | | | | “58.6994”; |
| chr20 | exon | 16225622 | 16229187 | − | gene_id “LGFS_NG39”; transcript_id |
| | | | | | “LGFS_NG39.2”; exon “1”; |
| chr20 | exon | 16237581 | 16238481 | − | gene_id “LGFS_NG39”; transcript_id |
| | | | | | “LGFS_NG39.2”; exon “2”; |
| chr20 | transcript | 16225622 | 16238481 | − | gene_id “LGFS_NG39”; transcript_id |
| | | | | | “LGFS_NG39.3”; RPKM “1.6825”; cov |
| | | | | | “21.7252”; |
| chr20 | exon | 16225622 | 16229187 | − | gene_id “LGFS_NG39”; transcript_id |
| | | | | | “LGFS_NG39.3”; exon “1”; |
| chr20 | exon | 16235951 | 16236245 | − | gene_id “LGFS_NG39”; transcript_id |
| | | | | | “LGFS_NG39.3”; exon “2”; |
| chr20 | exon | 16237581 | 16238481 | − | gene_id “LGFS_NG39”; transcript_id |
| | | | | | “LGFS_NG39.3”; exon “3”; |
| chr20 | transcript | 16225622 | 16238481 | − | gene_id “LGFS_NG39”; transcript_id |
| | | | | | “LGFS_NG39.4”; RPKM “6.2200”; cov |
| | | | | | “80.3137”; |
| chr20 | exon | 16225622 | 16238481 | − | gene_id “LGFS_NG39”; transcript_id |
| | | | | | “LGFS_NG39.4”; exon “1”; |
| chr20 | transcript | 60948873 | 60954213 | − | gene_id “LGFS_NG40”; transcript_id |
| | | | | | “LGFS_NG40.1”; RPKM “2.5232”; cov |
| | | | | | “32.5794”; |
| chr20 | exon | 60948873 | 60954213 | − | gene_id “LGFS_NG40”; transcript_id |
| | | | | | “LGFS_NG40.1”; exon “1”; |
| chr21 | transcript | 45613809 | 45617644 | − | gene_id “LGFS_NG41”; transcript_id |
| | | | | | “LGFS_NG41.1”; RPKM “3.9217”; cov |
| | | | | | “50.6375”; |
| chr21 | exon | 45613809 | 45617644 | − | gene_id “LGFS_NG41”; transcript_id |
| | | | | | “LGFS_NG41.1”; exon “1”; |
| chr21 | transcript | 46436885 | 46438642 | − | gene_id “LGFS_NG42”; transcript_id |
| | | | | | “LGFS_NG42.1”; RPKM “7.6262”; cov |
| | | | | | “98.4709”; |
| chr21 | exon | 46436885 | 46438642 | − | gene_id “LGFS_NG42”; transcript_id |
| | | | | | “LGFS_NG42.1”; exon “1”; |
| chr21 | transcript | 46439040 | 46440703 | − | gene_id “LGFS_NG43”; transcript_id |
| | | | | | “LGFS_NG43.1”; RPKM “0.8014”; cov |
| | | | | | “10.3478”; |
| chr21 | exon | 46439040 | 46439772 | − | gene_id “LGFS_NG43”; transcript_id |
| | | | | | “LGFS_NG43.1”; exon “1”; |
| chr21 | exon | 46439865 | 46440703 | − | gene_id “LGFS_NG43”; transcript_id |
| | | | | | “LGFS_NG43.1”; exon “2”; |
| chr21 | transcript | 46439040 | 46440703 | − | gene_id “LGFS_NG43”; transcript_id |
| | | | | | “LGFS_NG43.2”; RPKM “1.0897”; cov |
| | | | | | “14.0706”; |
| chr21 | exon | 46439040 | 46439459 | − | gene_id “LGFS_NG43”; transcript_id |
| | | | | | “LGFS_NG43.2”; exon “1”; |
| chr21 | exon | 46439627 | 46440703 | − | gene_id “LGFS_NG43”; transcript_id |
| | | | | | “LGFS_NG43.2”; exon “2”; |
| chr21 | transcript | 46439040 | 46440703 | − | gene_id “LGFS_NG43”; transcript_id |
| | | | | | “LGFS_NG43.3”; RPKM “1.6567”; cov |
| | | | | | “21.3913”; |
| chr21 | exon | 46439040 | 46440703 | − | gene_id “LGFS_NG43”; transcript_id |
| | | | | | “LGFS_NG43.3”; exon “1”; |
| chr3 | transcript | 32856881 | 32857230 | + | gene_id “LGFS_NG4”; transcript_id |
| | | | | | “LGFS_NG4.1”; RPKM “2.1430”; cov |
| | | | | | “27.6714”; |
| chr3 | exon | 32856881 | 32857230 | + | gene_id “LGFS_NG4”; transcript_id |
| | | | | | “LGFS_NG4.1”; exon “1”; |
| chr3 | transcript | 77830044 | 77830414 | + | gene_id “LGFS_NG5”; transcript_id |
| | | | | | “LGFS_NG5.1”; RPKM “5.5118”; cov |
| | | | | | “71.1698”; |
| chr3 | exon | 77830044 | 77830414 | + | gene_id “LGFS_NG5”; transcript_id |
| | | | | | “LGFS_NG5.1”; exon “1”; |
| chr3 | transcript | 193991118 | 193993494 | + | gene_id “LGFS_NG6”; transcript_id |
| | | | | | “LGFS_NG6.1”; RPKM “3.4515”; cov |
| | | | | | “44.5670”; |
| chr3 | exon | 193991118 | 193993494 | + | gene_id “LGFS_NG6”; transcript_id |
| | | | | | “LGFS_NG6.1”; exon “1”; |
| chr3 | transcript | 193993562 | 193994357 | + | gene_id “LGFS_NG7”; transcript_id |
| | | | | | “LGFS_NG7.1”; RPKM “1.8492”; cov |
| | | | | | “23.8777”; |
| chr3 | exon | 193993562 | 193994357 | + | gene_id “LGFS_NG7”; transcript_id |
| | | | | | “LGFS_NG7.1”; exon “1”; |
| chr3 | transcript | 195546095 | 195546686 | + | gene_id “LGFS_NG8”; transcript_id |
| | | | | | “LGFS_NG8.1”; RPKM “2.5985”; cov |
| | | | | | “33.5524”; |
| chr3 | exon | 195546095 | 195546686 | + | gene_id “LGFS_NG8”; transcript_id |
| | | | | | “LGFS_NG8.1”; exon “1”; |
| chr4 | transcript | 1595164 | 1597919 | − | gene_id “LGFS_NG9”; transcript_id |
| | | | | | “LGFS_NG9.1”; RPKM “2.5235”; cov |
| | | | | | “32.5843”; |
| chr4 | exon | 1595164 | 1597919 | − | gene_id “LGFS_NG9”; transcript_id |
| | | | | | “LGFS_NG9.1”; exon “1”; |
| chr4 | transcript | 1563636 | 1565411 | − | gene_id “LGFS_NG10”; transcript_id |
| | | | | | “LGFS_NG10.1”; RPKM “2.1116”; cov |
| | | | | | “27.2658”; |
| chr4 | exon | 1563636 | 1565411 | − | gene_id “LGFS_NG10”; transcript_id |
| | | | | | “LGFS_NG10.1”; exon “1”; |
| chr4 | transcript | 1569202 | 1570351 | − | gene_id “LGFS_NG11”; transcript_id |
| | | | | | “LGFS_NG11.1”; RPKM “1.7834”; cov |
| | | | | | “23.0278”; |
| chr4 | exon | 1569202 | 1570351 | − | gene_id “LGFS_NG11”; transcript_id |
| | | | | | “LGFS_NG11.1”; exon “1”; |
| chr4 | transcript | 161467437 | 161747178 | − | gene_id “LGFS_NG12”; transcript_id |
| | | | | | “LGFS_NG12.1”; RPKM “2.0796”; cov |
| | | | | | “26.8517”; |
| chr4 | exon | 161467437 | 161475312 | − | gene_id “LGFS_NG12”; transcript_id |
| | | | | | “LGFS_NG12.1”; exon “1”; |
| chr4 | exon | 161477177 | 161477250 | − | gene_id “LGFS_NG12”; transcript_id |
| | | | | | “LGFS_NG12.1”; exon “2”; |
| chr4 | exon | 161480550 | 161480692 | − | gene_id “LGFS_NG12”; transcript_id |
| | | | | | “LGFS_NG12.1”; exon “3”; |
| chr4 | exon | 161590109 | 161590220 | − | gene_id “LGFS_NG12”; transcript_id |
| | | | | | “LGFS_NG12.1”; exon “4”; |
| chr4 | exon | 161746934 | 161747178 | − | gene_id “LGFS_NG12”; transcript_id |
| | | | | | “LGFS_NG12.1”; exon “5”; |
| chr4 | transcript | 161467437 | 161747178 | − | gene_id “LGFS_NG12”; transcript_id |
| | | | | | “LGFS_NG12.2”; RPKM “1.1556”; cov |
| | | | | | “14.9208”; |
| chr4 | exon | 161467437 | 161477250 | − | gene_id “LGFS_NG12”; transcript_id |
| | | | | | “LGFS_NG12.2”; exon “1”; |
| chr4 | exon | 161480550 | 161480692 | − | gene_id “LGFS_NG12”; transcript_id |
| | | | | | “LGFS_NG12.2”; exon “2”; |
| chr4 | exon | 161590109 | 161590220 | − | gene_id “LGFS_NG12”; transcript_id |
| | | | | | “LGFS_NG12.2”; exon “3”; |
| chr4 | exon | 161746934 | 161747178 | − | gene_id “LGFS_NG12”; transcript_id |
| | | | | | “LGFS_NG12.2”; exon “4”; |
| chr5 | transcript | 1748243 | 1752448 | − | gene_id “LGFS_NG13”; transcript_id |
| | | | | | “LGFS_NG13.1”; RPKM “1.4604”; cov |
| | | | | | “18.8576”; |
| chr5 | exon | 1748243 | 1748621 | − | gene_id “LGFS_NG13”; transcript_id |
| | | | | | “LGFS_NG13.1”; exon “1”; |
| chr5 | exon | 1751766 | 1751909 | − | gene_id “LGFS_NG13”; transcript_id |
| | | | | | “LGFS_NG13.1”; exon “2”; |
| chr5 | exon | 1752145 | 1752448 | − | gene_id “LGFS_NG13”; transcript_id |
| | | | | | “LGFS_NG13.1”; exon “3”; |
| chr5 | transcript | 1748243 | 1752448 | − | gene_id “LGFS_NG13”; transcript_id |
| | | | | | “LGFS_NG13.2”; RPKM “1.5637”; cov |
| | | | | | “20.1905”; |
| chr5 | exon | 1748243 | 1748621 | − | gene_id “LGFS_NG13”; transcript_id |
| | | | | | “LGFS_NG13.2”; exon “1”; |
| chr5 | exon | 1751766 | 1752448 | − | gene_id “LGFS_NG13”; transcript_id |
| | | | | | “LGFS_NG13.2”; exon “2”; |
| chr6 | transcript | 792810 | 797245 | + | gene_id “LGFS_NG14”; transcript_id |
| | | | | | “LGFS_NG14.1”; RPKM “3.4413”; cov |
| | | | | | “44.4351”; |
| chr6 | exon | 792810 | 797245 | + | gene_id “LGFS_NG14”; transcript_id |
| | | | | | “LGFS_NG14.1”; exon “1”; |
| chr7 | transcript | 149670553 | 149678196 | − | gene_id “LGFS_NG15”; transcript_id |
| | | | | | “LGFS_NG15.1”; RPKM “3.5145”; cov |
| | | | | | “45.3805”; |
| chr7 | exon | 149670553 | 149678196 | − | gene_id “LGFS_NG15”; transcript_id |
| | | | | | “LGFS_NG15.1”; exon “1”; |
| chr7 | transcript | 152246961 | 152263275 | − | gene_id “LGFS_NG16”; transcript_id |
| | | | | | “LGFS_NG16.1”; RPKM “1.3525”; cov |
| | | | | | “17.4633”; |
| chr7 | exon | 152246961 | 152247702 | − | gene_id “LGFS_NG16”; transcript_id |
| | | | | | “LGFS_NG16.1”; exon “1”; |
| chr7 | exon | 152247887 | 152247962 | − | gene_id “LGFS_NG16”; transcript_id |
| | | | | | “LGFS_NG16.1”; exon “2”; |
| chr7 | exon | 152256248 | 152256391 | − | gene_id “LGFS_NG16”; transcript_id |
| | | | | | “LGFS_NG16.1”; exon “3”; |
| chr7 | exon | 152262674 | 152263275 | − | gene_id “LGFS_NG16”; transcript_id |
| | | | | | “LGFS_NG16.1”; exon “4”; |
| chr7 | transcript | 152246961 | 152263275 | − | gene_id “LGFS_NG16”; transcript_id |
| | | | | | “LGFS_NG16.2”; RPKM “5.1906”; cov |
| | | | | | “67.0218”; |
| chr7 | exon | 152246961 | 152247702 | − | gene_id “LGFS_NG16”; transcript_id |
| | | | | | “LGFS_NG16.2”; exon “1”; |
| chr7 | exon | 152247887 | 152247962 | − | gene_id “LGFS_NG16”; transcript_id |
| | | | | | “LGFS_NG16.2”; exon “2”; |
| chr7 | exon | 152262674 | 152263275 | − | gene_id “LGFS_NG16”; transcript_id |
| | | | | | “LGFS_NG16.2”; exon “3”; |
| chr7 | transcript | 152246961 | 152263275 | − | gene_id “LGFS_NG16”; transcript_id |
| | | | | | “LGFS_NG16.3”; RPKM “1.6603”; cov |
| | | | | | “21.4379”; |
| chr7 | exon | 152246961 | 152247962 | − | gene_id “LGFS_NG16”; transcript_id |
| | | | | | “LGFS_NG16.3”; exon “1”; |
| chr7 | exon | 152262674 | 152263275 | − | gene_id “LGFS_NG16”; transcript_id |
| | | | | | “LGFS_NG16.3”; exon “2”; |
| chr7 | transcript | 152246961 | 152263275 | − | gene_id “LGFS_NG16”; transcript_id |
| | | | | | “LGFS_NG16.4”; RPKM “2.7724”; cov |
| | | | | | “35.7975”; |
| chr7 | exon | 152246961 | 152247649 | − | gene_id “LGFS_NG16”; transcript_id |
| | | | | | “LGFS_NG16.4”; exon “1”; |
| chr7 | exon | 152247887 | 152247962 | − | gene_id “LGFS_NG16”; transcript_id |
| | | | | | “LGFS_NG16.4”; exon “2”; |
| chr7 | exon | 152262674 | 152263275 | − | gene_id “LGFS_NG16”; transcript_id |
| | | | | | “LGFS_NG16.4”; exon “3”; |
| chr7 | transcript | 157652510 | 157659496 | − | gene_id “LGFS_NG17”; transcript_id |
| | | | | | “LGFS_NG17.1”; RPKM “4.4823”; cov |
| | | | | | “57.8762”; |
| chr7 | exon | 157652510 | 157659496 | − | gene_id “LGFS_NG17”; transcript_id |
| | | | | | “LGFS_NG17.1”; exon “1”; |
| chr7 | transcript | 157660018 | 157664994 | − | gene_id “LGFS_NG17”; transcript_id |
| | | | | | “LGFS_NG17.2”; RPKM “3.1830”; cov |
| | | | | | “41.0996”; |
| chr7 | exon | 157660018 | 157664994 | − | gene_id “LGFS_NG17”; transcript_id |
| | | | | | “LGFS_NG17.2”; exon “1”; |
| chr8 | transcript | 1258226 | 1261162 | + | gene_id “LGFS_NG18”; transcript_id |
| | | | | | “LGFS_NG18.1”; RPKM “1.6657”; cov |
| | | | | | “21.5073”; |
| chr8 | exon | 1258226 | 1261162 | + | gene_id “LGFS_NG18”; transcript_id |
| | | | | | “LGFS_NG18.1”; exon “1”; |
| chr9 | transcript | 138444994 | 138446508 | + | gene_id “LGFS_NG19”; transcript_id |
| | | | | | “LGFS_NG19.1”; RPKM “1.3338”; cov |
| | | | | | “17.2217”; |
| chr9 | exon | 138444994 | 138445373 | + | gene_id “LGFS_NG19”; transcript_id |
| | | | | | “LGFS_NG19.1”; exon “1”; |
| chr9 | exon | 138445507 | 138446508 | + | gene_id “LGFS_NG19”; transcript_id |
| | | | | | “LGFS_NG19.1”; exon “2”; |
| chrX | transcript | 569266 | 579257 | − | gene_id “LGFS_NG44”; transcript_id |
| | | | | | “LGFS_NG44.1”; RPKM “2.2858”; cov |
| | | | | | “29.5148”; |
| chrX | exon | 569266 | 569381 | − | gene_id “LGFS_NG44”; transcript_id |
| | | | | | “LGFS_NG44.1”; exon “1”; |
| chrX | exon | 574611 | 574729 | − | gene_id “LGFS_NG44”; transcript_id |
| | | | | | “LGFS_NG44.1”; exon “2”; |
| chrX | exon | 574840 | 575188 | − | gene_id “LGFS_NG44”; transcript_id |
| | | | | | “LGFS_NG44.1”; exon “3”; |
| chrX | exon | 578262 | 579257 | − | gene_id “LGFS_NG44”; transcript_id |
| | | | | | “LGFS_NG44.1”; exon “4”; |
| chrX | transcript | 569266 | 585408 | − | gene_id “LGFS_NG44”; transcript_id |
| | | | | | “LGFS_NG44.2”; RPKM “1.9361”; cov |
| | | | | | “24.9988”; |
| chrX | exon | 569266 | 569381 | − | gene_id “LGFS_NG44”; transcript_id |
| | | | | | “LGFS_NG44.2”; exon “1”; |
| chrX | exon | 574611 | 574729 | − | gene_id “LGFS_NG44”; transcript_id |
| | | | | | “LGFS_NG44.2”; exon “2”; |
| chrX | exon | 574840 | 575188 | − | gene_id “LGFS_NG44”; transcript_id |
| | | | | | “LGFS_NG44.2”; exon “3”; |
| chrX | exon | 578262 | 578401 | − | gene_id “LGFS_NG44”; transcript_id |
| | | | | | “LGFS_NG44.2”; exon “4”; |
| chrX | exon | 580400 | 580520 | − | gene_id “LGFS_NG44”; transcript_id |
| | | | | | “LGFS_NG44.2”; exon “5”; |
| chrX | exon | 580954 | 581495 | − | gene_id “LGFS_NG44”; transcript_id |
| | | | | | “LGFS_NG44.2”; exon “6”; |
| chrX | exon | 584233 | 585408 | − | gene_id “LGFS_NG44”; transcript_id |
| | | | | | “LGFS_NG44.2”; exon “7”; |
| chrX | transcript | 572821 | 577946 | − | gene_id “LGFS_NG44”; transcript_id |
| | | | | | “LGFS_NG44.3”; RPKM “1.4510”; cov |
| | | | | | “18.7352”; |
| chrX | exon | 572821 | 574261 | − | gene_id “LGFS_NG44”; transcript_id |
| | | | | | “LGFS_NG44.3”; exon “1”; |
| chrX | exon | 574521 | 577946 | − | gene_id “LGFS_NG44”; transcript_id |
| | | | | | “LGFS_NG44.3”; exon “2”; |
| chrX | transcript | 572821 | 577946 | − | gene_id “LGFS_NG44”; transcript_id |
| | | | | | “LGFS_NG44.4”; RPKM “2.2981”; cov |
| | | | | | “29.6733”; |
| chrX | exon | 572821 | 577946 | − | gene_id “LGFS_NG44”; transcript_id |
| | | | | | “LGFS_NG44.4”; exon “1”; |
| chr1 | exon | 34784807 | 34785347 | − | gene_id “MCS_NG1”; transcript_id |
| | | | | | “MCS_NG1.1”; exon “1”; |
| chr1 | exon | 34790517 | 34790702 | − | gene_id “MCS_NG1”; transcript_id |
| | | | | | “MCS_NG1.1”; exon “2”; |
| chr1 | exon | 34796309 | 34796495 | − | gene_id “MCS_NG1”; transcript_id |
| | | | | | “MCS_NG1.1”; exon “3”; |
| chr1 | transcript | 157180582 | 157185792 | − | gene_id “MCS_NG2”; transcript_id |
| | | | | | “MCS_NG2.1”; RPKM “1.4970”; cov |
| | | | | | “31.3598”; |
| chr1 | exon | 157180582 | 157185792 | − | gene_id “MCS_NG2”; transcript_id |
| | | | | | “MCS_NG2.1”; exon “1”; |
| chr1 | transcript | 157191437 | 157249328 | − | gene_id “MCS_NG3”; transcript_id |
| | | | | | “MCS_NG3.2”; RPKM “1.2674”; cov |
| | | | | | “26.5487”; |
| chr1 | exon | 157191437 | 157195390 | − | gene_id “MCS_NG3”; transcript_id |
| | | | | | “MCS_NG3.2”; exon “1”; |
| chr1 | exon | 157195973 | 157196072 | − | gene_id “MCS_NG3”; transcript_id |
| | | | | | “MCS_NG3.2”; exon “2”; |
| chr1 | exon | 157222196 | 157222493 | − | gene_id “MCS_NG3”; transcript_id |
| | | | | | “MCS_NG3.2”; exon “3”; |
| chr1 | exon | 157248992 | 157249328 | − | gene_id “MCS_NG3”; transcript_id |
| | | | | | “MCS_NG3.2”; exon “4”; |
| chr1 | transcript | 157191437 | 157249328 | − | gene_id “MCS_NG3”; transcript_id |
| | | | | | “MCS_NG3.3”; RPKM “1.7347”; cov |
| | | | | | “36.3377”; |
| chr1 | exon | 157191437 | 157196072 | − | gene_id “MCS_NG3”; transcript_id |
| | | | | | “MCS_NG3.3”; exon “1”; |
| chr1 | exon | 157222196 | 157222493 | − | gene_id “MCS_NG3”; transcript_id |
| | | | | | “MCS_NG3.3”; exon “2”; |
| chr1 | exon | 157248992 | 157249328 | − | gene_id “MCS_NG3”; transcript_id |
| | | | | | “MCS_NG3.3”; exon “3”; |
| chr1 | transcript | 157191437 | 157249328 | − | gene_id “MCS_NG3”; transcript_id |
| | | | | | “MCS_NG3.4”; RPKM “0.9720”; cov |
| | | | | | “20.3623”; |
| chr1 | exon | 157191437 | 157199369 | − | gene_id “MCS_NG3”; transcript_id |
| | | | | | “MCS_NG3.4”; exon “1”; |
| chr1 | exon | 157222196 | 157222493 | − | gene_id “MCS_NG3”; transcript_id |
| | | | | | “MCS_NG3.4”; exon “2”; |
| chr1 | exon | 157248992 | 157249328 | − | gene_id “MCS_NG3”; transcript_id |
| | | | | | “MCS_NG3.4”; exon “3”; |
| chr1 | transcript | 157219685 | 157249328 | − | gene_id “MCS_NG3”; transcript_id |
| | | | | | “MCS_NG3.1”; RPKM “1.4025”; cov |
| | | | | | “29.3786”; |
| chr1 | exon | 157219685 | 157222493 | − | gene_id “MCS_NG3”; transcript_id |
| | | | | | “MCS_NG3.1”; exon “1”; |
| chr1 | exon | 157248992 | 157249328 | − | gene_id “MCS_NG3”; transcript_id |
| | | | | | “MCS_NG3.1”; exon “2”; |
| chr11 | transcript | 16040881 | 16069090 | + | gene_id “MCS_NG8”; transcript_id |
| | | | | | “MCS_NG8.1”; RPKM “0.7233”; cov |
| | | | | | “15.1523”; |
| chr11 | exon | 16040881 | 16041006 | + | gene_id “MCS_NG8”; transcript_id |
| | | | | | “MCS_NG8.1”; exon “1”; |
| chr11 | exon | 16063432 | 16063634 | + | gene_id “MCS_NG8”; transcript_id |
| | | | | | “MCS_NG8.1”; exon “2”; |
| chr11 | exon | 16067794 | 16067836 | + | gene_id “MCS_NG8”; transcript_id |
| | | | | | “MCS_NG8.1”; exon “3”; |
| chr11 | exon | 16068784 | 16069090 | + | gene_id “MCS_NG8”; transcript_id |
| | | | | | “MCS_NG8.1”; exon “4”; |
| chr11 | transcript | 16040881 | 16069090 | + | gene_id “MCS_NG8”; transcript_id |
| | | | | | “MCS_NG8.2”; RPKM “0.9597”; cov |
| | | | | | “20.1041”; |
| chr11 | exon | 16040881 | 16041006 | + | gene_id “MCS_NG8”; transcript_id |
| | | | | | “MCS_NG8.2”; exon “1”; |
| chr11 | exon | 16063432 | 16063634 | + | gene_id “MCS_NG8”; transcript_id |
| | | | | | “MCS_NG8.2”; exon “2”; |
| chr11 | exon | 16068784 | 16069090 | + | gene_id “MCS_NG8”; transcript_id |
| | | | | | “MCS_NG8.2”; exon “3”; |
| chr12 | transcript | 54124477 | 54128954 | − | gene_id “MCS_NG9”; transcript_id |
| | | | | | “MCS_NG9.1”; RPKM “1.1933”; cov |
| | | | | | “24.9969”; |
| chr12 | exon | 54124477 | 54128954 | − | gene_id “MCS_NG9”; transcript_id |
| | | | | | “MCS_NG9.1”; exon “1”; |
| chr16 | transcript | 70005696 | 70006778 | + | gene_id “MCS_NG10”; transcript_id |
| | | | | | “MCS_NG10.1”; RPKM “2.7994”; cov |
| | | | | | “58.6408”; |
| chr16 | exon | 70005696 | 70006778 | + | gene_id “MCS_NG10”; transcript_id |
| | | | | | “MCS_NG10.1”; exon “1”; |
| chr16 | transcript | 85880130 | 85881773 | + | gene_id “MCS_NG11”; transcript_id |
| | | | | | “MCS_NG11.1”; RPKM “0.5942”; cov |
| | | | | | “12.4473”; |
| chr16 | exon | 85880130 | 85880230 | + | gene_id “MCS_NG11”; transcript_id |
| | | | | | “MCS_NG11.1”; exon “1”; |
| chr16 | exon | 85881283 | 85881773 | + | gene_id “MCS_NG11”; transcript_id |
| | | | | | “MCS_NG11.1”; exon “2”; |
| chr3 | transcript | 193981277 | 193982729 | − | gene_id “MCS_NG4”; transcript_id |
| | | | | | “MCS_NG4.1”; RPKM “1.0808”; cov |
| | | | | | “22.6401”; |
| chr3 | exon | 193981277 | 193982729 | − | gene_id “MCS_NG4”; transcript_id |
| | | | | | “MCS_NG4.1”; exon “1”; |
| chr4 | transcript | 145120948 | 145132678 | − | gene_id “MCS_NG5”; transcript_id |
| | | | | | “MCS_NG5.1”; RPKM “0.9690”; cov |
| | | | | | “20.2988”; |
| chr4 | exon | 145120948 | 145123131 | − | gene_id “MCS_NG5”; transcript_id |
| | | | | | “MCS_NG5.1”; exon “1”; |
| chr4 | exon | 145131926 | 145132678 | − | gene_id “MCS_NG5”; transcript_id |
| | | | | | “MCS_NG5.1”; exon “2”; |
| chr4 | transcript | 145120948 | 145132678 | − | gene_id “MCS_NG5”; transcript_id |
| | | | | | “MCS_NG5.2”; RPKM “0.6430”; cov |
| | | | | | “13.4695”; |
| chr4 | exon | 145120948 | 145123131 | − | gene_id “MCS_NG5”; transcript_id |
| | | | | | “MCS_NG5.2”; exon “1”; |
| chr4 | exon | 145127174 | 145127334 | − | gene_id “MCS_NG5”; transcript_id |
| | | | | | “MCS_NG5.2”; exon “2”; |
| chr4 | exon | 145131926 | 145132678 | − | gene_id “MCS_NG5”; transcript_id |
| | | | | | “MCS_NG5.2”; exon “3”; |
| chr4 | transcript | 145120948 | 145430613 | − | gene_id “MCS_NG5”; transcript_id |
| | | | | | “MCS_NG5.3”; RPKM “1.1879”; cov |
| | | | | | “24.8835”; |
| chr4 | exon | 145120948 | 145123131 | − | gene_id “MCS_NG5”; transcript_id |
| | | | | | “MCS_NG5.3”; exon “1”; |
| chr4 | exon | 145127174 | 145127334 | − | gene_id “MCS_NG5”; transcript_id |
| | | | | | “MCS_NG5.3”; exon “2”; |
| chr4 | exon | 145131926 | 145132166 | − | gene_id “MCS_NG5”; transcript_id |
| | | | | | “MCS_NG5.3”; exon “3”; |
| chr4 | exon | 145137246 | 145137327 | − | gene_id “MCS_NG5”; transcript_id |
| | | | | | “MCS_NG5.3”; exon “4”; |
| chr4 | exon | 145141856 | 145141925 | − | gene_id “MCS_NG5”; transcript_id |
| | | | | | “MCS_NG5.3”; exon “5”; |
| chr4 | exon | 145204248 | 145204339 | − | gene_id “MCS_NG5”; transcript_id |
| | | | | | “MCS_NG5.3”; exon “6”; |
| chr4 | exon | 145337055 | 145337130 | − | gene_id “MCS_NG5”; transcript_id |
| | | | | | “MCS_NG5.3”; exon “7”; |
| chr4 | exon | 145430393 | 145430613 | − | gene_id “MCS_NG5”; transcript_id |
| | | | | | “MCS_NG5.3”; exon “8”; |
| chr4 | transcript | 145120948 | 145430613 | − | gene_id “MCS_NG5”; transcript_id |
| | | | | | “MCS_NG5.4”; RPKM “0.5472”; cov |
| | | | | | “11.4633”; |
| chr4 | exon | 145120948 | 145123131 | − | gene_id “MCS_NG5”; transcript_id |
| | | | | | “MCS_NG5.4”; exon “1”; |
| chr4 | exon | 145127174 | 145127334 | − | gene_id “MCS_NG5”; transcript_id |
| | | | | | “MCS_NG5.4”; exon “2”; |
| chr4 | exon | 145131926 | 145132166 | − | gene_id “MCS_NG5”; transcript_id |
| | | | | | “MCS_NG5.4”; exon “3”; |
| chr4 | exon | 145137246 | 145137327 | − | gene_id “MCS_NG5”; transcript_id |
| | | | | | “MCS_NG5.4”; exon “4”; |
| chr4 | exon | 145141856 | 145141925 | − | gene_id “MCS_NG5”; transcript_id |
| | | | | | “MCS_NG5.4”; exon “5”; |
| chr4 | exon | 145204248 | 145204339 | − | gene_id “MCS_NG5”; transcript_id |
| | | | | | “MCS_NG5.4”; exon “6”; |
| chr4 | exon | 145337055 | 145337130 | − | gene_id “MCS_NG5”; transcript_id |
| | | | | | “MCS_NG5.4”; exon “7”; |
| chr4 | exon | 145420599 | 145420672 | − | gene_id “MCS_NG5”; transcript_id |
| | | | | | “MCS_NG5.4”; exon “8”; |
| chr4 | exon | 145430393 | 145430613 | − | gene_id “MCS_NG5”; transcript_id |
| | | | | | “MCS_NG5.4”; exon “9”; |
| chr4 | transcript | 145120948 | 145430613 | − | gene_id “MCS_NG5”; transcript_id |
| | | | | | “MCS_NG5.5”; RPKM “1.4518”; cov |
| | | | | | “30.4127”; |
| chr4 | exon | 145120948 | 145123131 | − | gene_id “MCS_NG5”; transcript_id |
| | | | | | “MCS_NG5.5”; exon “1”; |
| chr4 | exon | 145131926 | 145132166 | − | gene_id “MCS_NG5”; transcript_id |
| | | | | | “MCS_NG5.5”; exon “2”; |
| chr4 | exon | 145137246 | 145137327 | − | gene_id “MCS_NG5”; transcript_id |
| | | | | | “MCS_NG5.5”; exon “3”; |
| chr4 | exon | 145141856 | 145141925 | − | gene_id “MCS_NG5”; transcript_id |
| | | | | | “MCS_NG5.5”; exon “4”; |
| chr4 | exon | 145204248 | 145204339 | − | gene_id “MCS_NG5”; transcript_id |
| | | | | | “MCS_NG5.5”; exon “5”; |
| chr4 | exon | 145337055 | 145337130 | − | gene_id “MCS_NG5”; transcript_id |
| | | | | | “MCS_NG5.5”; exon “6”; |
| chr4 | exon | 145430393 | 145430613 | − | gene_id “MCS_NG5”; transcript_id |
| | | | | | “MCS_NG5.5”; exon “7”; |
| chr4 | transcript | 145120948 | 145430613 | − | gene_id “MCS_NG5”; transcript_id |
| | | | | | “MCS_NG5.6”; RPKM “0.6165”; cov |
| | | | | | “12.9149”; |
| chr4 | exon | 145120948 | 145123131 | − | gene_id “MCS_NG5”; transcript_id |
| | | | | | “MCS_NG5.6”; exon “1”; |
| chr4 | exon | 145131926 | 145132166 | − | gene_id “MCS_NG5”; transcript_id |
| | | | | | “MCS_NG5.6”; exon “2”; |
| chr4 | exon | 145137246 | 145137327 | − | gene_id “MCS_NG5”; transcript_id |
| | | | | | “MCS_NG5.6”; exon “3”; |
| chr4 | exon | 145141856 | 145141925 | − | gene_id “MCS_NG5”; transcript_id |
| | | | | | “MCS_NG5.6”; exon “4”; |
| chr4 | exon | 145204248 | 145204339 | − | gene_id “MCS_NG5”; transcript_id |
| | | | | | “MCS_NG5.6”; exon “5”; |
| chr4 | exon | 145337055 | 145337130 | − | gene_id “MCS_NG5”; transcript_id |
| | | | | | “MCS_NG5.6”; exon “6”; |
| chr4 | exon | 145420581 | 145420672 | − | gene_id “MCS_NG5”; transcript_id |
| | | | | | “MCS_NG5.6”; exon “7”; |
| chr4 | exon | 145430393 | 145430613 | − | gene_id “MCS_NG5”; transcript_id |
| | | | | | “MCS_NG5.6”; exon “8”; |
| chr4 | transcript | 145120948 | 145430613 | − | gene_id “MCS_NG5”; transcript_id |
| | | | | | “MCS_NG5.7”; RPKM “0.6688”; cov |
| | | | | | “14.0105”; |
| chr4 | exon | 145120948 | 145123131 | − | gene_id “MCS_NG5”; transcript_id |
| | | | | | “MCS_NG5.7”; exon “1”; |
| chr4 | exon | 145131926 | 145132166 | − | gene_id “MCS_NG5”; transcript_id |
| | | | | | “MCS_NG5.7”; exon “2”; |
| chr4 | exon | 145137246 | 145137327 | − | gene_id “MCS_NG5”; transcript_id |
| | | | | | “MCS_NG5.7”; exon “3”; |
| chr4 | exon | 145141856 | 145141925 | − | gene_id “MCS_NG5”; transcript_id |
| | | | | | “MCS_NG5.7”; exon “4”; |
| chr4 | exon | 145204248 | 145204339 | − | gene_id “MCS_NG5”; transcript_id |
| | | | | | “MCS_NG5.7”; exon “5”; |
| chr4 | exon | 145337055 | 145337130 | − | gene_id “MCS_NG5”; transcript_id |
| | | | | | “MCS_NG5.7”; exon “6”; |
| chr4 | exon | 145420599 | 145420672 | − | gene_id “MCS_NG5”; transcript_id |
| | | | | | “MCS_NG5.7”; exon “7”; |
| chr4 | exon | 145430393 | 145430613 | − | gene_id “MCS_NG5”; transcript_id |
| | | | | | “MCS_NG5.7”; exon “8”; |
| chr7 | transcript | 25627460 | 25627927 | − | gene_id “MCS_NG6”; transcript_id |
| | | | | | “MCS_NG6.1”; RPKM “2.8060”; cov |
| | | | | | “58.7799”; |
| chr7 | exon | 25627460 | 25627927 | − | gene_id “MCS_NG6”; transcript_id |
| | | | | | “MCS_NG6.1”; exon “1”; |
| chr9 | transcript | 98278808 | 98280219 | + | gene_id “MCS_NG7”; transcript_id |
| | | | | | “MCS_NG7.1”; RPKM “1.3822”; cov |
| | | | | | “28.9533”; |
| chr9 | exon | 98278808 | 98280219 | + | gene_id “MCS_NG7”; transcript_id |
| | | | | | “MCS_NG7.1”; exon “1”; |
| chr1 | transcript | 81733802 | 81740717 | − | gene_id “Midline_NG1”; transcript_id |
| | | | | | “Midline_NG1.1”; RPKM “3.4068”; cov |
| | | | | | “59.0412”; |
| chr1 | exon | 81733802 | 81740717 | − | gene_id “Midline_NG1”; transcript_id |
| | | | | | “Midline_NG1.1”; exon “1”; |
| chr1 | transcript | 81742914 | 81772404 | − | gene_id “Midline_NG1”; transcript_id |
| | | | | | “Midline_NG1.5”; RPKM “0.9581”; cov |
| | | | | | “16.6047”; |
| chr1 | exon | 81742914 | 81743799 | − | gene_id “Midline_NG1”; transcript_id |
| | | | | | “Midline_NG1.5”; exon “1”; |
| chr1 | exon | 81744759 | 81744956 | − | gene_id “Midline_NG1”; transcript_id |
| | | | | | “Midline_NG1.5”; exon “2”; |
| chr1 | exon | 81764288 | 81764443 | − | gene_id “Midline_NG1”; transcript_id |
| | | | | | “Midline_NG1.5”; exon “3”; |
| chr1 | exon | 81766365 | 81766583 | − | gene_id “Midline_NG1”; transcript_id |
| | | | | | “Midline_NG1.5”; exon “4”; |
| chr1 | exon | 81772176 | 81772404 | − | gene_id “Midline_NG1”; transcript_id |
| | | | | | “Midline_NG1.5”; exon “5”; |
| chr1 | transcript | 81742914 | 81772404 | − | gene_id “Midline_NG1”; transcript_id |
| | | | | | “Midline_NG1.6”; RPKM “0.6033”; cov |
| | | | | | “10.4558”; |
| chr1 | exon | 81742914 | 81743799 | − | gene_id “Midline_NG1”; transcript_id |
| | | | | | “Midline_NG1.6”; exon “1”; |
| chr1 | exon | 81744759 | 81744956 | − | gene_id “Midline_NG1”; transcript_id |
| | | | | | “Midline_NG1.6”; exon “2”; |
| chr1 | exon | 81764288 | 81764443 | − | gene_id “Midline_NG1”; transcript_id |
| | | | | | “Midline_NG1.6”; exon “3”; |
| chr1 | exon | 81765321 | 81765443 | − | gene_id “Midline_NG1”; transcript_id |
| | | | | | “Midline_NG1.6”; exon “4”; |
| chr1 | exon | 81766365 | 81766583 | − | gene_id “Midline_NG1”; transcript_id |
| | | | | | “Midline_NG1.6”; exon “5”; |
| chr1 | exon | 81772176 | 81772404 | − | gene_id “Midline_NG1”; transcript_id |
| | | | | | “Midline_NG1.6”; exon “6”; |
| chr1 | transcript | 81753786 | 81772404 | − | gene_id “Midline_NG1”; transcript_id |
| | | | | | “Midline_NG1.3”; RPKM “0.6470”; cov |
| | | | | | “11.2125”; |
| chr1 | exon | 81753786 | 81764443 | − | gene_id “Midline_NG1”; transcript_id |
| | | | | | “Midline_NG1.3”; exon “1”; |
| chr1 | exon | 81765321 | 81765443 | − | gene_id “Midline_NG1”; transcript_id |
| | | | | | “Midline_NG1.3”; exon “2”; |
| chr1 | exon | 81766089 | 81766583 | − | gene_id “Midline_NG1”; transcript_id |
| | | | | | “Midline_NG1.3”; exon “3”; |
| chr1 | exon | 81772176 | 81772404 | − | gene_id “Midline_NG1”; transcript_id |
| | | | | | “Midline_NG1.3”; exon “4”; |
| chr1 | transcript | 81762332 | 81772404 | − | gene_id “Midline_NG1”; transcript_id |
| | | | | | “Midline_NG1.4”; RPKM “1.2253”; cov |
| | | | | | “21.2346”; |
| chr1 | exon | 81762332 | 81764443 | − | gene_id “Midline_NG1”; transcript_id |
| | | | | | “Midline_NG1.4”; exon “1”; |
| chr1 | exon | 81765321 | 81766583 | − | gene_id “Midline_NG1”; transcript_id |
| | | | | | “Midline_NG1.4”; exon “2”; |
| chr1 | exon | 81772176 | 81772404 | − | gene_id “Midline_NG1”; transcript_id |
| | | | | | “Midline_NG1.4”; exon “3”; |
| chr1 | transcript | 81764518 | 81772404 | − | gene_id “Midline_NG1”; transcript_id |
| | | | | | “Midline_NG1.2”; RPKM “0.7967”; cov |
| | | | | | “13.8066”; |
| chr1 | exon | 81764518 | 81766583 | − | gene_id “Midline_NG1”; transcript_id |
| | | | | | “Midline_NG1.2”; exon “1”; |
| chr1 | exon | 81772176 | 81772404 | − | gene_id “Midline_NG1”; transcript_id |
| | | | | | “Midline_NG1.2”; exon “2”; |
| chr13 | transcript | 86001004 | 86111279 | − | gene_id “Midline_NG26”; transcript_id |
| | | | | | “Midline_NG26.1”; RPKM “1.2608”; cov |
| | | | | | “21.8503”; |
| chr13 | exon | 86001004 | 86003874 | − | gene_id “Midline_NG26”; transcript_id |
| | | | | | “Midline_NG26.1”; exon “1”; |
| chr13 | exon | 86042621 | 86042759 | − | gene_id “Midline_NG26”; transcript_id |
| | | | | | “Midline_NG26.1”; exon “2”; |
| chr13 | exon | 86065671 | 86065859 | − | gene_id “Midline_NG26”; transcript_id |
| | | | | | “Midline_NG26.1”; exon “3”; |
| chr13 | exon | 86105572 | 86105658 | − | gene_id “Midline_NG26”; transcript_id |
| | | | | | “Midline_NG26.1”; exon “4”; |
| chr13 | exon | 86111176 | 86111279 | − | gene_id “Midline_NG26”; transcript_id |
| | | | | | “Midline_NG26.1”; exon “5”; |
| chr13 | transcript | 86001004 | 86111279 | − | gene_id “Midline_NG26”; transcript_id |
| | | | | | “Midline_NG26.2”; RPKM “0.6895”; cov |
| | | | | | “11.9486”; |
| chr13 | exon | 86001004 | 86003874 | − | gene_id “Midline_NG26”; transcript_id |
| | | | | | “Midline_NG26.2”; exon “1”; |
| chr13 | exon | 86042621 | 86042759 | − | gene_id “Midline_NG26”; transcript_id |
| | | | | | “Midline_NG26.2”; exon “2”; |
| chr13 | exon | 86065671 | 86065859 | − | gene_id “Midline_NG26”; transcript_id |
| | | | | | “Midline_NG26.2”; exon “3”; |
| chr13 | exon | 86103939 | 86104013 | − | gene_id “Midline_NG26”; transcript_id |
| | | | | | “Midline_NG26.2”; exon “4”; |
| chr13 | exon | 86105572 | 86105658 | − | gene_id “Midline_NG26”; transcript_id |
| | | | | | “Midline_NG26.2”; exon “5”; |
| chr13 | exon | 86111176 | 86111279 | − | gene_id “Midline_NG26”; transcript_id |
| | | | | | “Midline_NG26.2”; exon “6”; |
| chr13 | transcript | 86240716 | 86357493 | + | gene_id “Midline_NG27”; transcript_id |
| | | | | | “Midline_NG27.1”; RPKM “0.6024”; cov |
| | | | | | “10.4401”; |
| chr13 | exon | 86240716 | 86240895 | + | gene_id “Midline_NG27”; transcript_id |
| | | | | | “Midline_NG27.1”; exon “1”; |
| chr13 | exon | 86250665 | 86250748 | + | gene_id “Midline_NG27”; transcript_id |
| | | | | | “Midline_NG27.1”; exon “2”; |
| chr13 | exon | 86270269 | 86270364 | + | gene_id “Midline_NG27”; transcript_id |
| | | | | | “Midline_NG27.1”; exon “3”; |
| chr13 | exon | 86312972 | 86313040 | + | gene_id “Midline_NG27”; transcript_id |
| | | | | | “Midline_NG27.1”; exon “4”; |
| chr13 | exon | 86353687 | 86353776 | + | gene_id “Midline_NG27”; transcript_id |
| | | | | | “Midline_NG27.1”; exon “5”; |
| chr13 | exon | 86354607 | 86354649 | + | gene_id “Midline_NG27”; transcript_id |
| | | | | | “Midline_NG27.1”; exon “6”; |
| chr13 | exon | 86357201 | 86357493 | + | gene_id “Midline_NG27”; transcript_id |
| | | | | | “Midline_NG27.1”; exon “7”; |
| chr13 | transcript | 86240716 | 86349201 | + | gene_id “Midline_NG27”; transcript_id |
| | | | | | “Midline_NG27.2”; RPKM “3.6059”; cov |
| | | | | | “62.4921”; |
| chr13 | exon | 86240716 | 86240895 | + | gene_id “Midline_NG27”; transcript_id |
| | | | | | “Midline_NG27.2”; exon “1”; |
| chr13 | exon | 86250665 | 86250748 | + | gene_id “Midline_NG27”; transcript_id |
| | | | | | “Midline_NG27.2”; exon “2”; |
| chr13 | exon | 86270269 | 86270364 | + | gene_id “Midline_NG27”; transcript_id |
| | | | | | “Midline_NG27.2”; exon “3”; |
| chr13 | exon | 86312972 | 86313040 | + | gene_id “Midline_NG27”; transcript_id |
| | | | | | “Midline_NG27.2”; exon “4”; |
| chr13 | exon | 86348403 | 86349201 | + | gene_id “Midline_NG27”; transcript_id |
| | | | | | “Midline_NG27.2”; exon “5”; |
| chr13 | transcript | 86240716 | 86615294 | + | gene_id “Midline_NG27”; transcript_id |
| | | | | | “Midline_NG27.3”; RPKM “0.6617”; cov |
| | | | | | “11.4672”; |
| chr13 | exon | 86240716 | 86240895 | + | gene_id “Midline_NG27”; transcript_id |
| | | | | | “Midline_NG27.3”; exon “1”; |
| chr13 | exon | 86250665 | 86250748 | + | gene_id “Midline_NG27”; transcript_id |
| | | | | | “Midline_NG27.3”; exon “2”; |
| chr13 | exon | 86270269 | 86270364 | + | gene_id “Midline_NG27”; transcript_id |
| | | | | | “Midline_NG27.3”; exon “3”; |
| chr13 | exon | 86312972 | 86313040 | + | gene_id “Midline_NG27”; transcript_id |
| | | | | | “Midline_NG27.3”; exon “4”; |
| chr13 | exon | 86610794 | 86615294 | + | gene_id “Midline_NG27”; transcript_id |
| | | | | | “Midline_NG27.3”; exon “5”; |
| chr13 | transcript | 86310504 | 86314041 | + | gene_id “Midline_NG27”; transcript_id |
| | | | | | “Midline_NG27.4”; RPKM “1.6177”; cov |
| | | | | | “28.0357”; |
| chr13 | exon | 86310504 | 86314041 | + | gene_id “Midline_NG27”; transcript_id |
| | | | | | “Midline_NG27.4”; exon “1”; |
| chr17_gl000205_random | transcript | 6725 | 14470 | + | gene_id “Midline_NG29”; transcript_id |
| | | | | | “Midline_NG29.1”; RPKM “0.6211”; cov |
| | | | | | “10.7632”; |
| chr17_gl000205_random | exon | 6725 | 7257 | + | gene_id “Midline_NG29”; transcript_id |
| | | | | | “Midline_NG29.1”; exon “1”; |
| chr17_gl000205_random | exon | 10851 | 14470 | + | gene_id “Midline_NG29”; transcript_id |
| | | | | | “Midline_NG29.1”; exon “2”; |
| chr2 | transcript | 57436798 | 57442143 | − | gene_id “Midline_NG2”; transcript_id |
| | | | | | “Midline_NG2.1”; RPKM “3.0087”; cov |
| | | | | | “52.1431”; |
| chr2 | exon | 57436798 | 57442143 | − | gene_id “Midline_NG2”; transcript_id |
| | | | | | “Midline_NG2.1”; exon “1”; |
| chr2 | transcript | 59063403 | 59091410 | − | gene_id “Midline_NG3”; transcript_id |
| | | | | | “Midline_NG3.1”; RPKM “2.9486”; cov |
| | | | | | “51.1015”; |
| chr2 | exon | 59063403 | 59065418 | − | gene_id “Midline_NG3”; transcript_id |
| | | | | | “Midline_NG3.1”; exon “1”; |
| chr2 | exon | 59069232 | 59069283 | − | gene_id “Midline_NG3”; transcript_id |
| | | | | | “Midline_NG3.1”; exon “2”; |
| chr2 | exon | 59091060 | 59091410 | − | gene_id “Midline_NG3”; transcript_id |
| | | | | | “Midline_NG3.1”; exon “3”; |
| chr2 | transcript | 59063403 | 59091410 | − | gene_id “Midline_NG3”; transcript_id |
| | | | | | “Midline_NG3.2”; RPKM “1.9007”; cov |
| | | | | | “32.9400”; |
| chr2 | exon | 59063403 | 59065418 | − | gene_id “Midline_NG3”; transcript_id |
| | | | | | “Midline_NG3.2”; exon “1”; |
| chr2 | exon | 59069232 | 59069283 | − | gene_id “Midline_NG3”; transcript_id |
| | | | | | “Midline_NG3.2”; exon “2”; |
| chr2 | exon | 59076762 | 59077439 | − | gene_id “Midline_NG3”; transcript_id |
| | | | | | “Midline_NG3.2”; exon “3”; |
| chr2 | exon | 59091060 | 59091410 | − | gene_id “Midline_NG3”; transcript_id |
| | | | | | “Midline_NG3.2”; exon “4”; |
| chr2 | transcript | 59063403 | 59091410 | − | gene_id “Midline_NG3”; transcript_id |
| | | | | | “Midline_NG3.3”; RPKM “0.9661”; cov |
| | | | | | “16.7422”; |
| chr2 | exon | 59063403 | 59065418 | − | gene_id “Midline_NG3”; transcript_id |
| | | | | | “Midline_NG3.3”; exon “1”; |
| chr2 | exon | 59069232 | 59069283 | − | gene_id “Midline_NG3”; transcript_id |
| | | | | | “Midline_NG3.3”; exon “2”; |
| chr2 | exon | 59076762 | 59077439 | − | gene_id “Midline_NG3”; transcript_id |
| | | | | | “Midline_NG3.3”; exon “3”; |
| chr2 | exon | 59090231 | 59091410 | − | gene_id “Midline_NG3”; transcript_id |
| | | | | | “Midline_NG3.3”; exon “4”; |
| chr2 | transcript | 60781021 | 60783180 | + | gene_id “Midline_NG4”; transcript_id |
| | | | | | “Midline_NG4.1”; RPKM “3.1323”; cov |
| | | | | | “54.2843”; |
| chr2 | exon | 60781021 | 60783180 | + | gene_id “Midline_NG4”; transcript_id |
| | | | | | “Midline_NG4.1”; exon “1”; |
| chr2 | transcript | 60783193 | 60784227 | + | gene_id “Midline_NG5”; transcript_id |
| | | | | | “Midline_NG5.1”; RPKM “1.9665”; cov |
| | | | | | “34.0812”; |
| chr2 | exon | 60783193 | 60784227 | + | gene_id “Midline_NG5”; transcript_id |
| | | | | | “Midline_NG5.1”; exon “1”; |
| chr2 | transcript | 117511281 | 117536452 | + | gene_id “Midline_NG6”; transcript_id |
| | | | | | “Midline_NG6.2”; RPKM “0.8703”; cov |
| | | | | | “15.0835”; |
| chr2 | exon | 117511281 | 117511916 | + | gene_id “Midline_NG6”; transcript_id |
| | | | | | “Midline_NG6.2”; exon “1”; |
| chr2 | exon | 117528080 | 117528151 | + | gene_id “Midline_NG6”; transcript_id |
| | | | | | “Midline_NG6.2”; exon “2”; |
| chr2 | exon | 117531621 | 117536452 | + | gene_id “Midline_NG6”; transcript_id |
| | | | | | “Midline_NG6.2”; exon “3”; |
| chr2 | transcript | 117511281 | 117568649 | + | gene_id “Midline_NG6”; transcript_id |
| | | | | | “Midline_NG6.3”; RPKM “0.7225”; cov |
| | | | | | “12.5214”; |
| chr2 | exon | 117511281 | 117511916 | + | gene_id “Midline_NG6”; transcript_id |
| | | | | | “Midline_NG6.3”; exon “1”; |
| chr2 | exon | 117528080 | 117528151 | + | gene_id “Midline_NG6”; transcript_id |
| | | | | | “Midline_NG6.3”; exon “2”; |
| chr2 | exon | 117566452 | 117566494 | + | gene_id “Midline_NG6”; transcript_id |
| | | | | | “Midline_NG6.3”; exon “3”; |
| chr2 | exon | 117568280 | 117568649 | + | gene_id “Midline_NG6”; transcript_id |
| | | | | | “Midline_NG6.3”; exon “4”; |
| chr2 | transcript | 117511281 | 117556188 | + | gene_id “Midline_NG6”; transcript_id |
| | | | | | “Midline_NG6.4”; RPKM “0.6393”; cov |
| | | | | | “11.0791”; |
| chr2 | exon | 117511281 | 117511916 | + | gene_id “Midline_NG6”; transcript_id |
| | | | | | “Midline_NG6.4”; exon “1”; |
| chr2 | exon | 117528080 | 117528151 | + | gene_id “Midline_NG6”; transcript_id |
| | | | | | “Midline_NG6.4”; exon “2”; |
| chr2 | exon | 117548537 | 117556188 | + | gene_id “Midline_NG6”; transcript_id |
| | | | | | “Midline_NG6.4”; exon “3”; |
| chr2 | transcript | 117544112 | 117556188 | + | gene_id “Midline_NG6”; transcript_id |
| | | | | | “Midline_NG6.1”; RPKM “1.8155”; cov |
| | | | | | “31.4633”; |
| chr2 | exon | 117544112 | 117556188 | + | gene_id “Midline_NG6”; transcript_id |
| | | | | | “Midline_NG6.1”; exon “1”; |
| chr3 | transcript | 172340093 | 172349793 | + | gene_id “Midline_NG7”; transcript_id |
| | | | | | “Midline_NG7.1”; RPKM “0.8464”; cov |
| | | | | | “14.6681”; |
| chr3 | exon | 172340093 | 172340226 | + | gene_id “Midline_NG7”; transcript_id |
| | | | | | “Midline_NG7.1”; exon “1”; |
| chr3 | exon | 172349186 | 172349793 | + | gene_id “Midline_NG7”; transcript_id |
| | | | | | “Midline_NG7.1”; exon “2”; |
| chr3 | transcript | 181943552 | 181954028 | − | gene_id “Midline_NG8”; transcript_id |
| | | | | | “Midline_NG8.1”; RPKM “0.6069”; cov |
| | | | | | “10.5179”; |
| chr3 | exon | 181943552 | 181944257 | − | gene_id “Midline_NG8”; transcript_id |
| | | | | | “Midline_NG8.1”; exon “1”; |
| chr3 | exon | 181947388 | 181947459 | − | gene_id “Midline_NG8”; transcript_id |
| | | | | | “Midline_NG8.1”; exon “2”; |
| chr3 | exon | 181950019 | 181950090 | − | gene_id “Midline_NG8”; transcript_id |
| | | | | | “Midline_NG8.1”; exon “3”; |
| chr3 | exon | 181951884 | 181954028 | − | gene_id “Midline_NG8”; transcript_id |
| | | | | | “Midline_NG8.1”; exon “4”; |
| chr3 | transcript | 181981092 | 181984367 | + | gene_id “Midline_NG9”; transcript_id |
| | | | | | “Midline_NG9.1”; RPKM “3.7999”; cov |
| | | | | | “65.8538”; |
| chr3 | exon | 181981092 | 181984367 | + | gene_id “Midline_NG9”; transcript_id |
| | | | | | “Midline_NG9.1”; exon “1”; |
| chr3 | transcript | 182296463 | 182410116 | − | gene_id “Midline_NG10”; transcript_id |
| | | | | | “Midline_NG10.4”; RPKM “1.4826”; cov |
| | | | | | “25.6944”; |
| chr3 | exon | 182296463 | 182297846 | − | gene_id “Midline_NG10”; transcript_id |
| | | | | | “Midline_NG10.4”; exon “1”; |
| chr3 | exon | 182335300 | 182335385 | − | gene_id “Midline_NG10”; transcript_id |
| | | | | | “Midline_NG10.4”; exon “2”; |
| chr3 | exon | 182338171 | 182338412 | − | gene_id “Midline_NG10”; transcript_id |
| | | | | | “Midline_NG10.4”; exon “3”; |
| chr3 | exon | 182406874 | 182406991 | − | gene_id “Midline_NG10”; transcript_id |
| | | | | | “Midline_NG10.4”; exon “4”; |
| chr3 | exon | 182409868 | 182410116 | − | gene_id “Midline_NG10”; transcript_id |
| | | | | | “Midline_NG10.4”; exon “5”; |
| chr3 | transcript | 182402789 | 182410116 | − | gene_id “Midline_NG10”; transcript_id |
| | | | | | “Midline_NG10.2”; RPKM “1.1113”; cov |
| | | | | | “19.2601”; |
| chr3 | exon | 182402789 | 182406991 | − | gene_id “Midline_NG10”; transcript_id |
| | | | | | “Midline_NG10.2”; exon “1”; |
| chr3 | exon | 182409868 | 182410116 | − | gene_id “Midline_NG10”; transcript_id |
| | | | | | “Midline_NG10.2”; exon “2”; |
| chr3 | transcript | 182410454 | 182415565 | − | gene_id “Midline_NG10”; transcript_id |
| | | | | | “Midline_NG10.3”; RPKM “3.7889”; cov |
| | | | | | “65.6631”; |
| chr3 | exon | 182410454 | 182411045 | − | gene_id “Midline_NG10”; transcript_id |
| | | | | | “Midline_NG10.3”; exon “1”; |
| chr3 | exon | 182414828 | 182414948 | − | gene_id “Midline_NG10”; transcript_id |
| | | | | | “Midline_NG10.3”; exon “2”; |
| chr3 | exon | 182415159 | 182415565 | − | gene_id “Midline_NG10”; transcript_id |
| | | | | | “Midline_NG10.3”; exon “3”; |
| chr3 | transcript | 182410454 | 182412434 | − | gene_id “Midline_NG10”; transcript_id |
| | | | | | “Midline_NG10.1”; RPKM “1.6169”; cov |
| | | | | | “28.0225”; |
| chr3 | exon | 182410454 | 182412434 | − | gene_id “Midline_NG10”; transcript_id |
| | | | | | “Midline_NG10.1”; exon “1”; |
| chr3 | transcript | 182281415 | 182288803 | − | gene_id “Midline_NG11”; transcript_id |
| | | | | | “Midline_NG11.1”; RPKM “3.1229”; cov |
| | | | | | “54.1210”; |
| chr3 | exon | 182281415 | 182288803 | − | gene_id “Midline_NG11”; transcript_id |
| | | | | | “Midline_NG11.1”; exon “1”; |
| chr3 | transcript | 182421165 | 182503431 | + | gene_id “Midline_NG12”; transcript_id |
| | | | | | “Midline_NG12.3”; RPKM “0.8552”; cov |
| | | | | | “14.8209”; |
| chr3 | exon | 182421165 | 182421493 | + | gene_id “Midline_NG12”; transcript_id |
| | | | | | “Midline_NG12.3”; exon “1”; |
| chr3 | exon | 182499690 | 182499807 | + | gene_id “Midline_NG12”; transcript_id |
| | | | | | “Midline_NG12.3”; exon “2”; |
| chr3 | exon | 182502139 | 182503431 | + | gene_id “Midline_NG12”; transcript_id |
| | | | | | “Midline_NG12.3”; exon “3”; |
| chr3 | transcript | 182421165 | 182503431 | + | gene_id “Midline_NG12”; transcript_id |
| | | | | | “Midline_NG12.2”; RPKM “1.4405”; cov |
| | | | | | “24.9639”; |
| chr3 | exon | 182421165 | 182421493 | + | gene_id “Midline_NG12”; transcript_id |
| | | | | | “Midline_NG12.2”; exon “1”; |
| chr3 | exon | 182499690 | 182499807 | + | gene_id “Midline_NG12”; transcript_id |
| | | | | | “Midline_NG12.2”; exon “2”; |
| chr3 | exon | 182501056 | 182501148 | + | gene_id “Midline_NG12”; transcript_id |
| | | | | | “Midline_NG12.2”; exon “3”; |
| chr3 | exon | 182502139 | 182503431 | + | gene_id “Midline_NG12”; transcript_id |
| | | | | | “Midline_NG12.2”; exon “4”; |
| chr3 | transcript | 182421165 | 182473642 | + | gene_id “Midline_NG12”; transcript_id |
| | | | | | “Midline_NG12.1”; RPKM “1.2159”; cov |
| | | | | | “21.0714”; |
| chr3 | exon | 182421165 | 182421493 | + | gene_id “Midline_NG12”; transcript_id |
| | | | | | “Midline_NG12.1”; exon “1”; |
| chr3 | exon | 182469939 | 182472111 | + | gene_id “Midline_NG12”; transcript_id |
| | | | | | “Midline_NG12.1”; exon “2”; |
| chr3 | exon | 182472132 | 182473642 | + | gene_id “Midline_NG12”; transcript_id |
| | | | | | “Midline_NG12.1”; exon “3”; |
| chr4 | transcript | 74531148 | 74553199 | − | gene_id “Midline_NG13”; transcript_id |
| | | | | | “Midline_NG13.1”; RPKM “0.9354”; cov |
| | | | | | “16.2116”; |
| chr4 | exon | 74531148 | 74536640 | − | gene_id “Midline_NG13”; transcript_id |
| | | | | | “Midline_NG13.1”; exon “1”; |
| chr4 | exon | 74552851 | 74553199 | − | gene_id “Midline_NG13”; transcript_id |
| | | | | | “Midline_NG13.1”; exon “2”; |
| chr4 | transcript | 74531148 | 74553199 | − | gene_id “Midline_NG13”; transcript_id |
| | | | | | “Midline_NG13.2”; RPKM “1.2050”; cov |
| | | | | | “20.8835”; |
| chr4 | exon | 74531148 | 74532256 | − | gene_id “Midline_NG13”; transcript_id |
| | | | | | “Midline_NG13.2”; exon “1”; |
| chr4 | exon | 74536467 | 74536640 | − | gene_id “Midline_NG13”; transcript_id |
| | | | | | “Midline_NG13.2”; exon “2”; |
| chr4 | exon | 74552851 | 74553199 | − | gene_id “Midline_NG13”; transcript_id |
| | | | | | “Midline_NG13.2”; exon “3”; |
| chr4 | transcript | 74746644 | 74754291 | + | gene_id “Midline_NG14”; transcript_id |
| | | | | | “Midline_NG14.2”; RPKM “1.1574”; cov |
| | | | | | “20.0575”; |
| chr4 | exon | 74746644 | 74746813 | + | gene_id “Midline_NG14”; transcript_id |
| | | | | | “Midline_NG14.2”; exon “1”; |
| chr4 | exon | 74747504 | 74747631 | + | gene_id “Midline_NG14”; transcript_id |
| | | | | | “Midline_NG14.2”; exon “2”; |
| chr4 | exon | 74753866 | 74754291 | + | gene_id “Midline_NG14”; transcript_id |
| | | | | | “Midline_NG14.2”; exon “3”; |
| chr4 | transcript | 74746644 | 74749249 | + | gene_id “Midline_NG14”; transcript_id |
| | | | | | “Midline_NG14.3”; RPKM “1.5361”; cov |
| | | | | | “26.6207”; |
| chr4 | exon | 74746644 | 74746813 | + | gene_id “Midline_NG14”; transcript_id |
| | | | | | “Midline_NG14.3”; exon “1”; |
| chr4 | exon | 74747504 | 74747631 | + | gene_id “Midline_NG14”; transcript_id |
| | | | | | “Midline_NG14.3”; exon “2”; |
| chr4 | exon | 74748469 | 74749249 | + | gene_id “Midline_NG14”; transcript_id |
| | | | | | “Midline_NG14.3”; exon “3”; |
| chr4 | transcript | 74751694 | 74754291 | + | gene_id “Midline_NG14”; transcript_id |
| | | | | | “Midline_NG14.1”; RPKM “1.1381”; cov |
| | | | | | “19.7246”; |
| chr4 | exon | 74751694 | 74751921 | + | gene_id “Midline_NG14”; transcript_id |
| | | | | | “Midline_NG14.1”; exon “1”; |
| chr4 | exon | 74753866 | 74754291 | + | gene_id “Midline_NG14”; transcript_id |
| | | | | | “Midline_NG14.1”; exon “2”; |
| chr5 | transcript | 82056215 | 82057217 | + | gene_id “Midline_NG15”; transcript_id |
| | | | | | “Midline_NG15.1”; RPKM “4.1334”; cov |
| | | | | | “71.6332”; |
| chr5 | exon | 82056215 | 82057217 | + | gene_id “Midline_NG15”; transcript_id |
| | | | | | “Midline_NG15.1”; exon “1”; |
| chr5 | transcript | 102908913 | 102909803 | + | gene_id “Midline_NG16”; transcript_id |
| | | | | | “Midline_NG16.1”; RPKM “1.1635”; cov |
| | | | | | “20.1642”; |
| chr5 | exon | 102908913 | 102909803 | + | gene_id “Midline_NG16”; transcript_id |
| | | | | | “Midline_NG16.1”; exon “1”; |
| chr5 | transcript | 103011592 | 103286410 | + | gene_id “Midline_NG16”; transcript_id |
| | | | | | “Midline_NG16.7”; RPKM “1.2463”; cov |
| | | | | | “21.5988”; |
| chr5 | exon | 103011592 | 103012133 | + | gene_id “Midline_NG16”; transcript_id |
| | | | | | “Midline_NG16.7”; exon “1”; |
| chr5 | exon | 103014213 | 103014267 | + | gene_id “Midline_NG16”; transcript_id |
| | | | | | “Midline_NG16.7”; exon “2”; |
| chr5 | exon | 103154863 | 103154935 | + | gene_id “Midline_NG16”; transcript_id |
| | | | | | “Midline_NG16.7”; exon “3”; |
| chr5 | exon | 103249096 | 103249187 | + | gene_id “Midline_NG16”; transcript_id |
| | | | | | “Midline_NG16.7”; exon “4”; |
| chr5 | exon | 103251007 | 103251132 | + | gene_id “Midline_NG16”; transcript_id |
| | | | | | “Midline_NG16.7”; exon “5”; |
| chr5 | exon | 103269266 | 103286410 | + | gene_id “Midline_NG16”; transcript_id |
| | | | | | “Midline_NG16.7”; exon “6”; |
| chr5 | transcript | 103011592 | 103286410 | + | gene_id “Midline_NG16”; transcript_id |
| | | | | | “Midline_NG16.6”; RPKM “1.2780”; cov |
| | | | | | “22.1488”; |
| chr5 | exon | 103011592 | 103012133 | + | gene_id “Midline_NG16”; transcript_id |
| | | | | | “Midline_NG16.6”; exon “1”; |
| chr5 | exon | 103014213 | 103014267 | + | gene_id “Midline_NG16”; transcript_id |
| | | | | | “Midline_NG16.6”; exon “2”; |
| chr5 | exon | 103154863 | 103154935 | + | gene_id “Midline_NG16”; transcript_id |
| | | | | | “Midline_NG16.6”; exon “3”; |
| chr5 | exon | 103249096 | 103249187 | + | gene_id “Midline_NG16”; transcript_id |
| | | | | | “Midline_NG16.6”; exon “4”; |
| chr5 | exon | 103269266 | 103286410 | + | gene_id “Midline_NG16”; transcript_id |
| | | | | | “Midline_NG16.6”; exon “5”; |
| chr5 | transcript | 103011592 | 103286410 | + | gene_id “Midline_NG16”; transcript_id |
| | | | | | “Midline_NG16.5”; RPKM “1.0633”; cov |
| | | | | | “18.4269”; |
| chr5 | exon | 103011592 | 103012133 | + | gene_id “Midline_NG16”; transcript_id |
| | | | | | “Midline_NG16.5”; exon “1”; |
| chr5 | exon | 103014213 | 103014267 | + | gene_id “Midline_NG16”; transcript_id |
| | | | | | “Midline_NG16.5”; exon “2”; |
| chr5 | exon | 103154863 | 103154935 | + | gene_id “Midline_NG16”; transcript_id |
| | | | | | “Midline_NG16.5”; exon “3”; |
| chr5 | exon | 103249096 | 103249187 | + | gene_id “Midline_NG16”; transcript_id |
| | | | | | “Midline_NG16.5”; exon “4”; |
| chr5 | exon | 103251007 | 103251132 | + | gene_id “Midline_NG16”; transcript_id |
| | | | | | “Midline_NG16.5”; exon “5”; |
| chr5 | exon | 103267968 | 103268119 | + | gene_id “Midline_NG16”; transcript_id |
| | | | | | “Midline_NG16.5”; exon “6”; |
| chr5 | exon | 103269266 | 103269673 | + | gene_id “Midline_NG16”; transcript_id |
| | | | | | “Midline_NG16.5”; exon “7”; |
| chr5 | exon | 103281981 | 103286410 | + | gene_id “Midline_NG16”; transcript_id |
| | | | | | “Midline_NG16.5”; exon “8”; |
| chr5 | transcript | 103011592 | 103286410 | + | gene_id “Midline_NG16”; transcript_id |
| | | | | | “Midline_NG16.4”; RPKM “1.4194”; cov |
| | | | | | “24.5985”; |
| chr5 | exon | 103011592 | 103012133 | + | gene_id “Midline_NG16”; transcript_id |
| | | | | | “Midline_NG16.4”; exon “1”; |
| chr5 | exon | 103014213 | 103014267 | + | gene_id “Midline_NG16”; transcript_id |
| | | | | | “Midline_NG16.4”; exon “2”; |
| chr5 | exon | 103154863 | 103154935 | + | gene_id “Midline_NG16”; transcript_id |
| | | | | | “Midline_NG16.4”; exon “3”; |
| chr5 | exon | 103249096 | 103249187 | + | gene_id “Midline_NG16”; transcript_id |
| | | | | | “Midline_NG16.4”; exon “4”; |
| chr5 | exon | 103251007 | 103251132 | + | gene_id “Midline_NG16”; transcript_id |
| | | | | | “Midline_NG16.4”; exon “5”; |
| chr5 | exon | 103266433 | 103266724 | + | gene_id “Midline_NG16”; transcript_id |
| | | | | | “Midline_NG16.4”; exon “6”; |
| chr5 | exon | 103267968 | 103268119 | + | gene_id “Midline_NG16”; transcript_id |
| | | | | | “Midline_NG16.4”; exon “7”; |
| chr5 | exon | 103269266 | 103269673 | + | gene_id “Midline_NG16”; transcript_id |
| | | | | | “Midline_NG16.4”; exon “8”; |
| chr5 | exon | 103281981 | 103286410 | + | gene_id “Midline_NG16”; transcript_id |
| | | | | | “Midline_NG16.4”; exon “9”; |
| chr5 | transcript | 103011592 | 103286410 | + | gene_id “Midline_NG16”; transcript_id |
| | | | | | “Midline_NG16.2”; RPKM “0.8690”; cov |
| | | | | | “15.0606”; |
| chr5 | exon | 103011592 | 103012133 | + | gene_id “Midline_NG16”; transcript_id |
| | | | | | “Midline_NG16.2”; exon “1”; |
| chr5 | exon | 103014213 | 103014267 | + | gene_id “Midline_NG16”; transcript_id |
| | | | | | “Midline_NG16.2”; exon “2”; |
| chr5 | exon | 103154863 | 103154935 | + | gene_id “Midline_NG16”; transcript_id |
| | | | | | “Midline_NG16.2”; exon “3”; |
| chr5 | exon | 103269266 | 103269673 | + | gene_id “Midline_NG16”; transcript_id |
| | | | | | “Midline_NG16.2”; exon “4”; |
| chr5 | exon | 103281981 | 103286410 | + | gene_id “Midline_NG16”; transcript_id |
| | | | | | “Midline_NG16.2”; exon “5”; |
| chr5 | transcript | 103264884 | 103286410 | + | gene_id “Midline_NG16”; transcript_id |
| | | | | | “Midline_NG16.3”; RPKM “0.6171”; cov |
| | | | | | “10.6942”; |
| chr5 | exon | 103264884 | 103266724 | + | gene_id “Midline_NG16”; transcript_id |
| | | | | | “Midline_NG16.3”; exon “1”; |
| chr5 | exon | 103267968 | 103268119 | + | gene_id “Midline_NG16”; transcript_id |
| | | | | | “Midline_NG16.3”; exon “2”; |
| chr5 | exon | 103269266 | 103269673 | + | gene_id “Midline_NG16”; transcript_id |
| | | | | | “Midline_NG16.3”; exon “3”; |
| chr5 | exon | 103281981 | 103286410 | + | gene_id “Midline_NG16”; transcript_id |
| | | | | | “Midline_NG16.3”; exon “4”; |
| chr5 | transcript | 103047381 | 103048443 | + | gene_id “Midline_NG17”; transcript_id |
| | | | | | “Midline_NG17.1”; RPKM “5.0516”; cov |
| | | | | | “87.5475”; |
| chr5 | exon | 103047381 | 103048443 | + | gene_id “Midline_NG17”; transcript_id |
| | | | | | “Midline_NG17.1”; exon “1”; |
| chr5 | transcript | 103090181 | 103092255 | + | gene_id “Midline_NG18”; transcript_id |
| | | | | | “Midline_NG18.1”; RPKM “3.5904”; cov |
| | | | | | “62.2231”; |
| chr5 | exon | 103090181 | 103092255 | + | gene_id “Midline_NG18”; transcript_id |
| | | | | | “Midline_NG18.1”; exon “1”; |
| chr5 | transcript | 103292001 | 103308839 | + | gene_id “Midline_NG19”; transcript_id |
| | | | | | “Midline_NG19.2”; RPKM “3.9691”; cov |
| | | | | | “68.7869”; |
| chr5 | exon | 103292001 | 103308839 | + | gene_id “Midline_NG19”; transcript_id |
| | | | | | “Midline_NG19.2”; exon “1”; |
| chr5 | transcript | 103324512 | 103335038 | + | gene_id “Midline_NG19”; transcript_id |
| | | | | | “Midline_NG19.1”; RPKM “2.6366”; cov |
| | | | | | “45.6942”; |
| chr5 | exon | 103324512 | 103335038 | + | gene_id “Midline_NG19”; transcript_id |
| | | | | | “Midline_NG19.1”; exon “1”; |
| chr5 | transcript | 103310277 | 103324457 | + | gene_id “Midline_NG20”; transcript_id |
| | | | | | “Midline_NG20.1”; RPKM “1.1678”; cov |
| | | | | | “20.2378”; |
| chr5 | exon | 103310277 | 103324457 | + | gene_id “Midline_NG20”; transcript_id |
| | | | | | “Midline_NG20.1”; exon “1”; |
| chr5 | transcript | 103335061 | 103337734 | + | gene_id “Midline_NG21”; transcript_id |
| | | | | | “Midline_NG21.1”; RPKM “3.7553”; cov |
| | | | | | “65.0819”; |
| chr5 | exon | 103335061 | 103337734 | + | gene_id “Midline_NG21”; transcript_id |
| | | | | | “Midline_NG21.1”; exon “1”; |
| chr6 | transcript | 93621815 | 93625011 | − | gene_id “Midline_NG22”; transcript_id |
| | | | | | “Midline_NG22.1”; RPKM “10.2269”; cov |
| | | | | | “177.2371”; |
| chr6 | exon | 93621815 | 93625011 | − | gene_id “Midline_NG22”; transcript_id |
| | | | | | “Midline_NG22.1”; exon “1”; |
| chr7 | transcript | 7755069 | 7768011 | + | gene_id “Midline_NG23”; transcript_id |
| | | | | | “Midline_NG23.3”; RPKM “5.6055”; cov |
| | | | | | “97.1468”; |
| chr7 | exon | 7755069 | 7768011 | + | gene_id “Midline_NG23”; transcript_id |
| | | | | | “Midline_NG23.3”; exon “1”; |
| chr7 | transcript | 7755069 | 7768011 | + | gene_id “Midline_NG23”; transcript_id |
| | | | | | “Midline_NG23.2”; RPKM “1.9633”; cov |
| | | | | | “34.0243”; |
| chr7 | exon | 7755069 | 7757168 | + | gene_id “Midline_NG23”; transcript_id |
| | | | | | “Midline_NG23.2”; exon “1”; |
| chr7 | exon | 7760544 | 7768011 | + | gene_id “Midline_NG23”; transcript_id |
| | | | | | “Midline_NG23.2”; exon “2”; |
| chr7 | transcript | 7755069 | 7768011 | + | gene_id “Midline_NG23”; transcript_id |
| | | | | | “Midline_NG23.1”; RPKM “12.3499”; cov |
| | | | | | “214.0304”; |
| chr7 | exon | 7755069 | 7757081 | + | gene_id “Midline_NG23”; transcript_id |
| | | | | | “Midline_NG23.1”; exon “1”; |
| chr7 | exon | 7760544 | 7768011 | + | gene_id “Midline_NG23”; transcript_id |
| | | | | | “Midline_NG23.1”; exon “2”; |
| chr7_gl000195_random | transcript | 103067 | 110237 | − | gene_id “Midline_NG28”; transcript_id |
| | | | | | “Midline_NG28.1”; RPKM “3.4060”; cov |
| | | | | | “59.0282”; |
| chr7_gl000195_random | exon | 103067 | 110237 | − | gene_id “Midline_NG28”; transcript_id |
| | | | | | “Midline_NG28.1”; exon “1”; |
| chr8 | transcript | 76014971 | 76016954 | − | gene_id “Midline_NG24”; transcript_id |
| | | | | | “Midline_NG24.1”; RPKM “1.4732”; cov |
| | | | | | “25.5312”; |
| chr8 | exon | 76014971 | 76016954 | − | gene_id “Midline_NG24”; transcript_id |
| | | | | | “Midline_NG24.1”; exon “1”; |
| chr8 | transcript | 128120178 | 128148188 | − | gene_id “Midline_NG25”; transcript_id |
| | | | | | “Midline_NG25.1”; RPKM “3.8473”; cov |
| | | | | | “66.6760”; |
| chr8 | exon | 128120178 | 128148188 | − | gene_id “Midline_NG25”; transcript_id |
| | | | | | “Midline_NG25.1”; exon “1”; |
| chr8 | transcript | 128120178 | 128159468 | − | gene_id “Midline_NG25”; transcript_id |
| | | | | | “Midline_NG25.3”; RPKM “2.1451”; cov |
| | | | | | “37.1756”; |
| chr8 | exon | 128120178 | 128129210 | − | gene_id “Midline_NG25”; transcript_id |
| | | | | | “Midline_NG25.3”; exon “1”; |
| chr8 | exon | 128152989 | 128153103 | − | gene_id “Midline_NG25”; transcript_id |
| | | | | | “Midline_NG25.3”; exon “2”; |
| chr8 | exon | 128153719 | 128153816 | − | gene_id “Midline_NG25”; transcript_id |
| | | | | | “Midline_NG25.3”; exon “3”; |
| chr8 | exon | 128156008 | 128159468 | − | gene_id “Midline_NG25”; transcript_id |
| | | | | | “Midline_NG25.3”; exon “4”; |
| chr8 | transcript | 128151294 | 128159468 | − | gene_id “Midline_NG25”; transcript_id |
| | | | | | “Midline_NG25.2”; RPKM “2.5375”; cov |
| | | | | | “43.9754”; |
| chr8 | exon | 128151294 | 128153103 | − | gene_id “Midline_NG25”; transcript_id |
| | | | | | “Midline_NG25.2”; exon “1”; |
| chr8 | exon | 128153719 | 128159468 | − | gene_id “Midline_NG25”; transcript_id |
| | | | | | “Midline_NG25.2”; exon “2”; |
| chr8 | transcript | 128151294 | 128163711 | − | gene_id “Midline_NG25”; transcript_id |
| | | | | | “Midline_NG25.4”; RPKM “8.8818”; cov |
| | | | | | “153.9259”; |
| chr8 | exon | 128151294 | 128153103 | − | gene_id “Midline_NG25”; transcript_id |
| | | | | | “Midline_NG25.4”; exon “1”; |
| chr8 | exon | 128153719 | 128153816 | − | gene_id “Midline_NG25”; transcript_id |
| | | | | | “Midline_NG25.4”; exon “2”; |
| chr8 | exon | 128156008 | 128163711 | − | gene_id “Midline_NG25”; transcript_id |
| | | | | | “Midline_NG25.4”; exon “3”; |
| chr8 | transcript | 128163752 | 128182015 | − | gene_id “Midline_NG25”; transcript_id |
| | | | | | “Midline_NG25.5”; RPKM “0.9976”; cov |
| | | | | | “17.2894”; |
| chr8 | exon | 128163752 | 128165589 | − | gene_id “Midline_NG25”; transcript_id |
| | | | | | “Midline_NG25.5”; exon “1”; |
| chr8 | exon | 128174167 | 128174329 | − | gene_id “Midline_NG25”; transcript_id |
| | | | | | “Midline_NG25.5”; exon “2”; |
| chr8 | exon | 128181143 | 128182015 | − | gene_id “Midline_NG25”; transcript_id |
| | | | | | “Midline_NG25.5”; exon “3”; |
| chr10 | transcript | 131033896 | 131047803 | − | gene_id “mLPS_NG2”; transcript_id |
| | | | | | “mLPS_NG2.1”; RPKM “10.8022”; cov |
| | | | | | “151.6608”; |
| chr10 | exon | 131033896 | 131047803 | − | gene_id “mLPS_NG2”; transcript_id |
| | | | | | “mLPS_NG2.1”; exon “1”; |
| chr10 | transcript | 131033896 | 131054561 | − | gene_id “mLPS_NG2”; transcript_id |
| | | | | | “mLPS_NG2.2”; RPKM “1.6978”; cov |
| | | | | | “23.8367”; |
| chr10 | exon | 131033896 | 131041247 | − | gene_id “mLPS_NG2”; transcript_id |
| | | | | | “mLPS_NG2.2”; exon “1”; |
| chr10 | exon | 131053471 | 131053599 | − | gene_id “mLPS_NG2”; transcript_id |
| | | | | | “mLPS_NG2.2”; exon “2”; |
| chr10 | exon | 131053975 | 131054561 | − | gene_id “mLPS_NG2”; transcript_id |
| | | | | | “mLPS_NG2.2”; exon “3”; |
| chr10 | transcript | 131033896 | 131056347 | − | gene_id “mLPS_NG2”; transcript_id |
| | | | | | “mLPS_NG2.4”; RPKM “3.9750”; cov |
| | | | | | “55.8079”; |
| chr10 | exon | 131033896 | 131041247 | − | gene_id “mLPS_NG2”; transcript_id |
| | | | | | “mLPS_NG2.4”; exon “1”; |
| chr10 | exon | 131053471 | 131053599 | − | gene_id “mLPS_NG2”; transcript_id |
| | | | | | “mLPS_NG2.4”; exon “2”; |
| chr10 | exon | 131056007 | 131056347 | − | gene_id “mLPS_NG2”; transcript_id |
| | | | | | “mLPS_NG2.4”; exon “3”; |
| chr10 | transcript | 131047971 | 131056347 | − | gene_id “mLPS_NG2”; transcript_id |
| | | | | | “mLPS_NG2.3”; RPKM “1.4198”; cov |
| | | | | | “19.9331”; |
| chr10 | exon | 131047971 | 131053599 | − | gene_id “mLPS_NG2”; transcript_id |
| | | | | | “mLPS_NG2.3”; exon “1”; |
| chr10 | exon | 131056007 | 131056347 | − | gene_id “mLPS_NG2”; transcript_id |
| | | | | | “mLPS_NG2.3”; exon “2”; |
| chr13 | transcript | 53519925 | 53527015 | − | gene_id “mLPS_NG3”; transcript_id |
| | | | | | “mLPS_NG3.1”; RPKM “2.0584”; cov |
| | | | | | “28.9002”; |
| chr13 | exon | 53519925 | 53520170 | − | gene_id “mLPS_NG3”; transcript_id |
| | | | | | “mLPS_NG3.1”; exon “1”; |
| chr13 | exon | 53526864 | 53527015 | − | gene_id “mLPS_NG3”; transcript_id |
| | | | | | “mLPS_NG3.1”; exon “2”; |
| chr13 | transcript | 71563535 | 71574912 | + | gene_id “mLPS_NG5”; transcript_id |
| | | | | | “mLPS_NG5.3”; RPKM “1.5553”; cov |
| | | | | | “21.8360”; |
| chr13 | exon | 71563535 | 71564222 | + | gene_id “mLPS_NG5”; transcript_id |
| | | | | | “mLPS_NG5.3”; exon “1”; |
| chr13 | exon | 71572233 | 71574912 | + | gene_id “mLPS_NG5”; transcript_id |
| | | | | | “mLPS_NG5.3”; exon “2”; |
| chr13 | transcript | 71567950 | 71574912 | + | gene_id “mLPS_NG5”; transcript_id |
| | | | | | “mLPS_NG5.2”; RPKM “3.2833”; cov |
| | | | | | “46.0963”; |
| chr13 | exon | 71567950 | 71574912 | + | gene_id “mLPS_NG5”; transcript_id |
| | | | | | “mLPS_NG5.2”; exon “1”; |
| chr13 | transcript | 71894318 | 71900319 | + | gene_id “mLPS_NG5”; transcript_id |
| | | | | | “mLPS_NG5.1”; RPKM “5.2827”; cov |
| | | | | | “74.1679”; |
| chr13 | exon | 71894318 | 71900319 | + | gene_id “mLPS_NG5”; transcript_id |
| | | | | | “mLPS_NG5.1”; exon “1”; |
| chr13 | transcript | 71885454 | 71885954 | + | gene_id “mLPS_NG4”; transcript_id |
| | | | | | “mLPS_NG4.1”; RPKM “3.4751”; cov |
| | | | | | “48.7904”; |
| chr13 | exon | 71885454 | 71885954 | + | gene_id “mLPS_NG4”; transcript_id |
| | | | | | “mLPS_NG4.1”; exon “1”; |
| chr13 | transcript | 112869718 | 112871768 | + | gene_id “mLPS_NG6”; transcript_id |
| | | | | | “mLPS_NG6.1”; RPKM “1.9196”; cov |
| | | | | | “26.9502”; |
| chr13 | exon | 112869718 | 112871768 | + | gene_id “mLPS_NG6”; transcript_id |
| | | | | | “mLPS_NG6.1”; exon “1”; |
| chr14 | transcript | 59543239 | 59544797 | − | gene_id “mLPS_NG7”; transcript_id |
| | | | | | “mLPS_NG7.1”; RPKM “2.7626”; cov |
| | | | | | “38.7858”; |
| chr14 | exon | 59543239 | 59544797 | − | gene_id “mLPS_NG7”; transcript_id |
| | | | | | “mLPS_NG7.1”; exon “1”; |
| chr17 | transcript | 3323408 | 3327044 | − | gene_id “mLPS_NG8”; transcript_id |
| | | | | | “mLPS_NG8.1”; RPKM “1.9124”; cov |
| | | | | | “26.8499”; |
| chr17 | exon | 3323408 | 3327044 | − | gene_id “mLPS_NG8”; transcript_id |
| | | | | | “mLPS_NG8.1”; exon “1”; |
| chr2 | transcript | 5876719 | 5888324 | − | gene_id “mLPS_NG1”; transcript_id |
| | | | | | “mLPS_NG1.1”; RPKM “1.8575”; cov |
| | | | | | “26.0783”; |
| chr2 | exon | 5876719 | 5879421 | − | gene_id “mLPS_NG1”; transcript_id |
| | | | | | “mLPS_NG1.1”; exon “1”; |
| chr2 | exon | 5885881 | 5885959 | − | gene_id “mLPS_NG1”; transcript_id |
| | | | | | “mLPS_NG1.1”; exon “2”; |
| chr2 | exon | 5887739 | 5888324 | − | gene_id “mLPS_NG1”; transcript_id |
| | | | | | “mLPS_NG1.1”; exon “3”; |
| chr21 | transcript | 33679555 | 33681865 | + | gene_id “mLPS_NG9”; transcript_id |
| | | | | | “mLPS_NG9.1”; RPKM “1.9122”; cov |
| | | | | | “26.8473”; |
| chr21 | exon | 33679555 | 33681865 | + | gene_id “mLPS_NG9”; transcript_id |
| | | | | | “mLPS_NG9.1”; exon “1”; |
| chr1 | transcript | 1212163 | 1214780 | − | gene_id “PATZ1_NG1”; transcript_id |
| | | | | | “PATZ1_NG1.1”; RPKM “2.9763”; cov |
| | | | | | “31.3120”; |
| chr1 | exon | 1212163 | 1214780 | − | gene_id “PATZ1_NG1”; transcript_id |
| | | | | | “PATZ1_NG1.1”; exon “1”; |
| chr10 | transcript | 134208979 | 134210252 | + | gene_id “PATZ1_NG9”; transcript_id |
| | | | | | “PATZ1_NG9.1”; RPKM “2.8520”; cov |
| | | | | | “30.0039”; |
| chr10 | exon | 134208979 | 134210252 | + | gene_id “PATZ1_NG9”; transcript_id |
| | | | | | “PATZ1_NG9.1”; exon “1”; |
| chr11 | transcript | 2170575 | 2170814 | + | gene_id “PATZ1_NG10”; transcript_id |
| | | | | | “PATZ1_NG10.1”; RPKM “2.6742”; cov |
| | | | | | “28.1333”; |
| chr11 | exon | 2170575 | 2170814 | + | gene_id “PATZ1_NG10”; transcript_id |
| | | | | | “PATZ1_NG10.1”; exon “1”; |
| chr11 | transcript | 69634253 | 69635027 | − | gene_id “PATZ1_NG11”; transcript_id |
| | | | | | “PATZ1_NG11.1”; RPKM “7.6575”; cov |
| | | | | | “80.5587”; |
| chr11 | exon | 69634253 | 69635027 | − | gene_id “PATZ1_NG11”; transcript_id |
| | | | | | “PATZ1_NG11.1”; exon “1”; |
| chr14 | transcript | 104776845 | 104786942 | − | gene_id “PATZ1_NG12”; transcript_id |
| | | | | | “PATZ1_NG12.2”; RPKM “3.3659”; cov |
| | | | | | “35.4104”; |
| chr14 | exon | 104776845 | 104781165 | − | gene_id “PATZ1_NG12”; transcript_id |
| | | | | | “PATZ1_NG12.2”; exon “1”; |
| chr14 | exon | 104782698 | 104786942 | − | gene_id “PATZ1_NG12”; transcript_id |
| | | | | | “PATZ1_NG12.2”; exon “2”; |
| chr14 | transcript | 104776845 | 104786942 | − | gene_id “PATZ1_NG12”; transcript_id |
| | | | | | “PATZ1_NG12.3”; RPKM “4.2419”; cov |
| | | | | | “44.6260”; |
| chr14 | exon | 104776845 | 104786942 | − | gene_id “PATZ1_NG12”; transcript_id |
| | | | | | “PATZ1_NG12.3”; exon “1”; |
| chr14 | transcript | 104787023 | 104787463 | − | gene_id “PATZ1_NG12”; transcript_id |
| | | | | | “PATZ1_NG12.1”; RPKM “2.2179”; cov |
| | | | | | “23.3332”; |
| chr14 | exon | 104787023 | 104787463 | − | gene_id “PATZ1_NG12”; transcript_id |
| | | | | | “PATZ1_NG12.1”; exon “1”; |
| chr15 | transcript | 62982408 | 62983234 | + | gene_id “PATZ1_NG13”; transcript_id |
| | | | | | “PATZ1_NG13.1”; RPKM “21.6819”; cov |
| | | | | | “228.0992”; |
| chr15 | exon | 62982408 | 62983234 | + | gene_id “PATZ1_NG13”; transcript_id |
| | | | | | “PATZ1_NG13.1”; exon “1”; |
| chr17 | transcript | 45922563 | 45922901 | + | gene_id “PATZ1_NG14”; transcript_id |
| | | | | | “PATZ1_NG14.1”; RPKM “3.1071”; cov |
| | | | | | “32.6873”; |
| chr17 | exon | 45922563 | 45922901 | + | gene_id “PATZ1_NG14”; transcript_id |
| | | | | | “PATZ1_NG14.1”; exon “1”; |
| chr17 | transcript | 75783410 | 75785600 | + | gene_id “PATZ1_NG15”; transcript_id |
| | | | | | “PATZ1_NG15.2”; RPKM “2.6370”; cov |
| | | | | | “27.7421”; |
| chr17 | exon | 75783410 | 75785600 | + | gene_id “PATZ1_NG15”; transcript_id |
| | | | | | “PATZ1_NG15.2”; exon “1”; |
| chr17 | transcript | 75783410 | 75785600 | + | gene_id “PATZ1_NG15”; transcript_id |
| | | | | | “PATZ1_NG15.3”; RPKM “2.3067”; cov |
| | | | | | “24.2669”; |
| chr17 | exon | 75783410 | 75783627 | + | gene_id “PATZ1_NG15”; transcript_id |
| | | | | | “PATZ1_NG15.3”; exon “1”; |
| chr17 | exon | 75785376 | 75785600 | + | gene_id “PATZ1_NG15”; transcript_id |
| | | | | | “PATZ1_NG15.3”; exon “2”; |
| chr17 | transcript | 75785653 | 75792293 | + | gene_id “PATZ1_NG15”; transcript_id |
| | | | | | “PATZ1_NG15.1”; RPKM “2.1543”; cov |
| | | | | | “22.6639”; |
| chr17 | exon | 75785653 | 75792293 | + | gene_id “PATZ1_NG15”; transcript_id |
| | | | | | “PATZ1_NG15.1”; exon “1”; |
| chr19 | transcript | 1773302 | 1780143 | − | gene_id “PATZ1_NG16”; transcript_id |
| | | | | | “PATZ1_NG16.1”; RPKM “1.0012”; cov |
| | | | | | “10.5324”; |
| chr19 | exon | 1773302 | 1773539 | − | gene_id “PATZ1_NG16”; transcript_id |
| | | | | | “PATZ1_NG16.1”; exon “1”; |
| chr19 | exon | 1773633 | 1773726 | − | gene_id “PATZ1_NG16”; transcript_id |
| | | | | | “PATZ1_NG16.1”; exon “2”; |
| chr19 | exon | 1774035 | 1774166 | − | gene_id “PATZ1_NG16”; transcript_id |
| | | | | | “PATZ1_NG16.1”; exon “3”; |
| chr19 | exon | 1775229 | 1775362 | − | gene_id “PATZ1_NG16”; transcript_id |
| | | | | | “PATZ1_NG16.1”; exon “4”; |
| chr19 | exon | 1776262 | 1777195 | − | gene_id “PATZ1_NG16”; transcript_id |
| | | | | | “PATZ1_NG16.1”; exon “5”; |
| chr19 | exon | 1779040 | 1779111 | − | gene_id “PATZ1_NG16”; transcript_id |
| | | | | | “PATZ1_NG16.1”; exon “6”; |
| chr19 | exon | 1779204 | 1780143 | − | gene_id “PATZ1_NG16”; transcript_id |
| | | | | | “PATZ1_NG16.1”; exon “7”; |
| chr19 | transcript | 51060154 | 51060950 | + | gene_id “PATZ1_NG17”; transcript_id |
| | | | | | “PATZ1 JMG17.1”; RPKM “4.5928”; cov |
| | | | | | “48.3174”; |
| chr1 9 | exon | 51060154 | 51060950 | + | gene_id “PATZ1_NG17”; transcript_id |
| | | | | | “PATZ1_NG17.1”; exon “1”; |
| chr19 | transcript | 51060968 | 51065241 | + | gene_id “PATZ1_NG18”; transcript_id |
| | | | | | “PATZ1_NG18.1”; RPKM “2.0394”; cov |
| | | | | | “21.4553”; |
| chr19 | exon | 51060968 | 51065241 | + | gene_id “PATZ1_NG18”; transcript_id |
| | | | | | “PATZ1_NG18.1”; exon “1”; |
| chr2 | transcript | 239478138 | 239505638 | + | gene_id “PATZ1_NG2”; transcript_id |
| | | | | | “PATZ1_NG2.1”; RPKM “2.1141”; cov |
| | | | | | “22.2409”; |
| chr2 | exon | 239478138 | 239479055 | + | gene_id “PATZ1_NG2”; transcript_id |
| | | | | | “PATZ1_NG2.1”; exon “1”; |
| chr2 | exon | 239479357 | 239479457 | + | gene_id “PATZ1_NG2”; transcript_id |
| | | | | | “PATZ1_NG2.1”; exon “2”; |
| chr2 | exon | 239503583 | 239505638 | + | gene_id “PATZ1_NG2”; transcript_id |
| | | | | | “PATZ1_NG2.1”; exon “3”; |
| chr2 | transcript | 239478138 | 239505638 | + | gene_id “PATZ1_NG2”; transcript_id |
| | | | | | “PATZ1_NG2.2”; RPKM “1.3301”; cov |
| | | | | | “13.9935”; |
| chr2 | exon | 239478138 | 239479055 | + | gene_id “PATZ1_NG2”; transcript_id |
| | | | | | “PATZ1_NG2.2”; exon “1”; |
| chr2 | exon | 239479357 | 239479457 | + | gene_id “PATZ1_NG2”; transcript_id |
| | | | | | “PATZ1_NG2.2”; exon “2”; |
| chr2 | exon | 239504098 | 239505638 | + | gene_id “PATZ1_NG2”; transcript_id |
| | | | | | “PATZ1_NG2.2”; exon “3”; |
| chr2 | transcript | 239478138 | 239483992 | + | gene_id “PATZ1_NG2”; transcript_id |
| | | | | | “PATZ1_NG2.3”; RPKM “2.5665”; cov |
| | | | | | “27.0000”; |
| chr2 | exon | 239478138 | 239479055 | + | gene_id “PATZ1_NG2”; transcript_id |
| | | | | | “PATZ1_NG2.3”; exon “1”; |
| chr2 | exon | 239479357 | 239483992 | + | gene_id “PATZ1_NG2”; transcript_id |
| | | | | | “PATZ1_NG2.3”; exon “2”; |
| chr2 | transcript | 239620076 | 239621883 | + | gene_id “PATZ1_NG3”; transcript_id |
| | | | | | “PATZ1_NG3.1”; RPKM “3.6585”; cov |
| | | | | | “38.4889”; |
| chr2 | exon | 239620076 | 239621883 | + | gene_id “PATZ1_NG3”; transcript_id |
| | | | | | “PATZ1_NG3.1”; exon “1”; |
| chr2 | transcript | 239621988 | 239624075 | + | gene_id “PATZ1_NG4”; transcript_id |
| | | | | | “PATZ1_NG4.1”; RPKM “4.1061”; cov |
| | | | | | “43.1968”; |
| chr2 | exon | 239621988 | 239624075 | + | gene_id “PATZ1_NG4”; transcript_id |
| | | | | | “PATZ1_NG4.1”; exon “1”; |
| chr20 | transcript | 52809662 | 52831410 | − | gene_id “PATZ1_NG19”; transcript_id |
| | | | | | “PATZ1_NG19.3”; RPKM “0.9689”; cov |
| | | | | | “10.1932”; |
| chr20 | exon | 52809662 | 52810276 | − | gene_id “PATZ1_NG19”; transcript_id |
| | | | | | “PATZ1_NG19.3”; exon “1”; |
| chr20 | exon | 52813950 | 52814792 | − | gene_id “PATZ1_NG19”; transcript_id |
| | | | | | “PATZ1_NG19.3”; exon “2”; |
| chr20 | exon | 52829635 | 52829702 | − | gene_id “PATZ1_NG19”; transcript_id |
| | | | | | “PATZ1_NG19.3”; exon “3”; |
| chr20 | exon | 52831173 | 52831410 | − | gene_id “PATZ1_NG19”; transcript_id |
| | | | | | “PATZ1_NG19.3”; exon “4”; |
| chr20 | transcript | 52813414 | 52831410 | − | gene_id “PATZ1_NG19”; transcript_id |
| | | | | | “PATZ1_NG19.2”; RPKM “2.5514”; cov |
| | | | | | “26.8412”; |
| chr20 | exon | 52813414 | 52814792 | − | gene_id “PATZ1_NG19”; transcript_id |
| | | | | | “PATZ1NG19.2”; exon “1”; |
| chr20 | exon | 52829635 | 52829702 | − | gene_id “PATZ1_NG19”; transcript_id |
| | | | | | “PATZ1_NG19.2”; exon “2”; |
| chr20 | exon | 52831173 | 52831410 | − | gene_id “PATZ1_NG19”; transcript_id |
| | | | | | “PATZ1_NG19.2”; exon “3”; |
| chr20 | transcript | 52823374 | 52831410 | − | gene_id “PATZ1_NG19”; transcript_id |
| | | | | | “PATZ1_NG19.1”; RPKM “3.2138”; cov |
| | | | | | “33.8104”; |
| chr20 | exon | 52823374 | 52823595 | − | gene_id “PATZ1_NG19”; transcript_id |
| | | | | | “PATZ1_NG19.1”; exon “1”; |
| chr20 | exon | 52829635 | 52829702 | − | gene_id “PATZ1_NG19”; transcript_id |
| | | | | | “PATZ1_NG19.1”; exon “2”; |
| chr20 | exon | 52831173 | 52831410 | − | gene_id “PATZ1_NG19”; transcript_id |
| | | | | | “PATZ1_NG19.1”; exon “3”; |
| chr21 | transcript | 47453262 | 47453630 | + | gene_id “PATZ1_NG20”; transcript_id |
| | | | | | “PATZ1_NG20.1”; RPKM “3.7321”; cov |
| | | | | | “39.2624”; |
| chr21 | exon | 47453262 | 47453630 | + | gene_id “PATZ1_NG20”; transcript_id |
| | | | | | “PATZ1_NG20.1”; exon “1”; |
| chr21 | transcript | 47453666 | 47454344 | + | gene_id “PATZ1_NG21”; transcript_id |
| | | | | | “PATZ1_NG21.1”; RPKM “4.0558”; cov |
| | | | | | “42.6686”; |
| chr21 | exon | 47453666 | 47454344 | + | gene_id “PATZ1_NG21”; transcript_id |
| | | | | | “PATZ1_NG21.1”; exon “1”; |
| chr22 | transcript | 41084712 | 41087359 | − | gene_id “PATZ1_NG22”; transcript_id |
| | | | | | “PATZ1_NG22.1”; RPKM “3.6400”; cov |
| | | | | | “38.2938”; |
| chr22 | exon | 41084712 | 41087359 | − | gene_id “PATZ1_NG22”; transcript_id |
| | | | | | “PATZ1_NG22.1”; exon “1”; |
| chr3 | transcript | 195488341 | 195494410 | + | gene_id “PATZ1_NG5”; transcript_id |
| | | | | | “PATZ1_NG5.1”; RPKM “15.0300”; cov |
| | | | | | “158.1202”; |
| chr3 | exon | 195488341 | 195494410 | + | gene_id “PATZ1_NG5”; transcript_id |
| | | | | | “PATZ1_NG5.1”; exon “1”; |
| chr3 | transcript | 195494780 | 195497907 | + | gene_id “PATZ1_NG6”; transcript_id |
| | | | | | “PATZ1_NG6.1”; RPKM “9.4405”; cov |
| | | | | | “99.3168”; |
| chr3 | exon | 195494780 | 195497907 | + | gene_id “PATZ1_NG6”; transcript_id |
| | | | | | “PATZ1_NG6.1”; exon “1”; |
| chr6 | transcript | 168764116 | 168764737 | + | gene_id “PATZ1_NG7”; transcript_id |
| | | | | | “PATZ1_NG7.1”; RPKM “2.8197”; cov |
| | | | | | “29.6640”; |
| chr6 | exon | 168764116 | 168764737 | + | gene_id “PATZ1_NG7”; transcript_id |
| | | | | | “PATZ1_NG7.1”; exon “1”; |
| chr8 | transcript | 143697338 | 143700950 | − | gene_id “PATZ1_NG8”; transcript_id |
| | | | | | “PATZ1_NG8.1”; RPKM “4.1842”; cov |
| | | | | | “44.0190”; |
| chr8 | exon | 143697338 | 143700950 | − | gene_id “PATZ1_NG8”; transcript_id |
| | | | | | “PATZ1_NG8.1”; exon “1”; |
| chr1 | transcript | 142683230 | 142683763 | − | gene_id “SFT_NG1”; transcript_id |
| | | | | | “SFT_NG1.1”; RPKM “3.9685”; cov |
| | | | | | “50.9663”; |
| chr1 | exon | 142683230 | 142683763 | − | gene_id “SFT_NG1”; transcript_id |
| | | | | | “SFT_NG1.1”; exon “1”; |
| chr10 | transcript | 30411990 | 30412288 | + | gene_id “SFT_NG17”; transcript_id |
| | | | | | “SFT_NG17.1”; RPKM “5.9000”; cov |
| | | | | | “75.7726”; |
| chr10 | exon | 30411990 | 30412288 | + | gene_id “SFT_NG17”; transcript_id |
| | | | | | “SFT_NG17.1”; exon “1”; |
| chr11 | transcript | 113929287 | 113930169 | + | gene_id “SFT_NG18”; transcript_id |
| | | | | | “SFT_NG18.1”; RPKM “1.6975”; cov |
| | | | | | “21.8011”; |
| chr11 | exon | 113929287 | 113930169 | + | gene_id “SFT_NG18”; transcript_id |
| | | | | | “SFT_NG18.1”; exon “1”; |
| chr11 | transcript | 125991911 | 126010530 | − | gene_id “SFT_NG19”; transcript_id |
| | | | | | “SFT_NG19.1”; RPKM “0.8982”; cov |
| | | | | | “11.5350”; |
| chr11 | exon | 125991911 | 125994379 | − | gene_id “SFT_NG19”; transcript_id |
| | | | | | “SFT_NG19.1”; exon “1”; |
| chr11 | exon | 125994529 | 125994716 | − | gene_id “SFT_NG19”; transcript_id |
| | | | | | “SFT_NG19.1”; exon “2”; |
| chr11 | exon | 125997891 | 125998088 | − | gene_id “SFT_NG19”; transcript_id |
| | | | | | “SFT_NG19.1”; exon “3”; |
| chr11 | exon | 126010014 | 126010530 | − | gene_id “SFT_NG19”; transcript_id |
| | | | | | “SFT_NG19.1”; exon “4”; |
| chr11 | transcript | 125991156 | 125991810 | − | gene_id “SFT_NG20”; transcript_id |
| | | | | | “SFT_NG20.1”; RPKM “2.2721”; cov |
| | | | | | “29.1802”; |
| chr11 | exon | 125991156 | 125991810 | − | gene_id “SFT_NG20”; transcript_id |
| | | | | | “SFT_NG20.1”; exon “1”; |
| chr12 | transcript | 52219697 | 52220242 | − | gene_id “SFT_NG21”; transcript_id |
| | | | | | “SFT_NG21.1”; RPKM “3.7953”; cov |
| | | | | | “48.7418”; |
| chr12 | exon | 52219697 | 52220242 | − | gene_id “SFT_NG21”; transcript_id |
| | | | | | “SFT_NG21.1”; exon “1”; |
| chr14 | transcript | 19959511 | 19960440 | − | gene_id “SFT_NG22”; transcript_id |
| | | | | | “SFT_NG22.1”; RPKM “2.8731”; cov |
| | | | | | “36.8978”; |
| chr14 | exon | 19959511 | 19960440 | − | gene_id “SFT_NG22”; transcript_id |
| | | | | | “SFT_NG22.1”; exon “1”; |
| chr15 | transcript | 70415043 | 70439551 | + | gene_id “SFT_NG23”; transcript_id |
| | | | | | “SFT_NG23.3”; RPKM “1.2002”; cov |
| | | | | | “15.4142”; |
| chr15 | exon | 70415043 | 70416063 | + | gene_id “SFT_NG23”; transcript_id |
| | | | | | “SFT_NG23.3”; exon “1”; |
| chr15 | exon | 70418208 | 70426082 | + | gene_id “SFT_NG23”; transcript_id |
| | | | | | “SFT_NG23.3”; exon “2”; |
| chr15 | exon | 70428193 | 70428354 | + | gene_id “SFT_NG23”; transcript_id |
| | | | | | “SFT_NG23.3”; exon “3”; |
| chr15 | exon | 70434123 | 70439551 | + | gene_id “SFT_NG23”; transcript_id |
| | | | | | “SFT_NG23.3”; exon “4”; |
| chr15 | transcript | 70415043 | 70439551 | + | gene_id “SFT_NG23”; transcript_id |
| | | | | | “SFT_NG23.2”; RPKM “1.5012”; cov |
| | | | | | “19.2789”; |
| chr15 | exon | 70415043 | 70416063 | + | gene_id “SFT_NG23”; transcript_id |
| | | | | | “SFT_NG23.2”; exon “1”; |
| chr15 | exon | 70418208 | 70422746 | + | gene_id “SFT_NG23”; transcript_id |
| | | | | | “SFT_NG23.2”; exon “2”; |
| chr15 | exon | 70434123 | 70439551 | + | gene_id “SFT_NG23”; transcript_id |
| | | | | | “SFT_NG23.2”; exon “3”; |
| chr15 | transcript | 70415043 | 70417653 | + | gene_id “SFT_NG23”; transcript_id |
| | | | | | “SFT_NG23.1”; RPKM “7.2379”; cov |
| | | | | | “92.9542”; |
| chr15 | exon | 70415043 | 70416063 | + | gene_id “SFT_NG23”; transcript_id |
| | | | | | “SFT_NG23.1”; exon “1”; |
| chr15 | exon | 70416370 | 70417653 | + | gene_id “SFT_NG23”; transcript_id |
| | | | | | “SFT_NG23.1”; exon “2”; |
| chr15 | transcript | 70415043 | 70439551 | + | gene_id “SFT_NG23”; transcript_id |
| | | | | | “SFT_NG23.5”; RPKM “0.9600”; cov |
| | | | | | “12.3285”; |
| chr15 | exon | 70415043 | 70416063 | + | gene_id “SFT_NG23”; transcript_id |
| | | | | | “SFT_NG23.5”; exon “1”; |
| chr15 | exon | 70418208 | 70429114 | + | gene_id “SFT_NG23”; transcript_id |
| | | | | | “SFT_NG23.5”; exon “2”; |
| chr15 | exon | 70434123 | 70439551 | + | gene_id “SFT_NG23”; transcript_id |
| | | | | | “SFT_NG23.5”; exon “3”; |
| chr15 | transcript | 70415043 | 70439551 | + | gene_id “SFT_NG23”; transcript_id |
| | | | | | “SFT_NG23.4”; RPKM “0.8231”; cov |
| | | | | | “10.5715”; |
| chr15 | exon | 70415043 | 70416063 | + | gene_id “SFT_NG23”; transcript_id |
| | | | | | “SFT_NG23.4”; exon “1”; |
| chr15 | exon | 70418208 | 70426082 | + | gene_id “SFT_NG23”; transcript_id |
| | | | | | “SFT_NG23.4”; exon “2”; |
| chr15 | exon | 70428193 | 70429114 | + | gene_id “SFT_NG23”; transcript_id |
| | | | | | “SFT_NG23.4”; exon “3”; |
| chr15 | exon | 70434123 | 70439551 | + | gene_id “SFT_NG23”; transcript_id |
| | | | | | “SFT_NG23.4”; exon “4”; |
| chr15 | transcript | 70911175 | 70916508 | − | gene_id “SFT_NG24”; transcript_id |
| | | | | | “SFT_NG24.1”; RPKM “4.0989”; cov |
| | | | | | “52.6406”; |
| chr15 | exon | 70911175 | 70916508 | − | gene_id “SFT_NG24”; transcript_id |
| | | | | | “SFT_NG24.1”; exon “1”; |
| chr15 | transcript | 71101764 | 71115313 | + | gene_id “SFT_NG25”; transcript_id |
| | | | | | “SFT_NG25.1”; RPKM “2.0099”; cov |
| | | | | | “25.8122”; |
| chr15 | exon | 71101764 | 71102098 | + | gene_id “SFT_NG25”; transcript_id |
| | | | | | “SFT_NG25.1”; exon “1”; |
| chr15 | exon | 71102735 | 71103060 | + | gene_id “SFT_NG25”; transcript_id |
| | | | | | “SFT_NG25.1”; exon “2”; |
| chr15 | exon | 71109090 | 71109264 | + | gene_id “SFT_NG25”; transcript_id |
| | | | | | “SFT_NG25.1”; exon “3”; |
| chr15 | exon | 71109406 | 71115313 | + | gene_id “SFT_NG25”; transcript_id |
| | | | | | “SFT_NG25.1”; exon “4”; |
| chr15 | transcript | 71101764 | 71115313 | + | gene_id “SFT_NG25”; transcript_id |
| | | | | | “SFT_NG25.2”; RPKM “6.9654”; cov |
| | | | | | “89.4548”; |
| chr15 | exon | 71101764 | 71106777 | + | gene_id “SFT_NG25”; transcript_id |
| | | | | | “SFT_NG25.2”; exon “1”; |
| chr15 | exon | 71106824 | 71109264 | + | gene_id “SFT_NG25”; transcript_id |
| | | | | | “SFT_NG25.2”; exon “2”; |
| chr15 | exon | 71109406 | 71115313 | + | gene_id “SFT_NG25”; transcript_id |
| | | | | | “SFT_NG25.2”; exon “3”; |
| chr15 | transcript | 71101764 | 71115313 | + | gene_id “SFT_NG25”; transcript_id |
| | | | | | “SFT_NG25.3”; RPKM “5.8781”; cov |
| | | | | | “75.4908”; |
| chr15 | exon | 71101764 | 71109264 | + | gene_id “SFT_NG25”; transcript_id |
| | | | | | “SFT_NG25.3”; exon “1”; |
| chr15 | exon | 71109406 | 71115313 | + | gene_id “SFT_NG25”; transcript_id |
| | | | | | “SFT_NG25.3”; exon “2”; |
| chr16 | transcript | 85363530 | 85364961 | + | gene_id “SFT_NG26”; transcript_id |
| | | | | | “SFT_NG26.1”; RPKM “4.2046”; cov |
| | | | | | “53.9986”; |
| chr16 | exon | 85363530 | 85364961 | + | gene_id “SFT_NG26”; transcript_id |
| | | | | | “SFT_NG26.1”; exon “1”; |
| chr17 | transcript | 48035752 | 48037167 | − | gene_id “SFT_NG27”; transcript_id |
| | | | | | “SFT_NG27.1”; RPKM “6.0701”; cov |
| | | | | | “77.9562”; |
| chr17 | exon | 48035752 | 48037167 | − | gene_id “SFT_NG27”; transcript_id |
| | | | | | “SFT_NG27.1exon “1”; |
| chr17 | transcript | 48037188 | 48037448 | − | gene_id “SFT_NG28”; transcript_id |
| | | | | | “SFT_NG28.1”; RPKM “4.9350”; cov |
| | | | | | “63.3793”; |
| chr17 | exon | 48037188 | 48037448 | − | gene_id “SFT_NG28”; transcript_id |
| | | | | | “SFT_NG28.1”; exon “1”; |
| chr18 | transcript | 45935913 | 45943415 | + | gene_id “SFT_NG29”; transcript_id |
| | | | | | “SFT_NG29.1”; RPKM “2.5746”; cov |
| | | | | | “33.0651”; |
| chr18 | exon | 45935913 | 45943415 | + | gene_id “SFT_NG29”; transcript_id |
| | | | | | “SFT_NG29.1”; exon “1”; |
| chr18 | transcript | 46491779 | 46493562 | − | gene_id “SFT_NG30”; transcript_id |
| | | | | | “SFT_NG30.1”; RPKM “1.7619”; cov |
| | | | | | “22.6277”; |
| chr18 | exon | 46491779 | 46493562 | − | gene_id “SFT_NG30”; transcript_id |
| | | | | | “SFT_NG30.1”; exon “1”; |
| chr18 | transcript | 46493740 | 46496736 | − | gene_id “SFT_NG30”; transcript_id |
| | | | | | “SFT_NG30.2”; RPKM “1.9060”; cov |
| | | | | | “24.4788”; |
| chr18 | exon | 46493740 | 46496736 | − | gene_id “SFT_NG30”; transcript_id |
| | | | | | “SFT_NG30.2”; exon “1”; |
| chr19 | transcript | 1860130 | 1862091 | − | gene_id “SFT_NG31”; transcript_id |
| | | | | | “SFT_NG31.1”; RPKM “1.9486”; cov |
| | | | | | “25.0257”; |
| chr19 | exon | 1860130 | 1862091 | − | gene_id “SFT_NG31”; transcript_id |
| | | | | | “SFT_NG31.1”; exon “1”; |
| chr19 | transcript | 15217570 | 15218137 | + | gene_id “SFT_NG32”; transcript_id |
| | | | | | “SFT_NG32.1”; RPKM “1.0936”; cov |
| | | | | | “14.0444”; |
| chr19 | exon | 15217570 | 15217705 | + | gene_id “SFT_NG32”; transcript_id |
| | | | | | “SFT_NG32.1”; exon “1”; |
| chr19 | exon | 15217818 | 15218137 | + | gene_id “SFT_NG32”; transcript_id |
| | | | | | “SFT_NG32.1”; exon “2”; |
| chr19 | transcript | 46603199 | 46621713 | − | gene_id “SFT_NG33”; transcript_id |
| | | | | | “SFT_NG33.1”; RPKM “2.2762”; cov |
| | | | | | “29.2330”; |
| chr19 | exon | 46603199 | 46605136 | − | gene_id “SFT_NG33”; transcript_id |
| | | | | | “SFT_NG33.1”; exon “1”; |
| chr19 | exon | 46612199 | 46612353 | − | gene_id “SFT_NG33”; transcript_id |
| | | | | | “SFT_NG33.1”; exon “2”; |
| chr19 | exon | 46619074 | 46619278 | − | gene_id “SFT_NG33”; transcript_id |
| | | | | | “SFT_NG33.1”; exon “3”; |
| chr19 | exon | 46621562 | 46621713 | − | gene_id “SFT_NG33”; transcript_id |
| | | | | | “SFT_NG33.1”; exon “4”; |
| chr2 | transcript | 40841005 | 40875354 | − | gene_id “SFT_NG2”; transcript_id |
| | | | | | “SFT_NG2.1”; RPKM “1.0623”; cov |
| | | | | | “13.6430”; |
| chr2 | exon | 40841005 | 40845769 | − | gene_id “SFT_NG2”; transcript_id |
| | | | | | “SFT_NG2.1”; exon “1”; |
| chr2 | exon | 40845916 | 40846062 | − | gene_id “SFT_NG2”; transcript_id |
| | | | | | “SFT_NG2.1”; exon “2”; |
| chr2 | exon | 40847405 | 40847459 | − | gene_id “SFT_NG2”; transcript_id |
| | | | | | “SFT_NG2.1”; exon “3”; |
| chr2 | exon | 40847806 | 40847858 | − | gene_id “SFT_NG2”; transcript_id |
| | | | | | “SFT_NG2.1”; exon “4”; |
| chr2 | exon | 40866233 | 40866285 | − | gene_id “SFT_NG2”; transcript_id |
| | | | | | “SFT_NG2.1”; exon “5”; |
| chr2 | exon | 40875231 | 40875354 | − | gene_id “SFT_NG2”; transcript_id |
| | | | | | “SFT_NG2.1”; exon “6”; |
| chr2 | transcript | 42274184 | 42274859 | + | gene_id “SFT_NG3”; transcript_id |
| | | | | | “SFT_NG3.1”; RPKM “1.7380”; cov |
| | | | | | “22.3210”; |
| chr2 | exon | 42274184 | 42274859 | + | gene_id “SFT_NG3”; transcript_id |
| | | | | | “SFT_NG3.1”; exon “1”; |
| chr2 | transcript | 78485784 | 78605970 | + | gene_id “SFT_NG4”; transcript_id |
| | | | | | “SFT_NG4.5”; RPKM “0.7852”; cov |
| | | | | | “10.0836”; |
| chr2 | exon | 78485784 | 78486086 | + | gene_id “SFT_NG4”; transcript_id |
| | | | | | “SFT_NG4.5”; exon “1”; |
| chr2 | exon | 78545547 | 78545619 | + | gene_id “SFT_NG4”; transcript_id |
| | | | | | “SFT_NG4.5”; exon “2”; |
| chr2 | exon | 78545877 | 78545971 | + | gene_id “SFT_NG4”; transcript_id |
| | | | | | “SFT_NG4.5”; exon “3”; |
| chr2 | exon | 78603272 | 78605970 | + | gene_id “SFT_NG4”; transcript_id |
| | | | | | “SFT_NG4.5”; exon “4”; |
| chr2 | transcript | 78485784 | 78593232 | + | gene_id “SFT_NG4”; transcript_id |
| | | | | | “SFT_NG4.6”; RPKM “1.8657”; cov |
| | | | | | “23.9610”; |
| chr2 | exon | 78485784 | 78486086 | + | gene_id “SFT_NG4”; transcript_id |
| | | | | | “SFT_NG4.6”; exon “1”; |
| chr2 | exon | 78545547 | 78545619 | + | gene_id “SFT_NG4”; transcript_id |
| | | | | | “SFT_NG4.6”; exon “2”; |
| chr2 | exon | 78545877 | 78545971 | + | gene_id “SFT_NG4”; transcript_id |
| | | | | | “SFT_NG4.6”; exon “3”; |
| chr2 | exon | 78580161 | 78580252 | + | gene_id “SFT_NG4”; transcript_id |
| | | | | | “SFT_NG4.6”; exon “4”; |
| chr2 | exon | 78588656 | 78593232 | + | gene_id “SFT_NG4”; transcript_id |
| | | | | | “SFT_NG4.6”; exon “5”; |
| chr2 | transcript | 78485784 | 78593232 | + | gene_id “SFT_NG4”; transcript_id |
| | | | | | “SFT_NG4.7”; RPKM “1.1276”; cov |
| | | | | | “14.4812”; |
| chr2 | exon | 78485784 | 78486086 | + | gene_id “SFT_NG4”; transcript_id |
| | | | | | “SFT_NG4.7”; exon “1”; |
| chr2 | exon | 78545547 | 78545619 | + | gene_id “SFT_NG4”; transcript_id |
| | | | | | “SFT_NG4.7”; exon “2”; |
| chr2 | exon | 78545877 | 78545971 | + | gene_id “SFT_NG4”; transcript_id |
| | | | | | “SFT_NG4.7”; exon “3”; |
| chr2 | exon | 78588656 | 78593232 | + | gene_id “SFT_NG4”; transcript_id |
| | | | | | “SFT_NG4.7”; exon “4”; |
| chr2 | transcript | 78485784 | 78593232 | + | gene_id “SFT_NG4”; transcript_id |
| | | | | | “SFT_NG4.8”; RPKM “1.5300”; cov |
| | | | | | “19.6487”; |
| chr2 | exon | 78485784 | 78486086 | + | gene_id “SFT_NG4”; transcript_id |
| | | | | | “SFT_NG4.8”; exon “1”; |
| chr2 | exon | 78545547 | 78545619 | + | gene_id “SFT_NG4”; transcript_id |
| | | | | | “SFT_NG4.8”; exon “2”; |
| chr2 | exon | 78545877 | 78545971 | + | gene_id “SFT_NG4”; transcript_id |
| | | | | | “SFT_NG4.8”; exon “3”; |
| chr2 | exon | 78560741 | 78560859 | + | gene_id “SFT_NG4”; transcript_id |
| | | | | | “SFT_NG4.8”; exon “4”; |
| chr2 | exon | 78588656 | 78593232 | + | gene_id “SFT_NG4”; transcript_id |
| | | | | | “SFT_NG4.8”; exon “5”; |
| chr2 | transcript | 78485784 | 78582099 | + | gene_id “SFT_NG4”; transcript_id |
| | | | | | “SFT_NG4.9”; RPKM “0.8169”; cov |
| | | | | | “10.4915”; |
| chr2 | exon | 78485784 | 78486086 | + | gene_id “SFT_NG4”; transcript_id |
| | | | | | “SFT_NG4.9”; exon “1”; |
| chr2 | exon | 78545547 | 78545619 | + | gene_id “SFT_NG4”; transcript_id |
| | | | | | “SFT_NG4.9”; exon “2”; |
| chr2 | exon | 78545877 | 78545971 | + | gene_id “SFT_NG4”; transcript_id |
| | | | | | “SFT_NG4.9”; exon “3”; |
| chr2 | exon | 78560741 | 78560859 | + | gene_id “SFT_NG4”; transcript_id |
| | | | | | “SFT_NG4.9”; exon “4”; |
| chr2 | exon | 78580161 | 78582099 | + | gene_id “SFT_NG4”; transcript_id |
| | | | | | “SFT_NG4.9”; exon “5”; |
| chr2 | transcript | 78485784 | 78553796 | + | gene_id “SFT_NG4”; transcript_id |
| | | | | | “SFT_NG4.3”; RPKM “1.0557”; cov |
| | | | | | “13.5578”; |
| chr2 | exon | 78485784 | 78486086 | + | gene_id “SFT_NG4”; transcript_id |
| | | | | | “SFT_NG4.3”; exon “1”; |
| chr2 | exon | 78545547 | 78545652 | + | gene_id “SFT_NG4”; transcript_id |
| | | | | | “SFT_NG4.3”; exon “2”; |
| chr2 | exon | 78545877 | 78545971 | + | gene_id “SFT_NG4”; transcript_id |
| | | | | | “SFT_NG4.3”; exon “3”; |
| chr2 | exon | 78550761 | 78553796 | + | gene_id “SFT_NG4”; transcript_id |
| | | | | | “SFT_NG4.3”; exon “4”; |
| chr2 | transcript | 78485784 | 78545759 | + | gene_id “SFT_NG4”; transcript_id |
| | | | | | “SFT_NG4.2”; RPKM “5.6629”; cov |
| | | | | | “72.7268”; |
| chr2 | exon | 78485784 | 78486086 | + | gene_id “SFT_NG4”; transcript_id |
| | | | | | “SFT_NG4.2”; exon “1”; |
| chr2 | exon | 78545547 | 78545759 | + | gene_id “SFT_NG4”; transcript_id |
| | | | | | “SFT_NG4.2”; exon “2”; |
| chr2 | transcript | 78543617 | 78553796 | + | gene_id “SFT_NG4”; transcript_id |
| | | | | | “SFT_NG4.4”; RPKM “1.4526”; cov |
| | | | | | “18.6547”; |
| chr2 | exon | 78543617 | 78545619 | + | gene_id “SFT_NG4”; transcript_id |
| | | | | | “SFT_NG4.4”; exon “1”; |
| chr2 | exon | 78545877 | 78545971 | + | gene_id “SFT_NG4”; transcript_id |
| | | | | | “SFT_NG4.4”; exon “2”; |
| chr2 | exon | 78550761 | 78553796 | + | gene_id “SFT_NG4”; transcript_id |
| | | | | | “SFT_NG4.4”; exon “3”; |
| chr2 | transcript | 78585803 | 78593232 | + | gene_id “SFT_NG4”; transcript_id |
| | | | | | “SFT_NG4.1”; RPKM “1.8700”; cov |
| | | | | | “24.0155”; |
| chr2 | exon | 78585803 | 78593232 | + | gene_id “SFT_NG4”; transcript_id |
| | | | | | “SFT_NG4.1”; exon “1”; |
| chr2 | transcript | 157492668 | 157511012 | − | gene_id “SFT_NG5”; transcript_id |
| | | | | | “SFT_NG5.1”; RPKM “2.3180”; cov |
| | | | | | “29.7699”; |
| chr2 | exon | 157492668 | 157496164 | − | gene_id “SFT_NG5”; transcript_id |
| | | | | | “SFT_NG5.1”; exon “1”; |
| chr2 | exon | 157498197 | 157498338 | − | gene_id “SFT_NG5”; transcript_id |
| | | | | | “SFT_NG5.1”; exon “2”; |
| chr2 | exon | 157503898 | 157504173 | − | gene_id “SFT_NG5”; transcript_id |
| | | | | | “SFT_NG5.1”; exon “3”; |
| chr2 | exon | 157507476 | 157507593 | − | gene_id “SFT_NG5”; transcript_id |
| | | | | | “SFT_NG5.1”; exon “4”; |
| chr2 | exon | 157510573 | 157511012 | − | gene_id “SFT_NG5”; transcript_id |
| | | | | | “SFT_NG5.1”; exon “5”; |
| chr2 | transcript | 157492668 | 157511012 | − | gene_id “SFT_NG5”; transcript_id |
| | | | | | “SFT_NG5.2”; RPKM “3.3591”; cov |
| | | | | | “43.1400”; |
| chr2 | exon | 157492668 | 157496164 | − | gene_id “SFT_NG5”; transcript_id |
| | | | | | “SFT_NG5.2”; exon “1”; |
| chr2 | exon | 157498197 | 157498338 | − | gene_id “SFT_NG5”; transcript_id |
| | | | | | “SFT_NG5.2”; exon “2”; |
| chr2 | exon | 157503898 | 157504173 | − | gene_id “SFT_NG5”; transcript_id |
| | | | | | “SFT_NG5.2”; exon “3”; |
| chr2 | exon | 157507645 | 157508107 | − | gene_id “SFT_NG5”; transcript_id |
| | | | | | “SFT_NG5.2”; exon “4”; |
| chr2 | exon | 157509640 | 157509729 | − | gene_id “SFT_NG5”; transcript_id |
| | | | | | “SFT_NG5.2”; exon “5”; |
| chr2 | exon | 157510573 | 157511012 | − | gene_id “SFT_NG5”; transcript_id |
| | | | | | “SFT_NG5.2”; exon “6”; |
| chr22 | transcript | 16288123 | 16288552 | − | gene_id “SFT_NG34”; transcript_id |
| | | | | | “SFT_NG34.1”; RPKM “8.7515”; cov |
| | | | | | “112.3930”; |
| chr22 | exon | 16288123 | 16288552 | − | gene_id “SFT_NG34”; transcript_id |
| | | | | | “SFT_NG34.1”; exon “1”; |
| chr22 | transcript | 30723059 | 30723431 | − | gene_id “SFT_NG35”; transcript_id |
| | | | | | “SFT_NG35.1”; RPKM “3.0376”; cov |
| | | | | | “39.0107”; |
| chr22 | exon | 30723059 | 30723431 | − | gene_id “SFT_NG35”; transcript_id |
| | | | | | “SFT_NG35.1”; exon “1”; |
| chr3 | transcript | 52056479 | 52058737 | + | gene_id “SFT_NG6”; transcript_id |
| | | | | | “SFT_NG6.1”; RPKM “1.6012”; cov |
| | | | | | “20.5642”; |
| chr3 | exon | 52056479 | 52058737 | + | gene_id “SFT_NG6”; transcript_id |
| | | | | | “SFT_NG6.1”; exon “1”; |
| chr3 | transcript | 52488135 | 52489443 | − | gene_id “SFT_NG7”; transcript_id |
| | | | | | “SFT_NG7.1”; RPKM “1.6606”; cov |
| | | | | | “21.3270”; |
| chr3 | exon | 52488135 | 52489443 | − | gene_id “SFT_NG7”; transcript_id |
| | | | | | “SFT_NG7.1”; exon “1”; |
| chr3 | transcript | 87235969 | 87258938 | + | gene_id “SFT_NG8”; transcript_id |
| | | | | | “SFT_NG8.1”; RPKM “2.3137”; cov |
| | | | | | “29.7144”; |
| chr3 | exon | 87235969 | 87236116 | + | gene_id “SFT_NG8”; transcript_id |
| | | | | | “SFT_NG8.1”; exon “1”; |
| chr3 | exon | 87238393 | 87238579 | + | gene_id “SFT_NG8”; transcript_id |
| | | | | | “SFT_NG8.1”; exon “2”; |
| chr3 | exon | 87239325 | 87239534 | + | gene_id “SFT_NG8”; transcript_id |
| | | | | | “SFT_NG8.1”; exon “3”; |
| chr3 | exon | 87255460 | 87258938 | + | gene_id “SFT_NG8”; transcript_id |
| | | | | | “SFT_NG8.1”; exon “4”; |
| chr3 | transcript | 87235969 | 87258938 | + | gene_id “SFT_NG8”; transcript_id |
| | | | | | “SFT_NG8.2”; RPKM “4.1247”; cov |
| | | | | | “52.9719”; |
| chr3 | exon | 87235969 | 87236116 | + | gene_id “SFT_NG8”; transcript_id |
| | | | | | “SFT_NG8.2”; exon “1”; |
| chr3 | exon | 87239325 | 87239534 | + | gene_id “SFT_NG8”; transcript_id |
| | | | | | “SFT_NG8.2”; exon “2”; |
| chr3 | exon | 87255460 | 87258938 | + | gene_id “SFT_NG8”; transcript_id |
| | | | | | “SFT_NG8.2”; exon “3”; |
| chr3 | transcript | 87235969 | 87258938 | + | gene_id “SFT_NG8”; transcript_id |
| | | | | | “SFT_NG8.3”; RPKM “2.2814”; cov |
| | | | | | “29.2990”; |
| chr3 | exon | 87235969 | 87236116 | + | gene_id “SFT_NG8”; transcript_id |
| | | | | | “SFT_NG8.3”; exon “1”; |
| chr3 | exon | 87239325 | 87239534 | + | gene_id “SFT_NG8”; transcript_id |
| | | | | | “SFT_NG8.3”; exon “2”; |
| chr3 | exon | 87255460 | 87255548 | + | gene_id “SFT_NG8”; transcript_id |
| | | | | | “SFT_NG8.3”; exon “3”; |
| chr3 | exon | 87255968 | 87258938 | + | gene_id “SFT_NG8”; transcript_id |
| | | | | | “SFT_NG8.3”; exon “4”; |
| chr3 | transcript | 184429880 | 184430804 | + | gene_id “SFT_NG9”; transcript_id |
| | | | | | “SFT_NG9.1”; RPKM “4.2790”; cov |
| | | | | | “54.9540”; |
| chr3 | exon | 184429880 | 184430804 | + | gene_id “SFT_NG9”; transcript_id |
| | | | | | “SFT_NG9.1”; exon “1”; |
| chr3 | transcript | 184442119 | 184446869 | + | gene_id “SFT_NG9”; transcript_id |
| | | | | | “SFT_NG9.2”; RPKM “1.1517”; cov |
| | | | | | “14.7911”; |
| chr3 | exon | 184442119 | 184443993 | + | gene_id “SFT_NG9”; transcript_id |
| | | | | | “SFT_NG9.2”; exon “1”; |
| chr3 | exon | 184445188 | 184445368 | + | gene_id “SFT_NG9”; transcript_id |
| | | | | | “SFT_NG9.2”; exon “2”; |
| chr3 | exon | 184446622 | 184446869 | + | gene_id “SFT_NG9”; transcript_id |
| | | | | | “SFT_NG9.2”; exon “3”; |
| chr5 | transcript | 115117574 | 115125147 | + | gene_id “SFT_NG10”; transcript_id |
| | | | | | “SFT_NG10.1”; RPKM “0.8285”; cov |
| | | | | | “10.6399”; |
| chr5 | exon | 115117574 | 115117698 | + | gene_id “SFT_NG10”; transcript_id |
| | | | | | “SFT_NG10.1”; exon “1”; |
| chr5 | exon | 115121124 | 115125147 | + | gene_id “SFT_NG10”; transcript_id |
| | | | | | “SFT_NG10.1”; exon “2”; |
| chr6 | transcript | 41420105 | 41457322 | − | gene_id “SFT_NG11”; transcript_id |
| | | | | | “SFT_NG11.1”; RPKM “1.7938”; cov |
| | | | | | “23.0371”; |
| chr6 | exon | 41420105 | 41421058 | − | gene_id “SFT_NG11”; transcript_id |
| | | | | | “SFT_NG11.1”; exon “1”; |
| chr6 | exon | 41454803 | 41454867 | − | gene_id “SFT_NG11”; transcript_id |
| | | | | | “SFT_NG11.1”; exon “2”; |
| chr6 | exon | 41457219 | 41457322 | − | gene_id “SFT_NG11”; transcript_id |
| | | | | | “SFT_NG11.1”; exon “3”; |
| chr6 | transcript | 41471782 | 41472769 | + | gene_id “SFT_NG12”; transcript_id |
| | | | | | “SFT_NG12.1”; RPKM “2.3489”; cov |
| | | | | | “30.1660”; |
| chr6 | exon | 41471782 | 41472769 | + | gene_id “SFT_NG12”; transcript_id |
| | | | | | “SFT_NG12.1”; exon “1”; |
| chr6 | transcript | 43706320 | 43736422 | + | gene_id “SFT_NG13”; transcript_id |
| | | | | | “SFT_NG13.1”; RPKM “1.7937”; cov |
| | | | | | “23.0357”; |
| chr6 | exon | 43706320 | 43706643 | + | gene_id “SFT_NG13”; transcript_id |
| | | | | | “SFT_NG13.1”; exon “1”; |
| chr6 | exon | 43735802 | 43736422 | + | gene_id “SFT_NG13”; transcript_id |
| | | | | | “SFT_NG13.1”; exon “2”; |
| chr8 | transcript | 49313376 | 49396211 | + | gene_id “SFT_NG14”; transcript_id |
| | | | | | “SFT_NG14.1”; RPKM “2.0826”; cov |
| | | | | | “26.7458”; |
| chr8 | exon | 49313376 | 49314160 | + | gene_id “SFT_NG14”; transcript_id |
| | | | | | “SFT_NG14.1”; exon “1”; |
| chr8 | exon | 49315862 | 49315985 | + | gene_id “SFT_NG14”; transcript_id |
| | | | | | “SFT_NG14.1”; exon “2”; |
| chr8 | exon | 49336913 | 49337075 | + | gene_id “SFT_NG14”; transcript_id |
| | | | | | “SFT_NG14.1”; exon “3”; |
| chr8 | exon | 49386058 | 49396211 | + | gene_id “SFT_NG14”; transcript_id |
| | | | | | “SFT_NG14.1”; exon “4”; |
| chr9 | transcript | 70838941 | 70842110 | + | gene_id “SFT_NG15”; transcript_id |
| | | | | | “SFT_NG15.1”; RPKM “1.6529”; cov |
| | | | | | “21.2274”; |
| chr9 | exon | 70838941 | 70842110 | + | gene_id “SFT_NG15”; transcript_id |
| | | | | | “SFT_NG15.1”; exon “1”; |
| chr9 | transcript | 126761041 | 126761848 | + | gene_id “SFT_NG16”; transcript_id |
| | | | | | “SFT_NG16.1”; RPKM “2.2860”; cov |
| | | | | | “29.3584”; |
| chr9 | exon | 126761041 | 126761848 | + | gene_id “SFT_NG16”; transcript_id |
| | | | | | “SFT_NG16.1”; exon “1”; |
| chrUn_gl000224 | transcript | 32128 | 32740 | − | gene_id “SFT_NG36”; transcript_id |
| | | | | | “SFT_NG36.1”; RPKM “1.7292”; cov |
| | | | | | “22.2072”; |
| chrUn_gl000224 | exon | 32128 | 32740 | − | gene_id “SFT_NG36”; transcript_id |
| | | | | | “SFT_NG36.1”; exon “1”; |
| chr1 | transcript | 197928257 | 197975079 | + | gene_id “TFE3_NG1”; transcript_id |
| | | | | | “TFE3_NG1.1”; RPKM “1.1747”; cov |
| | | | | | “17.0491”; |
| chr1 | exon | 197928257 | 197928347 | + | gene_id “TFE3_NG1”; transcript_id |
| | | | | | “TFE3_NG1.1”; exon “1”; |
| chr1 | exon | 197966750 | 197966879 | + | gene_id “TFE3_NG1”; transcript_id |
| | | | | | “TFE3_NG1.1”; exon “2”; |
| chr1 | exon | 197969301 | 197975079 | + | gene_id “TFE3_NG1”; transcript_id |
| | | | | | “TFE3_NG1.1”; exon “3”; |
| chr1 | transcript | 197928257 | 198018376 | + | gene_id “TFE3_NG1”; transcript_id |
| | | | | | “TFE3_NG1.2”; RPKM “0.9345”; cov |
| | | | | | “13.5628”; |
| chr1 | exon | 197928257 | 197928347 | + | gene_id “TFE3_NG1”; transcript_id |
| | | | | | “TFE3_NG1.2”; exon “1”; |
| chr1 | exon | 198004177 | 198004334 | + | gene_id “TFE3_NG1”; transcript_id |
| | | | | | “TFE3_NG1.2”; exon “2”; |
| chr1 | exon | 198006277 | 198006464 | + | gene_id “TFE3_NG1”; transcript_id |
| | | | | | “TFE3_NG1.2”; exon “3”; |
| chr1 | exon | 198010742 | 198010799 | + | gene_id “TFE3_NG1”; transcript_id |
| | | | | | “TFE3_NG1.2”; exon “4”; |
| chr1 | exon | 198017865 | 198018376 | + | gene_id “TFE3_NG1”; transcript_id |
| | | | | | “TFE3_NG1.2”; exon “5”; |
| chr1 | transcript | 197928257 | 198014754 | + | gene_id “TFE3_NG1”; transcript_id |
| | | | | | “TFE3_NG1.3”; RPKM “1.9316”; cov |
| | | | | | “28.0349”; |
| chr1 | exon | 197928257 | 197928347 | + | gene_id “TFE3_NG1”; transcript_id |
| | | | | | “TFE3_NG1.3”; exon “1”; |
| chr1 | exon | 198004177 | 198004334 | + | gene_id “TFE3_NG1”; transcript_id |
| | | | | | “TFE3_NG1.3”; exon “2”; |
| chr1 | exon | 198006277 | 198014754 | + | gene_id “TFE3_NG1”; transcript_id |
| | | | | | “TFE3_NG1.3”; exon “3”; |
| chr1 | transcript | 197928257 | 198014754 | + | gene_id “TFE3_NG1”; transcript_id |
| | | | | | “TFE3_NG1.4”; RPKM “0.9085”; cov |
| | | | | | “13.1861”; |
| chr1 | exon | 197928257 | 197928347 | + | gene_id “TFE3_NG1”; transcript_id |
| | | | | | “TFE3_NG1.4”; exon “1”; |
| chr1 | exon | 198004177 | 198004334 | + | gene_id “TFE3_NG1”; transcript_id |
| | | | | | “TFE3_NG1.4”; exon “2”; |
| chr1 | exon | 198006277 | 198006464 | + | gene_id “TFE3_NG1”; transcript_id |
| | | | | | “TFE3_NG1.4”; exon “3”; |
| chr1 | exon | 198010742 | 198010799 | + | gene_id “TFE3_NG1”; transcript_id |
| | | | | | “TFE3_NG1.4”; exon “4”; |
| chr1 | exon | 198013482 | 198014754 | + | gene_id “TFE3_NG1”; transcript_id |
| | | | | | “TFE3_NG1.4”; exon “5”; |
| chr1 | transcript | 198103357 | 198104387 | + | gene_id “TFE3_NG2”; transcript_id |
| | | | | | “TFE3_NG2.1”; RPKM “2.6741”; cov |
| | | | | | “38.8118”; |
| chr1 | exon | 198103357 | 198104387 | + | gene_id “TFE3_NG2”; transcript_id |
| | | | | | “TFE3_NG2.1”; exon “1”; |
| chr12 | transcript | 265578 | 272387 | − | gene_id “TFE3_NG5”; transcript_id |
| | | | | | “TFE3_NG5.1”; RPKM “14.3596”; cov |
| | | | | | “208.4113”; |
| chr12 | exon | 265578 | 272387 | − | gene_id “TFE3_NG5”; transcript_id |
| | | | | | “TFE3_NG5.1”; exon “1”; |
| chr13 | transcript | 113322717 | 113334602 | − | gene_id “TFE3_NG6”; transcript_id |
| | | | | | “TFE3_NG6.1”; RPKM “12.1953”; cov |
| | | | | | “176.9991”; |
| chr13 | exon | 113322717 | 113333337 | − | gene_id “TFE3_NG6”; transcript_id |
| | | | | | “TFE3_NG6.1”; exon “1”; |
| chr13 | exon | 113333577 | 113334602 | − | gene_id “TFE3_NG6”; transcript_id |
| | | | | | “TFE3_NG6.1”; exon “2”; |
| chr13 | transcript | 113322717 | 113334602 | − | gene_id “TFE3_NG6”; transcript_id |
| | | | | | “TFE3_NG6.2”; RPKM “7.8941”; cov |
| | | | | | “114.5728”; |
| chr13 | exon | 113322717 | 113325637 | − | gene_id “TFE3_NG6”; transcript_id |
| | | | | | “TFE3_NG6.2”; exon “1”; |
| chr13 | exon | 113326747 | 113333337 | − | gene_id “TFE3_NG6”; transcript_id |
| | | | | | “TFE3_NG6.2”; exon “2”; |
| chr13 | exon | 113333577 | 113334602 | − | gene_id “TFE3_NG6”; transcript_id |
| | | | | | “TFE3_NG6.2”; exon “3”; |
| chr13 | transcript | 113322717 | 113334602 | − | gene_id “TFE3_NG6”; transcript_id |
| | | | | | “TFE3_NG6.3”; RPKM “6.3242”; cov |
| | | | | | “91.7887”; |
| chr13 | exon | 113322717 | 113325637 | − | gene_id “TFE3_NG6”; transcript_id |
| | | | | | “TFE3_NG6.3”; exon “1”; |
| chr13 | exon | 113326747 | 113326811 | − | gene_id “TFE3_NG6”; transcript_id |
| | | | | | “TFE3_NG6.3”; exon “2”; |
| chr13 | exon | 113329517 | 113329587 | − | gene_id “TFE3_NG6”; transcript_id |
| | | | | | “TFE3_NG6.3”; exon “3”; |
| chr13 | exon | 113332861 | 113333337 | − | gene_id “TFE3_NG6”; transcript_id |
| | | | | | “TFE3_NG6.3”; exon “4”; |
| chr13 | exon | 113333513 | 113334602 | − | gene_id “TFE3_NG6”; transcript_id |
| | | | | | “TFE3_NG6.3”; exon “5”; |
| chr13 | transcript | 113322717 | 113334602 | − | gene_id “TFE3_NG6”; transcript_id |
| | | | | | “TFE3_NG6.4”; RPKM “5.0608”; cov |
| | | | | | “73.4520”; |
| chr13 | exon | 113322717 | 113325637 | − | gene_id “TFE3_NG6”; transcript_id |
| | | | | | “TFE3_NG6.4”; exon “1”; |
| chr13 | exon | 113329517 | 113329587 | − | gene_id “TFE3_NG6”; transcript_id |
| | | | | | “TFE3_NG6.4”; exon “2”; |
| chr13 | exon | 113332861 | 113333337 | − | gene_id “TFE3_NG6”; transcript_id |
| | | | | | “TFE3_NG6.4”; exon “3”; |
| chr13 | exon | 113333513 | 113334602 | − | gene_id “TFE3_NG6”; transcript_id |
| | | | | | “TFE3_NG6.4”; exon “4”; |
| chr16 | transcript | 20547676 | 20548684 | + | gene_id “TFE3_NG7”; transcript_id |
| | | | | | “TFE3_NG7.1”; RPKM “9.1675”; cov |
| | | | | | “133.0555”; |
| chr1 6 | exon | 20547676 | 20548684 | + | gene_id “TFE3_NG7”; transcript_id |
| | | | | | “TFE3_NG7.1”; exon “1”; |
| chr17 | transcript | 17336544 | 17344732 | + | gene_id “TFE3_NG8”; transcript_id |
| | | | | | “TFE3_NG8.1”; RPKM “24.3878”; cov |
| | | | | | “353.9585”; |
| chr17 | exon | 17336544 | 17336592 | + | gene_id “TFE3_NG8”; transcript_id |
| | | | | | “TFE3_NG8.1”; exon “1”; |
| chr17 | exon | 17338690 | 17338847 | + | gene_id “TFE3_NG8”; transcript_id |
| | | | | | “TFE3_NG8.1”; exon “2”; |
| chr17 | exon | 17342707 | 17344732 | + | gene_id “TFE3_NG8”; transcript_id |
| | | | | | “TFE3_NG8.1”; exon “3”; |
| chr17 | transcript | 17336544 | 17344732 | + | gene_id “TFE3_NG8”; transcript_id |
| | | | | | “TFE3_NG8.2”; RPKM “5.9317”; cov |
| | | | | | “86.0914”; |
| chr17 | exon | 17336544 | 17336628 | + | gene_id “TFE3_NG8”; transcript_id |
| | | | | | “TFE3_NG8.2”; exon “1”; |
| chr17 | exon | 17338690 | 17338847 | + | gene_id “TFE3_NG8”; transcript_id |
| | | | | | “TFE3_NG8.2”; exon “2”; |
| chr17 | exon | 17342707 | 17344732 | + | gene_id “TFE3_NG8”; transcript_id |
| | | | | | “TFE3_NG8.2”; exon “3”; |
| chr17 | transcript | 17336544 | 17344732 | + | gene_id “TFE3_NG8”; transcript_id |
| | | | | | “TFE3_NG8.3”; RPKM “3.4443”; cov |
| | | | | | “49.9897”; |
| chr17 | exon | 17336544 | 17336592 | + | gene_id “TFE3_NG8”; transcript_id |
| | | | | | “TFE3_NG8.3”; exon “1”; |
| chr17 | exon | 17338690 | 17344732 | + | gene_id “TFE3_NG8”; transcript_id |
| | | | | | “TFE3_NG8.3”; exon “2”; |
| chr17 | transcript | 17336544 | 17344732 | + | gene_id “TFE3_NG8”; transcript_id |
| | | | | | “TFE3_NG8.4”; RPKM “1.1033”; cov |
| | | | | | “16.0131”; |
| chr17 | exon | 17336544 | 17336628 | + | gene_id “TFE3_NG8”; transcript_id |
| | | | | | “TFE3_NG8.4”; exon “1”; |
| chr17 | exon | 17338690 | 17344732 | + | gene_id “TFE3_NG8”; transcript_id |
| | | | | | “TFE3_NG8.4”; exon “2”; |
| chr17 | transcript | 17336544 | 17344732 | + | gene_id “TFE3_NG8”; transcript_id |
| | | | | | “TFE3_NG8.5”; RPKM “1.1118”; cov |
| | | | | | “16.1358”; |
| chr17 | exon | 17336544 | 17338847 | + | gene_id “TFE3_NG8”; transcript_id |
| | | | | | “TFE3_NG8.5”; exon “1”; |
| chr17 | exon | 17342707 | 17344732 | + | gene_id “TFE3_NG8”; transcript_id |
| | | | | | “TFE3_NG8.5”; exon “2”; |
| chr17 | transcript | 17336544 | 17344732 | + | gene_id “TFE3_NG8”; transcript_id |
| | | | | | “TFE3_NG8.6”; RPKM “4.2478”; cov |
| | | | | | “61.6512”; |
| chr17 | exon | 17336544 | 17344732 | + | gene_id “TFE3_NG8”; transcript_id |
| | | | | | “TFE3_NG8.6”; exon “1”; |
| chr17 | transcript | 80292461 | 80297873 | − | gene_id “TFE3_NG9”; transcript_id |
| | | | | | “TFE3_NG9.1”; RPKM “23.1051”; cov |
| | | | | | “335.3425”; |
| chr17 | exon | 80292461 | 80297873 | − | gene_id “TFE3_NG9”; transcript_id |
| | | | | | “TFE3_NG9.1”; exon “1”; |
| chr17 | transcript | 80298264 | 80304802 | − | gene_id “TFE3_NG10”; transcript_id |
| | | | | | “TFE3_NG10.1”; RPKM “28.2359”; cov |
| | | | | | “409.8092”; |
| chr17 | exon | 80298264 | 80299053 | − | gene_id “TFE3_NG10”; transcript_id |
| | | | | | “TFE3_NG10.1”; exon “1”; |
| chr17 | exon | 80300274 | 80304802 | − | gene_id “TFE3_NG10”; transcript_id |
| | | | | | “TFE3_NG10.1”; exon “2”; |
| chr17 | transcript | 80298264 | 80304802 | − | gene_id “TFE3_NG10”; transcript_id |
| | | | | | “TFE3_NG10.2”; RPKM “12.7802”; cov |
| | | | | | “185.4884”; |
| chr17 | exon | 80298264 | 80304802 | − | gene_id “TFE3_NG10”; transcript_id |
| | | | | | “TFE3_NG10.2”; exon “1”; |
| chr18 | transcript | 33444806 | 33446227 | − | gene_id “TFE3_NG11”; transcript_id |
| | | | | | “TFE3_NG11.1”; RPKM “5.8396”; cov |
| | | | | | “84.7553”; |
| chr18 | exon | 33444806 | 33446227 | − | gene_id “TFE3_NG11”; transcript_id |
| | | | | | “TFE3_NG11.1”; exon “1”; |
| chr18 | transcript | 60755402 | 60759211 | + | gene_id “TFE3_NG12”; transcript_id |
| | | | | | “TFE3_NG12.1”; RPKM “1.3508”; cov |
| | | | | | “19.6058”; |
| chr18 | exon | 60755402 | 60756131 | + | gene_id “TFE3_NG12”; transcript_id |
| | | | | | “TFE3_NG12.1”; exon “1”; |
| chr18 | exon | 60757568 | 60759211 | + | gene_id “TFE3_NG12”; transcript_id |
| | | | | | “TFE3_NG12.1”; exon “2”; |
| chr18 | transcript | 60755402 | 60769670 | + | gene_id “TFE3_NG12”; transcript_id |
| | | | | | “TFE3_NG12.2”; RPKM “4.9223”; cov |
| | | | | | “71.4404”; |
| chr18 | exon | 60755402 | 60756131 | + | gene_id “TFE3_NG12”; transcript_id |
| | | | | | “TFE3_NG12.2”; exon “1”; |
| chr18 | exon | 60757568 | 60757670 | + | gene_id “TFE3_NG12”; transcript_id |
| | | | | | “TFE3_NG12.2”; exon “2”; |
| chr18 | exon | 60763246 | 60769670 | + | gene_id “TFE3_NG12”; transcript_id |
| | | | | | “TFE3_NG12.2”; exon “3”; |
| chr2 | transcript | 41605975 | 41606187 | + | gene_id “TFE3_NG3”; transcript_id |
| | | | | | “TFE3_NG3.1”; RPKM “14.8287”; cov |
| | | | | | “215.2207”; |
| chr2 | exon | 41605975 | 41606187 | + | gene_id “TFE3_NG3”; transcript_id |
| | | | | | “TFE3_NG3.1”; exon “1”; |
| chr2 | transcript | 41606478 | 41606776 | + | gene_id “TFE3_NG4”; transcript_id |
| | | | | | “TFE3_NG4.1”; RPKM “16.3155”; cov |
| | | | | | “236.7987”; |
| chr2 | exon | 41606478 | 41606776 | + | gene_id “TFE3_NG4”; transcript_id |
| | | | | | “TFE3_NG4.1”; exon “1”; |
| chr20 | transcript | 7391956 | 7400603 | + | gene_id “TFE3_NG13”; transcript_id |
| | | | | | “TFE3_NG13.1”; RPKM “1.7134”; cov |
| | | | | | “24.8678”; |
| chr20 | exon | 7391956 | 7392062 | + | gene_id “TFE3_NG13”; transcript_id |
| | | | | | “TFE3_NG13.1”; exon “1”; |
| chr20 | exon | 7397768 | 7400603 | + | gene_id “TFE3_NG13”; transcript_id |
| | | | | | “TFE3_NG13.1”; exon “2”; |
| chr20 | transcript | 7391956 | 7400603 | + | gene_id “TFE3_NG13”; transcript_id |
| | | | | | “TFE3_NG13.2”; RPKM “7.7831”; cov |
| | | | | | “112.9625”; |
| chr20 | exon | 7391956 | 7392057 | + | gene_id “TFE3_NG13”; transcript_id |
| | | | | | “TFE3_NG13.2”; exon “1”; |
| chr20 | exon | 7397768 | 7400603 | + | gene_id “TFE3_NG13”; transcript_id |
| | | | | | “TFE3_NG13.2”; exon “2”; |
| chr20 | transcript | 7391956 | 7410806 | + | gene_id “TFE3_NG13”; transcript_id |
| | | | | | “TFE3_NG13.3”; RPKM “1.8293”; cov |
| | | | | | “26.5495”; |
| chr20 | exon | 7391956 | 7392057 | + | gene_id “TFE3_NG13”; transcript_id |
| | | | | | “TFE3_NG13.3”; exon “1”; |
| chr20 | exon | 7397768 | 7397871 | + | gene_id “TFE3_NG13”; transcript_id |
| | | | | | “TFE3_NG13.3”; exon “2”; |
| chr20 | exon | 7401921 | 7410806 | + | gene_id “TFE3_NG13”; transcript_id |
| | | | | | “TFE3_NG13.3”; exon “3”; |
| chr22 | transcript | 50920076 | 50923291 | − | gene_id “TFE3_NG14”; transcript_id |
| | | | | | “TFE3_NG14.1”; RPKM “0.7608”; cov |
| | | | | | “11.0419”; |
| chr22 | exon | 50920076 | 50922308 | − | gene_id “TFE3_NG14”; transcript_id |
| | | | | | “TFE3_NG14.1”; exon “1”; |
| chr22 | exon | 50922639 | 50923291 | − | gene_id “TFE3_NG14”; transcript_id |
| | | | | | “TFE3_NG14.1”; exon “2”; |
| chrX | transcript | 74846065 | 74857308 | − | gene_id “TFE3_NG15”; transcript_id |
| | | | | | “TFE3_NG15.1”; RPKM “0.7409”; cov |
| | | | | | “10.7529”; |
| chrX | exon | 74846065 | 74851850 | − | gene_id “TFE3_NG15”; transcript_id |
| | | | | | “TFE3_NG15.1”; exon “1”; |
| chrX | exon | 74856976 | 74857308 | − | gene_id “TFE3_NG15”; transcript_id |
| | | | | | “TFE3_NG15.1”; exon “2”; |
| chrX | transcript | 74846065 | 74857308 | − | gene_id “TFE3_NG15”; transcript_id |
| | | | | | “TFE3_NG15.2”; RPKM “3.0723”; cov |
| | | | | | “44.5905”; |
| chrX | exon | 74846065 | 74851854 | − | gene_id “TFE3_NG15”; transcript_id |
| | | | | | “TFE3_NG15.2”; exon “1”; |
| chrX | exon | 74856976 | 74857308 | − | gene_id “TFE3_NG15”; transcript_id |
| | | | | | “TFE3_NG15.2”; exon “2”; |
| chrX | transcript | 74846065 | 74857308 | − | gene_id “TFE3_NG15”; transcript_id |
| | | | | | “TFE3_NG15.3”; RPKM “13.9975”; cov |
| | | | | | “203.1562”; |
| chrX | exon | 74846065 | 74857308 | − | gene_id “TFE3_NG15”; transcript_id |
| | | | | | “TFE3_NG15.3”; exon “1”; |
| chr1 | transcript | 9158343 | 9163247 | + | gene_id “TMFI_NG1”; transcript_id |
| | | | | | “TMFI_NG1.1”; RPKM “3.0798”; cov |
| | | | | | “53.0276”; |
| chr1 | exon | 9158343 | 9163247 | + | gene_id “TMFI_NG1”; transcript_id |
| | | | | | “TMFI_NG1.1”; exon “1”; |
| chr10 | transcript | 44280423 | 44289869 | − | gene_id “TMFI_NG5”; transcript_id |
| | | | | | “TMFI_NG5.1”; RPKM “1.1567”; cov |
| | | | | | “19.9166”; |
| chr10 | exon | 44280423 | 44280632 | − | gene_id “TMFI_NG5”; transcript_id |
| | | | | | “TMFI_NG5.1”; exon “1”; |
| chr10 | exon | 44280757 | 44281632 | − | gene_id “TMFI_NG5”; transcript_id |
| | | | | | “TMFI_NG5.1”; exon “2”; |
| chr10 | exon | 44289425 | 44289869 | − | gene_id “TMFI_NG5”; transcript_id |
| | | | | | “TMFI_NG5.1”; exon “3”; |
| chr10 | transcript | 44280423 | 44289869 | − | gene_id “TMFI_NG5”; transcript_id |
| | | | | | “TMFI_NG5.2”; RPKM “1.3729”; cov |
| | | | | | “23.6389”; |
| chr10 | exon | 44280423 | 44280632 | − | gene_id “TMFI_NG5”; transcript_id |
| | | | | | “TMFI_NG5.2”; exon “1”; |
| chr10 | exon | 44280757 | 44281632 | − | gene_id “TMFI_NG5”; transcript_id |
| | | | | | “TMFI_NG5.2”; exon “2”; |
| chr10 | exon | 44289161 | 44289869 | − | gene_id “TMFI_NG5”; transcript_id |
| | | | | | “TMFI_NG5.2”; exon “3”; |
| chr14 | transcript | 23355271 | 23356086 | + | gene_id “TMFI_NG1.1”; transcript_id |
| | | | | | “TMFI_gene.21900.1.0”; RPKM |
| | | | | | “0.1602”; cov “2.7582”; |
| chr14 | exon | 23355271 | 23355440 | + | gene_id “TMFI_NG1.1”; transcript_id |
| | | | | | “TMFI_gene.21900.1.0”; exon “1”; |
| chr14 | exon | 23355823 | 23356086 | + | gene_id “TMFI_NG1.1”; transcript_id |
| | | | | | “TMFI_gene.21900.1.0”; exon “2”; |
| chr14 | transcript | 23743275 | 23754199 | − | gene_id “TMFI_NG1.1”; transcript_id |
| | | | | | “TMFI_gene.21920.1.0”; RPKM |
| | | | | | “0.3807”; cov “6.5557”; |
| chr14 | exon | 23743275 | 23746396 | − | gene_id “TMFI_NG1.1”; transcript_id |
| | | | | | “TMFI_gene.21920.1.0”; exon “1”; |
| chr14 | exon | 23754107 | 23754199 | − | gene_id “TMFI_NG1.1”; transcript_id |
| | | | | | “TMFI_gene.21920.1.0”; exon “2”; |
| chr14 | transcript | 23743275 | 23755321 | − | gene_id “TMFI_NG1.1”; transcript_id |
| | | | | | “TMFI_gene.21920.1.2”; RPKM |
| | | | | | “0.5820”; cov “10.0210”; |
| chr14 | exon | 23743275 | 23746396 | − | gene_id “TMFI_NG1.1”; transcript_id |
| | | | | | “TMFI_gene.21920.1.2”; exon “1”; |
| chr14 | exon | 23755122 | 23755321 | − | gene_id “TMFI_NG1.1”; transcript_id |
| | | | | | “TMFI_gene.21920.1.2”; exon “2”; |
| chr18 | transcript | 27452992 | 27474842 | + | gene_id “TMFI_NG6”; transcript_id |
| | | | | | “TMFI_NG6.1”; RPKM “21.5395”; cov |
| | | | | | “370.8664”; |
| chr18 | exon | 27452992 | 27453200 | + | gene_id “TMFI_NG6”; transcript_id |
| | | | | | “TMFI_NG6.1”; exon “1”; |
| chr18 | exon | 27453299 | 27474842 | + | gene_id “TMFI_NG6”; transcript_id |
| | | | | | “TMFI_NG6.1”; exon “2”; |
| chr18 | transcript | 27452992 | 27474842 | + | gene_id “TMFI_NG6”; transcript_id |
| | | | | | “TMFI_NG6.2”; RPKM “19.4384”; cov |
| | | | | | “334.6911”; |
| chr18 | exon | 27452992 | 27474842 | + | gene_id “TMFI_NG6”; transcript_id |
| | | | | | “TMFI_NG6.2”; exon “1”; |
| chr2 | transcript | 30393847 | 30406047 | − | gene_id “TMFI_NG2”; transcript_id |
| | | | | | “TMFI_NG2.2”; RPKM “2.6920”; cov |
| | | | | | “46.3504”; |
| chr2 | exon | 30393847 | 30406047 | − | gene_id “TMFI_NG2”; transcript_id |
| | | | | | “TMFI_NG2.2”; exon “1”; |
| chr2 | transcript | 30393847 | 30407124 | − | gene_id “TMFI_NG2”; transcript_id |
| | | | | | “TMFI_NG2.3”; RPKM “1.3154”; cov |
| | | | | | “22.6486”; |
| chr2 | exon | 30393847 | 30404344 | − | gene_id “TMFI_NG2”; transcript_id |
| | | | | | “TMFI_NG2.3”; exon “1”; |
| chr2 | exon | 30406850 | 30407124 | − | gene_id “TMFI_NG2”; transcript_id |
| | | | | | “TMFI_NG2.3”; exon “2”; |
| chr2 | transcript | 30406201 | 30407124 | − | gene_id “TMFI_NG2”; transcript_id |
| | | | | | “TMFI_NG2.1”; RPKM “2.6356”; cov |
| | | | | | “45.3806”; |
| chr2 | exon | 30406201 | 30407124 | − | gene_id “TMFI_NG2”; transcript_id |
| | | | | | “TMFI_NG2.1”; exon “1”; |
| chr9 | transcript | 137513888 | 137514341 | − | gene_id “TMFI_NG3”; transcript_id |
| | | | | | “TMFI_NG3.1”; RPKM “3.1922”; cov |
| | | | | | “54.9626”; |
| chr9 | exon | 137513888 | 137514341 | − | gene_id “TMFI_NG3”; transcript_id |
| | | | | | “TMFI_NG3.1”; exon “1”; |
| chr9 | transcript | 137514674 | 137516981 | − | gene_id “TMFI_NG4”; transcript_id |
| | | | | | “TMFI_NG4.1”; RPKM “3.4731”; cov |
| | | | | | “59.7998”; |
| chr9 | exon | 137514674 | 137516981 | − | gene_id “TMFI_NG4”; transcript_id |
| | | | | | “TMFI_NG4.1”; exon “1”; |

Legends are as follow. Chromosome (number of the chromosome); source: GTF data from reference-based transcript assembler “Scallop”; feature: transcript or exon (each transcript is composed of one or more exons, the standard order in the table is the transcript and then its one or more exon components); start; beginning of the transcript or exon; end: end of the transcript or exon; DNA strand + or −; attributes:
for the transcripts: gene_id (gene name), transcript_id (name/reference of the transcript), RPKM (expression value as defined in the present application), coverage (coverage value)
for the exons: gene_id (gene name), transcript_id (name of the transcript), exon (exon No).
It is to be noted that the source are GTF data from reference-based transcript assembler “Scallop” for all transcripts and the score is 1000 for all.

### FIGURES

FIG. 1. EWS-FLI1 regulates Ewing specific neogenes.

(A) Scheme of the genomic region of the JUTE 1 gene. From top to bottom: a XX GGAA microsatellite is indicated, ChiP-seq of EWS-FLI1 in EWS-FLI1high and EWS-FLI1low conditions, H3K27ac and H3K4me3 in the same EWS-FLI1high and EWS-FLI1low conditions as well as short read RNA-seq alignments of the A673/TR/shEF in + and − DOX conditions and of three different Ewing tumors.

(B) RT-PCR analysis of the expression of the four neo-transcripts in the A673/TR/shEF cell line in EWS-FLI1high (DOX−) and EWS-FLI1low (DOX+) conditions.

(C) RT-PCR analysis of the expression of the four neo-transcripts in wild type and EWS-FLI1-expressing MSCs.

(D) Expression of JUJE 1 in cancers (TCGA and institutional databases). TPM: transcripts per million.

(E) Expression of JUJE 1 in tissues (GTEx, TCGA, HPA databases).

FIG. 2. Discovery of Ewing specific neogenes from short-read RNA-seq data.

(A) Overview of the neogene discovery pipeline (cf Methods for details).

(B) Modulation of neogene expression by EWS-FLI1 in 10 Ewing cell lines and MSCs. Size of the bubbles shows mean expression level in log 10 TPM of neogenes in Ewing cell lines with baseline EWS-FLI1 expression or MSCs expressing EWS-FLI1 (EF high), capped at 100 TPM. Color represents the log 2 fold-change (capped at 6) of mean neogene expression levels in EF high cell lines versus in corresponding cell lines with downregulated expression of EWS-FLI1, or MSCs not expressing EWS-FLI1 (EF low). Number of replicates per condition are: n=4 for TC71; n=3 for ASP14, MSC; n=2 for A673, CHLA10, EW1, EW24 and MHH-ES1; n=1 for EW7, POE and SK-N-MC.

FIG. 3. Discovery of neogenes specific for alveolar rhabdomyosarcoma (aRMS) and desmoplastic small round cell tumor (DSRCT).

(A) Modulation of aRMS-specific neogene expression by PAX3-FOXO1 in aRMS RH4 cell line. Size of the bubbles shows expression level in log 10 TPM of neogenes in RH4 with baseline PAX3-FOXO1 expression (PF high). Color represents the log 2 fold-change of neogene expression level in PF high RH4 versus in RH4 with downregulated expression of PAX3-FOXO1 (PF low). n=1 replicate per condition.

(B) Modulation of DSRCT-specific neogene expression by EWS-WT1 in DSRCT cell lines. Size of the bubbles shows mean expression level in log 10 TPM of neogenes in cell lines with baseline EWS-WT1 expression (EW high). Color represents the log 2 fold-change of neogene expression level in EW high cell lines versus in cell lines with downregulated expression of EWS-WT1 (EW low). n=3 replicates per condition for both cell lines.

FIG. 4: Exemple of tetramer analysis and phenotypic characterization of a peptide derived from an EWS-FLI1-regulated lincRNA in lymphocyte from a healthy donor. A, principle of the teramer analysis; B, identification of tetramer-bound T cells; C, characterization of the naïve/memory phenotype.

FIG. 5: T cell clone sensibility. Cytokine secretion of CD8 T cells specific for peptides encoded by EWS-FLI1-regulated lincRNAs (initially identified by PacBio sequencing) in response to increasing concentration of their specific antigen presented by the K562 cell antigen presenting cells.

### EXAMPLES

### Experimental Procedures

Cell Culture

A673/TR/shEF (also called ASP14) cell line (Carrillo et al., 2007) was routinely checked by PCR for the absence of mycoplasma. A673/TR/shEF cell line was cultured at 37° C., in 5% CO² with Dulbecco's Modified Eagle Medium DMEM with High Glucose, 4 mM of L-Glutamine, 4500 mg/L Glucose and sodium pyruvate (HyClone) supplemented with 10% FBS (Eurobio) and 1% antibiotics (v/v) (penicillin and streptomycin (Gibco)). Induction of EWS-FLI1 specific shRNA was performed by adding 1 μg/mL of doxycycline in the medium ex-tempo. After seven days of treatment, doxycycline was removed and cells were washed three times to stop the shRNA induction, thus enabling re-expression of EWS-FLI1.

RNA from MSCs expressing or not EWS-FLI1 has been obtained from Stamenkovic lab.

RNA Extraction and Reverse Transcriptase

From cell extract: Total RNA was isolated using the Nucleospin II kit (Macherey-Nagel) and reverse-transcribed using the High-Capacity cDNA Reverse Transcription kit (Applied Biosystems). Next, cDNA molecules were amplified by PCR performed using the AmpliTaqGold DNA Polymerase kit with Gold Buffer and MgCl2 (Applied Biosystems). One microgram of template total RNA was used for each reaction.

From polysome profiling fraction: gradient fractions were collected in 16 tubes. Equal RNA volumes (300 μL) of each gradient fraction were used for RT using the iScript cDNA Synthesis Kit (BioRad).

Next, cDNA molecules were amplified by qPCR performed using SYBR Green (Applied Biosystems). Reactions were run on 7500 QPCR instrument and analyzed using the 7500 system SDS software (Applied Biosystems). Relative quantification of neotranscripts was normalized to an endogenous control (RPLPO) and was performed using the comparative Ct method. Error bars indicate SD. Oligonucleotides were purchased from MWG Eurofins Genomics.

EWS-FLI1 primers:
F: GCCAAGCTCCAAGTCAATATAGC/
R: GAGGCCAGAATTCATGTTATTGC
RPLP0 primers:
F: GAAACTCTGCATTCTCGCTTC/
R: GGTGTAATCCGTCTCCACAG.
Primer JUJE1:
F: ATACTGGGCTTGCAACTGGAG/
R: TCCTCCTTGGAATGAATGGGC.
Primer JUJE2:
F: TGGCCAATTGGAGGGTCTTC/
R: GCAAATTACATCCTCATTTCTCCA.
Primer JUJE3:
F: ACAGCACTGCTAGATTAGGGAA/
R: CAGTGCAAGCCCTCGAATGTC.
Primer JUJE10:
F: ACTTTCCTTTCTCCTCACCACC/
R: AGACATCCTAAAGTAACAGAGGCA.

RNA Seq (Illumina & PacBio) & Data Processing

Illumina:

Every RNA samples were evaluated for integrity using BioAnalyzer instrument (Agilent). All samples displayed excellent quality (RNA Integrity Number above 9). Libraries were performed using the TruSeq Stranded mRNA Library Preparation Kit. Equimolar pools of libraries were sequenced on an Illumina HiSeq 2500 machine using paired-ends reads (PE, 2×101 bp) and High Output run mode allowing to get around 200 million of raw reads per sample. Raw reads were mapped on the human reference genome hg19 using the STAR aligner (v.2.5.0a). PCR-duplicated reads and low mapping quality reads (MQ<20) were removed using Picard tools and SAMtools, respectively.

Pacbio:

Libraries have been prepared following the protocol from Pacific Biosciences: “Procedure & Checklist—Iso-Seg™ Template Preparation for Sequel® Systems—Version 5 (November 2017)”

1 ug of totalRNA has been used as input. The cDNA has been synthetized with the SMARTer PCR cDNA Synthesis Kit from Clontech, following manufacturer's recommendations. Then the cDNA was amplified with the PrimeSTAR GXL DNA Polymerase from Clontech with 12 cycles of PCR. This number has been set up after PCR optimization, in order to obtain enough yield, and to avoid PCR bias. The amplified cDNA was then split into only 2 fractions to perfom 2 different purifications using AMPure beads (ratio of 0.4× and 1×). No 3rd fraction was isolated to perform a size-selection step using Blue Pippin system. Then an equimolar pool was made from the 2 fractions. The SMRTbell has been prepared from 2.8 ug of this equimolar pool of cDNA using the SMRTbell Template Prep Kit from Pacific Biosciences, following manufacturer's recommendations. The sequencing was performed on Sequel system, using V2.1 chemistry and Magbead loading. 4 SMRTcells have been used for the ASP14 sample and 3 SMRTcells for the ASP14+DOX sample. The sequencing runs were set up with a pre-extension step of 240 min and 10 hours of movie. We used the implemented pbsmrtpipe pipeline to perform read processing.

To annotate IsoSeq reads, we used MatchAnnot script (https://github.com/TomSkelly/MatchAnnot). MatchAnnot assign each read to transcripts annotation using score base-pair matching. Reads that have no match on the Gencode v19 reference are annotated as NA. We manually curated the list of NA reads (n=145) using Integrative Genomics Viewer (IGV). We also used Illumina RNAseq data, ChIPseq data (EWS-FLI1, H3K27me3, H3K27ac, H3K4me3) and GGAA repeats track at the same time in order to identify EWS-FLI1-regulated reads from intergenic region. After applying these filters, we found 4 clusters corresponding to four distinct expressed-intergenic regions.

JUJE Neotranscripts Quantification

Public fastq files from TCGA, GTEx and HPA dataset were downloaded and aligned to the hg19 genome assembly using STAR version 2.7.0e (Dobin et al, 2013). The GTF file used for alignment and quantification of gene expression was based on evidence-based annotation of the human genome (GRCh37), version 19 (Ensembl 74) provided by GENCODE, to which was added the information for the 4 neotranscripts, previously described, in GTF format. Gene expression was quantified using the GeneCounts procedure from STAR. Raw counts were then normalized to Transcripts Per Million (TPM).

Tumor RNA-Seq Used for Discovery of Neogenes

Paired-end RNA-seq from the institutional database (Institut Curie) of fresh-frozen patient tumor tissue were used to search for tumor-specific neogenes. RNA-seq sequencing was performed using established protocols on Illumina instruments. 31 types of cancers, for which the inventors had at least 4 RNA-seq samples were tested, including 20 fusion-driven cancers. All diagnoses were made by pathological examination, confirmed by fusion gene detection in the case of fusion-driven cancers and independently reviewed by an expert clinician. In detail, the inventors assessed the following diseases (abbreviations for neogene nomenclature in parentheses): 20 fusion-driven cancers including Ewing sarcoma (Ew), alveolar rhabdomyosarcoma (aRMS), desmoplastic small round cell tumor (DSRCT), BCOR-rearranged sarcoma (BCOR), CIC-fused sarcoma (CIC), clear cell sarcoma (CCS), EWSR1-NFATC2 sarcoma (NFAT), synovial sarcoma (SS), angiomatoid fibrous histiocytoma (AFH), alveolar soft part sarcoma (ASPS), congenital fibrosarcoma (CFS), extraskeletal myxoid chondrosarcoma (emCS), low-grade fibromyxoid sarcoma (LGFS), mesenchymal chondrosarcoma (MCS), midline carcinoma (Midline), myxoid liposarcoma (mLPS), EWSR1-PATZ1 sarcoma (PATZ1), solitary fibrous tumor (SFT), TFE3 renal cell carcinoma (TFE3), inflammatory myofibroblastic tumor (TMFI); 11 non-fusion driven cancers including atypical teratoid rhabdoid tumor (ATRT), desmoid tumor (Desmoid), embryonal rhabdomyosarcoma (eRMS), leiomyosarcoma (LMS), dedifferentiated liposarcoma (LPS), malignant peripheral nerve sheath tumor (MPNST), nephroblastoma (NEP), neuroblastoma (NEU), osteosarcoma (OST), small cell carcinoma of the ovary hypercalcemic type (SCCOHT) and undifferentiated pleomorphic sarcoma (UPS). For discovery of neogenes, the inventors initially ran the pipeline on the first 8 fusion-driven sarcomas in the list («discovery batch 1») before testing the 23 other cancers («discovery batch 2»).

RNA-Seq Alignment, Transcript Assembly and Detection of Unannotated Transcripts

The inventors used Scallop (Shao M, Kingsford C. Accurate assembly of transcripts through phase-preserving graph decomposition. Nat Biotechnol. 2017 December; 35(12):1167-1169), a reference-based transcript assembler, to predict all transcript sequences based on aligned RNA-seq reads, independently of a reference transcriptome annotation. First, paired-end FASTQ files were aligned to the hg19 human reference genome using STAR v2.7.0 (Dobin et al., Bioinformatics 2013). They then ran Scallop on the resulting BAM file with default parameters to assemble all expressed transcript sequences. To conserve only unannotated transcripts, they used Gffcompare (Pertea and Pertea, F1000 Research 2020) to compare the Scallop output GTF file with the reference GENCODE v19 GTF file, and conserved only transcripts labeled by Gffcompare as «u» (unknown, intergenic), «y» (contains a reference within its introns) and «x» (exonic overlap on the opposite strand). Finally, to remove lowly expressed transcripts and decrease the rate of false positives, they removed all transcripts with coverage less than 10 as output by Scallop.

Selection of Tumor-Specific Unannotated Transcripts

To discover tumor-specific neogenes and discard all other transcripts assembled by Scallop, three steps of selection were used.

1. First, the inventors ran Scallop as described previously on one RNA-seq sample of the cancer of interest to generate a first set of candidate unannotated transcripts (Candidate set 1).

2. Then they applied a first filtering process based on high and tumor-specific expression as compared to a limited set of other tumors: for this they quantified the expression of Candidate set 1 neogenes on 3 samples from each cancer type (24 samples of 8 types in «discovery batch 1» and 69 samples of 23 types in «discovery batch 2») by re-aligning each sample with STAR and quantifying expression using the GeneCounts procedure with the GENCODE v19 reference GTF file to which they added the Candidate set 1 neotranscripts. Raw counts were converted to transcripts per million (TPM) before the filtering process. To retain only tumor-specific and highly expressed candidates, they selected transcripts with:

(i) mean expression in the disease of interest of more than 20 TPM,

(ii) log-fold change of mean expression in samples of other diagnoses versus mean expression in disease of interest of less than −2,

(iii) mean expression in samples of other diagnoses of less than 3 TPM,

(iv) maximum expression in samples of other diagnoses of less than 10 TPM,

resulting in a second set of candidate neogenes (Candidate set 2). As expression in this process was quantified with a unique GTF file containing all Candidate set 1 neogenes for batch 2 (23 tumor types), the first threshold for the filtering process in batch 2 was adapted to (i) mean expression in the disease of interest of more than 10 TPM (instead of 20). As more samples were used in the filtering process for batch 2 (69 versus 24 in batch 1), the fourth threshold was adapted to (iv) maximum expression in samples of another diagnosis of less than 15 TPM (instead of 10). Other thresholds were left unchanged for batch 2. For Ewing sarcoma, we also ran Scallop as described previously on one RNA-seq sample from a cell line (ASP14) and applied the same above filters to the resulting Candidate set 1 of neogenes. The resulting Candidate set 2 neogenes not overlapping Candidate set 2 neogenes from the Ewing tumor sample were included in the final Candidate set 2 neogenes used for the subsequent round of filtering.

3. Finally, a second filtering step was applied based on expression levels across a wide range of cancers and normal tissues: for this we quantified expression of Candidate set 2 neogenes in all tumors from our institutional database, all cancer types from TCGA (either all the samples from one type or 50 samples if number of samples exceeded 50), all normal tissue samples from TCGA, all normal tissues from GTEx (either all the samples from one type or 50 samples if number of samples exceeded 50) and all normal tissue samples from the Human Protein Atlas. Every sample was re-aligned with STAR and expression quantified by the GeneCounts procedure with the use of a GTF file including GENCODE v19 and Candidate set 2 neotranscripts (one for each discovery batch). Raw counts were converted to TPM before filtering.

To retain tumor-specific candidates with a relatively high expression level (accounting for potentially lower tumor content in some samples the inventors diminished the first threshold (i) as compared to the first filter) and quasi-null expression in other cancers and normal tissues, they selected transcripts with:

(i) mean expression in the disease of interest of more than 7.5 TPM,

(ii) log-fold change of mean expression in other samples versus mean expression in disease of interest of less than −3,

(iii) mean expression in other samples of less than 2 TPM,

(iv) 99% quantile of expression in other samples of less than 10 TPM,

(v) maximum mean expression in another cancer or tissue of less than 10 TPM (excluding testis and placenta),

resulting in a final set of tumor-specific neogenes.

During the procedure they found that a large part of candidate neogenes were mutually and exclusively expressed in the following pairs of diagnoses: angiomatoid fibrous histiocytoma (AFH) and clear cell sarcoma (CCS), alveolar soft part sarcoma (ASPS) and TFE3 renal cell carcinoma (TFE3), alveolar rhabdomyosarcoma (aRMS) and embryonal rhabdomyosarcoma (eRMS). They did not remove those neogenes (cf Results) to account for neogenes driven by the same fusion gene in two different diseases (AFH/CCS, ASPS/TFE3) and disease-associated neogenes common to both types of rhabdomyosarcoma (aRMS/eRMS).

Analysis of Fusion Transcription Factor Binding and Histone Marks Around Neogenes

The inventors analyzed ChIP-seq data to explore the epigenetic landscape of neogenes in 3 fusion-driven sarcomas (Ewing, aRMS and DSRCT). For Ewing sarcoma, they used ChIP-seq data from our laboratory for EWS-FLI1, H3K27ac and H3K4me3 in the ASP14 cell line. For alveolar rhabdomyosarcoma, ChIP-seq from public data was used: H3K27ac, H3K4me3 (GSE83726 from Gryder et al., Cancer Discov 2017) and PAX3-FOXO1 (GSE19063 from Cao et al., Cancer Res 2010) for the RH4 cell line. For DSRCT, ChIP-seq from public data was used: EWS-WT1 and PolII (GSE156277 from Hingorani et al., Sci Rep 2020) for the JN-DSRCT-1 cell line. For public data, FASTQ files were downloaded from SRA and aligned with Bowtie2 v2.2.9 (Langmead and Salzberg, Nature Methods 2012), duplicates and multi-mapped reads were removed with Samtools (Li et al., Bioinformatics 2009).

Analysis of Expression of Neogenes in Cell Lines

For Ewing sarcoma, aRMS and DSRCT, the inventors quantified expression of neogenes in cell lines having normal or downregulated expression of the fusion transcript (respectively EWS-FLI1, PAX3-FOXO1 and EWS-WT1). RNA-seq reads were aligned with STAR using a GTF file containing GENCODE v19 and the corresponding neogenes, quantification was done using GeneCounts. For Ewing sarcoma, they used RNA-seq data from their laboratory for 10 cell lines having either normal expression of EWS-FLI1 at day 0 or downregulated expression by a doxycyclin-inducible system or siRNA after 7 days. They also used RNA-seq data for MSCs, some of which were induced to express EWS-FLI1. For aRMS, the inventors used RNA-seq from public data (GSE83724 from Gryder et al., Cancer Discov 2017) for the RH4 cell line treated with an shRNA against PAX3-FOXO1 for 48 hours or a control shRNA. For DSRCT, they used RNA-seq from public data (GSE137561 from Gedminas et al., Oncogenesis 2020) for BER and JN-DSRCT-1 cell lines treated with siRNA against EWS-WT1 for 48 hours or control siRNA. All FASTQ files from public data were downloaded from SRA.

Generation of Genomic-Browser Style Figures for Neogene Loci

Genomic browser-style figures showing neotranscript sequences, RNA-seq read alignments, and ChIP-seq data were generated with a custom script written in R (R Core Team, 2017). RNA-seq reads in FASTQ format were aligned to the hg19 human reference genome with STAR using a GENCODE v19 reference GTF annotation and visualized in BAM format. ChIP-seq BAM files used for visualization were generated as described previously. For Ewing sarcoma, GGAA repeats (EWS-FLI1 canonical binding sites) were also displayed in the same figure.

Peptide Prediction

For each transcript, open reading frames were identified using ORFfinder (https://www.ncbi.nlm.nih.gov/orffinder) in the three frames, with parameters “minimal ORF length”=75 nucleotides and “ORF start codon”=any.

Peptides binding to MHC Class 1 molecules were predicted using the NetMHCpan 4.1 suite (http://www.cbs.dtu.dk/services/NetMHCpan/), using “HLA allele”=A2, “peptide length”=8-11 and “rank threshold for strong binding”=0.5%.

Tetramer Preparation and Immunological Analyses

All experiments were conducted at the immunology department of Institut Curie. Peptides were synthetized by the GeneCust company and HLA-A*02:01/peptide monomers were prepared using the easYmer kit (immunAware, Denmark). After control of the affinity of monomers, tetramers were incubated with human CD8+ prepared from healthy controls (EasySep kit from STEMCELL technologies). Tetramer-CD8 cell binding and analysis of the bound CD8 populations (naïve or memory) was assessed by flow cytometry.

Results:

The inventors initially performed long-read RNA sequencing using the PacBio Iso-Seq protocol to investigate the full-length transcriptomic profile of the A673 Ewing cell line. A total of 15,576,646 raw sequence reads were generated, representing 40.3 Gbp. Using the pbsmtpipe pipeline (REF), 56,051 high-quality Circular Consensus Sequence (CCS) were kept for downstream analysis. Surprisingly, they identified 145 high-quality CCS (0.5%) that could be aligned to the human genome but had no match on the RefSeq database of human transcripts.

Manual inspection of each CCS identified 80 robust candidate neotranscripts. Other sequences were classified as mis-annotated genes (e.g. read-through transcription) or had low coverage support. They hypothesized that some of these transcripts may be generated by transcriptional activation by EWS-FLI1, the aberrant transcription factor specific for Ewing sarcoma. Under this hypothesis, such transcripts should fulfill the following criteria:
i) be regulated by EWS-FLI,
ii) demonstrate the presence of an EWS-FLI1 ChIP-seq peak as well as H3K27ac and H3K4me3 histone marks around their transcription start sites (TSS) and
iii) be expressed specifically in Ewing tumors.

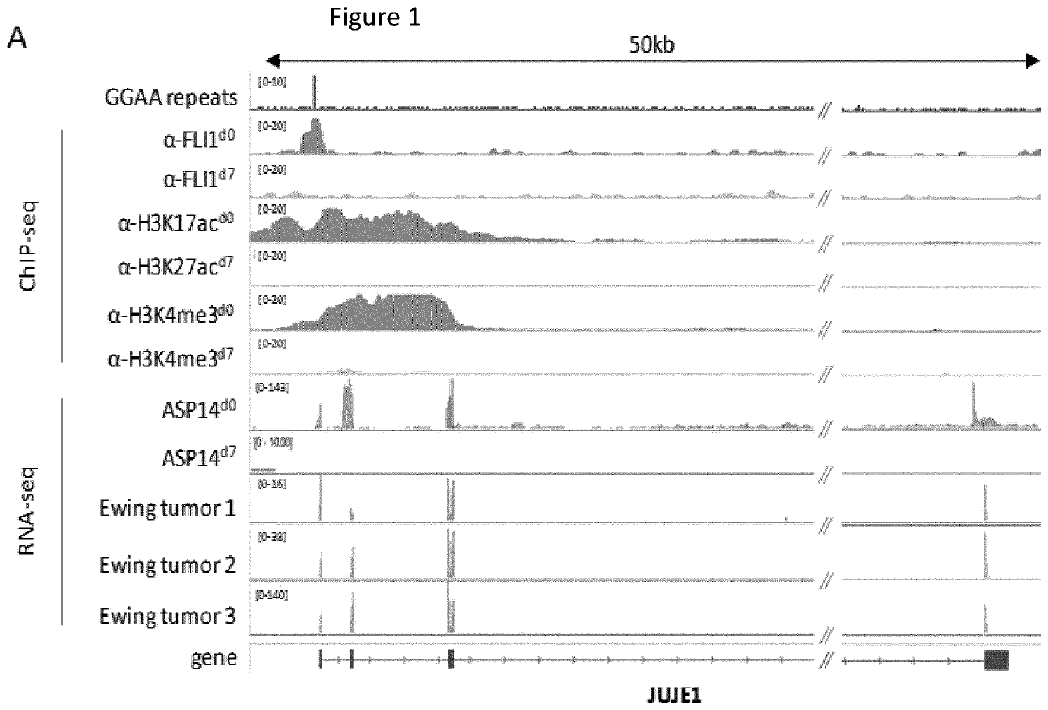

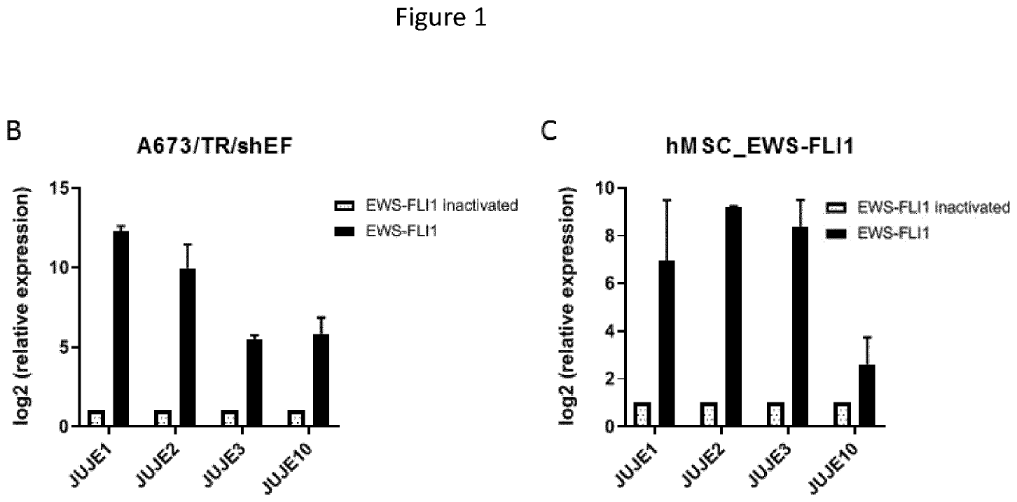

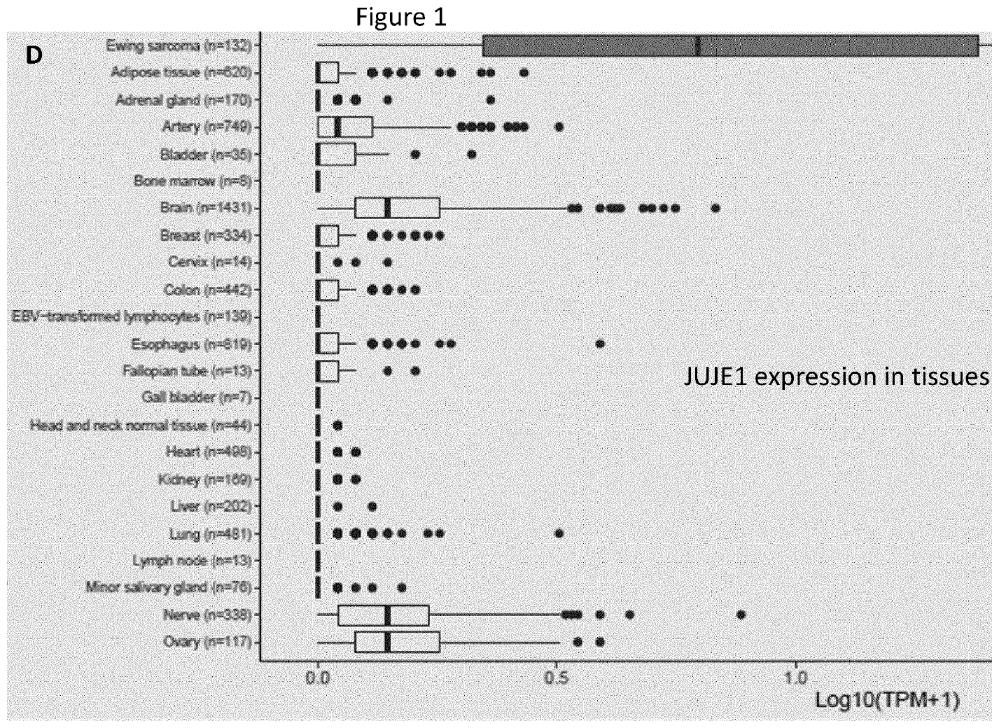

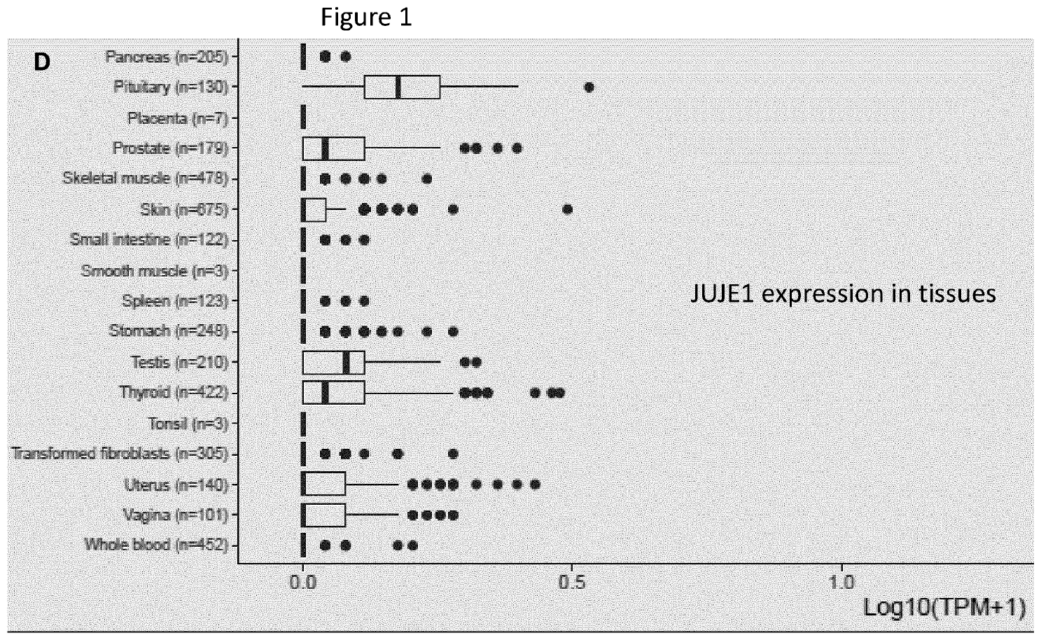

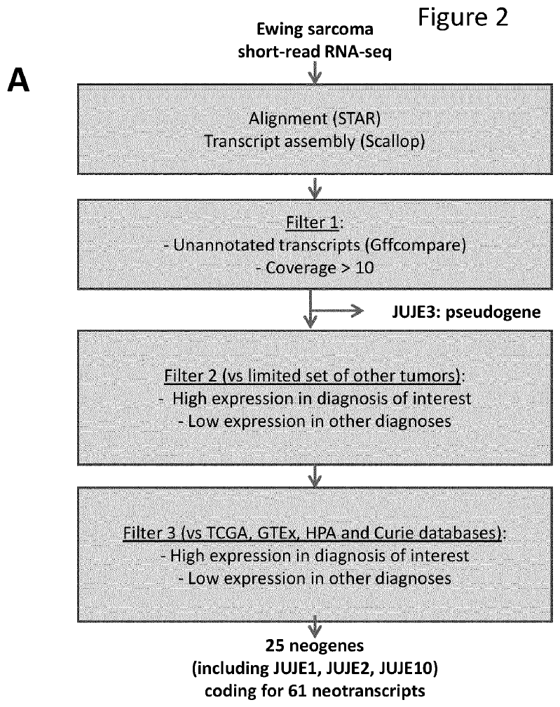

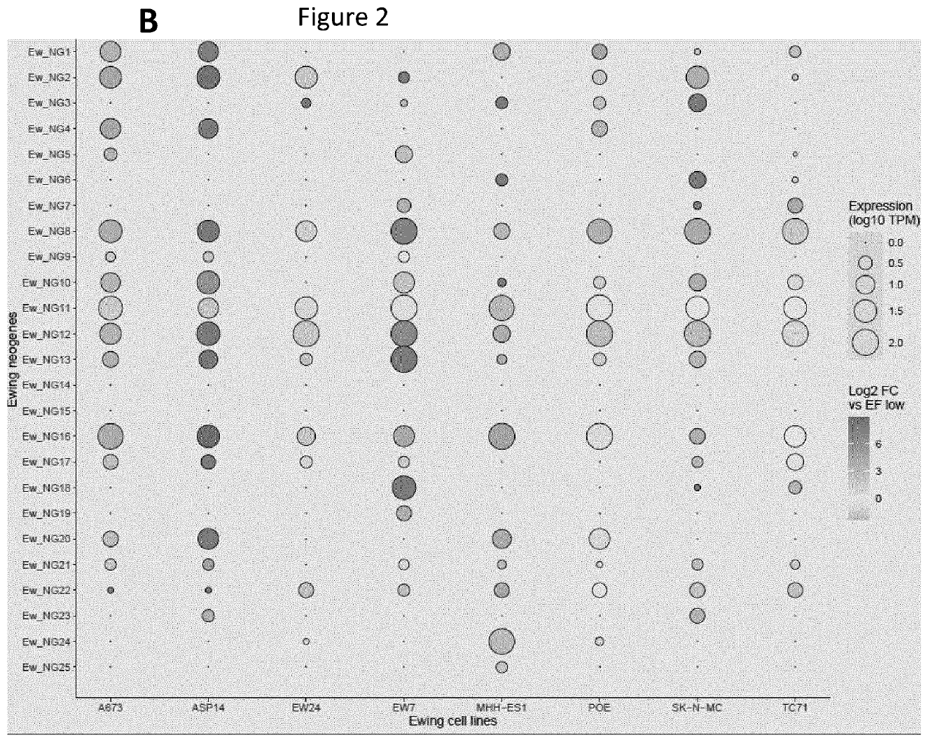

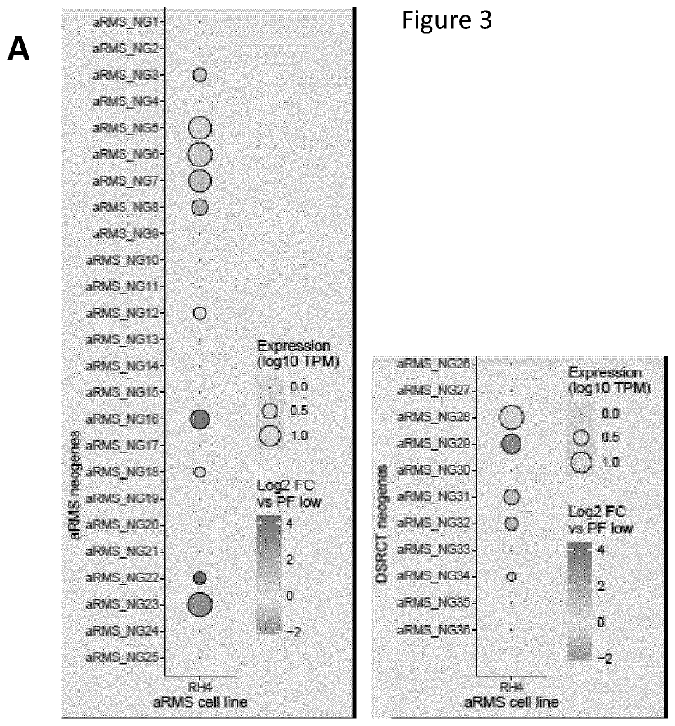

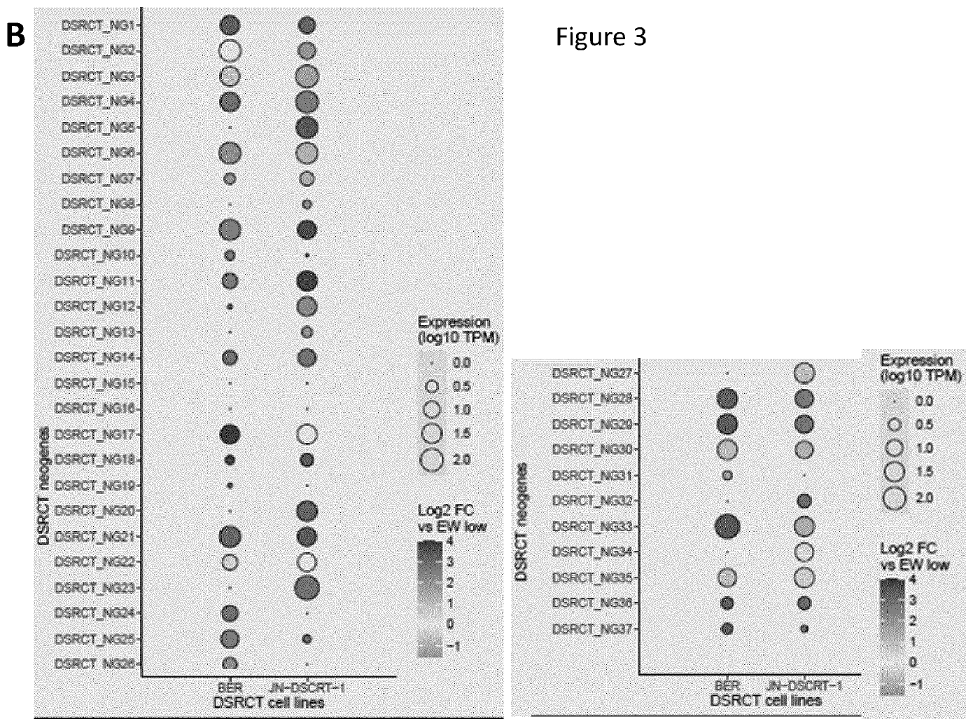

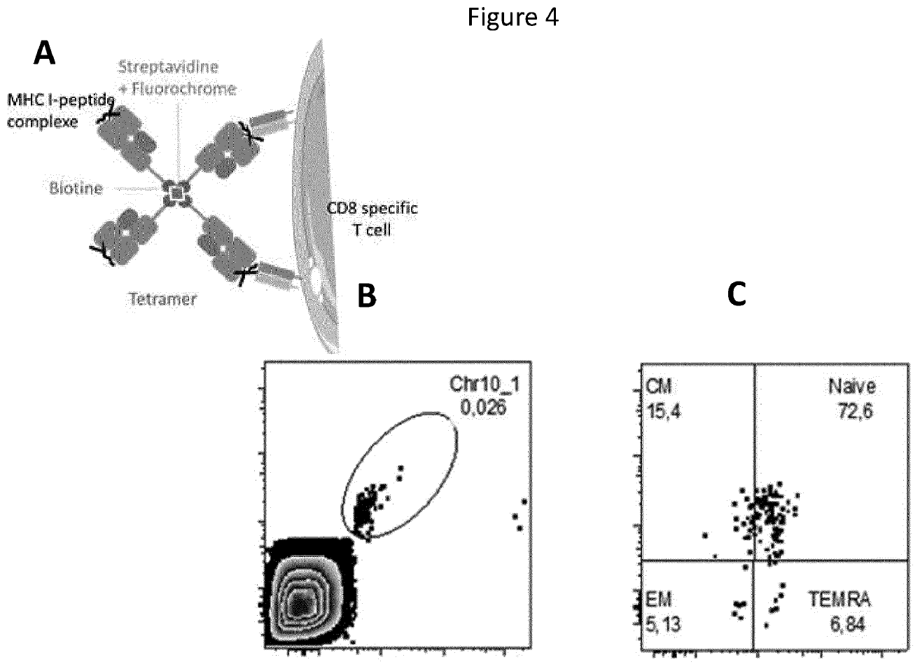

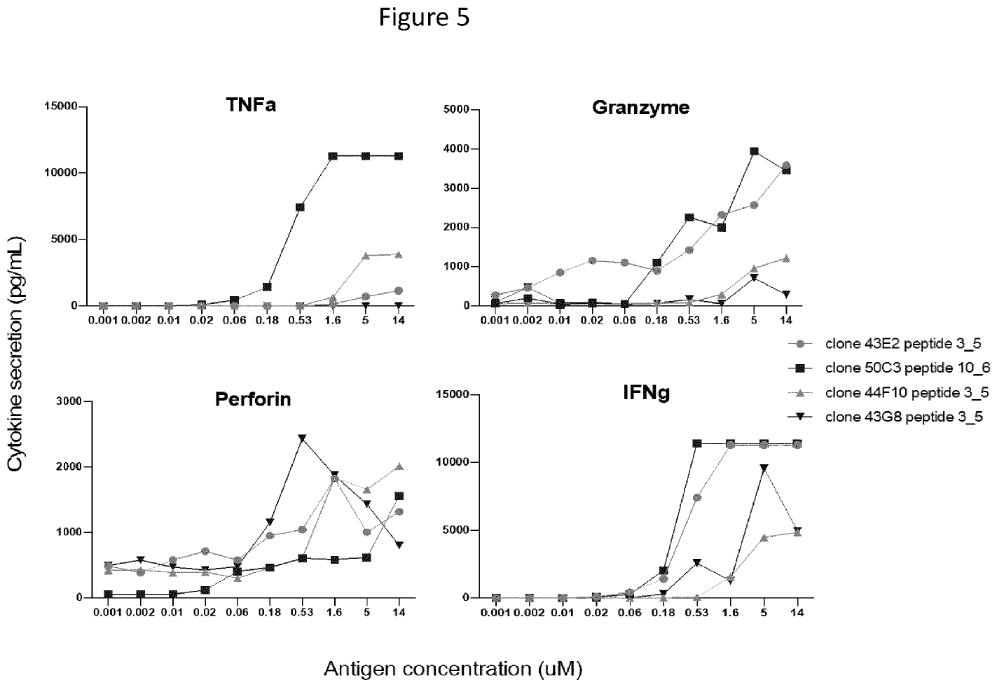

Four highly confident neotranscripts (JUJE1, JUJE2, JUJE3 and JUJE10) were identified. The inventors found that EWS-FLI1 binds GGAA microsatellites located in close vicinity to their TSS 5 kb). This binding was furthermore associated with the presence of H3K27ac and H3K4me3 activation marks. Both marks were considerably decreased upon down-regulation of EWS-FLI1 by DOX treatment in the inducible A673/TR/shEF cell line (FIG. 1A). A specific RT-PCR assay further showed that these transcripts were correctly spliced and induced by EWS-FLI1 both in A673/TR/shEF cells (FIG. 1B) and in mesenchymal stem cells (MSC) stably expressing EWS-FLI1 (FIG. 1C).

These transcripts were highly expressed in Ewing sarcoma tumor biospecimens as quantified by Illumina short-read RNA-seq data (FIG. 1A). To more thoroughly investigate the specificity for Ewing sarcoma, the inventors appended these transcripts to the RefSeq dataset and realigned the GTEx, HPA and TCGA cohorts as well as their in-house collection of 132 Ewing sarcomas and 862 tumors of diverse diagnoses, mainly sarcomas. As shown in FIGS. 1D & E, while sporadic low-level expression of these transcripts could be observed in a limited number of tissues or tumors, these levels were much lower than the mean expression level observed in Ewing sarcoma.

These data show that Ewing sarcoma expresses specific transcripts induced by the binding of EWS-FLI1 within otherwise transcriptionally silent regions. As long-read PacBio sequencing has technical limitations with respect to clinical samples, the inventors designed a short-read strategy to explore in more depth the neotranscriptome of Ewing sarcoma based on genome-guided assembly (Shao and Kingsford, Nat Biotech 2017). Transcripts that were not annotated in the reference GENCODE transcriptome were retrieved. The expression of transcripts that were detected in three different Ewing sarcomas but not in 21 non-Ewing tumors was thoroughly explored across various databases of normal and tumor tissues (FIG. 2A, see expression thresholds in the Materials and Methods section). A total 61 Ewing-specific transcripts were thus identified. As some of these were derived from the same genomic loci through alternative splicing, they could be further assigned to 25 Ewing-specific neo-genes (Ew_NG)(data not shown). This set included the JUTE genes described above, except for JUJE3 which was filtered out as it corresponds to an annotated pseudogene in GENCODE, but not in RefSeq, which was used for filtering in the PacBio strategy. The inventors noted during this procedure (cf below) that some neogenes could be moderately expressed (most less than 10 TPM) in germinal tissues (testis and placenta), reflecting known higher transcriptomic diversity and exclusivity there (e.g. for cancer-testis antigens), and therefore allowed the few genes (less than 1.5% of neogenes in this study) expressed in these tissues at more than 10 TPM to pass the filter nonetheless. A total of 25 neogenes, corresponding to 61 neotranscripts were identified in Ewing sarcoma (data not shown).

The inventors then explored the suspected role of EWS-FLI1 in the expression of these Ew_NGs. They first used 10 separate Ewing cell lines with si- or shRNA targeting EWS-FLI to show that most Ew_NG were expressed in EWS-FLI1high conditions and down-regulated in EWS-FLI1low conditions (FIG. 2B). Similarly, while these genes were not expressed in MSC, the suspected cell-of-origin of Ewing sarcoma, most were strongly induced in EWS-FLI1-expressing MSC (FIG. 2C). They also took advantage of available EWS-FLI1 ChIP-seq data for Ewing cell lines to show that such EWS-FLI1 peaks situated on GGAA microsatellites were strongly enriched around TSS of several Ew_NGs (12/25) along with H2K27ac and H3K4me3 marks (data not shown). Altogether, these data strongly suggest that the aberrant transcription factor of Ewing sarcoma can bind specific regions in the genome and induce transcription of Ewing-specific neogenes.

The identification of EWS-FLI1-regulated neogenes in Ewing sarcoma and the validation of the short-read approach prompted the inventors to investigate other sarcomas characterized by the expression of chimeric transcription factors. Alveolar rhabdomyosarcoma (aRMS) and desmoplastic small round cell tumor (DSRCT) are sarcomas characterized by the expression of PAX3/7-FOXO1 and EWS-WT1 fusions, respectively. Using the same strategy of genome-guided assembly of RNA-seq from clinical samples (FIG. 2A) they identified 36 aRMS-specific neogenes (aRMS NG) corresponding to 72 aRMS-specific neotranscripts, and 37 DSRCT-specific neogenes (DSRCT NG) corresponding to 105 DSRCT-specific neotranscripts (data not shown).

Similarly to Ewing sarcoma, they explored the potential roles of chimeric transcription factors PAX3/7-FOXO1 and EWS-WT1 in the expression of these neogenes. For aRMS, they used RNA-seq data (Gryder et al., Cancer Discov 2017) performed in the RH4 cell line after treatment with an shRNA against PAX3-FOXO1 to show that 15/36 of the neogenes are expressed in this cell line and 7/15 are downregulated (log 2FC>2 in PAX-FOXO high versus low) upon knock-down of PAX3-FOXO1. They also explored ChIP-seq data for PAX3-FOXO1, H3K27ac and H3K4me3 in the same cell line (Gryder et al., Cancer Discov 2017) and found that PAX3-FOXO1 peaks were enriched at the TSS of 13/36 of these neogenes (FIG. 3A).

For DSRCT, the inventors took advantage of recent work using an siRNA against EWS-WT1 in BER and JN-DSRCT-1 cell lines (Gedminas et al., Oncogenesis 2020) to show that all but 2 neogenes were expressed in at least one cell line and that most (28/36) were strongly downregulated (log 2FC>2 in EWS-WT1 high versus low) upon repression of EWS-WT1 (FIG. 3B). They also analyzed ChIP-seq data for EWS-WT1 in JN-DSRCT-1 (Hingorani et al., Sci Rep 2020) to show a high enrichment of EWS-WT1 peaks around TSS of neogenes (25/37), as well as RNA Polymerase II modifications showing active transcription (data not shown).

Based on their observations in Ewing sarcoma, aRMS and DSRCT, the inventors propose the concept of fusion-driven neogenes, i.e. tumor-specific neogenes that

1) are specifically associated with the fusion-driven tumor type,

2) depend on the chimeric transcription factors, as shown by regulation of expression in cell lines with activity of the chimeric transcription factor, and

3) have evidence of physical binding of the chimeric transcription factor near their TSS. Additional evidence is available for Ewing fusion-driven neogenes with presence of GGAA microsatellites in the binding sites for EWS-FLI1, as well as decreased chromatin activation marks in EWS-FLI low condition. Numbers of fusion-driven genes satisfying all criteria are 12/25 in Ewing sarcoma, 3/36 in aRMS and 16/37 in DSRCT (see Table 5 for details of classification).

A subset of neogenes depends on the chimeric transcription factor for expression in knock-down or over-expression experiments but lack clear evidence of binding by ChIP-seq. This may be due to many factors, including sensitivity of ChIP-seq, long-range regulatory interaction, or indirect regulation. The inventors thus consider such neogenes fusion-dependent.

Based on the detailed analysis in these three fusion-driven sarcomas, the inventors hypothesized that this mechanism of chimeric transcription factor-driven (or dependent) neogenes could be a recurrent phenomenon in fusion-driven sarcomas and other cancers. To test this hypothesis, they took advantage of our large institutional database of RNA-seq for sarcomas (FIG. 2A, Methods). They studied 238 cases of 17 additional types of fusion-driven cancer (all sarcomas except for midline carcinoma and TFE3 renal cell carcinoma). Analysis of this cohort (cf Methods), identified specific neogenes for all but congenital fibrosarcoma (data not shown), with between 2 and 47 tumor-specific neogenes per tumor type (table 6). Overall, the neogenes we identified are mostly multi-exonic, typically have consensus splice sites and do not show evidence of sequence conservation across vertebrates. As the inventors have only clinical samples for these types of cancer they cannot distinguish between fusion-driven, -dependent, and -associated neogenes. Interestingly, in their selection of tumor-specific neogenes, the inventors found that 5/15 neogenes found in clear cell sarcoma (CCS) were also highly expressed (mean TPM>10) in angiomatoid fibrous histiocytoma (AFH), and conversely 15/20 AFH neogenes were highly expressed in CCS. This sharing of neogenes in two types of tumors was also found for 7 neogenes in alveolar soft part sarcoma (ASPS) and TFE3 renal cell carcinoma (TFE3). These pairs of fusion-driven cancers share the same chimeric transcription factors in most cases (EWSR1-ATF1/CREB1 for AFH/CCS and ASPSCR1-TFE3 for ASPS/TFE3), strongly suggesting that the chimeric transcription factor is indeed a determining influence in the induction of these neogenes. Finally, it is noteworthy that fusion-driven cancers characterized by fusion genes not expressing chimeric sequence specific DNA-binding transcription factors, such as synovial sarcoma (SSX-SS18) and congenital fibrosarcoma (ETV6-NTRK3), have only 3 and 0 tumor-specific neogenes respectively, potentially because the chimeric transcription factor is not able to directly induce strictly fusion-driven neogenes in these cases.

The inventors believe that as in their 3 model sarcomas, part of the neogenes may be tumor-associated lncRNAs induced by a mechanism independent of the chimeric transcription factor (cf MiTranscriptome). However for fusion-driven cancers which have significantly more of these specific neogenes, they hypothesize that part of these neogenes may be chimeric transcription factor-driven or dependent, paralleling their observations in the 3 model sarcomas and extending this concept to all other fusion-driven cancers, at least those with chimeric DNA-binding transcription

Finally, open reading frames of the identified transcripts as above described were predicted and peptides predicted to be presented by the most MEW class 1 complex were synthetized for the JUJE transcripts (see tables 6-8).

Those neopeptides predicted to be presented in the context of the most frequent HLA-A2 molecule (tables 6-8) were further tested using the tetramer technology with synthetic peptides. Soluble dye-labelled tetramers.

Combined with synthesized candidate peptides were generated, assessed by flow-cytometry and then incubated with CD3+, CD8+ T cells. Double-positive T cells were further analysed using staining with CCR7 and CD45RA. This analysis showed that double positive naïve T-cells could be detected for most of the tested peptides.

FIG. 4 shows the principle of the tetramer approach and preliminary results obtained with a peptide derived from an ORF of a chromosome 10 EWS-FLI1-regulated lincRNA (i.e., an unannotated transcript specifically regulated by the EWS-FLI1 transcription factor fusion). Selected T cells were then expanded and tested in co-culture with professional Antigen Presenting cells (APCs) presenting the synthetized neopeptides. FIG. 5 shows cytokine secretion of CD8 T cells specific for the peptides (SEQ ID XX-XX) encoded by EWS-FLI1-regulated lincRNAs (initially identified by PacBio sequencing) in response to increasing concentration of their specific antigen, presented by the K562 cell antigen presenting cells. The results presented herein show that clones have responded to the synthetic neopeptide presentation (FIG. 5) at different concentrations.

Therefore the results provide evidence that neopeptides encoded by unannotated transcripts which expression is (i) regulated by a transcription factor fusion and (ii) specifically associated with the fusion-driven tumor type can not only be recognized by naïve T cells but that they can further drive their activation when presented by antigen presenting cells.

1-19. (canceled)

20. A method for identifying tumor specific neoantigenic peptides which comprises:
i. identifying transcripts from one or more samples isolated from a tumor driven by a transcription factor fusion and obtained from one or more subjects,
which transcription is specifically positively regulated by said transcription factor fusion,
which are specifically associated with the transcription-fusion tumor type, and optionally
which are encoded by neogenes that originate from intergenic or intronic regions of the genome,
ii. identifying open reading frame (ORF) sequences from the transcripts of step (i), optionally wherein said ORF sequences are specifically expressed in a tissue (or cell) sample from said transcription factor fusion-driven tumor.

21. A tumor specific neoantigenic peptide, wherein said tumor is associated with a transcription factor fusion, and wherein said peptide
i) is encoded by a part of an (ORF) sequence from a neotranscript wherein:
a. its expression is regulated by a transcription factor fusion as evidenced by expression in cell line wherein the expression of said transcription factor fusion is made inducible,
b. it is specifically associated with the fusion-driven tumor type,
c. it is are encoded by genome regions having binding motifs involved in promoter regulation such as a poly GGAA motif and/or histone marks activation, such as H3K27ac and H3K4me3 histone, located at 5 kb or less of the TSS.
ii) is expressed at a higher level or frequency in a sample from said tumor compared to normal tissue sample.

22. The tumor specific neoantigenic peptide according to claim 21 wherein the aberrant fusion transcription factor is encoded by any one of the genes selected from PAX3-FOXO1, PAX7-FOXO1, ASPSCR1-TFE3, AHRR-NCOA2, EWSR1-CREB1, EWSR1-ATF1, FUS-ATF1, EWSR1-CREB1, COL1A1-PDGFB, EWSR1-WT1, WWTR1-CAMTA1, TFE3-YAP1, EWSR1-FLI1, EWSR1-ERG, EWSR1 fusion with various ETS partners such as ETV1 FEV and ETV4, FUS-ERG, EWSR1-NFATC2, CIC-DUX4, BCOR-CCNB3, EWSR1-NR4A3, TAF15-NR4A3, TCF12-NR4A3, TFG-NR4A3, ETV6-NTRK3, ALK-TPM4, ALK-TPM3, ALK-CLTC, ALK-RANBP2, ALK-ATIC, ALK-SEC31A, ALK-CARS, PLAF fusions, HMGA2 fusions, HMGA1 fusions, C-MKL2, f95-MKL2, FUS-CREB3L2, EWSR1-ZNF444, EWSR1-PBX1, EWSR1-POU5F1, FUS-DDIT3, EWSR1-DDIT3, EWS-CHOP, EWS-CHN, TGFBR3-MGEA5, TGFBR3-MGEA5, MYH9-USP6, PHF1 fusions, ACTB-GLI1, FUS-CREB3L1, NAB2-STAT6, NCOA2-SRF, NCOA2-TEAD1, SS18-SSX1, SS18-SSX2, SS18-SSX4, and CSF1-COL6A3, preferably wherein the aberrant fusion transcription factor is encoded by EWSR1-FLI1.

23. The tumor specific neoantigenic peptide according to claim 21, which is encoded by a part of an open reading frame (ORF) of one of the sequences selected from the group comprising SEQ ID No 1-145 and the transcripts identified in table 9.

24. The tumor specific neoantigenic peptide according to claim 21, wherein said peptide comprises at least 8 amino acids, in particular 8 or 9 amino acids and binds at least one MHC class I molecule of a subject or in particular from 13 to 25 amino acids and binds at least one MHC class II of a subject; optionally wherein the neoantigenic peptides are defined in SEQ ID NO:166-201.

25. A population of autologous dendritic cells or antigen presenting cells that have been pulsed with one or more of the peptides according to claim 21 or transfected with a polynucleotide encoding one or more of the peptides according to claim 21.

26. A vaccine or immunogenic composition capable of inducing a specific T-cell response comprising
a) one or more neoantigenic peptides according to claim 21; and/or
b) one or more polynucleotides encoding a neoantigenic peptide according to claim 21, optionally linked to a heterologous regulatory control nucleotide sequence.

27. An antibody, or an antigen-binding fragment thereof, a T cell receptor (TCR), or a chimeric antigen receptor (CAR) that specifically binds a neoantigenic peptide according to claim 21, optionally in association with an MHC molecule, with a Kd affinity of about 10⁻⁶ M or less.

28. A T cell receptor according to claim 27, wherein said T cell receptor is made soluble and fused to an antibody fragment directed to a T cell antigen, optionally wherein the targeted antigen is CD3 or CD16.

29. An antibody according to claim 27, wherein said antibody is a multispecific antibody that further targets at least an immune cell antigen, optionally wherein the immune cell is a T cell, a NK cell or a dendritic cell, optionally wherein the targeted antigen is CD3, CD16, CD30 or a TCR.

30. A polynucleotide encoding the neoantigenic peptide according to claim 21.

31. A vector comprising the polynucleotide of claim 30.

32. An immune cell that specifically binds to one or more neoantigenic peptides according to claim 21, optionally wherein the immune cell is an allogenic or autologous cell selected from T cell, NK cell, CD4+/CD8+, TILs/tumor derived CD8 T cells, central memory CD8+ T cells, Treg, MAIT, and Yδ T cell.

33. A T cell, which comprises a T cell receptor that specifically binds one or more neoantigenic peptides according to claim 21.

34. A method of treatment of a cancer associated with a transcription factor fusion in a subject in need thereof, comprising the administration of a vaccine or immunogenic composition comprising:
a) one or more neoantigenic peptides according to claim 21 or one or more polynucleotides encoding said neoantigenic peptides, optionally linked to a heterologous regulatory control nucleotide sequence.

35. The method according to claim 34, wherein the cancer is Ewing sarcoma.

36. The method according to claim 34, comprising the administration of the vaccine or immunogenic composition in combination with at least one further therapeutic agent, optionally wherein the therapeutic agent is a chemotherapeutic agent or an immunotherapeutic agent.

37. A method of treatment of a cancer associated with a transcription factor fusion comprising the administration of the antibody or the antigen-binding fragment thereof, the multispecific antibody, the TCR or the CAR according to claim 27 to a subject in need thereof.

38. A method of treatment of a cancer associated with a transcription factor fusion comprising the administration of the polynucleotide according to claim 30.

39. A method of treatment of a cancer associated with a transcription factor fusion comprising the administration of a population of the immune cells according to claim 32 to a subject in need thereof.

40. A vaccine or immunogenic composition capable of inducing a specific T-cell response comprising
a) a population of antigen presenting cells according to claim 25.

41. A polynucleotide encoding the antibody, the CAR or the TCR according to claim 27.

42. A T cell, which comprises a TCR or a CAR according to claim 27.

43. A method of treatment of a cancer associated with a transcription factor fusion in a subject in need thereof, comprising the administration of a vaccine or immunogenic composition comprising:
a) a population of antigen presenting cells according to claim 25.
